SEQUENCE LISTING (322 sequences)

SEQ ID NO: 1 - DNA, 51 bp, Artificial Sequence
  Description of Artificial Sequence Synthetic oligonucleotide
ctctcgggacgaccgtgtgtgttgctctgtaacagtgtccattgtcgtccc

SEQ ID NO: 2 - DNA, 51 bp, Artificial Sequence
  Description of Artificial Sequence Synthetic oligonucleotide
ctctcgggacgaccgcgtttcccaagaaagcaagtattggttggtcgtccc

SEQ ID NO: 3 - DNA, 51 bp, Artificial Sequence
  Description of Artificial Sequence Synthetic oligonucleotide
ctctcgggacgaccggtgggggttctttttcaggggaggtacggtcgtccc

SEQ ID NO: 4 - DNA, 51 bp, Artificial Sequence
  Description of Artificial Sequence Synthetic oligonucleotide
ctctcgggacgacgaggctggatgcattcgccggatgttcgatgtcgtccc

SEQ ID NO: 5 - DNA, 51 bp, Artificial Sequence
  Description of Artificial Sequence Synthetic oligonucleotide
ctctcgggacgacgcccgcatgttccatggatagtcttgactagtcgtccc

SEQ ID NO: 6 - DNA, 51 bp, Artificial Sequence
  Description of Artificial Sequence Synthetic oligonucleotide
ctctcgggacgactagcgtatgcgccagaagtatacgaggatagtcgtccc

SEQ ID NO: 7 - DNA, 51 bp, Artificial Sequence
  Description of Artificial Sequence Synthetic oligonucleotide
ctctcgggacgacgccagaagtttacgaggatatggtaacatagtcgtccc

SEQ ID NO: 8 - DNA, 52 bp, Artificial Sequence
  Description of Artificial Sequence Synthetic oligonucleotide
ctctcgggacgacggggattttcccaattggttctttcaatttagtcgtccc

SEQ ID NO: 9 - DNA, 51 bp, Artificial Sequence
  Description of Artificial Sequence Synthetic oligonucleotide
ctctcgggacgacgggggtggcatagggtaggctagggtcactgtcgtccc

SEQ ID NO: 10 - DNA, 51 bp, Artificial Sequence
  Description of Artificial Sequence Synthetic oligonucleotide
ctctcgggacgacgtggctaggtaggttgcatgcggcataggggtcgtccc

SEQ ID NO: 11 - DNA, 51 bp, Artificial Sequence
  Description of Artificial Sequence Synthetic oligonucleotide
ctctcgggacgacgtgacggtgtgtagttgggttgtggcaggagtcgtccc

SEQ ID NO: 12 - DNA, 52 bp, Artificial Sequence
  Description of Artificial Sequence Synthetic oligonucleotide
ctctcgggacgacccggattttccgagtggaactagctgtggcggtcgtccc

SEQ ID NO: 13 - DNA, 51 bp, Artificial Sequence
  Description of Artificial Sequence Synthetic oligonucleotide
ctctcgggacgacgggatgtccggggtacggtggttgcagttcgtcgtccc

SEQ ID NO: 14 - DNA, 51 bp, Artificial Sequence
  Description of Artificial Sequence Synthetic oligonucleotide
ctctcgggacgaccaggtgccattagcgtcagtgtgctacgatgtcgtccc

SEQ ID NO: 15 - DNA, 51 bp, Artificial Sequence
  Description of Artificial Sequence Synthetic oligonucleotide
ctctcgggacgacccgttcgatctaacccttgttagccgtgatgtcgtccc

SEQ ID NO: 16 - DNA, 51 bp, Artificial Sequence
  Description of Artificial Sequence Synthetic oligonucleotide
ctctcgggacgaccccttcgatcttcaaccaaagccgttggatgtcgtccc

SEQ ID NO: 17 - DNA, 51 bp, Artificial Sequence
  Description of Artificial Sequence Synthetic oligonucleotide
ctctcgggacgacgggtggtcattgagtggtcttaggcaggtagtcgtccc

SEQ ID NO: 18 - DNA, 57 bp, Artificial Sequence
  Description of Artificial Sequence Synthetic oligonucleotide
ctctcgggacgacgtggtgtgggagaaagaattttcattggggtagggggtcgtccc

SEQ ID NO: 19 - DNA, 57 bp, Artificial Sequence
  Description of Artificial Sequence Synthetic oligonucleotide
ctctcgggacgaccacttcagacgctcaacgtttggggaggcacggcaggtcgtccc

SEQ ID NO: 20 - DNA, 57 bp, Artificial Sequence
  Description of Artificial Sequence Synthetic oligonucleotide
ctctcgggacgacggggaggagttagcatgacggcaactttagtacttcgtcgtccc

SEQ ID NO: 21 - DNA, 57 bp, Artificial Sequence
  Description of Artificial Sequence Synthetic oligonucleotide
ctctcgggacgacgccagtttgaaggttcgttcgcaggtgtggagtgacgtcgtccc

SEQ ID NO: 22 - DNA, 57 bp, Artificial Sequence
  Description of Artificial Sequence Synthetic oligonucleotide
ctctcgggacgactgcagcctggggttgtggggggtaggggaggtctgagtcgtccc

SEQ ID NO: 23 - DNA, 57 bp, Artificial Sequence
  Description of Artificial Sequence Synthetic oligonucleotide
ctctcgggacgaccacacagaggcacaactcgcaggagcaaagcggcaggtcgtccc

SEQ ID NO: 24 - DNA, 57 bp, Artificial Sequence
  Description of Artificial Sequence Synthetic oligonucleotide
ctctcgggacgacaggggcatatatagtctagggtttggtgtgggtagtgtcgtccc

SEQ ID NO: 25 - DNA, 57 bp, Artificial Sequence
  Description of Artificial Sequence Synthetic oligonucleotide
ctctcgggacgactggtaggcagataggggaagctgattcgatgcgtgggtcgtccc

SEQ ID NO: 26 - DNA, 57 bp, Artificial Sequence
  Description of Artificial Sequence Synthetic oligonucleotide
ctctcgggacgactggtaggcagcaggggaagtaggcgtgtcctcgtgggtcgtccc

SEQ ID NO: 27 - DNA, 57 bp, Artificial Sequence
  Description of Artificial Sequence Synthetic oligonucleotide
ctctcgggacgaccagtaggggatccacagtgaggggtttgtatgggtggtcgtccc

SEQ ID NO: 28 - DNA, 58 bp, Artificial Sequence
  Description of Artificial Sequence Synthetic oligonucleotide
ctctcgggacgactggtaggcaacaggggaagggagttctgcgtacgtgggtcgtccc

SEQ ID NO: 29 - DNA, 57 bp, Artificial Sequence
  Description of Artificial Sequence Synthetic oligonucleotide
ctctcgggacgacggaggtggtgtcttggacagtggtattcgcagttgcgtcgtccc

SEQ ID NO: 30 - DNA, 57 bp, Artificial Sequence
  Description of Artificial Sequence Synthetic oligonucleotide
ctctcgggacgacagagacggggtgcttacttggttcaggggagtcgacgtcgtccc

SEQ ID NO: 31 - DNA, 57 bp, Artificial Sequence
  Description of Artificial Sequence Synthetic oligonucleotide
ctctcgggacgacagccaaggtcgtaaggtacggtcagtgtactcggttgtcgtccc

SEQ ID NO: 32 - DNA, 57 bp, Artificial Sequence
  Description of Artificial Sequence Synthetic oligonucleotide
ctctcgggacgacgtcttgggggtggtgggtttggctggtacttagggcgtcgtccc

SEQ ID NO: 33 - DNA, 38 bp, Artificial Sequence
  Description of Artificial Sequence Synthetic oligonucleotide
ctctcgggacgacggcccgatctcagagtagtcgtccc

SEQ ID NO: 34 - DNA, 63 bp, Artificial Sequence
  Description of Artificial Sequence Synthetic oligonucleotide
tctgtgtcatgtggcgcctcgaccccagccttggtagctgttggccacacaagagcacatgac

SEQ ID NO: 35 - DNA, 63 bp, Artificial Sequence
  Description of Artificial Sequence Synthetic oligonucleotide
tctgtgtcatgtgacgagacaaggagaacggaaggcgaccggataaatcggatatcacatgac

SEQ ID NO: 36 - DNA, 58 bp, Artificial Sequence
  Description of Artificial Sequence Synthetic oligonucleotide
gtcatgtggattgtgacttgcccacaacgattttgccaaacatgcatagccacatgac

SEQ ID NO: 37 - DNA, 57 bp, Artificial Sequence; identical to SEQ ID NO: 24
  Description of Artificial Sequence Synthetic oligonucleotide

SEQ ID NO: 38 - DNA, 14 bp, Artificial Sequence
  Description of Artificial Sequence Synthetic oligonucleotide
tgtcgtcccgagag

SEQ ID NO: 39 - DNA, 38 bp, Artificial Sequence
  Description of Artificial Sequence Synthetic oligonucleotide
  modified_base (1)..(1): a, c, t or g
  modified_base (38)..(38): a, c, t or g
naggggcatatatagtctagggtttggtgtgggtagtn

SEQ ID NO: 40 - DNA, 57 bp, Artificial Sequence; identical to SEQ ID NO: 25
  Description of Artificial Sequence Synthetic oligonucleotide

SEQ ID NO: 41 - DNA, 13 bp, Artificial Sequence
  Description of Artificial Sequence Synthetic oligonucleotide
gtcgtcccgagag

SEQ ID NO: 42 - DNA, 39 bp, Artificial Sequence
  Description of Artificial Sequence Synthetic oligonucleotide
  modified_base (1)..(1): a, c, t or g
  modified_base (8)..(10): a, c, t or g
  modified_base (18)..(21): a, c, t or g
  modified_base (27)..(29): a, c, t or g
  modified_base (32)..(34): a, c, t or g
  modified_base (39)..(39): a, c, t or g
ntggtagnnngatagggnnnngctgannngannngtggn

SEQ ID NO: 43 - DNA, 57 bp, Artificial Sequence; identical to SEQ ID NO: 26
  Description of Artificial Sequence Synthetic oligonucleotide

SEQ ID NO: 44 - DNA, 13 bp, Artificial Sequence
  Description of Artificial Sequence Synthetic oligonucleotide
gtcgtcccgagag

SEQ ID NO: 45 - DNA, 38 bp, Artificial Sequence
  Description of Artificial Sequence Synthetic oligonucleotide
  modified_base (1)..(1): a, c, t or g
  modified_base (38)..(38): a, c, t or g
ntggtaggcagcaggggaagtaggcgtgtcctcgtggn

SEQ ID NO: 46 - DNA, 57 bp, Artificial Sequence; identical to SEQ ID NO: 27
  Description of Artificial Sequence Synthetic oligonucleotide

SEQ ID NO: 47 - DNA, 14 bp, Artificial Sequence
  Description of Artificial Sequence Synthetic oligonucleotide
ggtcgtcccgagag

SEQ ID NO: 48 - DNA, 35 bp, Artificial Sequence
  Description of Artificial Sequence Synthetic oligonucleotide
  modified_base (1)..(1): a, c, t or g
  modified_base (11)..(13): a, c, t or g
  modified_base (30)..(33): a, c, t or g
  modified_base (35)..(35): a, c, t or g
nagtaggggannncagtgaggggtttgtannnntn

SEQ ID NO: 49 - DNA, 58 bp, Artificial Sequence; identical to SEQ ID NO: 28
  Description of Artificial Sequence Synthetic oligonucleotide

SEQ ID NO: 50 - DNA, 13 bp, Artificial Sequence
  Description of Artificial Sequence Synthetic oligonucleotide
gtcgtcccgagag

SEQ ID NO: 51 - DNA, 38 bp, Artificial Sequence
  Description of Artificial Sequence Synthetic oligonucleotide
  modified_base (1)..(1): a, c, t or g
  modified_base (5)..(5): a, c, t or g
  modified_base (9)..(9): a, c, t or g
  modified_base (18)..(18): a, c, t or g
  modified_base (24)..(25): a, c, t or g
  modified_base (28)..(28): a, c, t or g
  modified_base (32)..(32): a, c, t or g
  modified_base (38)..(38): a, c, t or g
ntggnaggnaacaggggngggagnnctncgtncgtggn

SEQ ID NO: 52 - DNA, 57 bp, Artificial Sequence; identical to SEQ ID NO: 29
  Description of Artificial Sequence Synthetic oligonucleotide

SEQ ID NO: 53 - DNA, 14 bp, Artificial Sequence
  Description of Artificial Sequence Synthetic oligonucleotide
cgtcgtcccgagag

SEQ ID NO: 54 - DNA, 38 bp, Artificial Sequence
  Description of Artificial Sequence Synthetic oligonucleotide
  modified_base (1)..(1): a, c, t or g
  modified_base (10)..(20): a, c, t or g
  modified_base (38)..(38): a, c, t or g
nggaggtggnnnnnnnnnnngtggtattcgcagttgcn

SEQ ID NO: 55 - DNA, 57 bp, Artificial Sequence; identical to SEQ ID NO: 30
  Description of Artificial Sequence Synthetic oligonucleotide

SEQ ID NO: 56 - DNA, 14 bp, Artificial Sequence
  Description of Artificial Sequence Synthetic oligonucleotide
tgtcgtcccgagag

SEQ ID NO: 57 - DNA, 40 bp, Artificial Sequence
  Description of Artificial Sequence Synthetic oligonucleotide
  modified_base (1)..(2): a, c, t or g
  modified_base (6)..(8): a, c, t or g
  modified_base (33)..(35): a, c, t or g
  modified_base (39)..(40): a, c, t or g
nnagannnggggtgcttacttggttcaggggannngacnn

SEQ ID NO: 58 - DNA, 44 bp, Artificial Sequence
  Description of Artificial Sequence Synthetic oligonucleotide
cgactggtaggcagataggggaagctgattcgatgcgtgggtcg

SEQ ID NO: 59 - DNA, 63 bp, Artificial Sequence
  Description of Artificial Sequence Synthetic oligonucleotide
tctgtgtcatgtggattgtgacttgcccacaacgattttgccaaacatgcatagccacatgac

SEQ ID NO: 60 - DNA, 54 bp, Artificial Sequence
  Description of Artificial Sequence Synthetic oligonucleotide
catgtggattgtgacttgcccacaacgattttgccaaacatgcatagccacatg

SEQ ID NO: 61 - DNA, 13 bp, Artificial Sequence
  Description of Artificial Sequence Synthetic oligonucleotide
cacatgacacaga

SEQ ID NO: 62 - DNA, 39 bp, Artificial Sequence
  Description of Artificial Sequence Synthetic oligonucleotide
cgacccggattttccgagtggaactagctgtggcggtcg

SEQ ID NO: 63 - DNA, 38 bp, Artificial Sequence
  Description of Artificial Sequence Synthetic oligonucleotide
cgacagatagttgttcttagcgatgttcagcgttgtcg

SEQ ID NO: 64 - DNA, 42 bp, Artificial Sequence
  Description of Artificial Sequence Synthetic oligonucleotide
gacgacgcccgcatgttccatggatagtcttgactagtcgtc

SEQ ID NO: 65 - DNA, 34 bp, Artificial Sequence
  Description of Artificial Sequence Synthetic oligonucleotide
acgggatgtccggggtacggtggttgcagttcgt

SEQ ID NO: 66 - DNA, 38 bp, Artificial Sequence
  Description of Artificial Sequence Synthetic oligonucleotide
cgacacagcgtgagccaactaattagtgcgtattgtcg

SEQ ID NO: 67 - DNA, 36 bp, Artificial Sequence
  Description of Artificial Sequence Synthetic oligonucleotide
gaccggtgggggttctttttcaggggaggtacggtc

SEQ ID NO: 68 - DNA, 40 bp, Artificial Sequence
  Description of Artificial Sequence Synthetic oligonucleotide
acgaccgtgtgtgttgctctgtaacagtgtccattgtcgt

SEQ ID NO: 69 - DNA, 39 bp, Artificial Sequence
  Description of Artificial Sequence Synthetic oligonucleotide
cgaccgccacagctagttccactcggaaaatccgggtcg

SEQ ID NO: 70 - DNA, 5 bp, Artificial Sequence
  Description of Artificial Sequence Synthetic oligonucleotide
cgacc

SEQ ID NO: 71 - DNA, 5 bp, Artificial Sequence
  Description of Artificial Sequence Synthetic oligonucleotide
cgaca

SEQ ID NO: 72 - DNA, 6 bp, Artificial Sequence
  Description of Artificial Sequence Synthetic oligonucleotide
gacgac

SEQ ID NO: 73 - DNA, 5 bp, Artificial Sequence
  Description of Artificial Sequence Synthetic oligonucleotide
acggg

SEQ ID NO: 74 - DNA, 5 bp, Artificial Sequence
  Description of Artificial Sequence Synthetic oligonucleotide
cgaca

SEQ ID NO: 75 - DNA, 5 bp, Artificial Sequence
  Description of Artificial Sequence Synthetic oligonucleotide
gaccg

SEQ ID NO: 76 - DNA, 52 bp, Artificial Sequence; identical to SEQ ID NO: 12
  Description of Artificial Sequence Synthetic oligonucleotide

SEQ ID NO: 77 - DNA, 52 bp, Artificial Sequence
  Description of Artificial Sequence Synthetic oligonucleotide
gggacgaccgccacagctagttccactcggaaaatccgggtcgtcccgagag

SEQ ID NO: 78 - DNA, 52 bp, Artificial Sequence; identical to SEQ ID NO: 12
  Description of Artificial Sequence Synthetic oligonucleotide

SEQ ID NO: 79 - DNA, 51 bp, Artificial Sequence
  Description of Artificial Sequence Synthetic oligonucleotide
ctctcgggacgacagatagttgttcttagcgatgttcagcgttgtcgtccc

SEQ ID NO: 80 - DNA, 51 bp, Artificial Sequence
  Description of Artificial Sequence Synthetic oligonucleotide
gggacgacaacgctgaacatcgctaagaacaactatctgtcgtcccgagag

SEQ ID NO: 81 - DNA, 51 bp, Artificial Sequence; identical to SEQ ID NO: 79
  Description of Artificial Sequence Synthetic oligonucleotide

SEQ ID NO: 82 - DNA, 14 bp, Artificial Sequence
  Description of Artificial Sequence Synthetic oligonucleotide
ggtcgtcccgagag

SEQ ID NO: 83 - DNA, 23 bp, Artificial Sequence
  Description of Artificial Sequence Synthetic oligonucleotide
  modified_base (1)..(2): a, c, t, g, unknown or other
  modified_base (9)..(12): a, c, t, g, unknown or other
  modified_base (22)..(23): a, c, t, g, unknown or other
nncgtgtgnnnngtgtccattnn

SEQ ID NO: 84 - DNA, 14 bp, Artificial Sequence
  Description of Artificial Sequence Synthetic oligonucleotide
ggtcgtcccgagag

SEQ ID NO: 85 - DNA, 30 bp, Artificial Sequence
  Description of Artificial Sequence Synthetic oligonucleotide
  modified_base (1)..(2): a, c, t, g, unknown or other
  modified_base (5)..(8): a, c, t, g, unknown or other
  modified_base (13)..(16): a, c, t, g, unknown or other
  modified_base (18)..(20): a, c, t, g, unknown or other
  modified_base (24)..(26): a, c, t, g, unknown or other
  modified_base (29)..(30): a, c, t, g, unknown or other
nncgnnnnccaannnngnnngtannngtnn

SEQ ID NO: 86 - DNA, 13 bp, Artificial Sequence
  Description of Artificial Sequence Synthetic oligonucleotide
gtcgtcccgagag

SEQ ID NO: 87 - DNA, 30 bp, Artificial Sequence
  Description of Artificial Sequence Synthetic oligonucleotide
  modified_base (1)..(2): a, c, t, g, unknown or other
  modified_base (10)..(13): a, c, t, g, unknown or other
  modified_base (19)..(22): a, c, t, g, unknown or other
  modified_base (29)..(30): a, c, t, g, unknown or other
nngtgggggnnnnttttcnnnngaggtann

SEQ ID NO: 88 - DNA, 13 bp, Artificial Sequence
  Description of Artificial Sequence Synthetic oligonucleotide
gtcgtcccgagag

SEQ ID NO: 89 - DNA, 34 bp, Artificial Sequence
  Description of Artificial Sequence Synthetic oligonucleotide
  modified_base (1)..(3): a, c, t, g, unknown or other
  modified_base (32)..(34): a, c, t, g, unknown or other
nnnaggctggatgcattcgccggatgttcgannn

SEQ ID NO: 90 - DNA, 13 bp, Artificial Sequence
  Description of Artificial Sequence Synthetic oligonucleotide
gtcgtcccgagag

SEQ ID NO: 91 - DNA, 34 bp, Artificial Sequence
  Description of Artificial Sequence Synthetic oligonucleotide
  modified_base (1)..(2): a, c, t, g, unknown or other
  modified_base (8)..(11): a, c, t, g, unknown or other
  modified_base (15)..(18): a, c, t, g, unknown or other
  modified_base (21)..(21): a, c, t, g, unknown or other
  modified_base (23)..(24): a, c, t, g, unknown or other
  modified_base (29)..(34): a, c, t, g, unknown or other
nngcccgnnnnttcnnnngananncttgnnnnnn

SEQ ID NO: 92 - DNA, 13 bp, Artificial Sequence
  Description of Artificial Sequence Synthetic oligonucleotide
gtcgtcccgagag

SEQ ID NO: 93 - DNA, 30 bp, Artificial Sequence
  Description of Artificial Sequence Synthetic oligonucleotide
  modified_base (1)..(2): a, c, t, g, unknown or other
  modified_base (4)..(10): a, c, t, g, unknown or other
  modified_base (18)..(24): a, c, t, g, unknown or other
  modified_base (29)..(30): a, c, t, g, unknown or other
nngnnnnnnngccagaannnnnnnaggann

SEQ ID NO: 94 - DNA, 13 bp, Artificial Sequence
  Description of Artificial Sequence Synthetic oligonucleotide
gtcgtcccgagag

SEQ ID NO: 95 - DNA, 34 bp, Artificial Sequence
  Description of Artificial Sequence Synthetic oligonucleotide
acgccagaagtttacgaggatatggtaacatagt

SEQ ID NO: 96 - DNA, 14 bp, Artificial Sequence
  Description of Artificial Sequence Synthetic oligonucleotide
cgtcgtcccgagag

SEQ ID NO: 97 - DNA, 35 bp, Artificial Sequence
  Description of Artificial Sequence Synthetic oligonucleotide
  modified_base (1)..(1): a, c, t, g, unknown or other
  modified_base (5)..(7): a, c, t, g, unknown or other
  modified_base (11)..(13): a, c, t, g, unknown or other
  modified_base (16)..(19): a, c, t, g, unknown or other
  modified_base (27)..(30): a, c, t, g, unknown or other
  modified_base (35)..(35): a, c, t, g, unknown or other
ncggnnntttnnncannnngttctttnnnnttagn

SEQ ID NO: 98 - DNA, 13 bp, Artificial Sequence
  Description of Artificial Sequence Synthetic oligonucleotide
gtcgtcccgagag

SEQ ID NO: 99 - DNA, 32 bp, Artificial Sequence
  Description of Artificial Sequence Synthetic oligonucleotide
  modified_base (1)..(1): a, c, t, g, unknown or other
  modified_base (6)..(10): a, c, t, g, unknown or other
  modified_base (12)..(14): a, c, t, g, unknown or other
  modified_base (21)..(23): a, c, t, g, unknown or other
  modified_base (26)..(30): a, c, t, g, unknown or other
  modified_base (32)..(32): a, c, t, g, unknown or other
nggggnnnnnannnggtaggnnnggnnnnntn

SEQ ID NO: 100 - DNA, 13 bp, Artificial Sequence
  Description of Artificial Sequence Synthetic oligonucleotide
gtcgtcccgagag

SEQ ID NO: 101 - DNA, 34 bp, Artificial Sequence
  Description of Artificial Sequence Synthetic oligonucleotide
  modified_base (1)..(1): a, c, t, g, unknown or other
  modified_base (7)..(9): a, c, t, g, unknown or other
  modified_base (17)..(19): a, c, t, g, unknown or other
  modified_base (25)..(30): a, c, t, g, unknown or other
  modified_base (34)..(34): a, c, t, g, unknown or other
ncgtggnnnggtaggtnnnatgcgnnnnnngggn

SEQ ID NO: 102 - DNA, 14 bp, Artificial Sequence
  Description of Artificial Sequence Synthetic oligonucleotide
cgtcgtcccgagag

SEQ ID NO: 103 - DNA, 34 bp, Artificial Sequence
  Description of Artificial Sequence Synthetic oligonucleotide
  modified_base (1)..(2): a, c, t, g, unknown or other
  modified_base (33)..(34): a, c, t, g, unknown or other
nngtgacggtgtgtagttgggttgtggcaggann

SEQ ID NO: 104 - DNA, 13 bp, Artificial Sequence
  Description of Artificial Sequence Synthetic oligonucleotide
gtcgtcccgagag

SEQ ID NO: 105 - DNA, 33 bp, Artificial Sequence
  Description of Artificial Sequence Synthetic oligonucleotide
  modified_base (1)..(6): a, c, t, g, unknown or other
  modified_base (10)..(16): a, c, t, g, unknown or other
  modified_base (20)..(22): a, c, t, g, unknown or other
  modified_base (32)..(33): a, c, t, g, unknown or other
nnnnnntttnnnnnnnggannnagctgtggcnn

SEQ ID NO: 106 - DNA, 13 bp, Artificial Sequence
  Description of Artificial Sequence Synthetic oligonucleotide
gtcgtcccgagag

SEQ ID NO: 107 - DNA, 34 bp, Artificial Sequence
  Description of Artificial Sequence Synthetic oligonucleotide
  modified_base (1)..(2): a, c, t, g, unknown or other
  modified_base (33)..(34): a, c, t, g, unknown or other
nngggatgtccggggtacggtggttgcagttcnn

SEQ ID NO: 108 - DNA, 14 bp, Artificial Sequence
  Description of Artificial Sequence Synthetic oligonucleotide
ggtcgtcccgagag

SEQ ID NO: 109 - DNA, 36 bp, Artificial Sequence
  Description of Artificial Sequence Synthetic oligonucleotide
  modified_base (1)..(3): a, c, t, g, unknown or other
  modified_base (14)..(18): a, c, t, g, unknown or other
  modified_base (25)..(29): a, c, t, g, unknown or other
  modified_base (34)..(36): a, c, t, g, unknown or other
nnncaggtgccatnnnnntcagtgnnnnncgatnnn

SEQ ID NO: 110 - DNA, 14 bp, Artificial Sequence
  Description of Artificial Sequence Synthetic oligonucleotide
ggtcgtcccgagag

SEQ ID NO: 111 - DNA, 36 bp, Artificial Sequence
  Description of Artificial Sequence Synthetic oligonucleotide
  modified_base (1)..(3): a, c, t, g, unknown or other
  modified_base (14)..(16): a, c, t, g, unknown or other
  modified_base (23)..(25): a, c, t, g, unknown or other
  modified_base (34)..(36): a, c, t, g, unknown or other
nnnccgttcgatcnnncccttgnnngccgtgatnnn

SEQ ID NO: 112 - DNA, 13 bp, Artificial Sequence
  Description of Artificial Sequence Synthetic oligonucleotide
gtcgtcccgagag

SEQ ID NO: 113 - DNA, 36 bp, Artificial Sequence
  Description of Artificial Sequence Synthetic oligonucleotide
  modified_base (1)..(3): a, c, t, g, unknown or other
  modified_base (34)..(36): a, c, t, g, unknown or other
nnncccttcgatcttcaaccaaagccgttggatnnn

SEQ ID NO: 114 - DNA, 14 bp, Artificial Sequence
  Description of Artificial Sequence Synthetic oligonucleotide
cgtcgtcccgagag

SEQ ID NO: 115 - DNA, 38 bp, Artificial Sequence
  Description of Artificial Sequence Synthetic oligonucleotide
  modified_base (1)..(4): a, c, t, g, unknown or other
  modified_base (35)..(37): a, c, t, g, unknown or other
nnnngggtggtcattgagtggtcttaggcaggtannng

SEQ ID NO: 116 - DNA, 13 bp, Artificial Sequence
  Description of Artificial Sequence Synthetic oligonucleotide
gtcgtcccgagag

SEQ ID NO: 117 - DNA, 32 bp, Artificial Sequence
  Description of Artificial Sequence Synthetic oligonucleotide
  modified_base (1)..(2): a, c, t, g, unknown or other
  modified_base (15)..(16): a, c, t, g, unknown or other
  modified_base (31)..(32): a, c, t, g, unknown or other
nngtggtgtgggagnncattggggtaggggnn

SEQ ID NO: 118 - DNA, 14 bp, Artificial Sequence
  Description of Artificial Sequence Synthetic oligonucleotide
ggtcgtcccgagag

SEQ ID NO: 119 - DNA, 38 bp, Artificial Sequence
  Description of Artificial Sequence Synthetic oligonucleotide
  modified_base (1)..(1): a, c, t, g, unknown or other
  modified_base (5)..(11): a, c, t, g, unknown or other
  modified_base (19)..(25): a, c, t, g, unknown or other
  modified_base (38)..(38): a, c, t, g, unknown or other
ncacnnnnnnngctcaacnnnnnnngaggcacggcagn

SEQ ID NO: 120 - DNA, 14 bp, Artificial Sequence
  Description of Artificial Sequence Synthetic oligonucleotide
cgtcgtcccgagag

SEQ ID NO: 121 - DNA, 23 bp, Artificial Sequence
  Description of Artificial Sequence Synthetic oligonucleotide
  modified_base (1)..(1): a, c, t, g, unknown or other
  modified_base (10)..(11): a, c, t, g, unknown or other
  modified_base (23)..(23): a, c, t, g, unknown or other
nggggagganntttagtacttcn

SEQ ID NO: 122 - DNA, 13 bp, Artificial Sequence
  Description of Artificial Sequence Synthetic oligonucleotide
gtcgtcccgagag

SEQ ID NO: 123 - DNA, 38 bp, Artificial Sequence
  Description of Artificial Sequence Synthetic oligonucleotide
  modified_base (1)..(1): a, c, t, g, unknown or other
  modified_base (10)..(12): a, c, t, g, unknown or other
  modified_base (19)..(21): a, c, t, g, unknown or other
  modified_base (38)..(38): a, c, t, g, unknown or other
ngccagtttnnnggttcgnnngcaggtgtggagtgacn

SEQ ID NO: 124 - DNA, 13 bp, Artificial Sequence
  Description of Artificial Sequence Synthetic oligonucleotide
gtcgtcccgagag

SEQ ID NO: 125 - DNA, 38 bp, Artificial Sequence
  Description of Artificial Sequence Synthetic oligonucleotide
  modified_base (1)..(1): a, c, t, g, unknown or other
  modified_base (38)..(38): a, c, t, g, unknown or other
ntgcagcctggggttgtggggggtaggggaggtctgan

SEQ ID NO: 126 - DNA, 13 bp, Artificial Sequence
  Description of Artificial Sequence Synthetic oligonucleotide
gtcgtcccgagag

SEQ ID NO: 127 - DNA, 38 bp, Artificial Sequence
  Description of Artificial Sequence Synthetic oligonucleotide
  modified_base (1)..(2): a, c, t, g, unknown or other
  modified_base (19)..(21): a, c, t, g, unknown or other
  modified_base (31)..(33): a, c, t, g, unknown or other
  modified_base (37)..(38): a, c, t, g, unknown or other
nnacacagaggcacaactnnnaggagcaaannngcann

SEQ ID NO: 128 - DNA, 14 bp, Artificial Sequence
  Description of Artificial Sequence Synthetic oligonucleotide
tgtcgtcccgagag

SEQ ID NO: 129 - DNA, 38 bp, Artificial Sequence; identical to SEQ ID NO: 39
  Description of Artificial Sequence Synthetic oligonucleotide
  modified_base (1)..(1): a, c, t, g, unknown or other
  modified_base (38)..(38): a, c, t, g, unknown or other

SEQ ID NO: 130 - DNA, 13 bp, Artificial Sequence
  Description of Artificial Sequence Synthetic oligonucleotide
gtcgtcccgagag

SEQ ID NO: 131 - DNA, 38 bp, Artificial Sequence
  Description of Artificial Sequence Synthetic oligonucleotide
  modified_base (1)..(1): a, c, t, g, unknown or other
  modified_base (8)..(10): a, c, t, g, unknown or other
  modified_base (18)..(20): a, c, t, g, unknown or other
  modified_base (26)..(28): a, c, t, g, unknown or other
  modified_base (31)..(33): a, c, t, g, unknown or other
  modified_base (38)..(38): a, c, t, g, unknown or other
ntggtagnnngatagggnnngctgannngannngtggn

SEQ ID NO: 132 - DNA, 13 bp, Artificial Sequence
  Description of Artificial Sequence Synthetic oligonucleotide
gtcgtcccgagag

SEQ ID NO: 133 - DNA, 38 bp, Artificial Sequence; identical to SEQ ID NO: 45
  Description of Artificial Sequence Synthetic oligonucleotide
  modified_base (1)..(1): a, c, t, g, unknown or other
  modified_base (38)..(38): a, c, t, g, unknown or other

SEQ ID NO: 134 - DNA, 14 bp, Artificial Sequence
  Description of Artificial Sequence Synthetic oligonucleotide
ggtcgtcccgagag

SEQ ID NO: 135 - DNA, 36 bp, Artificial Sequence
  Description of Artificial Sequence Synthetic oligonucleotide
  modified_base (1)..(1): a, c, t, g, unknown or other
  modified_base (11)..(14): a, c, t, g, unknown or other
  modified_base (31)..(34): a, c, t, g, unknown or other
  modified_base (36)..(36): a, c, t, g, unknown or other
nagtaggggannnncagtgaggggtttgtannnntn

SEQ ID NO: 136 - DNA, 13 bp, Artificial Sequence
  Description of Artificial Sequence Synthetic oligonucleotide
gtcgtcccgagag

SEQ ID NO: 137 - DNA, 39 bp, Artificial Sequence
  Description of Artificial Sequence Synthetic oligonucleotide
  modified_base (1)..(1): a, c, t, g, unknown or other
  modified_base (5)..(5): a, c, t, g, unknown or other
  modified_base (9)..(9): a, c, t, g, unknown or other
  modified_base (18)..(19): a, c, t, g, unknown or other
  modified_base (25)..(26): a, c, t, g, unknown or other
  modified_base (29)..(29): a, c, t, g, unknown or other
  modified_base (33)..(33): a, c, t, g, unknown or other
  modified_base (39)..(39): a, c, t, g, unknown or other
ntggnaggnaacaggggnngggagnnctncgtncgtggn

SEQ ID NO: 138 - DNA, 14 bp, Artificial Sequence
  Description of Artificial Sequence Synthetic oligonucleotide
cgtcgtcccgagag

SEQ ID NO: 139 - DNA, 38 bp, Artificial Sequence; identical to SEQ ID NO: 54
  Description of Artificial Sequence Synthetic oligonucleotide
  modified_base (1)..(1): a, c, t, g, unknown or other
  modified_base (10)..(20): a, c, t, g, unknown or other
  modified_base (38)..(38): a, c, t, g, unknown or other

SEQ ID NO: 140 - DNA, 14 bp, Artificial Sequence
  Description of Artificial Sequence Synthetic oligonucleotide
tgtcgtcccgagag

SEQ ID NO: 141 - DNA, 40 bp, Artificial Sequence; identical to SEQ ID NO: 57
  Description of Artificial Sequence Synthetic oligonucleotide
  modified_base (1)..(2): a, c, t, g, unknown or other
  modified_base (6)..(8): a, c, t, g, unknown or other
  modified_base (33)..(35): a, c, t, g, unknown or other
  modified_base (39)..(40): a, c, t, g, unknown or other

SEQ ID NO: 142 - DNA, 15 bp, Artificial Sequence
  Description of Artificial Sequence Synthetic oligonucleotide
ctgtcgtcccgagag

SEQ ID NO: 143 - DNA, 34 bp, Artificial Sequence
  Description of Artificial Sequence Synthetic oligonucleotide
  modified_base (1)..(2): a, c, t, g, unknown or other
  modified_base (7)..(7): a, c, t, g, unknown or other
  modified_base (10)..(11): a, c, t, g, unknown or other
  modified_base (17)..(18): a, c, t, g, unknown or other
  modified_base (29)..(29): a, c, t, g, unknown or other
  modified_base (33)..(34): a, c, t, g, unknown or other
nncaagntcnnaaggtnnggtcagtgtantcgnn

SEQ ID NO: 144 - DNA, 14 bp, Artificial Sequence
  Description of Artificial Sequence Synthetic oligonucleotide
cgtcgtcccgagag

SEQ ID NO: 145 - DNA, 36 bp, Artificial Sequence
  Description of Artificial Sequence Synthetic oligonucleotide
  modified_base (1)..(2): a, c, t, g, unknown or other
  modified_base (24)..(25): a, c, t, g, unknown or other
  modified_base (31)..(32): a, c, t, g, unknown or other
  modified_base (35)..(36): a, c, t, g, unknown or other
nncttgggggtggtgggtttggcnngtactnnggnn

SEQ ID NO: 146 - DNA, 13 bp, Artificial Sequence
  Description of Artificial Sequence Synthetic oligonucleotide
gtcgtcccgagag

SEQ ID NO: 147 - DNA, 21 bp, Artificial Sequence
  Description of Artificial Sequence Synthetic oligonucleotide
acggcccgatctcagagtagt

SEQ ID NO: 148 - DNA, 38 bp, Artificial Sequence
  Description of Artificial Sequence Synthetic oligonucleotide
cgacgcgcgcatgttccatggatagtcttgactagtcg

SEQ ID NO: 149 - DNA, 38 bp, Artificial Sequence
  Description of Artificial Sequence Synthetic oligonucleotide
cgacctggtgtgttgctctgtaacagtgtctattgtcg

SEQ ID NO: 150 - DNA, 59 bp, Artificial Sequence
  Description of Artificial Sequence Synthetic oligonucleotide
ccggattttccgagtactaaggcgccttagtactcggaaaatccgggtcgtcccgagag

SEQ ID NO: 151 - DNA, 60 bp, Artificial Sequence
  Description of Artificial Sequence Synthetic oligonucleotide
gtgtgtgttgctctgagttattggccttagttttcttgggaaacgcggtcgtcccgagag

SEQ ID NO: 152 - DNA, 60 bp, Artificial Sequence
  Description of Artificial Sequence Synthetic oligonucleotide
gtgtgtgttgctctgagttattgcaataactcagagcaacacacacggtcgtcccgagag

SEQ ID NO: 153 - DNA, 46 bp, Artificial Sequence
  Description of Artificial Sequence Synthetic oligonucleotide
gcccgatcaggcttggccaagcctgatcgggccgtcgtcccgagag

SEQ ID NO: 154 - DNA, 59 bp, Artificial Sequence; identical to SEQ ID NO: 150
  Description of Artificial Sequence Synthetic oligonucleotide

SEQ ID NO: 155 - DNA, 60 bp, Artificial Sequence; identical to SEQ ID NO: 151
  Description of Artificial Sequence Synthetic oligonucleotide

SEQ ID NO: 156 - DNA, 13 bp, Artificial Sequence
  Description of Artificial Sequence Synthetic oligonucleotide
agacacagtacac

SEQ ID NO: 157 - DNA, 8 bp, Artificial Sequence
  Description of Artificial Sequence Synthetic oligonucleotide
ccgtgtgt

SEQ ID NO: 158 - DNA, 11 bp, Artificial Sequence
  Description of Artificial Sequence Synthetic oligonucleotide
agtgtccattg

SEQ ID NO: 159 - DNA, 11 bp, Artificial Sequence
  Description of Artificial Sequence Synthetic oligonucleotide
agtgtcctttg

SEQ ID NO: 160 - DNA, 51 bp, Artificial Sequence; identical to SEQ ID NO: 1
  Description of Artificial Sequence Synthetic oligonucleotide

SEQ ID NO: 161 - DNA, 60 bp, Artificial Sequence
  Description of Artificial Sequence Synthetic oligonucleotide
ctctcgggacgaccgtgtgtggtagagtcgtcgggctctaacagtgtcctttgtcgtccc

SEQ ID NO: 162 - DNA, 60 bp, Artificial Sequence
  Description of Artificial Sequence Synthetic oligonucleotide
ctctcgggacgaccgtgtgtgacgtgcgccgtggggaacgtcagtgttctttgtcgtccc

SEQ ID NO: 163 - DNA, 51 bp, Artificial Sequence
  Description of Artificial Sequence Synthetic oligonucleotide
ctctcgggacgaccgtgtgtcgacttagagtcgagtgtcctttgtcgtccc

SEQ ID NO: 164 - DNA, 51 bp, Artificial Sequence
  Description of Artificial Sequence Synthetic oligonucleotide
ctctcgggacgaccgtgtgttgcaattcttgcaagtgttctttgtcgtccc

SEQ ID NO: 165 - DNA, 4 bp, Artificial Sequence
  Description of Artificial Sequence Synthetic oligonucleotide
gcgt

SEQ ID NO: 166 - DNA, 4 bp, Artificial Sequence
  Description of Artificial Sequence Synthetic oligonucleotide
ggtt

SEQ ID NO: 167 - DNA, 36 bp, Artificial Sequence
  Description of Artificial Sequence Synthetic oligonucleotide
gaccgcgtttcccaagaaagcaagtattggttggtc

SEQ ID NO: 168 - DNA, 5 bp, Artificial Sequence
  Description of Artificial Sequence Synthetic oligonucleotide
ggggg

SEQ ID NO: 169 - DNA, 4 bp, Artificial Sequence
  Description of Artificial Sequence Synthetic oligonucleotide
gggg

SEQ ID NO: 170 - DNA, 4 bp, Artificial Sequence
  Description of Artificial Sequence Synthetic oligonucleotide
gagg

SEQ ID NO: 171 - DNA, 4 bp, Artificial Sequence
  Description of Artificial Sequence Synthetic oligonucleotide
catt

SEQ ID NO: 172 - DNA, 4 bp, Artificial Sequence
  Description of Artificial Sequence Synthetic oligonucleotide
ccgg

SEQ ID NO: 173 - DNA, 4 bp, Artificial Sequence
  Description of Artificial Sequence Synthetic oligonucleotide
tgtt

SEQ ID NO: 174 - DNA, 38 bp, Artificial Sequence
  Description of Artificial Sequence Synthetic oligonucleotide
cgacgaggctggatgcattcgccggatgttcgatgtcg

SEQ ID NO: 175 - DNA, 4 bp, Artificial Sequence
  Description of Artificial Sequence Synthetic oligonucleotide
cgcc

SEQ ID NO: 176 - DNA, 6 bp, Artificial Sequence
  Description of Artificial Sequence Synthetic oligonucleotide
atgttc

SEQ ID NO: 177 - DNA, 7 bp, Artificial Sequence
  Description of Artificial Sequence Synthetic oligonucleotide
ggatagt

SEQ ID NO: 178 - DNA, 37 bp, Artificial Sequence
  Description of Artificial Sequence Synthetic oligonucleotide
gacgcccgcatgttccatggatagtcttgactagtcg

SEQ ID NO: 179 - DNA, 5 bp, Artificial Sequence
  Description of Artificial Sequence Synthetic oligonucleotide
tacga

SEQ ID NO: 180 - DNA, 5 bp, Artificial Sequence
  Description of Artificial Sequence Synthetic oligonucleotide
ggata

SEQ ID NO: 181 - DNA, 34 bp, Artificial Sequence
  Description of Artificial Sequence Synthetic oligonucleotide
actagcgtatgcgccagaagtatacgaggatagt

SEQ ID NO: 182 - DNA, 36 bp, Artificial Sequence
  Description of Artificial Sequence Synthetic oligonucleotide
gacgccagaagtttacgaggatatggtaacatagtc

SEQ ID NO: 183 - DNA, 36 bp, Artificial Sequence
  Description of Artificial Sequence Synthetic oligonucleotide
gacgggggtggcatagggtaggctagggtcactgtc

SEQ ID NO: 184 - DNA, 40 bp, Artificial Sequence
  Description of Artificial Sequence Synthetic oligonucleotide
acgacgtggctaggtaggttgcatgcggcataggggtcgt

SEQ ID NO: 185 - DNA, 40 bp, Artificial Sequence
  Description of Artificial Sequence Synthetic oligonucleotide
acgacgtgacggtgtgtagttgggttgtggcaggagtcgt

SEQ ID NO: 186 - DNA, 4 bp, Artificial Sequence
  Description of Artificial Sequence Synthetic oligonucleotide
agct

SEQ ID NO: 187 - DNA, 4 bp, Artificial Sequence
  Description of Artificial Sequence Synthetic oligonucleotide
gcgg

SEQ ID NO: 188 - DNA, 37 bp, Artificial Sequence
  Description of Artificial Sequence Synthetic oligonucleotide
gacccggattttccgagtggaactagctgtggcggtc

SEQ ID NO: 189 - DNA, 41 bp, Artificial Sequence
  Description of Artificial Sequence Synthetic oligonucleotide
acgacggggattttccagtgcaactagctgaaagcggtcgt

SEQ ID NO: 190 - DNA, 39 bp, Artificial Sequence
  Description of Artificial Sequence Synthetic oligonucleotide
cgaccaggattttccagtgtaactagctacagcgggtcg

SEQ ID NO: 191 - DNA, 36 bp, Artificial Sequence
  Description of Artificial Sequence Synthetic oligonucleotide
gacgggatgtccggggtacggtggttgcagttcgtc

SEQ ID NO: 192 - DNA, 40 bp, Artificial Sequence
  Description of Artificial Sequence Synthetic oligonucleotide
acgacgggtggtcattgagtggtcttaggcaggtagtcgt

SEQ ID NO: 193 - DNA, 46 bp, Artificial Sequence
  Description of Artificial Sequence Synthetic oligonucleotide
acgacgtggtgtgggagaaagaattttcattggggtagggggtcgt

SEQ ID NO: 194 - DNA, 5 bp, Artificial Sequence
  Description of Artificial Sequence Synthetic oligonucleotide
ccgat

SEQ ID NO: 195 - DNA, 5 bp, Artificial Sequence
  Description of Artificial Sequence Synthetic oligonucleotide
ggtgt

SEQ ID NO: 196 - DNA, 44 bp, Artificial Sequence
  Description of Artificial Sequence Synthetic oligonucleotide
cgacgccagtttgaaggttcgttcgcaggtgtggagtgacgtcg

SEQ ID NO: 197 - DNA, 44 bp, Artificial Sequence
  Description of Artificial Sequence Synthetic oligonucleotide
cgactgcagcctggggttgtggggggtaggggaggtctgagtcg

SEQ ID NO: 198 - DNA, 5 bp, Artificial Sequence
  Description of Artificial Sequence Synthetic oligonucleotide
cacag

SEQ ID NO: 199 - DNA, 5 bp, Artificial Sequence
  Description of Artificial Sequence Synthetic oligonucleotide
cacaa

SEQ ID NO: 200 - DNA, 44 bp, Artificial Sequence
  Description of Artificial Sequence Synthetic oligonucleotide
cgaccacacagaggcacaactcgcaggagcaaagcggcaggtcg

SEQ ID NO: 201 - DNA, 44 bp, Artificial Sequence
  Description of Artificial Sequence Synthetic oligonucleotide
cgacggggaggagttagcatgacggcaactttagtacttcgtcg

SEQ ID NO: 202 - DNA, 44 bp, Artificial Sequence
  Description of Artificial Sequence Synthetic oligonucleotide
cgaccacttcagacgctcaacgtttggggaggcacggcaggtcg

SEQ ID NO: 203 - DNA, 44 bp, Artificial Sequence; identical to SEQ ID NO: 58
  Description of Artificial Sequence Synthetic oligonucleotide

SEQ ID NO: 204 - DNA, 45 bp, Artificial Sequence
  Description of Artificial Sequence Synthetic oligonucleotide
cgactggtaggcaacaggggaagggagttctgcgtacgtgggtcg

SEQ ID NO: 205 - DNA, 44 bp, Artificial Sequence
  Description of Artificial Sequence Synthetic oligonucleotide
cgacaggggcatatatagtctagggtttggtgtgggtagtgtcg

SEQ ID NO: 206 - DNA, 44 bp, Artificial Sequence
  Description of Artificial Sequence Synthetic oligonucleotide
cgactggtaggcagcaggggaagtaggcgtgtcctcgtgggtcg

SEQ ID NO: 207 - DNA, 42 bp, Artificial Sequence
  Description of Artificial Sequence Synthetic oligonucleotide
gaccagtaggggatccacagtgaggggtttgtatgggtggtc

SEQ ID NO: 208 - DNA, 44 bp, Artificial Sequence
  Description of Artificial Sequence Synthetic oligonucleotide
cgacggaggtggtgtcttggacagtggtattcgcagttgcgtcg

SEQ ID NO: 209 - DNA, 46 bp, Artificial Sequence
  Description of Artificial Sequence Synthetic oligonucleotide
acgacagagacggggtgcttacttggttcaggggagtcgacgtcgt

SEQ ID NO: 210 - DNA, 40 bp, Artificial Sequence
  Description of Artificial Sequence Synthetic oligonucleotide
acgtcttgggggtggtgggtttggctggtacttagggcgt

SEQ ID NO: 211 - DNA, 38 bp, Artificial Sequence
  Description of Artificial Sequence Synthetic oligonucleotide
cagccaaggtcgtaaggtacggtcagtgtactcggttg

SEQ ID NO: 212 - DNA, 6 bp, Artificial Sequence
  Description of Artificial Sequence Synthetic oligonucleotide
gatagt

SEQ ID NO: 213 - DNA, 6 bp, Artificial Sequence
  Description of Artificial Sequence Synthetic oligonucleotide
atgttc

SEQ ID NO: 214 - DNA, 36 bp, Artificial Sequence
  Description of Artificial Sequence Synthetic oligonucleotide
gacagatagttgttcttagcgatgttcagcgttgtc

SEQ ID NO: 215 - DNA, 38 bp, Artificial Sequence
  Description of Artificial Sequence Synthetic oligonucleotide
cgacggtaggtaggccaactgggtatttactggtgtcg

SEQ ID NO: 216 - DNA, 5 bp, Artificial Sequence
  Description of Artificial Sequence Synthetic oligonucleotide
tgaaa

SEQ ID NO: 217 - DNA, 5 bp, Artificial Sequence
  Description of Artificial Sequence Synthetic oligonucleotide
aagtg

SEQ ID NO: 218 - DNA, 44 bp, Artificial Sequence
  Description of Artificial Sequence Synthetic oligonucleotide
cgacggccccgcaaggggtgaaatgacagagtcaaagtgcgtcg

SEQ ID NO: 219 - DNA, 6 bp, Artificial Sequence
  Description of Artificial Sequence Synthetic oligonucleotide
gtagtc

SEQ ID NO: 220 - DNA, 7 bp, Artificial Sequence
  Description of Artificial Sequence Synthetic oligonucleotide
tcggtag

SEQ ID NO: 221 - DNA, 44 bp, Artificial Sequence
  Description of Artificial Sequence Synthetic oligonucleotide
acgacgtagtcggaaacggtgtctcagttcctcggtagagtcgt

SEQ ID NO: 222 - DNA, 4 bp, Artificial Sequence
  Description of Artificial Sequence Synthetic oligonucleotide
gccc

SEQ ID NO: 223 - DNA, 7 bp, Artificial Sequence
  Description of Artificial Sequence Synthetic oligonucleotide
gtttaga

SEQ ID NO: 224 - DNA, 6 bp, Artificial Sequence
  Description of Artificial Sequence Synthetic oligonucleotide
agtctt

SEQ ID NO: 225 - DNA, 44 bp, Artificial Sequence
  Description of Artificial Sequence Synthetic oligonucleotide
cgaccgcttgccccctggcatgtttagagcagagtctttggtcg

SEQ ID NO: 226 - DNA, 7 bp, Artificial Sequence
  Description of Artificial Sequence Synthetic oligonucleotide
ggttcat

SEQ ID NO: 227 - DNA, 7 bp, Artificial Sequence
  Description of Artificial Sequence Synthetic oligonucleotide
atgtggg

SEQ ID NO: 228 - DNA, 44 bp, Artificial Sequence
  Description of Artificial Sequence Synthetic oligonucleotide
cgacgtccggttcatgacttcagtagtctagtgggggtctgtcg

SEQ ID NO: 229 - DNA, 15 bp, Artificial Sequence
  Description of Artificial Sequence Synthetic oligonucleotide
cgtctacggcttagc

SEQ ID NO: 230 - DNA, 44 bp, Artificial Sequence
  Description of Artificial Sequence Synthetic oligonucleotide
cgacgcaaccattccagtggcgtctacggcttagcttttcgtcg

SEQ ID NO: 231 - DNA, 46 bp, Artificial Sequence
  Description of Artificial Sequence Synthetic oligonucleotide
acgaccaggatgctgttccaccggggtacaggtaggtcgctgtcgt

SEQ ID NO: 232 - DNA, 40 bp, Artificial Sequence
  Description of Artificial Sequence Synthetic oligonucleotide
acgggcgtagcgatagaagagagcagggggagagacctgt

SEQ ID NO: 233 - DNA, 46 bp, Artificial Sequence
  Description of Artificial Sequence Synthetic oligonucleotide
acgacgggaaggagttccggggtacgcgggtaagggaaggagtcgt

SEQ ID NO: 234 - DNA, 44 bp, Artificial Sequence
  Description of Artificial Sequence Synthetic oligonucleotide
cgaccaacgagtatacgcttacgtcacgttgatgctgtgggtcg

SEQ ID NO: 235 - DNA, 44 bp, Artificial Sequence
  Description of Artificial Sequence Synthetic oligonucleotide
cgacgcattgatgtacaagctcgattcgtatcccttgatcgtcg

SEQ ID NO: 236 - DNA, 38 bp, Artificial Sequence
  Description of Artificial Sequence Synthetic oligonucleotide
ctgggctcgtgttctatggacaagggggagtgacctgg

SEQ ID NO: 237 - DNA, 5 bp, Artificial Sequence
  Description of Artificial Sequence Synthetic oligonucleotide
ctctc

SEQ ID NO: 238 - DNA, 8 bp, Artificial Sequence
  Description of Artificial Sequence Synthetic oligonucleotide
ctctcggg

SEQ ID NO: 239 - DNA, 44 bp, Artificial Sequence
  Description of Artificial Sequence Synthetic oligonucleotide
cgacggaagaggctcagtgctatcttatctgagagggtttgtcg

SEQ ID NO: 240 - DNA, 38 bp, Artificial Sequence
  Description of Artificial Sequence Synthetic oligonucleotide
cgggagtgtctcctaaggccttagtaagaagggtcctg

SEQ ID NO: 241 - DNA, 46 bp, Artificial Sequence
  Description of Artificial Sequence Synthetic oligonucleotide
acgacgggaagaggctcgtgagttgatggggagagggtccggtcgt

SEQ ID NO: 242 - DNA, 44 bp, Artificial Sequence
  Description of Artificial Sequence Synthetic oligonucleotide
cgacggaagggtcccgttgagtttgcaatggtgagggtttgtcg

SEQ ID NO: 243 - DNA, 46 bp, Artificial Sequence
  Description of Artificial Sequence Synthetic oligonucleotide
acgacgccgatggaaggggccctggtgggagggtcaaaggggtcgt

SEQ ID NO: 244 - DNA, 44 bp, Artificial Sequence
  Description of Artificial Sequence Synthetic oligonucleotide
cgacgggcaggtagatctacatgaatatgaaggaatgatcgtcg

SEQ ID NO: 245 - DNA, 45 bp, Artificial Sequence
  Description of Artificial Sequence Synthetic oligonucleotide
acgacggggagtagccgggtggttagtgtctcgcgaggaggtcgt

SEQ ID NO: 246 - DNA, 4 bp, Artificial Sequence
  Description of Artificial Sequence Synthetic oligonucleotide
tccc

SEQ ID NO: 247 - DNA, 40 bp, Artificial Sequence
  Description of Artificial Sequence Synthetic oligonucleotide
acgaccaggtggggctgctcaagtggaggttcctcgtcgt

SEQ ID NO: 248 - DNA, 40 bp, Artificial Sequence
  Description of Artificial Sequence Synthetic oligonucleotide
acgaccagaggggcctcaaatgtggggtgttgctcgtcgt

SEQ ID NO: 249 - DNA, 44 bp, Artificial Sequence
  Description of Artificial Sequence Synthetic oligonucleotide
cgaccggggtaggggttaggtgggaatggagctggaccgtgtcg

SEQ ID NO: 250 - DNA, 42 bp, Artificial Sequence
  Description of Artificial Sequence Synthetic oligonucleotide
gacggaccgttgccctggggtagtgcgcgcttcgtttacgtc

SEQ ID NO: 251 - DNA, 48 bp, Artificial Sequence
  Description of Artificial Sequence Synthetic oligonucleotide
gtggttagtaacttgcacgccgcccaattgctattcatgacaagccac

SEQ ID NO: 252 - DNA, 63 bp, Artificial Sequence; identical to SEQ ID NO: 59
  Description of Artificial Sequence Synthetic oligonucleotide

SEQ ID NO: 253 - DNA, 63 bp, Artificial Sequence; identical to SEQ ID NO: 34
  Description of Artificial Sequence Synthetic oligonucleotide

SEQ ID NO: 254 - DNA, 8 bp, Artificial Sequence
  Description of Artificial Sequence Synthetic oligonucleotide
ggtggcct

SEQ ID NO: 255 - DNA, 7 bp, Artificial Sequence
  Description of Artificial Sequence Synthetic oligonucleotide
aggggtg

SEQ ID NO: 256 - DNA, 57 bp, Artificial Sequence
  Description of Artificial Sequence Synthetic oligonucleotide
gacgacggtggccttaatagatagatgatattcttatatgtgtgaggggtggtcgtc

SEQ ID NO: 257 - DNA, 51 bp, Artificial Sequence
  Description of Artificial Sequence Synthetic oligonucleotide
gacggtggcctatattggtatgtatgaagaatagaactattagggggtgtc

SEQ ID NO: 258 - DNA, 53 bp, Artificial Sequence
  Description of Artificial Sequence Synthetic oligonucleotide
cgacggtggcctattaaatagctttagtttaagaaaagtaatagggggtgtcg

SEQ ID NO: 259 - DNA, 53 bp, Artificial Sequence
  Description of Artificial Sequence Synthetic oligonucleotide
cgacggtggcctattaagtagctttagttcaagaaaagtaatagggggtgtcg

SEQ ID NO: 260 - DNA, 10 bp, Artificial Sequence
  Description of Artificial Sequence Synthetic oligonucleotide
gtagtacgtt

SEQ ID NO: 261 - DNA, 51 bp, Artificial Sequence
  Description of Artificial Sequence Synthetic oligonucleotide
gacgtccaagtagtacgtttaattaggatttccgaattattggcatgcgtc

SEQ ID NO: 262 - DNA, 52 bp, Artificial Sequence
  Description of Artificial Sequence Synthetic oligonucleotide
ctctcgggacgacggggattttccagtgcaactagctgaaagcggtcgtccc

SEQ ID NO: 263 - DNA, 52 bp, Artificial Sequence
  Description of Artificial Sequence Synthetic oligonucleotide
ctctcgggacgaccaggattttccagtgtaactagctacagcgggtcgtccc

SEQ ID NO: 264 - DNA, 51 bp, Artificial Sequence; identical to SEQ ID NO: 79
  Description of Artificial Sequence Synthetic oligonucleotide

SEQ ID NO: 265 - DNA, 51 bp, Artificial Sequence
  Description of Artificial Sequence Synthetic oligonucleotide
ctctcgggacgacggtaggtaggccaactgggtatttactggtgtcgtccc

SEQ ID NO: 266 - DNA, 57 bp, Artificial Sequence
  Description of Artificial Sequence Synthetic oligonucleotide
ctctcgggacgacggccccgcaaggggtgaaatgacagagtcaaagtgcgtcgtccc

SEQ ID NO: 267 - DNA, 55 bp, Artificial Sequence
  Description of Artificial Sequence Synthetic oligonucleotide
ctctcgggacgacgtagtcggaaacggtgtctcagttcctcggtagagtcgtccc

SEQ ID NO: 268 - DNA, 57 bp, Artificial Sequence
  Description of Artificial Sequence Synthetic oligonucleotide
ctctcgggacgaccgcttgccccctggcatgtttagagcagagtctttggtcgtccc

SEQ ID NO: 269 - DNA, 57 bp, Artificial Sequence
  Description of Artificial Sequence Synthetic oligonucleotide
ctctcgggacgacgtccggttcatgacttcagtagtctagtgggggtctgtcgtccc

SEQ ID NO: 270 - DNA, 57 bp, Artificial Sequence
  Description of Artificial Sequence Synthetic oligonucleotide
ctctcgggacgacgcaaccattccagtggcgtctacggcttagcttttcgtcgtccc

SEQ ID NO: 271 - DNA, 57 bp, Artificial Sequence
  Description of Artificial Sequence Synthetic oligonucleotide
ctctcgggacgaccaggatgctgttccaccggggtacaggtaggtcgctgtcgtccc

SEQ ID NO: 272 - DNA, 57 bp, Artificial Sequence
  Description of Artificial Sequence Synthetic oligonucleotide
ctctcgggacgacgggcgtagcgatagaagagagcagggggagagacctgtcgtccc

SEQ ID NO: 273 - DNA, 57 bp, Artificial Sequence
  Description of Artificial Sequence Synthetic oligonucleotide
ctctcgggacgacgggaaggagttccggggtacgcgggtaagggaaggagtcgtccc

SEQ ID NO: 274 - DNA, 57 bp, Artificial Sequence
  Description of Artificial Sequence Synthetic oligonucleotide
ctctcgggacgaccaacgagtatacgcttacgtcacgttgatgctgtgggtcgtccc

SEQ ID NO: 275 - DNA, 57 bp, Artificial Sequence
  Description of Artificial Sequence Synthetic oligonucleotide
ctctcgggacgacgcattgatgtacaagctcgattcgtatcccttgatcgtcgtccc

SEQ ID NO: 276 - DNA, 57 bp, Artificial Sequence
  Description of Artificial Sequence Synthetic oligonucleotide
ctctcgggacgactgggctcgtgttctatggacaagggggagtgacctggtcgtccc

SEQ ID NO: 277 - DNA, 57 bp, Artificial Sequence
  Description of Artificial Sequence Synthetic oligonucleotide
ctctcgggacgacggaagaggctcagtgctatcttatctgagagggtttgtcgtccc

SEQ ID NO: 278 - DNA, 57 bp, Artificial Sequence
  Description of Artificial Sequence Synthetic oligonucleotide
ctctcgggacgacgggagtgtctcctaaggccttagtaagaagggtcctgtcgtccc

SEQ ID NO: 279 - DNA, 57 bp, Artificial Sequence
  Description of Artificial Sequence Synthetic oligonucleotide
ctctcgggacgacgggaagaggctcgtgagttgatggggagagggtccggtcgtccc

SEQ ID NO: 280 - DNA, 57 bp, Artificial Sequence
  Description of Artificial Sequence Synthetic oligonucleotide
ctctcgggacgacggaagggtcccgttgagtttgcaatggtgagggtttgtcgtccc

SEQ ID NO: 281 - DNA, 57 bp, Artificial Sequence
  Description of Artificial Sequence Synthetic oligonucleotide
ctctcgggacgacgccgatggaaggggccctggtgggagggtcaaaggggtcgtccc

SEQ ID NO: 282 - DNA, 57 bp, Artificial Sequence
  Description of Artificial Sequence Synthetic oligonucleotide
ctctcgggacgacgggcaggtagatctacatgaatatgaaggaatgatcgtcgtccc

SEQ ID NO: 283 - DNA, 56 bp, Artificial Sequence
  Description of Artificial Sequence Synthetic oligonucleotide
ctctcgggacgacggggagtagccgggtggttagtgtctcgcgaggaagtcgtccc

SEQ ID NO: 284 - DNA, 51 bp, Artificial Sequence
  Description of Artificial Sequence Synthetic oligonucleotide
ctctcgggacgaccaggtggggctgctcaagtggaggttcctcgtcgtccc

SEQ ID NO: 285 - DNA, 51 bp, Artificial Sequence
  Description of Artificial Sequence Synthetic oligonucleotide
ctctcgggacgaccagaggggcctcaaatgtggggtgttgctcgtcgtccc

SEQ ID NO: 286 - DNA, 57 bp, Artificial Sequence
  Description of Artificial Sequence Synthetic oligonucleotide
ctctcgggacgaccggggtaggggttaggtgggaatggagctggaccgtgtcgtccc

SEQ ID NO: 287 - DNA, 57 bp, Artificial Sequence
  Description of Artificial Sequence Synthetic oligonucleotide
ctctcgggacgacggaccgttgccctggggtagtgcgcgcttcgtttacgtcgtccc

SEQ ID NO: 288 - DNA, 54 bp, Artificial Sequence
  Description of Artificial Sequence Synthetic oligonucleotide
gacgacggtggcctatattggtatgtatgaagaatagaactattagggggtgtc

SEQ ID NO: 289 - DNA, 66 bp, Artificial Sequence
  Description of Artificial Sequence Synthetic oligonucleotide
ctctcgggacgacggtggcctattaagtagctttagttcaagaaaagtaatagggggtgtcgtccc

SEQ ID NO: 290 - DNA, 58 bp, Artificial Sequence
  Description of Artificial Sequence Synthetic oligonucleotide
gtcatgtggcgcctcgaccccagccttggtagctgttggccacacaagagcacatgac

SEQ ID NO: 291 - DNA, 58 bp, Artificial Sequence
  Description of Artificial Sequence Synthetic oligonucleotide
gtcatgtgacgagacaaggagaacggaaggcgaccggataaatcggatatcacatgac

SEQ ID NO: 292 - DNA, 39 bp, Artificial Sequence; identical to SEQ ID NO: 62
  Description of Artificial Sequence Synthetic oligonucleotide

SEQ ID NO: 293 - DNA, 39 bp, Artificial Sequence; identical to SEQ ID NO: 69
  Description of Artificial Sequence Synthetic oligonucleotide

SEQ ID NO: 294 - DNA, 52 bp, Artificial Sequence; identical to SEQ ID NO: 12
  Description of Artificial Sequence Synthetic oligonucleotide

SEQ ID NO: 295 - DNA, 52 bp, Artificial Sequence; identical to SEQ ID NO: 77
  Description of Artificial Sequence Synthetic oligonucleotide

SEQ ID NO: 296 - DNA, 38 bp, Artificial Sequence; identical to SEQ ID NO: 63
  Description of Artificial Sequence Synthetic oligonucleotide

SEQ ID NO: 297 - DNA, 38 bp, Artificial Sequence
  Description of Artificial Sequence Synthetic oligonucleotide
cgacaacgctgaacatcgctaagaacaactatctgtcg

SEQ ID NO: 298 - DNA, 51 bp, Artificial Sequence; identical to SEQ ID NO: 79
  Description of Artificial Sequence Synthetic oligonucleotide

SEQ ID NO: 299 - DNA, 51 bp, Artificial Sequence; identical to SEQ ID NO: 80
  Description of Artificial Sequence Synthetic oligonucleotide

SEQ ID NO: 300 - DNA, 42 bp, Artificial Sequence; identical to SEQ ID NO: 64
  Description of Artificial Sequence Synthetic oligonucleotide

SEQ ID NO: 301 - DNA, 42 bp, Artificial Sequence
  Description of Artificial Sequence Synthetic oligonucleotide
gacgactagtcaagactatccatggaacatgcgggcgtcgtc

SEQ ID NO: 302 - DNA, 34 bp, Artificial Sequence; identical to SEQ ID NO: 65
  Description of Artificial Sequence Synthetic oligonucleotide

SEQ ID NO: 303 - DNA, 34 bp, Artificial Sequence
  Description of Artificial Sequence Synthetic oligonucleotide
acgaactgcaaccaccgtaccccggacatcccgt

SEQ ID NO: 304 - DNA, 38 bp, Artificial Sequence; identical to SEQ ID NO: 66
  Description of Artificial Sequence Synthetic oligonucleotide

SEQ ID NO: 305 - DNA, 38 bp, Artificial Sequence
  Description of Artificial Sequence Synthetic oligonucleotide
cgacaatacgcactaattagttggctcacgctgtgtcg

SEQ ID NO: 306 - DNA, 36 bp, Artificial Sequence; identical to SEQ ID NO: 67
  Description of Artificial Sequence Synthetic oligonucleotide

SEQ ID NO: 307 - DNA, 36 bp, Artificial Sequence
  Description of Artificial Sequence Synthetic oligonucleotide
gaccgtacctcccctgaaaaagaacccccaccggtc

SEQ ID NO: 308 - DNA, 15 bp, Artificial Sequence
  Description of Artificial Sequence Synthetic oligonucleotide
gtcgtcccgagagcc

SEQ ID NO: 309 - DNA, 34 bp, Artificial Sequence
  Description of Artificial Sequence Synthetic oligonucleotide
  modified_base (16)..(26): a, c, t or g
  See specification as filed for detailed      description of substitutions and preferred embodiments
ggctctcgggacgacnnnnnnnnnnngtcgtccc

SEQ ID NO: 310 - DNA, 44 bp, Artificial Sequence; identical to SEQ ID NO: 58
  Description of Artificial Sequence Synthetic oligonucleotide

SEQ ID NO: 311 - DNA, 54 bp, Artificial Sequence; identical to SEQ ID NO: 60
  Description of Artificial Sequence Synthetic oligonucleotide

SEQ ID NO: 312 - DNA, 44 bp, Artificial Sequence; identical to SEQ ID NO: 58
  Description of Artificial Sequence Synthetic oligonucleotide

SEQ ID NO: 313 - DNA, 48 bp, Artificial Sequence; identical to SEQ ID NO: 251
  Description of Artificial Sequence Synthetic oligonucleotide

SEQ ID NO: 314 - DNA, 44 bp, Artificial Sequence; identical to SEQ ID NO: 58
  Description of Artificial Sequence Synthetic oligonucleotide

SEQ ID NO: 315 - DNA, 14 bp, Artificial Sequence
  Description of Artificial Sequence Synthetic oligonucleotide
ccacatgacacaga

SEQ ID NO: 316 - DNA, 63 bp, Artificial Sequence; identical to SEQ ID NO: 59
  Description of Artificial Sequence Synthetic oligonucleotide

SEQ ID NO: 317 - DNA, 44 bp, Artificial Sequence; identical to SEQ ID NO: 58
  Description of Artificial Sequence Synthetic oligonucleotide

SEQ ID NO: 318 - DNA, 54 bp, Artificial Sequence; identical to SEQ ID NO: 60
  Description of Artificial Sequence Synthetic oligonucleotide

SEQ ID NO: 319 - DNA, 12 bp, Artificial Sequence
  Description of Artificial Sequence Synthetic oligonucleotide
acatgacacaga

SEQ ID NO: 320 - DNA, 63 bp, Artificial Sequence; identical to SEQ ID NO: 34
  Description of Artificial Sequence Synthetic oligonucleotide

SEQ ID NO: 321 - DNA, 39 bp, Artificial Sequence; identical to SEQ ID NO: 62
  Description of Artificial Sequence Synthetic oligonucleotide

SEQ ID NO: 322 - DNA, 50 bp, Artificial Sequence
  Description of Artificial Sequence Synthetic oligonucleotide
ctctcgggacgaccgcgtttcccaagaaagcaagtattggttgtcgtccc

(19) United States
(12) Patent Application Publication
(10) Pub. No.: US 2019/0136241 A1
(43) Pub. Date: May 9, 2019

(54) APTAMER-BASED ANALYTE ASSAYS

(71) Applicant: THE TRUSTEES OF COLUMBIA UNIVERSITY IN THE CITY OF NEW YORK, New York, NY (US)

(72) Inventors: Milan Stojanovic, Fort Lee, NJ (US); Kyungae Yang, New York, NY (US); Steven Taylor, Jersey City, NJ (US); Nenad Milosavic, Hackensack, NJ (US)

(73) Assignee: THE TRUSTEES OF COLUMBIA UNIVERSITY IN THE CITY OF NEW YORK, New York, NY (US)

(21) Appl. No.: 16/306,485

(22) PCT Filed: Jun. 5, 2017
(86) PCT No.: PCT/US17/35958

§ 371 Date: Nov. 30, 2018

Related U.S. Application Data
(60) Provisional application No. 62/345,697, filed on Jun. 3, 2016.
(60) Provisional application No. 62/345,641, filed on Jun. 3, 2016.
(60) Provisional application No. 62/346,374, filed on Jun. 6, 2016.

(51) Int. Cl. C12N 15/115; G01N 33/52; A61P 7/02; C07K 16/18; G01N 33/541

(52) U.S. Cl. CPC C12N 15/115; G01N 33/523; A61P 7/02; G01N 33/526; C12N 2310/3517; G01N 33/541; C12N 2310/113; C12N 2310/16; C07K 16/18

(57) ABSTRACT

The present invention relates to aptamer-based assays to capture and/or detect analytes comprising primary and secondary aptamers, as well as compositions comprising such primary and secondary aptamers, wherein analytes comprise small molecules that offer limited mutually non-competitive epitopes to antibodies, that is, with limited ability to measure in non-competitive sandwich assays using primary and secondary antibodies or primary and secondary aptamers.

### PRIORITY CLAIM

This patent application is a national phase of International Application No. PCT/US2017/035958 filed Jun. 5, 2017 which claims priority to U.S. Provisional Applications Nos. 62/345,641 and 62/345,697, both filed Jun. 3, 2016, and U.S. Provisional Application No. 62/346,374 filed Jun. 6, 2016, the contents of each of which are hereby incorporated by reference in their entireties herein.

### GRANT INFORMATION

This invention was made with government support under grant GM104960 awarded by the National Institutes of Health and grant 1518715 awarded by the National Science Foundation. The government has certain rights in the invention.

### SEQUENCE LISTING

The instant application contains a Sequence Listing which has been submitted electronically in ASCII format and is hereby incorporated by reference in its entirety. Said ASCII copy, created on Jul. 12, 2017, is named 070050-5953_SL.txt and is 89,363 bytes in size.

### 1. INTRODUCTION

The present invention relates to aptamer-based assays to capture and/or detect analytes, including small molecules that offer limited mutually non-competitive epitopes to antibodies and are therefore difficult to detect or measure using traditional antibody sandwich-type assays.

### 2. BACKGROUND OF THE INVENTION

Non-competitive sandwich assays employ two different binding elements to capture and then detect an analyte¹⁻⁵. For example, a typical example of a sandwich assay format is an enzyme linked immunosorbent assay (ELISA) on plates, wherein one antibody (capture antibody), directed toward a target analyte (e.g., a protein), is bound to a solid support (e.g., a plate), the analyte is bound, and then a second antibody (the “detection antibody”) bearing a detectable moiety is introduced that binds to a different site on the captured analyte. Because alternative binding sites are typically present on the analyte, in the presence of excess reagent, these assays can be more sensitive than competitive assays, detect ligand with increasing signal, and have lower noise. The sandwich principle also allows more stringent washing protocols that minimizes non-specific interactions. The principle of two or more binding elements interacting with a target analyte (i.e., “sandwiching”) is also used in other assays beyond ELISA, such as lateral flow assays or latex-bead agglutination assays.

The sandwich approach becomes problematic with small analytes that can be too small to bind to two antibodies at once (e.g., steroids or catecholamines or even some small peptides such as vasopressin) or that are non-immunogenic (e.g., molecules such as phenylalanine or glucose). When antibodies against small molecules were available, several idiosyncratic approaches⁶⁻⁹ have produced ELISA-like assays for small molecules.

Aptamers are oligonucleotide-based receptors that can bind to small molecules⁹⁻¹⁵. Aptamers have been used before in traditional sandwich assays, however, traditional sandwich assays depend upon availability of more than one binding site in the analyte. Therefore, there is a need for means for detecting small molecules that overcome the problems due to the lack of multiple epitopes.

### 3. SUMMARY OF THE INVENTION

The present invention relates to aptamer-based assays to capture and detect analytes that may not lend themselves to antibody-based assays. Three different exemplary assays are provided, as well as a variety of aptamers comprising a core sequence and an operative sequence, where the operative sequence can be varied depending upon the assay to be used.

A first set of embodiments provide for an “anti-aptamer assay” in which a sample to be tested for the presence and/or amount of an analyte of interest is contacted with effective amounts of (1) a primary aptamer comprising a core sequence that binds to the analyte and (2) an “anti-aptamer” which is complementary to at least a portion of the primary aptamer, wherein the primary aptamer and/or anti-aptamer comprise a detectable moiety(ies) which detect whether the primary aptamer and anti-aptamer are bound to each other or unbound; and wherein a primary aptamer bound to the analyte does not bind to an anti-aptamer. The analyte competes with anti-aptamer for binding to primary aptamer, so that the amount of bound (or unbound) anti-aptamer correlates with the amount of analyte present.

A second set of embodiments provide for a “pseudo-sandwich assay” in which a sample to be tested for the presence and/or amount of an analyte of interest is contacted with effective amounts of (1) a primary aptamer comprising a core sequence that binds to the analyte as well as at least a portion that is complementary to a structure-switching “sensor oligonucleotide”; (2) a sensor oligonucleotide, optionally bound to a solid support, which optionally further comprises a portion complementary to a “comp” (for “complementary”) oligonucleotide; wherein the primary aptamer and/or sensor oligonucleotide and/or comp oligonucleotide comprise a detectable moiety(ies) which can detect whether the primary aptamer and sensor oligonucleotide are bound to each other or unbound; and wherein the primary aptamer and sensor oligonucleotide form a partially double stranded helix that detectably dissociates, or does not form, when the primary aptamer is bound to analyte. For example, a test sample may be added to an effective amount of complexes comprised of primary aptamer and sensor oligonucleotide and comp oligonucleotide; the amount of primary aptamer released from the complexes correlates with the amount of analyte in the test sample (and the comp and sensor oligonucleotides form an at least partially double-stranded structure). As a further example, and not by way of limitation, the complexes may be linked to a solid support, the test sample applied, and then the primary aptamer released may be detected. As a more specific non-limiting example, the sensor oligonucleotide may be linked to a solid support and complexed to primary aptamer, the resulting complexes may then be contacted with test sample, and the amount of released primary aptamer detected, for example, washed off the support.

A third set of embodiments provides for a “sandwich assay” in which a sample to be tested for the presence and/or amount of an analyte of interest is contacted with effective amounts of (1) a primary aptamer comprising a core sequence that binds to the analyte as well as at least a portion that binds to a secondary aptamer when it is bound to analyte; and (ii) a sandwich aptamer (also referred to herein as a “secondary aptamer”) which binds to primary aptamer provided that the primary aptamer is bound to analyte to form a ternary complex (the “sandwich”); wherein the primary aptamer and/or sandwich aptamer comprise a detectable moiety(ies) which can detect whether the primary aptamer and sandwich oligonucleotide are bound to each other or unbound. For example, a test sample may be added to effective amounts of primary aptamer and sandwich aptamer, and then the amounts of primary aptamer/sandwich aptamer complexes, or the amount of unbound primary aptamer or sandwich aptamer, may be detected.

Also provided herein are consensus core sequences, core sequences, and primary aptamers that can bind to primary aptamers of interest, including glucose, hydrocortisone, phenylalanine, dehydroisoandrosterone, deoxycortisone, testosterone, aldosterone, dopamine, sphingosine-1-phosphate, serotonin, melatonin, tyrosine, tobramycin, amikacin, methylene blue, ammonium ion, boronic acid, epinephrine, creatinine and vasopressin.

Further provided are associated anti-aptamer, sensor, comp and sandwich aptamers/oligonucleotides.

Further embodiments provide for kits comprising the aforementioned primary aptamers and/or oligonucleotides.

### 4. BRIEF DESCRIPTION OF THE FIGURES

FIG. 1A-G. Structures of primary aptamers (short forms) and corresponding target analytes. (A) Primary aptamer (SEQ ID NO:62, a subsequence of SEQ ID NO:12) and its target analyte, deoxycorticosterone 21-glucoside (“DOG”); (B) Primary aptamer (SEQ ID NO:63) and its target analyte, aldosterone (“ALDS”); (C) Primary aptamer (SEQ ID NO:64, a subsequence of SEQ ID NO:5) and its target analyte, cortison (“CS”); (D) Primary aptamer (SEQ ID NO:65, a subsequence of SEQ ID NO:13) and its target analyte, testosterone (“TES”); (E) Primary aptamer (SEQ ID NO:66) and its target analyte, Phe-CpRh; (F) Primary aptamer (SEQ ID NO:67) and its target analyte, L-Phenylalanine; (G) Primary aptamer (SEQ ID NO:68, a subsequence of SEQ ID NO:1) and its target analyte, glucose.

FIG. 2A-C. Design principle of “anti-aptamer assay.” (A) Effective amounts of primary aptamer (“aptamer”) labeled with fluorescent label (e.g. F=fluorescein) and its “complement” oligonucleotide (a.k.a. “anti-aptamer”) labeled with quencher (e.g., D=dabcyl quencher) can hybridize to form a duplex. Fluorescence of the label on the aptamer is quenched upon duplex formation. (B) In the presence of target analyte, primary aptamer binds to analyte instead of its complement anti-aptamer, and the fluorescence of its label is not quenched. (C) Shows increasing fluorescence with increasing concentrations of target analyte DOG in unheated samples but not heated samples (because heating interferes with analyte/aptamer binding).

FIG. 3A-L. Examples of results of anti-aptamer assays for various target analytes using “short-form” primary aptamers. (A) Primary aptamer for deoxycorticosterone 21-glucoside (“DOG”) (SEQ ID NO:62), linked to fluorescent label (e.g. F=fluorescein) and its complement anti-aptamer (SEQ ID NO:69) labeled with quencher (e.g., D=dabcyl quencher) can hybridize to form a duplex (schematically indicated by SEQ ID NO:70); less duplex forms in the presence of DOG and fluorescent signal increases. (B) 1:1 ratio of primary aptamer and anti-aptamer of (A) are combined with increasing concentrations of DOG, from 0 to 100 μM. The graph shows fluorescence over time. (C) Primary aptamer (SEQ ID NO:63) for aldosterone (“ALDS”), linked to fluorescent label (e.g. F=fluorescein) and its complement anti-aptamer labeled with quencher (e.g., D=dabcyl quencher) can hybridize to form a duplex (schematically indicated by SEQ ID NO:71); less duplex forms in the presence of ALDS and fluorescent signal increases. (D) 1:1 ratio of primary aptamer and anti-aptamer of (C) are combined with increasing concentrations of ALDS, from 0 to 100 μM. The graph shows fluorescence over time. (E) Primary aptamer for cortisol (“CS”) (SEQ ID NO:64), linked to fluorescent label (e.g. F=fluorescein) and its complement anti-aptamer labeled with quencher (e.g., D=dabcyl quencher) can hybridize to form a duplex (schematically indicated by SEQ ID NO:72); less duplex forms in the presence of CS and fluorescent signal increases. (F) 1:10 ratio of primary aptamer and anti-aptamer of (E) are combined with increasing concentrations of CS, from 0 to 125 μM. The graph shows fluorescence over time. (G) Primary aptamer for testosterone (“TES”) (SEQ ID NO:65), linked to fluorescent label (e.g. F=fluorescein) and its complement anti-aptamer labeled with quencher (e.g., D=dabcyl quencher) can hybridize to form a duplex (schematically indicated by SEQ ID NO:73); less duplex forms in the presence of TES and fluorescent signal increases. (H) 1:1 ratio of primary aptamer and anti-aptamer of (G) are combined with increasing concentrations of TES, from 0 to 100 μM. The graph shows fluorescence over time. (I) Primary aptamer for Phe-CpRh (SEQ ID NO:66), linked to fluorescent label (e.g. F=fluorescein) and its complement anti-aptamer labeled with quencher (e.g., D=dabcyl quencher) can hybridize to form a duplex (schematically indicated by SEQ ID NO:74); less duplex forms in the presence of DOG and fluorescent signal increases. (J) 1:1 ratio of primary aptamer and anti-aptamer of (I) are combined with increasing concentrations of Phe-CpRh, from 0 to 100 μM. The graph shows fluorescence over time. (K) Primary aptamer for phenylalanine (“Phe”) (SEQ ID NO:67), linked to fluorescent label (e.g. F=fluorescein) and its complement anti-aptamer labeled with quencher (e.g., D=dabcyl quencher) can hybridize to form a duplex (schematically indicated by SEQ ID NO:75); less duplex forms in the presence of Phe and fluorescent signal increases. (L) 1:1 ratio of primary aptamer and anti-aptamer of (K) are combined with increasing concentrations of Phe, from 0 to 2 mM. The graph shows fluorescence over time. These results demonstrate the target analyte-concentration dependent effect on hybridization of the aptamer and anti-aptamer strands.

FIG. 4A-C. Aptamer/anti-aptamer (short-form) results in plate-based assay format. Signal is from the reaction between TMB and HRP-tagged anti-aptamer complement. (A) DOG: anti-aptamer coated plate exposed to a solution of 1 μM DOG-aptamer and DOG target for 30 mins. (B) Glucose: a solution of 2 μM glucose-aptamer and glucose target exposed to 0.2 μM anti-aptamer for 10 mins, then “pulled-down” on to a streptavidin-coated plate. (C) Phenylalanine: a solution of 1 μM Phe-aptamer and phenylalanine target exposed to 0.2 μM anti-aptamer for 5 mins, then “pulled-down” on to a streptavidin-coated plate.

FIG. 5A-F. Examples of results of anti-aptamer assays for various target analytes using “long-form” primary aptamers. (A) A long-form of a primary aptamer for deoxycorticosterone 21-glucoside (“DOG”) (SEQ ID NO:66, which comprises SEQ ID NOS:62 and 12), linked to fluorescent label (e.g. FAM=fluorescein) and its complement anti-aptamer (SEQ ID NO:69) labeled with quencher (e.g., QFBkA13 quencher) can hybridize to form a duplex (B; SEQ ID NO:78). (C) Fluorescent signal over time after mixing aptamer and anti-aptamer in the presence of various concentrations of target. These results demonstrate the target-concentration dependent effect on hybridization of the aptamer and anti-aptamer strands. (D) A long-form of a primary aptamer for aldosterone (“ALDS”) (SEQ ID NO:79, which comprises SEQ ID NO:63), linked to fluorescent label (e.g. FAM=fluorescein) and its complement anti-aptamer (SEQ ID NO:80) labeled with quencher (e.g., QFBkA13 (“Iowa Black”; Integrated DNA Technologies) quencher) can hybridize to form a duplex (E; SEQ ID NO:81). (F) Fluorescent signal over time after mixing aptamer and anti-aptamer in the presence of various concentrations of target. These results demonstrate the target-concentration dependent effect on hybidization of the aptamer and anti-aptamer strands.

FIG. 6A-C. Design principles of pseudo-sandwich assays. (A) Primary aptamer (A^{P}) in complex with its ligand (L). (B) A^{P} is extended (A^{P}ₑₓₜ) and complementary oligonucleotide (C_{D}) is added to obtain a structure-switching sensor (shown here in a set-up to achieve fluorescent signaling; F=fluorescein and D=dabcyl quencher). This is an equilibrium process, and a system, if attached to a surface to achieve ELISA-like format, cannot be washed without removing components. (C) Pseudo-sandwich assay overcomes these problems and translate equilibrium reactions into stable double helical structure. To achieve this, C_{D} is extended into C_{Dext} and then C_{Dext}^{comp} is used to establish a different equilibrium. If C_{Dext} is attached to surface (one possible implementation, in the other one C_{Dext}^{comp} is attached to surface), upon binding of ligand, C_{Dext} and C_{Dext}^{comp} form a stable double helical complex that can be, if formed on surface, extensively washed.

FIG. 7. Selection of primary aptamers. In the depicted schematic representation of a solution-phase selection an oligonucleotide library (N_{M}, with randomized region M having 8 to 100 nucleotides, flanked by constant regions, i.e., primers for the PCR amplification, where N can be any one of A, T, G, or C) is attached to an agarose-streptavidin column via a biotinylated complementary oligonucleotide (C_{B}). Exposure to a target (blue shape; red shape is a counter-target that ensures specificity through elimination of cross-reactivity, causes elution of receptors in which a stem (S) is stabilized, and these sequences are then amplified. Figure discloses SEQ ID NOS 308, 309, 308, and 309, respectively, in order of appearance.

FIG. 8A-C. Design principles for sandwich assay for small molecules and concept of secondary “sandwich” aptamers. (A) A^{P} in complex with its L. (B) Secondary “sandwich” aptamer (A^{S}) binds to A^{P} only when it is in a complex with L to form a ternary complex. (C) If A^{P} is attached to surface, A^{S} will attach itself to a complex of A^{P} with its L, thus forming a sandwich.

FIG. 9A-B. Selection of secondary “sandwich” aptamers. (A) A solid-state selection: A target aptamer (A^{p}) is attached to a matrix (e.g., beads), incubated with a library (e.g., structured N₄₀, with partially self-complementary primers forming a stem; alternative is unstructured N_{M}) in the presence of L to isolate aptamer candidates with affinity for A^{P}*L complex. These oligonucleotides are PCR-amplified, single-stranded species regenerated, and then used in the next selection cycle. The process is repeated until convergence is reached, pool cloned and sequenced, leading to A^{S} candidates. The counter-selection is performed by elimination of binders to A^{P} in the absence of L. (B) The process in solution-phase uses pre-structured library attached to matrix via complementary oligonucleotides, and A^{S} candidates are selected because their loop is closed through binding to A^{P} and they get released from the solid surface. Counter-selection in this case is against A^{P} without ligand, and ligand itself.

FIG. 10A-C. Primary aptamer for glucose as used in pseudosandwich assay. (A) Primary aptamer (SEQ ID NO:1) bound to sensor oligonucleotide (SEQ ID NO:82). (B) Core/pocket (SEQ ID NO:83); left-most strand of hairpin is 5′ end. (C) Dose/fluorescent response curve.

FIG. 11A-C. Primary aptamer for phenylalanine as used in pseudosandwich assay. (A) Primary aptamer (SEQ ID NO:2) bound to sensor oligonucleotide (SEQ ID NO:84). (B) Core/pocket (SEQ ID NO:85); left-most strand of hairpin is 5′ end. (C) Dose/fluorescent response curve.

FIG. 12A-C. Primary aptamer for phenylalanine as used in pseudosandwich assay. (A) Primary aptamer (SEQ ID NO:3) bound to sensor oligonucleotide (SEQ ID NO:86). (B) Core/pocket (SEQ ID NO:87); left-most strand of hairpin is 5′ end. (C) Dose/fluorescent response curve.

FIG. 13A-C. Primary aptamer for phenylalanine as used in pseudosandwich assay. (A) Primary aptamer (SEQ ID NO:4) bound to sensor oligonucleotide (SEQ ID NO:88). (B) Core/pocket (SEQ ID NO:89); left-most strand of hairpin is 5′ end. (C) Dose/fluorescent response curve.

FIG. 14A-C. Primary aptamer for hydrocortisone as used in pseudosandwich assay. (A) Primary aptamer (SEQ ID NO:5) bound to sensor oligonucleotide (SEQ ID NO:90). (B) Core/pocket (SEQ ID NO:91); left-most strand of hairpin is 5′ end. (C) Dose/fluorescent response curve.

FIG. 15A-C. Primary aptamer for hydrocortisone as used in pseudosandwich assay. (A) Primary aptamer (SEQ ID NO:6) bound to sensor oligonucleotide (SEQ ID NO:92). (B) Core/pocket (SEQ ID NO:93); left-most strand of hairpin is 5′ end. (C) Dose/fluorescent response curve.

FIG. 16A-C. Primary aptamer for hydrocortisone as used in pseudosandwich assay. (A) Primary aptamer (SEQ ID NO:7) bound to sensor oligonucleotide (SEQ ID NO:94). (B) Core/pocket (SEQ ID NO:95); left-most strand of hairpin is 5′ end. (C) Dose/fluorescent response curve.

FIG. 17A-C. Primary aptamer for dehyrdroisoandrosterone and deoxycorticosterone as used in pseudosandwich assay. (A) Primary aptamer (SEQ ID NO:8) bound to sensor oligonucleotide (SEQ ID NO:96); left-most strand of hairpin is 5′ end. (B) Core/pocket (SEQ ID NO:97); left-most strand of hairpin is 5′ end. (C) Dose/fluorescent response curve.

FIG. 18A-C. Primary aptamer for dehydroisoandrosterone as used in pseudosandwich assay. (A) Primary aptamer (SEQ ID NO:9) bound to sensor oligonucleotide (SEQ ID NO:98). (B) Core/pocket (SEQ ID NO:99); left-most strand of hairpin is 5′ end. (C) Dose/fluorescent response curve.

FIG. 19A-C. Primary aptamer for dehydroisoandrosterone as used in pseudosandwich assay. (A) Primary aptamer (SEQ ID NO:10) bound to sensor oligonucleotide (SEQ ID NO:100). (B) Core/pocket (SEQ ID NO:101); left-most strand of hairpin is 5′ end. (C) Dose/fluorescent response curve.

FIG. 20A-C. Primary aptamer for dehydroisoandrosterone as used in pseudosandwich assay. (A) Primary aptamer (SEQ ID NO:11) bound to sensor oligonucleotide (SEQ ID NO:102). (B) Core/pocket (SEQ ID NO:103); left-most strand of hairpin is 5′ end. (C) Dose/fluorescent response curve.

FIG. 21A-C. Primary aptamer for deoxycorticosterone as used in pseudosandwich assay. (A) Primary aptamer (SEQ ID NO:12) bound to sensor oligonucleotide (SEQ ID NO:104). (B) Core/pocket (SEQ ID NO:105); left-most strand of hairpin is 5′ end. (C) Dose/fluorescent response curve.

FIG. 22A-C. Primary aptamer for testosterone as used in pseudosandwich assay. (A) Primary aptamer (SEQ ID NO:13) bound to sensor oligonucleotide (SEQ ID NO:106). (B) Core/pocket (SEQ ID NO:107); left-most strand of hairpin is 5′ end. (C) Dose/fluorescent response curve.

FIG. 23A-C. Primary aptamer for testosterone as used in pseudosandwich assay. (A) Primary aptamer (SEQ ID NO:14) bound to sensor oligonucleotide (SEQ ID NO:108). (B) Core/pocket (SEQ ID NO:109); left-most strand of hairpin is 5′ end. (C) Dose/fluorescent response curve.

FIG. 24A-C. Primary aptamer for testosterone as used in pseudosandwich assay. (A) Primary aptamer (SEQ ID NO:15) bound to sensor oligonucleotide (SEQ ID NO:110). (B) Core/pocket (SEQ ID NO:111); left-most strand of hairpin is 5′ end. (C) Dose/fluorescent response curve.

FIG. 25A-C. Primary aptamer for testosterone as used in pseudosandwich assay. (A) Primary aptamer (SEQ ID NO:16) bound to sensor oligonucleotide (SEQ ID NO:112). (B) Core/pocket (SEQ ID NO:113); left-most strand of hairpin is 5′ end. (C) Dose/fluorescent response curve.

FIG. 26A-C. Primary aptamer for testosterone as used in pseudosandwich assay. (A) Primary aptamer (SEQ ID NO:17) bound to sensor oligonucleotide (SEQ ID NO:114). (B) Core/pocket (SEQ ID NO:115); left-most strand of hairpin is 5′ end. (C) Dose/fluorescent response curve.

FIG. 27A-C. Primary aptamer for sphingosine-1-phosphate (d18:1) as used in pseudosandwich assay. (A) Primary aptamer (SEQ ID NO:18) bound to sensor oligonucleotide (SEQ ID NO:116). (B) Core/pocket (SEQ ID NO:117); left-most strand of hairpin is 5′ end. (C) Dose/fluorescent response curve.

FIG. 28A-C. Primary aptamer for dopamine as used in pseudosandwich assay. (A) Primary aptamer (SEQ ID NO:19) bound to sensor oligonucleotide (SEQ ID NO:118). (B) Core/pocket (SEQ ID NO:119); left-most strand of hairpin is 5′ end. (C) Dose/fluorescent response curve.

FIG. 29A-C. Primary aptamer for dopamine as used in pseudosandwich assay. (A) Primary aptamer (SEQ ID NO:20) bound to sensor oligonucleotide (SEQ ID NO:120). (B) Core/pocket (SEQ ID NO:121); left-most strand of hairpin is 5′ end. (C) Dose/fluorescent response curve.

FIG. 30A-C. Primary aptamer for dopamine as used in pseudosandwich assay. (A) Primary aptamer (SEQ ID NO:21) bound to sensor oligonucleotide (SEQ ID NO:122). (B) Core/pocket (SEQ ID NO:123); left-most strand of hairpin is 5′ end. (C) Dose/fluorescent response curve.

FIG. 30A-C. Primary aptamer for dopamine as used in pseudosandwich assay. (A) Primary aptamer (SEQ ID NO:21) bound to sensor oligonucleotide (SEQ ID NO:122). (B) Core/pocket (SEQ ID NO:123); left-most strand of hairpin is 5′ end. (C) Dose/fluorescent response curve.

FIG. 31A-C. Primary aptamer for dopamine as used in pseudosandwich assay. (A) Primary aptamer (SEQ ID NO:22) bound to sensor oligonucleotide (SEQ ID NO:124). (B) Core/pocket (SEQ ID NO:125); left-most strand of hairpin is 5′ end. (C) Dose/fluorescent response curve.

FIG. 32A-C. Primary aptamer for dopamine as used in pseudosandwich assay. (A) Primary aptamer (SEQ ID NO:23) bound to sensor oligonucleotide (SEQ ID NO:126). (B) Core/pocket (SEQ ID NO:127); left-most strand of hairpin is 5′ end. (C) Dose/fluorescent response curve.

FIG. 33A-C. Primary aptamer for dopamine as used in pseudosandwich assay. (A) Primary aptamer (SEQ ID NO:24) bound to sensor oligonucleotide (SEQ ID NO:128). (B) Core/pocket (SEQ ID NO:129); left-most strand of hairpin is 5′ end. (C) Dose/fluorescent response curve.

FIG. 34A-C. Primary aptamer for dopamine as used in pseudosandwich assay. (A) Primary aptamer (SEQ ID NO:25) bound to sensor oligonucleotide (SEQ ID NO:130). (B) Core/pocket (SEQ ID NO:131); left-most strand of hairpin is 5′ end. (C) Dose/fluorescent response curve.

FIG. 35A-C. Primary aptamer for dopamine as used in pseudosandwich assay. (A) Primary aptamer (SEQ ID NO:26) bound to sensor oligonucleotide (SEQ ID NO:132). (B) Core/pocket (SEQ ID NO:133); left-most strand of hairpin is 5′ end. (C) Dose/fluorescent response curve.

FIG. 36A-C. Primary aptamer for dopamine as used in pseudosandwich assay. (A) Primary aptamer (SEQ ID NO:27) bound to sensor oligonucleotide (SEQ ID NO:134). (B) Core/pocket (SEQ ID NO:135); left-most strand of hairpin is 5′ end. (C) Dose/fluorescent response curve.

FIG. 37A-C. Primary aptamer for dopamine as used in pseudosandwich assay. (A) Primary aptamer (SEQ ID NO:28) bound to sensor oligonucleotide (SEQ ID NO:136). (B) Core/pocket (SEQ ID NO:137); left-most strand of hairpin is 5′ end. (C) Dose/fluorescent response curve.

FIG. 38A-C. Primary aptamer for dopamine as used in pseudosandwich assay. (A) Primary aptamer (SEQ ID NO:29) bound to sensor oligonucleotide (SEQ ID NO:138). (B) Core/pocket (SEQ ID NO:139); left-most strand of hairpin is 5′ end. (C) Dose/fluorescent response curve.

FIG. 39A-C. Primary aptamer for dopamine as used in pseudosandwich assay. (A) Primary aptamer (SEQ ID NO:30) bound to sensor oligonucleotide (SEQ ID NO:140). (B) Core/pocket (SEQ ID NO:141); left-most strand of hairpin is 5′ end. (C) Dose/fluorescent response curve.

FIG. 40A-C. Primary aptamer for dopamine as used in pseudosandwich assay. (A) Primary aptamer (SEQ ID NO:31) bound to sensor oligonucleotide (SEQ ID NO:142). (B) Core/pocket (SEQ ID NO:143); left-most strand of hairpin is 5′ end. (C) Dose/fluorescent response curve.

FIG. 41A-C. Primary aptamer for dopamine as used in pseudosandwich assay. (A) Primary aptamer (SEQ ID NO:32) bound to sensor oligonucleotide (SEQ ID NO:144). (B) Core/pocket (SEQ ID NO:145); left-most strand of hairpin is 5′ end. (C) Dose/fluorescent response curve.

FIG. 42A-C. Primary aptamer for dopamine as used in pseudosandwich assay. (A) Primary aptamer (SEQ ID NO:33) bound to sensor oligonucleotide (SEQ ID NO:146). (B) Core/pocket (SEQ ID NO:147); left-most strand of hairpin is 5′ end. (C) Dose/fluorescent response curve.

FIG. 43A-B. Primary aptamer for deoxycorticosterone (SEQ ID NO: 62) as used in pseudosandwich assay. In certain non-limiting embodiments, the deoxycorticosterone-binding primary aptamer has a binding affinity (dissociation constant, Kd) for deoxycorticosterone that is about 30 nM (in an aqueous solution at room temperature or 25° C.).

FIG. 44A-B. Primary aptamer for cortisol (SEQ ID NO: 148) as used in pseudosandwich assay. In certain non-limiting embodiments, the cortisol-binding primary aptamer has a binding affinity (dissociation constant, Kd) for cortisol that is about 30 nM (in an aqueous solution at room temperature or 25° C.).

FIG. 45A-B. Primary aptamer for serotonin (SEQ ID NO: 58) as used in pseudosandwich assay. In certain non-limiting embodiments, the serotonin-binding primary aptamer has a binding affinity (dissociation constant, Kd) for serotonin that is about 25 nM (in an aqueous solution at room temperature or 25° C.).

FIG. 46A-B. Primary aptamer for glucose (SEQ ID NO: 149) as used in pseudosandwich assay. In certain non-limiting embodiments, the glucose-binding primary aptamer has a binding affinity (dissociation constant, Kd) for glucose that is about 8 nM (in an aqueous solution at room temperature or 25° C.) and binds selectively with glucose versus galactose.

FIG. 47A-F. Examples of pseudosandwich assays, as practiced in solution and on a plate. (A) Example of pseudosandwich assay in solution. Sequences that are participating in a pseudosandwich assays, A^{P} is primary aptamer for deoxycorticosterone 21-glucoside (DOG) (shown labeled with Fluorescein) (SEQ ID NO:12; (SEQ ID NO:150). Cₑₓₜ is competitor that is extended (labeled with quencher, Q), while Cₑₓₜ^{comp} is complementary oligonucleotide which forms stable double helix. Dose-fluorescence response curve showing formation of double helix between Cₑₓₜ and Cₑₓₜ^{comp}, and release of fluorescently labeled aptamer (FIG. 6B; in this case Cₑₓₜ was labeled with a quencher, Q, and A^{P} with fluorescein, F). (B) Example of pseudosandwich assay in solution. Sequences that are participating in a pseudosandwich assays, Ap is primary aptamer for L-phenylalanine (shown labeled with Fluorescein) (SEQ ID NO: 322; (SEQ ID NO:151). Cₑₓₜ is competitor that is extended (labeled with quencher, Q), while Cₑₓₜ^{comp} is complementary oligonucleotide which forms stable double helix. Dose-fluorescence response curve showing formation of double helix between Cₑₓₜ and Cₑₓₜ^{comp}, and release of fluorescently labeled aptamer (FIG. 6B; in this case Cₑₓₜ was labeled with a quencher, Q, and A^{P} with fluorescein, F). (C) Example of pseudosandwich assay in solution. Sequences that are participating in a pseudosandwich assays, Ap is primary aptamer for D-glucose (shown labeled with Fluorescein) (SEQ ID NO:1; (SEQ ID NO:152). Cₑₓₜ is competitor that is extended (labeled with quencher, Q), while Cₑₓₜ^{comp} is complementary oligonucleotide which forms stable double helix. Dose-fluorescence response curve showing formation of double helix between Cₑₓₜ and Cₑₓₜ^{comp}, and release of fluorescently labeled aptamer (FIG. 6B; in this case Cₑₓₜ was labeled with a quencher, Q, and A^{P} with fluorescein, F). (D) Example of pseudosandwich assay in solution. Sequences that are participating in a pseudosandwich assays, Ap is primary aptamer for L-tyrosine (shown labeled with Fluorescein) (SEQ ID NO:33; (SEQ ID NO:153). Cₑₓₜ is competitor that is extended (labeled with quencher, Q), while Cₑₓₜ^{comp} is complementary oligonucleotide which forms stable double helix. Dose-fluorescence response curve showing formation of double helix between Cₑₓₜ and Cₑₓₜ^{comp}, and release of fluorescently labeled aptamer (FIG. 6B; in this case Cₑₓₜ was labeled with a quencher, Q, and A^{P} with fluorescein, F). (E) Example of pseudosandwich assay plate. Sequences as used in assay on plates (SEQ ID NO:12; (SEQ ID NO:154); sequences are the same as in FIG. 47A but sandwich is adapted to be formed on a plate (horseradish peroxidase-streptavidin conjugate (HRP-STV) used to amplify sandwich). Dose-color intensity response indicating ligand-dose-dependent sandwich formation on the surface of plate. (F) Example of pseudosandwich assay plate. Sequences as used in assay on plates (SEQ ID NO: 322; (SEQ ID NO:155); sequences are the same as in FIG. 47B but sandwich is adapted to be formed on a plate (horseradish peroxidase-streptavidin conjugate (HRP-STV) used to amplify sandwich). Dose-color intensity response indicating ligand-dose-dependent sandwich formation on the surface of plate.

FIG. 48A-E. Examples of secondary aptamers and examples of sandwich assays in solution and on a plate. (A) Example of secondary aptamers and assays in solution. Deoxycorticosterone as the targeted molecule that was used to form a complex with a primary aptamer. Presumed secondary structure of a primary aptamer (A^{P}) as used in selection of secondary aptamers (SEQ ID NO:62; derived from SEQ ID NO:12). Presumed secondary structure of secondary aptamer (A^{S}) that binds to the structure (SEQ ID NO:34). Dose-fluorescence response curve to increasing quantities of target is shown as red curve, while blue is control in which primary aptamer is eliminated. For the purpose of this assay secondary aptamer was labeled with fluorescein, and Cₑₓₜ with quencher was added to it. (B) Example of secondary aptamers and assays in solution. Deoxycorticosterone as the targeted molecule that was used to form a complex with a primary aptamer. Presumed secondary structure of a primary aptamer (A^{P}) as used in selection of secondary aptamers (SEQ ID NO:62; derived from SEQ ID NO:12). Presumed secondary structure of secondary aptamer (A^{S}) that binds to the structure (SEQ ID NO:35). Dose-fluorescence response curve to increasing quantities of target is shown as red curve, while blue is control in which primary aptamer is eliminated. For the purpose of this assay secondary aptamer was labeled with fluorescein, and Cₑₓₜ with quencher was added to it. (C) Example of secondary aptamers and assays in solution. Serotonin as the targeted molecule that was used to form a complex with a primary aptamer. Presumed secondary structure of a primary aptamer (A^{P}) as used in selection of secondary aptamers (SEQ ID NO:58; derived from SEQ ID NO:25). Presumed secondary structure of secondary aptamer (A^{S}) that binds to the structure (SEQ ID NO:59). Dose-fluorescence response curve to increasing quantities of target is shown as red curve, while blue is control in which primary aptamer is eliminated. For the purpose of this assay secondary aptamer was labeled with fluorescein, and Cₑₓₜ with quencher was added to it. (D) Example of sandwich assay on plate. Serotonin as the targeted molecule that was used to form a complex with a primary aptamer. Presumed secondary structure of a primary aptamer (A^{P}) as used in selection of secondary aptamers (SEQ ID NO:58; derived from SEQ ID NO:25). Presumed secondary structure of secondary aptamer (A^{S}) that binds to the structure (SEQ ID NO:59; Derived from SEQ ID NO: 36). Dose-color intensity response curve to ligand; color is developed by HRP-STV reaction.

FIG. 49A-Q. Exemplary Sandwich assays. FIG. 49C discloses SEQ ID NOS 310-311, respectively, in order of appearance. FIG. 49H discloses SEQ ID NOS 312-313, respectively, in order of appearance. FIG. 49J discloses SEQ ID NOS 314-316, respectively, in order of appearance. FIG. 49L discloses SEQ ID NOS 317-318, respectively, in order of appearance. FIG. 49N discloses SEQ ID NOS 319-321, respectively, in order of appearance.

FIG. 50A-B. (A) Glucose-binding primary aptamer (SEQ ID NO:68) showing stem and loop structures. (B) Dose/fluorescent response curve resulting from a primary aptamer comprising SEQ ID NO:68 and additional operative sequence complementary to a sensor oligonucleotide analogous to FIG. 10A-C, showing selective binding to glucose (top curve) versus galactose (middle curve) or fructose (bottom curve).

FIG. 51A-B. (A) Phenylalanine-binding primary aptamer (SEQ ID NO:167) showing stem and loop structures. (B) Dose/fluorescent response curve resulting from a primary aptamer comprising SEQ ID NO:167 and additional operative sequence complementary to a sensor oligonucleotide analogous to FIG. 11A-C, showing binding to phenylalanine.

FIG. 52A-B. (A) Phenylalanine-binding primary aptamer (SEQ ID NO:67) showing stem and loop structures. (B) Dose/fluorescent response curve resulting from a primary aptamer comprising SEQ ID NO:67 and additional operative sequence complementary to a sensor oligonucleotide analogous to FIG. 12A-C, showing binding to phenylalanine.

FIG. 53A-B. (A) Phenylalanine-binding primary aptamer (SEQ ID NO:174) showing stem and loop structures. (B) Dose/fluorescent response curve resulting from a primary aptamer comprising SEQ ID NO:174 and additional operative sequence complementary to a sensor oligonucleotide analogous to FIG. 13A-C, showing binding to phenylalanine (top curve) relative to tyrosine, glycine and tryptophan.

FIG. 54A-B. (A) Hydrocortisone-binding primary aptamer (SEQ ID NO:178) showing stem and loop structures. (B) Dose/fluorescent response curve resulting from a primary aptamer comprising SEQ ID NO:178 and additional operative sequence complementary to a sensor oligonucleotide analogous to FIG. 14A-C, showing binding to hydrocortisone (top curve) relative to other steroids.

FIG. 55A-B. (A) Hydrocortisone-binding primary aptamer (SEQ ID NO:181) showing stem and loop structures. (B) Dose/fluorescent response curve resulting from a primary aptamer comprising SEQ ID NO:181 and additional operative sequence complementary to a sensor oligonucleotide analogous to FIG. 15A-C, showing binding to hydrocortisone (top curve) relative to other steroids.

FIG. 56A-B. (A) Hydrocortisone-binding primary aptamer (SEQ ID NO:182) showing stem and loop structures. (B) Dose/fluorescent response curve resulting from a primary aptamer comprising SEQ ID NO:182 and additional operative sequence complementary to a sensor oligonucleotide analogous to FIG. 16A-C, showing binding to hydrocortisone (top curve) relative to other steroids.

FIG. 57A-B. (A) Dehydroisoandrosterone-binding primary aptamer (SEQ ID NO:183) showing stem and loop structures. (B) Dose/fluorescent response curve resulting from a primary aptamer comprising SEQ ID NO:183 and additional operative sequence complementary to a sensor oligonucleotide analogous to FIG. 18A-C, showing binding to dehydroisoandrosterone (top curve) relative to other steroids.

FIG. 58A-B. (A) Dehydroisoandrosterone-binding primary aptamer (SEQ ID NO:184) showing stem and loop structures. (B) Dose/fluorescent response curve resulting from a primary aptamer comprising SEQ ID NO:184 and additional operative sequence complementary to a sensor oligonucleotide analogous to FIG. 19A-C, showing binding to dehydroisoandrosterone (top curve) relative to other steroids.

FIG. 59A-B. (A) Dehydroisoandrosterone-binding primary aptamer (SEQ ID NO:185) showing stem and loop structures. (B) Dose/fluorescent response curve resulting from a primary aptamer comprising SEQ ID NO:185 and additional operative sequence complementary to a sensor oligonucleotide analogous to FIG. 20A-C, showing binding to dehydroisoandrosterone (top curve) relative to other steroids.

FIG. 60A-B. (A) Deoxycorticosterone-binding primary aptamer (SEQ ID NO:188) showing stem and loop structures. (B) Dose/fluorescent response curve resulting from a primary aptamer comprising SEQ ID NO:188 and additional operative sequence complementary to a sensor oligonucleotide analogous to FIG. 21A-C, showing binding to deoxycorticosterone (top curve) relative to other steroids.

FIG. 61A-B. (A) Deoxycorticosterone-binding primary aptamers (SEQ ID NOS:189 and 190) showing stem and loop structures. (B) Gel analysis showing selective binding.

FIG. 62A-B. (A) Testosterone-binding primary aptamer (SEQ ID NO:191) showing stem and loop structures. (B) Dose/fluorescent response curve resulting from a primary aptamer comprising SEQ ID NO:191 and additional operative sequence complementary to a sensor oligonucleotide analogous to FIG. 22A-C, showing binding to testosterone (top curve) relative to other steroids.

FIG. 63A-B. (A) Testosterone-binding primary aptamer (SEQ ID NO:192) showing stem and loop structures. (B) Dose/fluorescent response curve resulting from a primary aptamer comprising SEQ ID NO:192 and additional operative sequence complementary to a sensor oligonucleotide analogous to FIG. 26A-C, showing binding to testosterone (top curve) relative to other steroids.

FIG. 64A-B. (A) Sphingosine-1-phosphate-binding primary aptamer (SEQ ID NO:193) showing stem and loop structures. (B) Dose/fluorescent response curve resulting from a primary aptamer comprising SEQ ID NO:13 and additional operative sequence complementary to a sensor oligonucleotide analogous to FIG. 27A-C, showing binding to sphingosine-1-phosphate.

FIG. 65A-B. (A) Dopamine-binding primary aptamer (SEQ ID NO:196) showing stem and loop structures. (B) Dose/fluorescent response curve resulting from a primary aptamer comprising SEQ ID NO:196 and additional operative sequence complementary to a sensor oligonucleotide analogous to FIG. 30A-C, showing binding to dopamine (top curve) relative to (curves in descending order) norephinephrine, serotonin and 5-HIAA.

FIG. 66A-B. (A) Dopamine-binding primary aptamer (SEQ ID NO:197) showing stem and loop structures. (B) Dose/fluorescent response curve resulting from a primary aptamer comprising SEQ ID NO:197 and additional operative sequence complementary to a sensor oligonucleotide analogous to FIG. 31A-C, showing binding to dopamine (top curve) relative to (curves in descending order) L-dopa, serotonin and tyrosine.

FIG. 67A-B. (A) Dopamine-binding primary aptamer (SEQ ID NO:200) showing stem and loop structures. (B) Dose/fluorescent response curve resulting from a primary aptamer comprising SEQ ID NO:200 and additional operative sequence complementary to a sensor oligonucleotide analogous to FIG. 32A-C, showing binding to dopamine (top curve) relative to (curves in descending order) serotonin and tyrosine.

FIG. 68A-B. (A) Dopamine-binding primary aptamer (SEQ ID NO:201) showing stem and loop structures. (B) Dose/fluorescent response curve resulting from a primary aptamer comprising SEQ ID NO:201 and additional operative sequence complementary to a sensor oligonucleotide analogous to FIG. 29A-C, showing binding to dopamine (top curve) relative to (curves in descending order) L-dopa, serotonin and then various others, including tyrosine and melatonin.

FIG. 69A-B. (A) Dopamine-binding primary aptamer (SEQ ID NO:202) showing stem and loop structures. (B) Dose/fluorescent response curve resulting from a primary aptamer comprising SEQ ID NO:202 and additional operative sequence complementary to a sensor oligonucleotide analogous to FIG. 28A-C, showing binding to dopamine (top curve) relative to (curves in descending order) serotonin and then various others, including tyrosine.

FIG. 70A-B. (A) Serotonin-binding primary aptamer (SEQ ID NO:203) showing stem and loop structures. (B) Dose/fluorescent response curve resulting from a primary aptamer comprising SEQ ID NO:203 and additional operative sequence complementary to a sensor oligonucleotide analogous to FIG. 34A-C, showing binding to serotonin (top curve) relative to (curves in descending order) H-IAA, norepinephrine and dopamine.

FIG. 71A-B. (A) Serotonin-binding primary aptamer (SEQ ID NO:204) showing stem and loop structures. (B) Dose/fluorescent response curve resulting from a primary aptamer comprising SEQ ID NO:204 and additional operative sequence complementary to a sensor oligonucleotide analogous to FIG. 37A-C, showing binding to serotonin (top curve) relative to (curves in descending order) dopamine and various other compounds.

FIG. 72A-B. (A) Serotonin-binding primary aptamer (SEQ ID NO:205) showing stem and loop structures. (B) Dose/fluorescent response curve resulting from a primary aptamer comprising SEQ ID NO:205 and additional operative sequence complementary to a sensor oligonucleotide analogous to FIG. 33A-C, showing binding to serotonin (top curve) relative to e.g., melatonin, 5-HIAA and tryptophan.

FIG. 73A-B. (A) Serotonin-binding primary aptamer (SEQ ID NO:206) showing stem and loop structures. (B) Dose/fluorescent response curve resulting from a primary aptamer comprising SEQ ID NO:206 and additional operative sequence complementary to a sensor oligonucleotide analogous to FIG. 35A-C, showing binding to serotonin (top curve) relative to e.g., melatonin, 5-HIAA and tryptophan.

FIG. 74A-B. (A) Serotonin-binding primary aptamer (SEQ ID NO:207) showing stem and loop structures. (B) Dose/fluorescent response curve resulting from a primary aptamer comprising SEQ ID NO:207 and additional operative sequence complementary to a sensor oligonucleotide analogous to FIG. 36A-C, showing binding to serotonin (top curve) relative to other compounds.

FIG. 75A-B. (A) Serotonin-binding primary aptamer (SEQ ID NO:208) showing stem and loop structures. (B) Dose/fluorescent response curve resulting from a primary aptamer comprising SEQ ID NO:208 and additional operative sequence complementary to a sensor oligonucleotide analogous to FIG. 38A-C, showing binding to serotonin (top curve) relative to other compounds.

FIG. 76A-B. (A) Serotonin-binding primary aptamer (SEQ ID NO:209) showing stem and loop structures. (B) Dose/fluorescent response curve resulting from a primary aptamer comprising SEQ ID NO:209 and additional operative sequence complementary to a sensor oligonucleotide analogous to FIG. 39A-C, showing binding to serotonin (top curve) relative to other compounds.

FIG. 77A-B. (A) Melatonin-binding primary aptamer (SEQ ID NO:210) showing stem and loop structures. (B) Dose/fluorescent response curve resulting from a primary aptamer comprising SEQ ID NO:210 and additional operative sequence complementary to a sensor oligonucleotide analogous to FIG. 42A-C, showing binding to melatonin (top curve) relative to other compounds.

FIG. 78A-B. (A) Melatonin-binding primary aptamer (SEQ ID NO:211) showing stem and loop structures. (B) Dose/fluorescent response curve resulting from a primary aptamer comprising SEQ ID NO:211 and additional operative sequence complementary to a sensor oligonucleotide analogous to FIG. 40A-C, showing binding to melatonin (top curve) relative to other compounds.

FIG. 79A-B. Aldosterone-binding primary aptamer (SEQ ID NO:214) showing stem and loop structures. (B) Dose/fluorescent response curve resulting from a primary aptamer comprising SEQ ID NO:214 and additional operative sequence complementary to a sensor oligonucleotide, showing binding to aldosterone (top curve) relative to other steroid compounds.

FIG. 80A-B. Aldosterone-binding primary aptamer (SEQ ID NO:215) showing stem and loop structures. (B) Dose/fluorescent response curve resulting from a primary aptamer comprising SEQ ID NO:215 and additional operative sequence complementary to a sensor oligonucleotide, showing binding to aldosterone (top curve) relative to other steroid compounds.

FIG. 81A-B. Tobramycin-binding primary aptamer (SEQ ID NO:218) showing stem and loop structures. (B) Dose/fluorescent response curve resulting from a primary aptamer comprising SEQ ID NO:218 and additional operative sequence complementary to a sensor oligonucleotide, showing binding to tobramycin (top curve) relative to amikacin and kanamycin.

FIG. 82A-B. Tobramycin-binding primary aptamer (SEQ ID NO:221) showing stem and loop structures. (B) Dose/fluorescent response curve resulting from a primary aptamer comprising SEQ ID NO:221 and additional operative sequence complementary to a sensor oligonucleotide, showing binding to tobramycin (top curve) relative to amikacin and kanamycin.

FIG. 83A-B. Amikacin-binding primary aptamer (SEQ ID NO:225) showing stem and loop structures. (B) Dose/fluorescent response curve resulting from a primary aptamer comprising SEQ ID NO:225 and additional operative sequence complementary to a sensor oligonucleotide, showing binding to amikacin (top curve) relative to tobramycin and kanamycin.

FIG. 84A-B. Amikacin-binding primary aptamer (SEQ ID NO:228) showing stem and loop structures. (B) Dose/fluorescent response curve resulting from a primary aptamer comprising SEQ ID NO:228 and additional operative sequence complementary to a sensor oligonucleotide, showing binding to amikacin (top curve) relative to tobramycin and kanamycin.

FIG. 85A-B. Amikacin-binding primary aptamer (SEQ ID NO:230) showing stem and loop structures. (B) Dose/fluorescent response curve resulting from a primary aptamer comprising SEQ ID NO:230 and additional operative sequence complementary to a sensor oligonucleotide, showing binding to amikacin (top curve) relative to tobramycin and kanamycin.

FIG. 86A-B. Methylene blue-binding primary aptamer (SEQ ID NO:231) showing stem and loop structures. (B) Dose/fluorescent response curve resulting from a primary aptamer comprising SEQ ID NO: 231 and additional operative sequence complementary to a sensor oligonucleotide, showing binding to methylene blue.

FIG. 87A-B. Methylene blue-binding primary aptamer (SEQ ID NO:232) showing stem and loop structures. (B) Dose/fluorescent response curve resulting from a primary aptamer comprising SEQ ID NO: 232 and additional operative sequence complementary to a sensor oligonucleotide, showing binding to methylene blue.

FIG. 88A-B. Methylene blue-binding primary aptamer (SEQ ID NO:233) showing stem and loop structures. (B) Dose/fluorescent response curve resulting from a primary aptamer comprising SEQ ID NO:233 and additional operative sequence complementary to a sensor oligonucleotide, showing binding to methylene blue.

FIG. 89A-B. Methylene blue-binding primary aptamer (SEQ ID NO:234) showing stem and loop structures. (B) Dose/fluorescent response curve resulting from a primary aptamer comprising SEQ ID NO:234 and additional operative sequence complementary to a sensor oligonucleotide, showing binding to methylene blue.

FIG. 90A-B. Methylene blue-binding primary aptamer (SEQ ID NO:235) showing stem and loop structures. (B) Dose/fluorescent response curve resulting from a primary aptamer comprising SEQ ID NO:235 and additional operative sequence complementary to a sensor oligonucleotide, showing binding to methylene blue.

FIG. 91A-B. Methylene blue-binding primary aptamer (SEQ ID NO:236) showing stem and loop structures. (B) Dose/fluorescent response curve resulting from a primary aptamer comprising SEQ ID NO:236 and additional operative sequence complementary to a sensor oligonucleotide, showing binding to methylene blue.

FIG. 92A-B. Ammonium-binding primary aptamer (SEQ ID NO:239) showing stem and loop structures. (B) Dose/fluorescent response curve resulting from a primary aptamer comprising SEQ ID NO:239 and additional operative sequence complementary to a sensor oligonucleotide, showing selective binding to ammonium versus glycine or ethanolamine or potassium ion.

FIG. 93A-B. Ammonium-binding primary aptamer (SEQ ID NO:240) showing stem and loop structures. (B) Dose/fluorescent response curve resulting from a primary aptamer comprising SEQ ID NO:240 and additional operative sequence complementary to a sensor oligonucleotide, showing selective binding to ammonium versus glycine or ethanolamine or potassium ion.

FIG. 94A-B. Ammonium-binding primary aptamer (SEQ ID NO:241) showing stem and loop structures. (B) Dose/fluorescent response curve resulting from a primary aptamer comprising SEQ ID NO:241 and additional operative sequence complementary to a sensor oligonucleotide, showing selective binding to ammonium versus glycine or ethanolamine or potassium ion.

FIG. 95A-B. Ammonium-binding primary aptamer (SEQ ID NO:242) showing stem and loop structures. (B) Dose/fluorescent response curve resulting from a primary aptamer comprising SEQ ID NO:242 and additional operative sequence complementary to a sensor oligonucleotide, showing selective binding to ammonium versus glycine or ethanolamine or potassium ion.

FIG. 96A-B. Ammonium-binding primary aptamer (SEQ ID NO:243) showing stem and loop structures. (B) Dose/fluorescent response curve resulting from a primary aptamer comprising SEQ ID NO:243 and additional operative sequence complementary to a sensor oligonucleotide, showing selective binding to ammonium versus glycine or ethanolamine or potassium ion.

FIG. 97A-B. Ammonium-binding primary aptamer (SEQ ID NO:244) showing stem and loop structures. (B) Dose/fluorescent response curve resulting from a primary aptamer comprising SEQ ID NO:244 and additional operative sequence complementary to a sensor oligonucleotide, showing selective binding to ammonium versus glycine or ethanolamine or potassium ion.

FIG. 98A-B. Ammonium-binding primary aptamer (SEQ ID NO:245) showing stem and loop structures. (B) Dose/fluorescent response curve resulting from a primary aptamer comprising SEQ ID NO:245 and additional operative sequence complementary to a sensor oligonucleotide, showing selective binding to ammonium versus glycine or ethanolamine or potassium ion.

FIG. 99A-B. Boronic acid-binding primary aptamer (SEQ ID NO:247) showing stem and loop structures. (B) Dose/fluorescent response curve resulting from a primary aptamer comprising SEQ ID NO:247 and additional operative sequence complementary to a sensor oligonucleotide, showing selective binding to boronic acid versus bisboronic acid, e.g., complexed to glucose.

FIG. 100A-B. Boronic acid-binding primary aptamer (SEQ ID NO:248) showing stem and loop structures. (B) Dose/fluorescent response curve resulting from a primary aptamer comprising SEQ ID NO:248 and additional operative sequence complementary to a sensor oligonucleotide, showing selective binding to boronic acid versus bisboronic acid, e.g., complexed to glucose.

FIG. 101A-B. Epinephrine-binding primary aptamer (SEQ ID NO:249) showing stem and loop structures. (B) Dose/fluorescent response curve resulting from a primary aptamer comprising SEQ ID NO:249 and additional operative sequence complementary to a sensor oligonucleotide, showing selective binding to epinephrine and, in descending order, serotonin, norepinephrine and dopamine.

FIG. 102A-B. Epinephrine-binding primary aptamer (SEQ ID NO:250) showing stem and loop structures. (B) Dose/fluorescent response curve resulting from a primary aptamer comprising SEQ ID NO:250 and additional operative sequence complementary to a sensor oligonucleotide, showing selective binding to epinephrine and, in descending order, serotonin, dopamine and norepinephrine.

FIG. 103A-B. Creatinine-binding primary aptamer (SEQ ID NO:256) showing stem and loop structures. (B) Dose/fluorescent response curve resulting from a primary aptamer comprising SEQ ID NO:256 showing binding to creatinine.

FIG. 104A-B. Creatinine-binding primary aptamer (SEQ ID NO:257) showing stem and loop structures. (B) Dose/fluorescent response curve resulting from a primary aptamer comprising SEQ ID NO:257 showing binding to creatinine.

FIG. 105A-B. Creatinine-binding primary aptamer (SEQ ID NO:258) showing stem and loop structures. (B) Dose/fluorescent response curve resulting from a primary aptamer comprising SEQ ID NO:258 showing binding to creatinine.

FIG. 106A-B. Creatinine-binding primary aptamer (SEQ ID NO:259) showing stem and loop structures. (B) Dose/fluorescent response curve resulting from a primary aptamer comprising SEQ ID NO:259 showing binding to creatinine.

FIG. 107A-B. Vasopressin-binding primary aptamer (SEQ ID NO:261) showing stem and loop structures. (B) Dose/fluorescent response curve resulting from a primary aptamer comprising SEQ ID NO:261 showing binding to vasopressin.

### 5. DETAILED DESCRIPTION OF THE INVENTION

The present invention relates to aptamer-based assays to capture and detect analytes. In addition to the primary aptamers, associated oligonucleotides and assay methods described herein, the methodology may be applied to design other aptamers, associated oligonucleotides, and analogous assays with aptamers or aptamer pairs, with adjustments related to the types of molecules, available reagents, and quantitative goals, for example established by the concentration in actual clinical samples and/or affinities of isolated reagents. Further, the assays, which utilize various mixtures of nucleic acids, can be combined with other nucleic acid elements such as those that form strand-displacement cascades.

For clarity of disclosure and not by way of limitation, this detailed description of the invention is divided into the following sections:

5.1. Primary aptamers;

5.2. Anti-Aptamer Assays

5.3. Pseudosandwich Assays; and

5.4. Sandwich Assays.

Where a sequence provided herein refers to nucleotide “N”, that position in the sequence may be filled by any natural or unnatural nucleotide, unless specified to the contrary.

The term “epitope” is used herein as referring to the binding site for the primary aptamer on the target analyte or, in the case of a sandwich assay, can be a binding site on the target analyte and a second aptamer.

In certain embodiments, where the invention provides for a sequence having a terminal CTCTC (SEQ ID NO:237) 5′ sequence, the invention also provides for an alternative version of that sequence lacking the initial CTCTC (SEQ ID NO:237) sequence.

In certain embodiments, where the invention provides for a sequence having a CTCTC GGG (SEQ ID NO:238) 5′ terminal sequence, the invention also provides for an alternative version of that sequence lacking the initial CTCTCGGG (SEQ ID NO:238) sequence. In certain embodiments, where the invention provides for a sequence having a TCCC (SEQ ID NO:246) 3′ terminal sequence, the invention also provides for an alternative version of that sequence lacking the final TCCC (SEQ ID NO:246) sequence. In certain embodiments, where the invention provides for a sequence having a CTCTC GGG (SEQ ID NO:238) 5′ terminal sequence and a TCCC (SEQ ID NO:246) 3′ terminal sequence, the invention also provides for an alternative version lacking both these sequences.

In the assays described herein, detectable labels are used. In certain embodiments, a fluorescent moiety is comprised in one partner of a binding pair, and a quencher moiety is comprised in the other member of the binding pair. Assays may be designed so that the detectable moiety—e.g. the fluorescent moiety—is on either member of the pair. Fluorescent/quencher compounds are known in the art, and see Mary Katherine Johansson, Methods in Molecular Biol. 335:Fluorescent Energy Transfer Nucleic Acid Probes: Designs and Protocols, 2006, Didenko, ed., Humana Press, Totowa, N.J., and Marras et al., 2002, Nucl. Acids Res. 30, e122 (both incorporated by reference herein). Further, moieties that result in an increase in detectable signal when in proximity of each other may be used as alternative labels in the assays described herein, for example, as a result of fluorescence resonance energy transfer (“FRET”); suitable pairs include but are not limited to fluoroscein and tetramethylrhodamine; rhodamine 6G and malachite green, and FITC and thiosemicarbazole, to name a few.

5.1. Primary Aptamers

A “primary aptamer” (A^{P}) binds an target analyte (ligand, L). A primary aptamer may be isolated (identified as binding to its target analyte) by solution-phase or solid-phase selection.

Primary aptamers (A^{p}) and associated oligonucleotides (e.g., anti-aptamers, sensor oligonucleotides, comp oligonucleotides, and sandwich oligonucleotides (also referred to as “secondary aptamers” or A^{s})), can have any size consistent with their intended function. In certain non-limiting embodiments, a primary aptamer or associated oligonucleotide is between about 20-250 nucleotides in length. For example, but not by way of limitation, the length can be between about 20-200 nucleotides, or between about 20-150 nucleotides, or between about 30 and 200 nucleotides, or between about 40-200 nucleotides, or between about 50-200 nucleotides, or between about 60-200 nucleotides, or between about 70-200 nucleotides, or between about 80-200 nucleotides, or between about 100-200 nucleotides, or between about 150-200 nucleotides, or between about 30-150 nucleotides, or between about 30-100 nucleotides, or between about 30-80 nucleotides, or between about 30-50 nucleotides, or between about 40-100 nucleotides; or at least about 20 nucleotides, or at least about 30 nucleotides, or up to about 100 nucleotides, or up to about 200 nucleotides (where “about” means plus or minus 20 percent), or between 20-250 nucleotides, or between 25 and 100 nucleotides. In certain non-limiting embodiments, primary aptamers and/or associated oligonucleotides can be spiegelmers or contain unnatural enantiomers of nucleic acids.²²⁻²⁵

In particular embodiments, a primary aptamer is provided comprising a core sequence that acts as a binding pocket for the target analyte. In certain embodiments, a primary aptamer comprising a particular core sequence, or a consensus core sequence, is provided. A primary aptamer may further comprise an operative sequence which plays a functional role in a particular assay, as will be described below. A primary aptamer may also optionally comprise additional sequence, other than core sequence or operative sequence, which does not substantially impact its functionality. A primary aptamer comprising a core sequence that binds to a target analyte of interest may be utilized in diverse assays, including but not limited to those exemplified herein.

5.1.1. Methods for Isolating Primary Aptamers

Primary aptamers can be identified that selectively bind to diverse target analytes, including, but not limited to amino acids, mono- and oligo-saccharides, steroids, catecholamines, serotonin, melatonin, lipids, hormones, and/or peptides. In certain non-limiting embodiments, the method can be used to produce primary aptamers that bind to spiegelmers for vasopressin, aminoglycosides and other antibiotics, immunosupressants, anti-tumor agents, pesticides, hormones, etc.

For example, and not by way of limitation, the isolation of primary aptamers can be performed using the SELEX process. In certain non-limiting embodiments, the primary aptamers are isolated by either solid- (traditional) or newer solution-phase selections¹⁶⁻²¹. Further, in certain non-limiting embodiments, the solution-phase selection has inherent advantages for small molecules, such as higher affinity and ease of screening of aptamers.

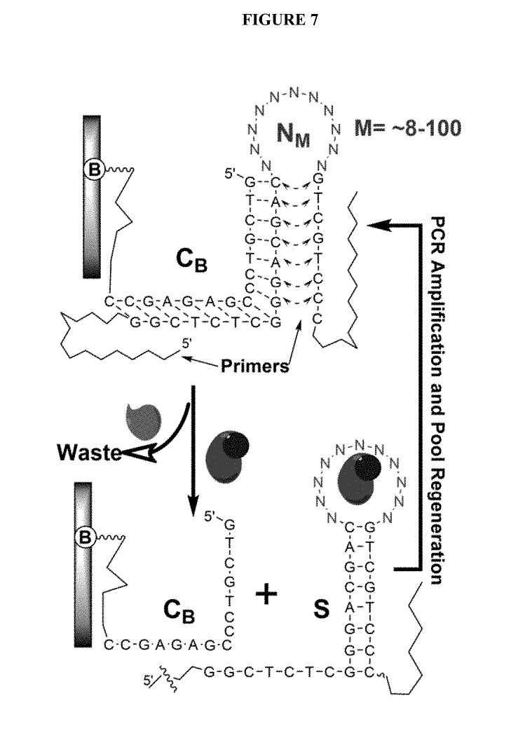

In certain non-limiting embodiments, the method comprises attaching a biotinylated strand complementary (C_{B}) to one of the PCR primers to agarose-streptavidin (FIG. 7) and attaching sequences from a library (e.g., but not limited to, N₈-N₁₀₀) through complementary interactions to C_{B} of a primer. Two primers on library, 5′- and 3′-, are also partially complementary; members of the library which interact with a target in a way that favors stem formation between complementary region of these primers are released from the agarose by displacing complement C_{B}, and used in PCR amplification that now creates an enriched pool of potential aptamers.

In the solution-phase selection, molecules are used without any attachment to a matrix, thus, no functional groups are “wasted”. This maximizes interactions with aptamers, leading to high affinities. Concentrations of compounds that can be used go up to the limit of solubility, allowing isolation of weak-affinity aptamers (e.g., against metabolites and glucose).

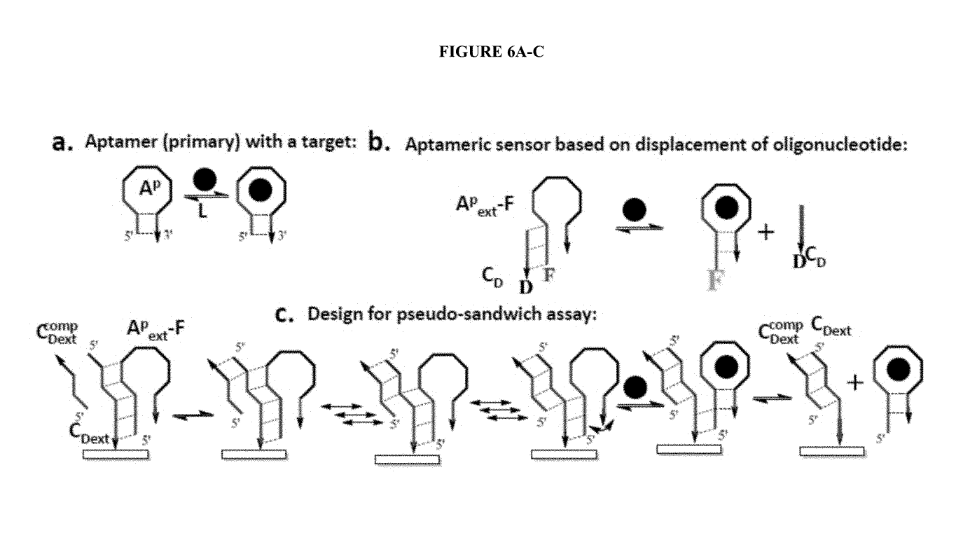

In certain non-limiting embodiments, fluorescent sensors can be directly obtained from this selection, by substituting biotin with dabcyl and attaching fluorescein to the aptamer (FIG. 6, C_{D}), confirming that aptamers bind, determining their K_{d} (this is a competitive assay, so half-response is shifted away from the K_{d}⁸⁰, and C_{D} is present in an excess), and establishing selectivity.

In certain non-limiting embodiments, the method comprises:

(1) Isolating primary aptamers (A^{p}s) by solution-phase (FIG. 7) or solid-phase selection, unless they are already available; use of enantiomeric aptamer (spiegelmers), if desired to minimize Watson-Crick base pairing (e.g., fusing aptamers or to minimize background interactions without analyte);

(2) Testing of the primary aptamer in its structure-switching form and modifying its structure switching form, which is then turned into pseudo-sandwich assay format (FIG. 6C);

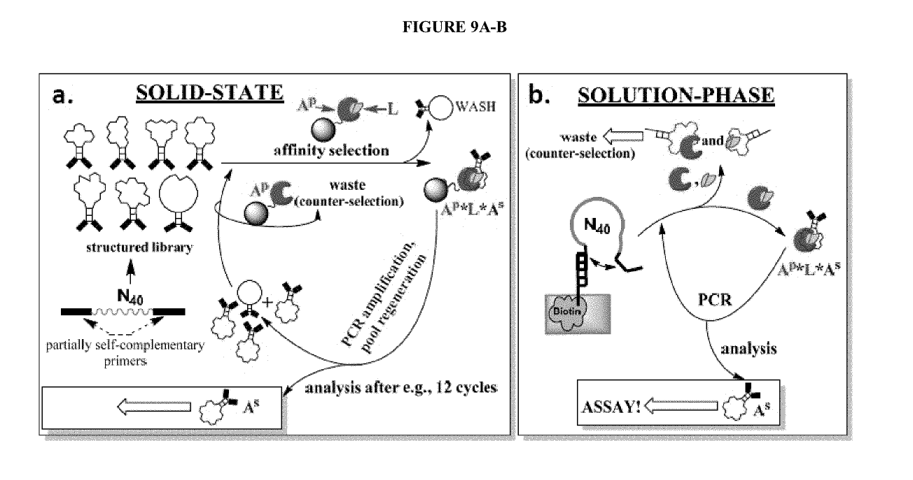

(3) Isolating secondary sandwich aptamers (A^{s}s) by either solution-phase or solid phase selections (FIG. 9A-B), using primary aptamers or spiegelmers in their complexes with targets;

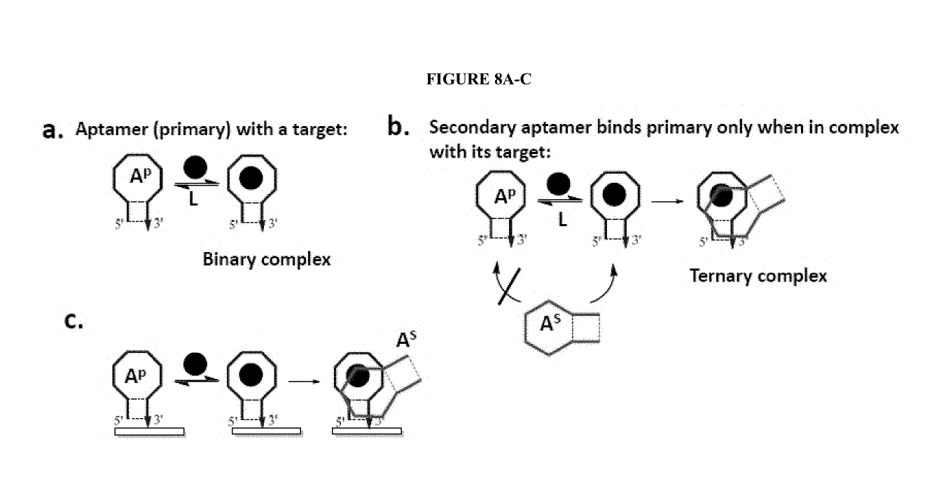

(4) Implementing sandwich assays for targets (FIG. 8A-C) and/or

(5) Preparing a shortened form of the primary aptamer originally isolated;

(6) Modifying the optionally shortened primary aptamer by introducing an operative sequence; and/or

(7) Modifying the optionally shortened primary aptamer by substituting one or more nucleotides in its binding pocket to improve binding properties in the intended assay.

5.1.2 Glucose-Binding Primary Aptamers

In certain non-limiting embodiments, a primary aptamer binds to glucose in an aqueous solution at room temperature or 25° C. with a dissociation constant of less than 10⁻¹ M (affinity 10) and binds selectively with glucose versus galactose.

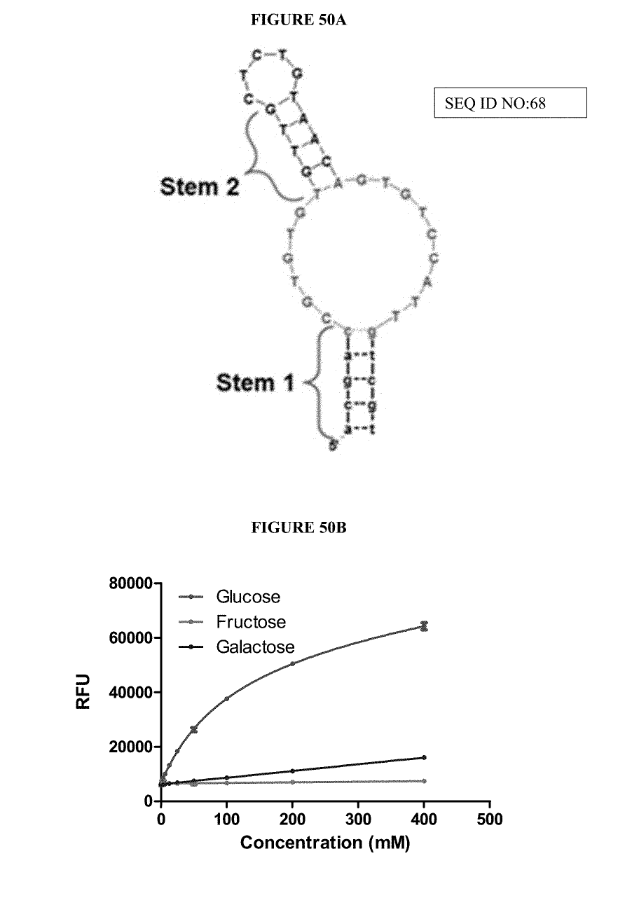

In certain non-limiting embodiments, a glucose-binding primary aptamer comprises the sequences CCGTGTGT (SEQ ID NO:157) and either AGTGTCCATTG (SEQ ID NO:158) or AGTGTCCTTTG (SEQ ID NO:159) or a variant of any of these sequences that differs in one or two bases by substitution, deletion, insertion or extension, where said primary aptamer binds to glucose in an aqueous solution at room temperature or 25° C. with a dissociation constant of less than 10⁻¹ M (affinity 10), and binds selectivity to glucose versus galactose or fructose (see, for example, FIG. 50B). In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, said glucose-binding primary aptamer comprising the sequences CCGTGTGT (SEQ ID NO:157) and either AGTGTCCATTG (SEQ ID NO:158) or AGTGTCCTTTG (SEQ ID NO:159) or a variant thereof has a binding affinity for glucose that is at least about 50 percent or at least about 75 percent the binding affinity of the primary aptamer having SEQ ID NO:68.). In certain non-limiting embodiments, said glucose-binding primary aptamer comprising the sequences CCGTGTGT (SEQ ID NO:157) and either AGTGTCCATTG (SEQ ID NO:158) or AGTGTCCTTTG (SEQ ID NO:159) or a variant thereof competes with primary aptamer having SEQ ID NO:68 for glucose binding. In certain non-limiting embodiments, a glucose-binding primary aptamer comprises the sequences CCGTGTGT (SEQ ID NO:157) and either AGTGTCCATTG (SEQ ID NO:158) or AGTGTCCTTTG (SEQ ID NO:159) or a variant thereof and further comprises at least one operative sequence. In certain non-limiting embodiments, a glucose-binding primary aptamer comprises the sequences CCGTGTGT (SEQ ID NO:157) and either AGTGTCCATTG (SEQ ID NO:158) or AGTGTCCTTTG (SEQ ID NO:159), or a variant thereof, and further comprises at least one operative sequence, said operative sequence complementary to a sequence comprised in a sensor oligonucleotide. In certain non-limiting embodiments, a glucose-binding primary aptamer comprises the sequences CCGTGTGT (SEQ ID NO:157) and either AGTGTCCATTG (SEQ ID NO:158) or AGTGTCCTTTG (SEQ ID NO:159), or a variant thereof, and at least one operative sequence on either side (flanking) these two sequences. In certain non-limiting embodiments, a glucose-binding primary aptamer comprises the sequences CCGTGTGT (SEQ ID NO:157) and either AGTGTCCATTG (SEQ ID NO:158) or AGTGTCCTTTG (SEQ ID NO:159), or a variant thereof, and at least one operative sequence on either side (flanking) these two sequences, where two of said operative sequences contain mutually complementary portions and can form a duplex.

In certain non-limiting embodiments, a glucose-binding primary aptamer has a predicted secondary structure that comprises two stems connected by sequences that bind to glucose (e.g., binding selectively to glucose versus galactose) and/or one or more of the following: a 4-O—R-glucose epitope, where R is hydrogen, an alkyl group, another carbohydrate or a protein; cellobiose; and/or maltose. See, for example, FIG. 50A-B.

For example, but not by way of limitation, a glucose-binding primary aptamer may comprise a sequence selected from the group consisting of:

>S-Glu01:
(SEQ ID NO:160)
CTCTCGGGACGACCGTGTGTGTTGCTCTGTAAC---------
AGTGTCCATTGTCGTCCC;
>S-Glu02:
(SEQ ID NO:161)
CTCTCGGGACGACCGTGTGTGGTAGAGTCGTCGGGCTCTAACAGTGTCCT
TTGTCGTCCC;
>S-Glu03:
(SEQ ID NO:162)
CTCTCGGGACGACCGTGTGTGACGTGCGCCGTGGGGAACGTCAGTGTTCT
TTGTCGTCCC;
>S-Glu04:
(SEQ ID NO:163)
CTCTCGGGACGACCGTGTGTCGACTTAGAGTCG---------
AGTGTCCTTTGTCGTCCC;
and
>S-Glu05:
(SEQ ID NO:164)
CTCTCGGGACGACCGTGTGTTGCAATTCTTGCA---------
AGTGTTCTTTGTCGTCCC.

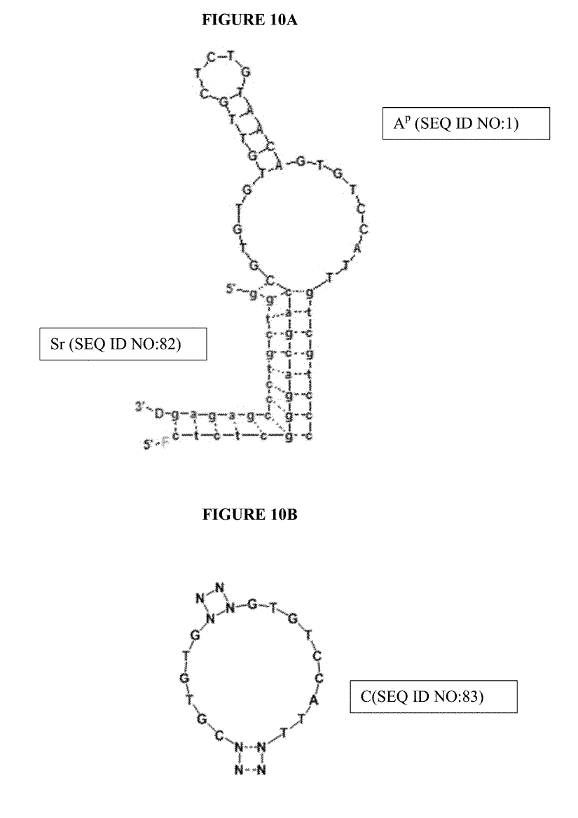

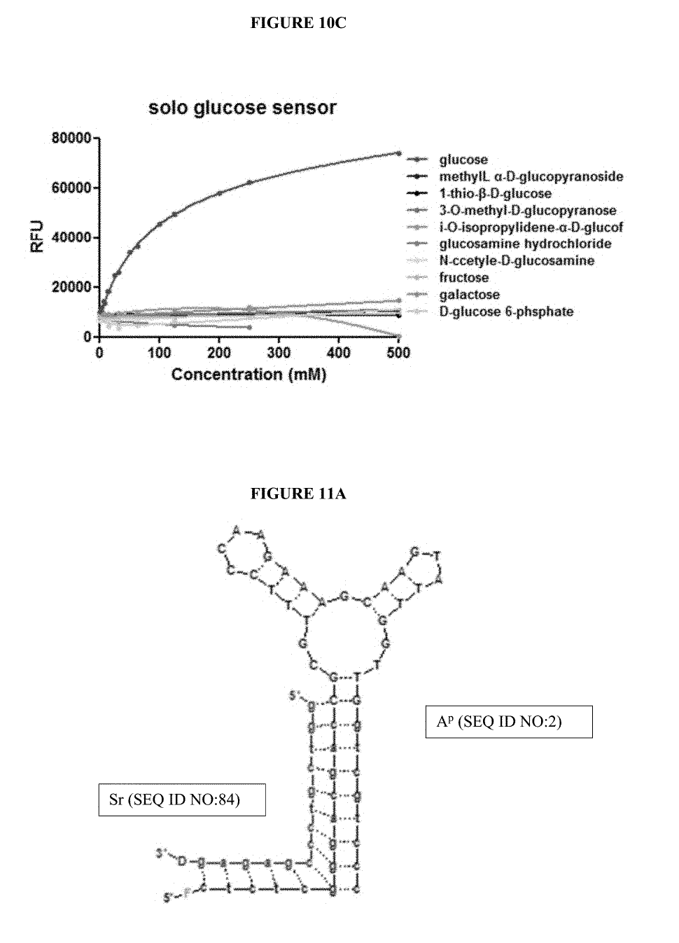

In certain non-limiting embodiments, a glucose-binding primary aptamer comprises a core sequence as set forth in SEQ ID NO:83 (FIG. 10B). In certain non-limiting embodiments, a glucose-binding primary aptamer comprising a core sequence as set forth in SEQ ID NO:83 (FIG. 10B) has a binding affinity for glucose that is at least about 50 percent or at least about 75 percent the binding affinity of the primary aptamer having SEQ ID NO:68). In certain non-limiting embodiments, a glucose-binding primary aptamer comprising a core sequence as set forth in SEQ ID NO:83 (FIG. 10B) competes with primary aptamer having SEQ ID NO:68 for glucose binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a glucose-binding primary aptamer comprises a core sequence as set forth in SEQ ID NO:83 (FIG. 10B) and further comprises at least one operative sequence. In certain non-limiting embodiments, a glucose-binding primary aptamer comprises a core sequence as set forth in SEQ ID NO:83 (FIG. 10B) and further comprises at least one operative sequence, said operative sequence complementary to a sequence comprised in a sensor oligonucleotide. In certain non-limiting embodiments, a glucose-binding primary aptamer comprises a core sequence as set forth in SEQ ID NO:83 (FIG. 10B) and at least one operative sequence on either side (flanking) the core sequence. In certain non-limiting embodiments, a glucose-binding primary aptamer comprises a core sequence as set forth in SEQ ID NO:83 (FIG. 10B) and at least one operative sequence on either side (flanking) the core sequence, where two of said operative sequences contain mutually complementary portions and can form a duplex.

In certain non-limiting embodiments, isolated glucose-binding primary aptamers comprise the nucleotide sequence of SEQ ID NO:1, SEQ ID NO:68, or SEQ ID NO:149 or variants of these sequences having at least about 80 percent, or at least about 85 percent, or at least about 90 percent, or at least about 95 percent, or at least about 98 percent homology to the original sequence, for example obtained by substitutions, deletions, and insertions of Watson-Crick base pairs or by mutations at non-conserved positions. Percent homology can be determined using standard software such as BLAST or FASTA. In certain non-limiting embodiments, isolated glucose-binding primary aptamers comprise the nucleotide sequence of SEQ ID NO:1. SEQ ID NO:68, or SEQ ID NO:149. Said aptamers can bind to glucose and in their structure-switching formats (for example, in an anti-aptamer assay, a pseudosandwich assay, or a sandwich assay) they can respond by an increase in fluorescence.

5.1.3 Phenylalanine-Binding Primary Aptamers

In certain non-limiting embodiments, a primary aptamer binds to phenylalanine in an aqueous solution at room temperature or 25° C. with a dissociation constant of less than 10⁻⁴ M and binds selectively with phenylalanine versus tyrosine (or hydroxyl-phenylalanine) or tryptophan.

In certain non-limiting embodiments, a phenylalanine-binding primary aptamer comprises the sequences GCGT (SEQ ID NO: 165) and AGC and GGTT (SEQ ID NO: 166) or a variant of any of these sequences that differs in one or two bases by substitution, deletion, insertion or extension, where said primary aptamer binds to phenylalanine in an aqueous solution at room temperature or 25° C. with a dissociation constant of less than 10⁻⁴ M and binds selectively to phenylalanine versus tyrosine (or hydroxyl-phenylalanine) or tryptophan (see, for example, FIG. 51A-B). In certain non-limiting embodiments, said phenylalanine-binding primary aptamer comprising the sequences GCGT (SEQ ID NO: 165) and AGC and GGTT (SEQ ID NO: 166), or a variant thereof, has a binding affinity for phenylalanine that is at least about 50 percent or at least about 75 percent the binding affinity of a primary aptamer having SEQ ID NO:2, SEQ ID NO:3, SEQ ID NO:4, or SEQ ID NO:67. In certain non-limiting embodiments, said phenylalanine-binding primary aptamer comprising the sequences GCGT (SEQ ID NO: 165) and AGC and GGTT (SEQ ID NO: 166), or a variant thereof, competes with a phenylalanine-binding primary aptamer having SEQ ID NO: SEQ ID NO:2, SEQ ID NO:3, SEQ ID NO:4, or SEQ ID NO:67. In certain non-limiting embodiments, a phenylalanine-binding primary aptamer comprises the sequences GCGT (SEQ ID NO: 165) and AGC and GGTT (SEQ ID NO: 166) or a variant thereof and further comprises at least one operative sequence. In certain non-limiting embodiments, a phenylalanine-binding primary aptamer comprises the sequences GCGT (SEQ ID NO: 165) and AGC and GGTT (SEQ ID NO: 166), or a variant thereof, and further comprises at least one operative sequence, said operative sequence complementary to a sequence comprised in a sensor oligonucleotide. In certain non-limiting embodiments, a phenylalanine-binding primary aptamer comprises the sequences GCGT (SEQ ID NO: 165) and AGC and GGTT (SEQ ID NO: 166), or a variant thereof, and at least one operative sequence on either side (flanking) these three sequences. In certain non-limiting embodiments, a phenylalanine-binding primary aptamer comprises the sequences GCGT (SEQ ID NO: 165) and AGC and GGTT (SEQ ID NO: 166), or a variant thereof, and at least one operative sequence on either side (flanking) these three sequences, where two of said operative sequences contain mutually complementary portions and can form a duplex.

For example, but not by way of limitation, a phenylalanine-binding primary aptamer may comprise the sequence: CTC TCG GGA CGA CCG CGT TTC CCA AGA AAG CAA GTA TTG GTT GGT CGT CCC (SEQ ID NO:2)

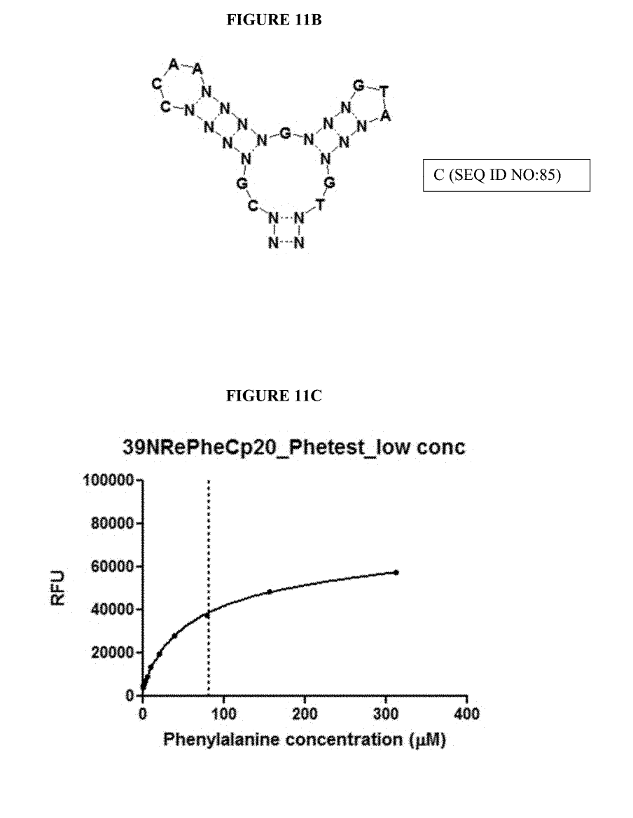

or a portion thereof comprising the core, SEQ ID NO:85) set forth in FIG. 11B, (see FIG. 51A (SEQ ID NO:167).

In certain non-limiting embodiments, a phenylalanine-binding primary aptamer comprises a core sequence as set forth in SEQ ID NO:85 (FIG. 11B). In certain non-limiting embodiments, a phenylalanine-binding primary aptamer comprising a core sequence as set forth in SEQ ID NO:85 (FIG. 11B) has a binding affinity for phenylalanine that is at least about 50 percent or at least about 75 percent the binding affinity of the primary aptamer having SEQ ID NO:167). In certain non-limiting embodiments, a phenylalanine-binding primary aptamer comprising a core sequence as set forth in SEQ ID NO:85 (FIG. 11B) competes with primary aptamer having SEQ ID NO:167 for phenylalanine binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a phenylalanine-binding primary aptamer comprises a core sequence as set forth in SEQ ID NO:85 (FIG. 11B) and further comprises at least one operative sequence. In certain non-limiting embodiments, a phenylalanine-binding primary aptamer comprises a core sequence as set forth in SEQ ID NO:85 (FIG. 11B) and further comprises at least one operative sequence, said operative sequence complementary to a sequence comprised in a sensor oligonucleotide. In certain non-limiting embodiments, a phenylalanine-binding primary aptamer comprises a core sequence as set forth in SEQ ID NO:85 (FIG. 11B) and at least one operative sequence on either side (flanking) the core sequence. In certain non-limiting embodiments, a phenylalanine-binding primary aptamer comprises a core sequence as set forth in SEQ ID NO:85 (FIG. 11B) and at least one operative sequence on either side (flanking) the core sequence, where two of said operative sequences contain mutually complementary portions and can form a duplex.

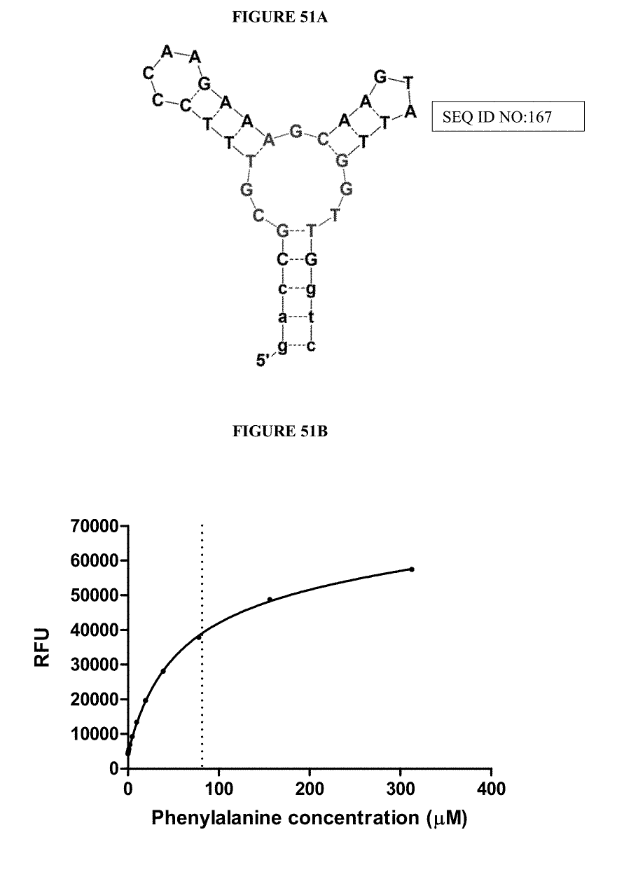

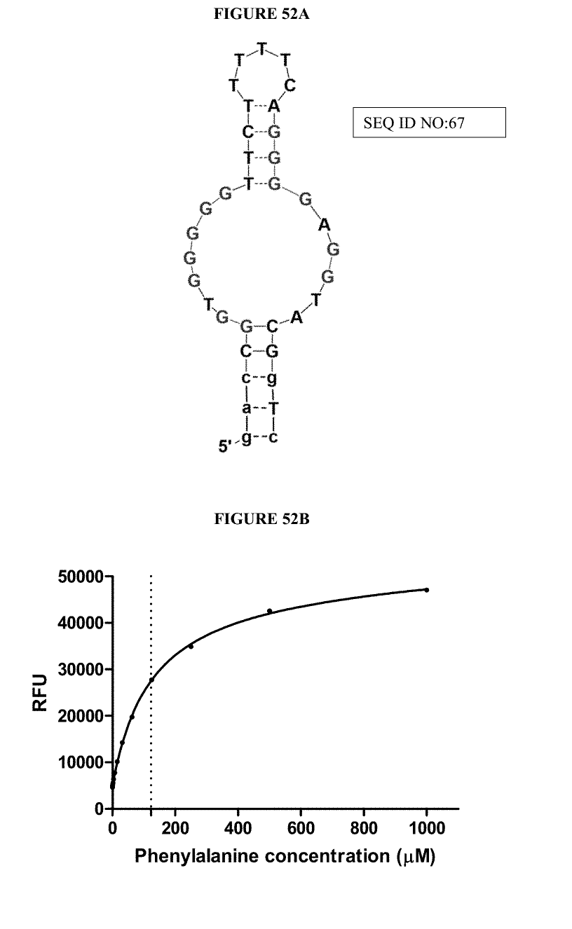

In certain non-limiting embodiments, a phenylalanine-binding primary aptamer comprises the sequences GG and GGGGG (SEQ ID NO:168) and GGGG (SEQ ID NO:169) or a variant of any of these sequences that differs in one or two bases by substitution, deletion, insertion or extension, where said primary aptamer binds to phenylalanine (see FIG. 52A-B) in an aqueous solution at room temperature or 25° C. with a dissociation constant of less than 10⁻⁴ M and binds to phenylalanine selectively versus tyrosine (or hydroxyl-phenylalanine) or tryptophan. In certain non-limiting embodiments, a phenylalanine-binding primary aptamer comprises the sequences GG and GGGGG (SEQ ID NO:168) and GGGG (SEQ ID NO:169) or a variant thereof and further comprises at least one operative sequence. In certain non-limiting embodiments, a phenylalanine-binding primary aptamer comprises the sequences GG and GGGGG (SEQ ID NO:168) and GGGG (SEQ ID NO:169), or a variant thereof, and further comprises at least one operative sequence, said operative sequence complementary to a sequence comprised in a sensor oligonucleotide. In certain non-limiting embodiments, a phenylalanine-binding primary aptamer comprises the sequences GG and GGGGG (SEQ ID NO:168) and GGGG (SEQ ID NO:169), or a variant thereof, and at least one operative sequence on either side (flanking) these three sequences. In certain non-limiting embodiments, a phenylalanine-binding primary aptamer comprises the sequences GG and GGGGG (SEQ ID NO:168) and GGGG (SEQ ID NO:169), or a variant thereof, and at least one operative sequence on either side (flanking) these three sequences, where two of said operative sequences contain mutually complementary portions and can form a duplex. For example, but not by way of limitation, a phenylalanine-binding aptamer may comprise the sequence:

(SEQ ID NO: 3)
CTC TCG GGA CGA CCG GTG GGG GTT CTT TTT CAG GGG
AGG TAC GGT CGT CCC.

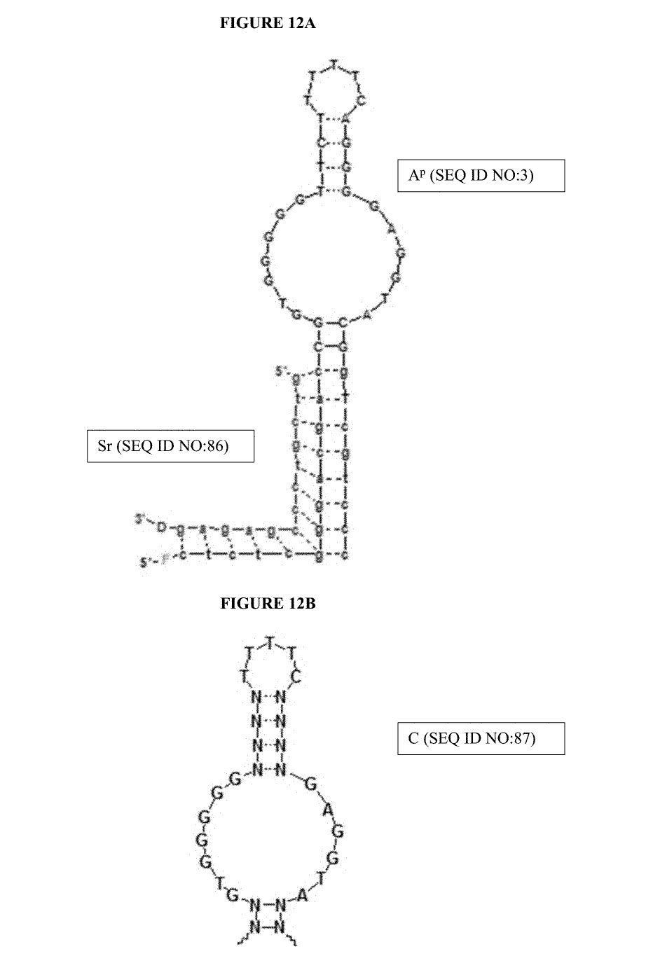

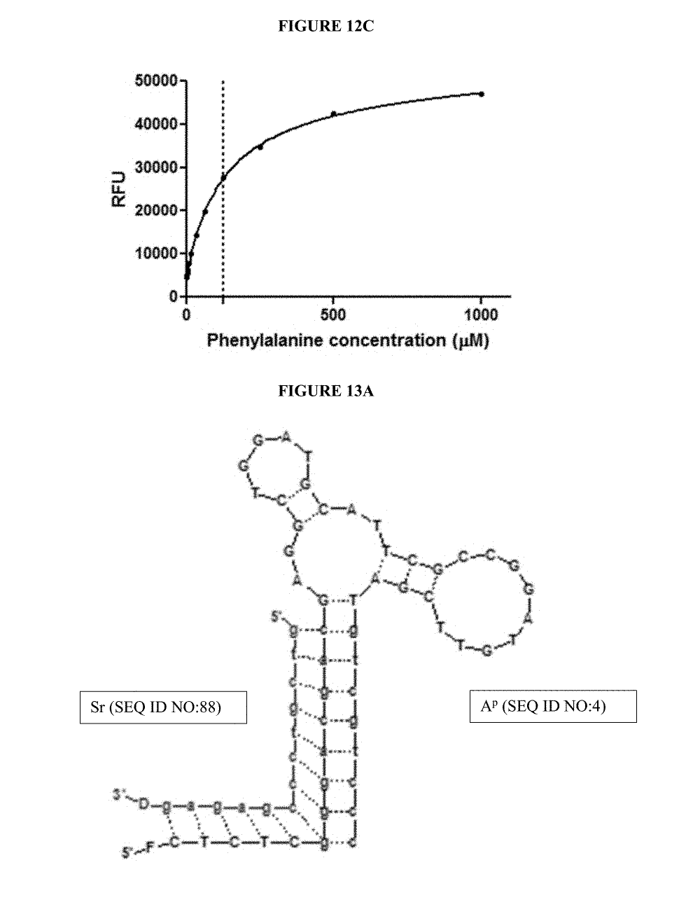

In certain non-limiting embodiments, a phenylalanine-binding primary aptamer comprises a core sequence as set forth in SEQ ID NO: 87 (FIG. 12B; FIG. 52A). In certain non-limiting embodiments, a phenylalanine-binding primary aptamer comprising a core sequence as set forth in SEQ ID NO:87 (FIG. 12B) has a binding affinity for phenylalanine that is at least about 50 percent or at least about 75 percent the binding affinity of the primary aptamer having SEQ ID NO:67). In certain non-limiting embodiments, a phenylalanine-binding primary aptamer comprising a core sequence as set forth in SEQ ID NO:87 (FIG. 12B) competes with primary aptamer having SEQ ID NO:67 for phenylalanine binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a phenylalanine-binding primary aptamer comprises a core sequence as set forth in SEQ ID NO:87 (FIG. 12B) and further comprises at least one operative sequence. In certain non-limiting embodiments, a phenylalanine-binding primary aptamer comprises a core sequence as set forth in SEQ ID NO:87 (FIG. 12B) and further comprises at least one operative sequence, said operative sequence complementary to a sequence comprised in a sensor oligonucleotide. In certain non-limiting embodiments, a phenylalanine-binding primary aptamer comprises a core sequence as set forth in SEQ ID NO: 87 (FIG. 12B) and at least one operative sequence on either side (flanking) the core sequence. In certain non-limiting embodiments, a phenylalanine-binding primary aptamer comprises a core sequence as set forth in SEQ ID NO:87 (FIG. 12B) and at least one operative sequence on either side (flanking) the core sequence, where two of said operative sequences contain mutually complementary portions and can form a duplex.

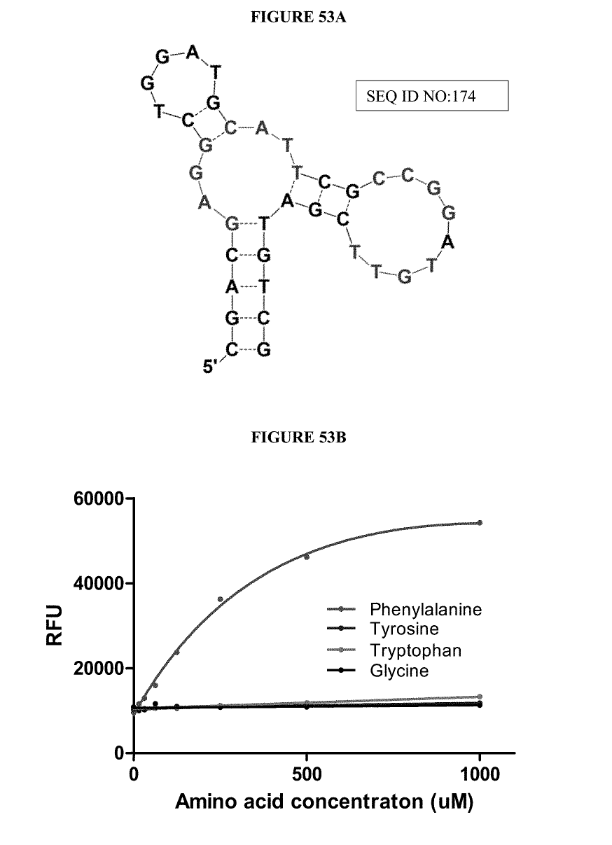

In certain non-limiting embodiments, a phenylalanine-binding primary aptamer comprises the sequences GAGG (SEQ ID NO:170) and CATT (SEQ ID NO:171) or CCGG (SEQ ID NO:172) and TGTT (SEQ ID NO:173) or a variant of any of these sequences that differs in one or two bases by substitution, deletion, insertion or extension, where said primary aptamer binds to phenylalanine in an aqueous solution at room temperature or 25° C. with a dissociation constant of less than 10⁻⁴ M and binds to phenylalanine selectively versus tyrosine (or hydroxyl-phenylalanine) or tryptophan. In certain non-limiting embodiments, a phenylalanine-binding primary aptamer comprises the sequences GAGG (SEQ ID NO:170) and CATT (SEQ ID NO:171) or CCGG (SEQ ID NO:172) and TGTT (SEQ ID NO:173) or a variant thereof and further comprises at least one operative sequence. In certain non-limiting embodiments, a phenylalanine-binding primary aptamer comprises the sequences GAGG (SEQ ID NO:170) and CATT (SEQ ID NO:171) or CCGG (SEQ ID NO:172) and TGTT (SEQ ID NO:173), or a variant thereof, and further comprises at least one operative sequence, said operative sequence complementary to a sequence comprised in a sensor oligonucleotide. In certain non-limiting embodiments, a phenylalanine-binding primary aptamer comprises the sequences GAGG (SEQ ID NO:170) and CATT (SEQ ID NO:171) or CCGG (SEQ ID NO:172) and TGTT (SEQ ID NO:173), or a variant thereof, and at least one operative sequence on either side (flanking) these four sequences. In certain non-limiting embodiments, a phenylalanine-binding primary aptamer comprises the sequences GAGG (SEQ ID NO:170) and CATT (SEQ ID NO:171) or CCGG (SEQ ID NO:172) and TGTT (SEQ ID NO:173), or a variant thereof, and at least one operative sequence on either side (flanking) these four sequences, where two of said operative sequences contain mutually complementary portions and can form a duplex. For example, but not by way of limitation, a phenylalanine-bidning aptamer may comprise the sequence: CTC TCG GGA CGA CGA GGC TGG ATG CAT TCG CCG GAT GTT CGA TGT CGT CCC (SEQ ID NO:4) or related sequence (SEQ ID NO:174, FIG. 53A).

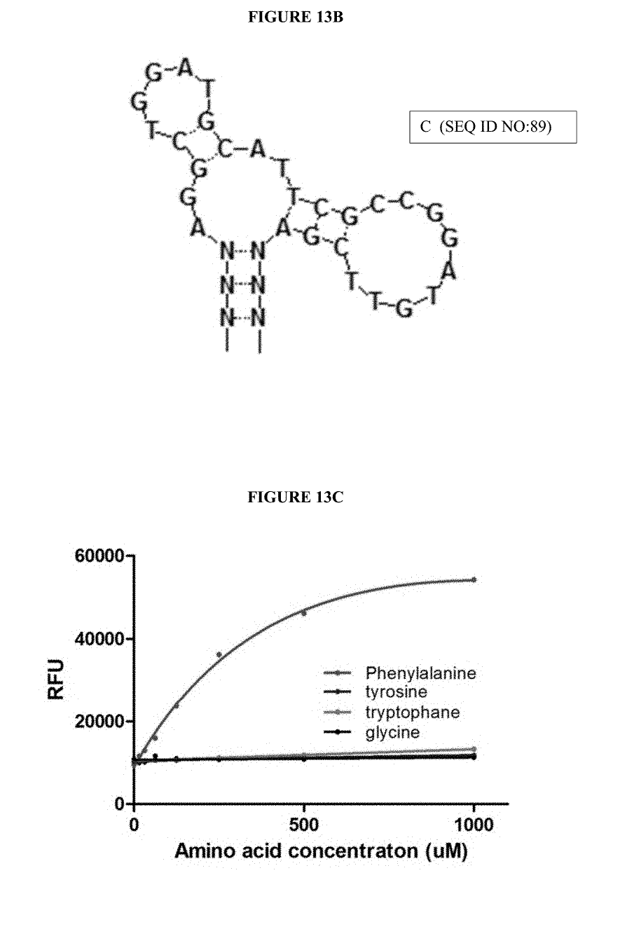

In certain non-limiting embodiments, a phenylalanine-binding primary aptamer comprises a core sequence as set forth in SEQ ID NO:89 (FIG. 13B; FIG. 53A). In certain non-limiting embodiments, a phenylalanine-binding primary aptamer comprising a core sequence as set forth in SEQ ID NO:89 (FIG. 13B) has a binding affinity for phenylalanine that is at least about 50 percent or at least about 75 percent the binding affinity of the primary aptamer having SEQ ID NO:174). In certain non-limiting embodiments, a phenylalanine-binding primary aptamer comprising a core sequence as set forth in SEQ ID NO:89 (FIG. 13B) competes with primary aptamer having SEQ ID NO:174 for phenylalanine binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a phenylalanine-binding primary aptamer comprises a core sequence as set forth in SEQ ID NO:89 (FIG. 13B) and further comprises at least one operative sequence. In certain non-limiting embodiments, a phenylalanine-binding primary aptamer comprises a core sequence as set forth in SEQ ID NO:89 (FIG. 13B) and further comprises at least one operative sequence, said operative sequence complementary to a sequence comprised in a sensor oligonucleotide. In certain non-limiting embodiments, a phenylalanine-binding primary aptamer comprises a core sequence as set forth in SEQ ID NO:89 (FIG. 13B) and at least one operative sequence on either side (flanking) the core sequence. In certain non-limiting embodiments, a phenylalanine-binding primary aptamer comprises a core sequence as set forth in SEQ ID NO:89 (FIG. 13B) and at least one operative sequence on either side (flanking) the core sequence, where two of said operative sequences contain mutually complementary portions and can form a duplex.

In certain non-limiting embodiments, isolated phenylalanine-binding primary aptamers comprise the nucleotide sequence of SEQ ID NO:2, SEQ ID NO:3, SEQ ID NO:4, SEQ ID NO: 67, SEQ ID NO: 167, SEQ ID NO:174 or variants of these sequences having at least about 80 percent, or at least about 85 percent, or at least about 90 percent, or at least about 95 percent, or at least about 98 percent homology to the original sequence, for example obtained by substitutions, deletions, and insertions of Watson-Crick base pairs or by mutations at non-conserved positions. Percent homology can be determined using standard software such as BLAST or FASTA. In certain non-limiting embodiments, isolated phenylalanine-binding primary aptamers comprise the nucleotide sequence of SEQ ID NO:2, SEQ ID NO:3, SEQ ID NO:4, SEQ ID NO: 67, SEQ ID NO: 167, or SEQ ID NO:174. Said aptamers can bind to phenylalanine and in their structure-switching formats (for example, in an anti-aptamer assay, a pseudosandwich assay, or a sandwich assay) they can respond by an increase in fluorescence.

5.1.4 Hydrocortisone-Binding Primary Aptamers

In certain non-limiting embodiments, a primary aptamer binds to hydrocortisone in an aqueous solution at room temperature or 25° C. with a dissociation constant of less than 10−4 M and binds selectively with corticosterone, 11-deoxycorticosterone and/or (17α,21-dihydroxyprogesterone, and/or that selectively binds to steroids with a C.17 carbon connected to one oxygen versus steroids that do not have an oxygen at C.17 position.

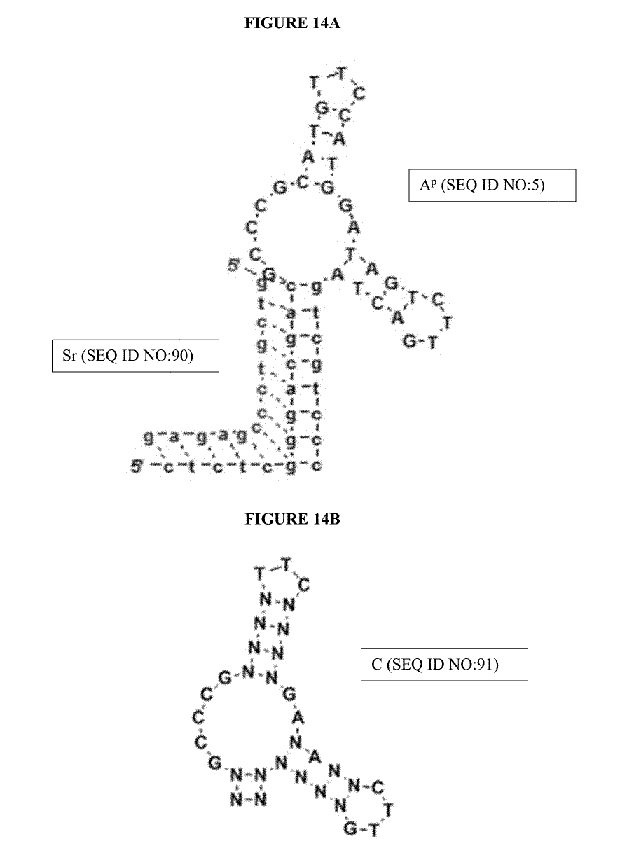

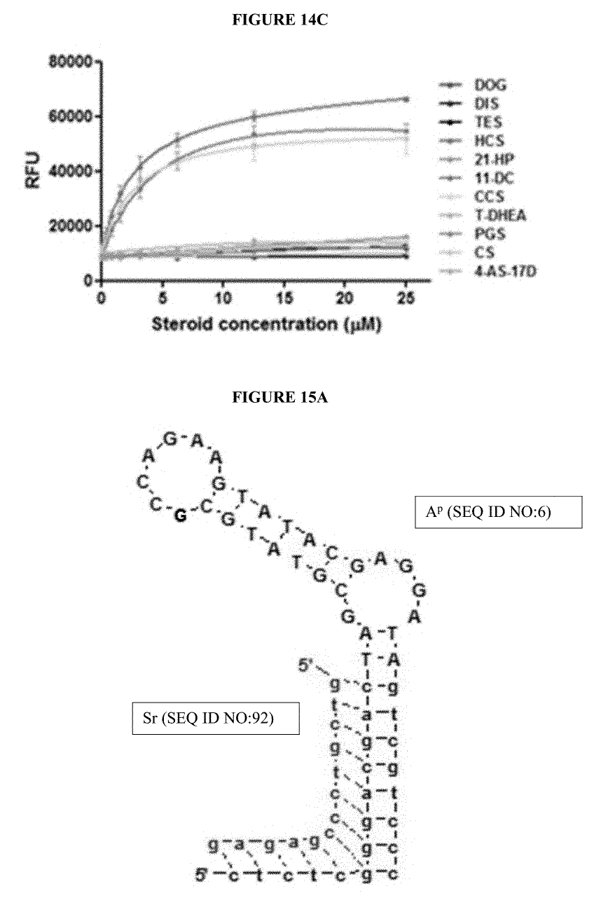

In certain non-limiting embodiments, a hydrocortisone-binding primary aptamer comprises the sequences CGCC (SEQ ID NO:175) and ATGTTC (SEQ ID NO:176) and GGATAGT (SEQ ID NO:177) or a variant of any of these sequences that differs in one or two bases by substitution, deletion, insertion or extension, where said primary aptamer binds to hydrocortisone in an aqueous solution at room temperature or 25° C. with a dissociation constant of less than 10−4 M and binds to hydrocortisone selectively versus steroids that do not have an oxygen at C.17 position (see FIG. 54B). In certain non-limiting embodiments, a hydrocortisone-binding primary aptamer comprises the sequences CGCC (SEQ ID NO:175) and ATGTTC (SEQ ID NO:176) and GGATAGT (SEQ ID NO:177) or a variant thereof and further comprises at least one operative sequence. In certain non-limiting embodiments, a hydrocortisone-binding primary aptamer comprises the sequences CGCC (SEQ ID NO:175) and ATGTTC (SEQ ID NO:176) and GGATAGT (SEQ ID NO:177), or a variant thereof, and further comprises at least one operative sequence, said operative sequence complementary to a sequence comprised in a sensor oligonucleotide. In certain non-limiting embodiments, a hydrocortisone-binding primary aptamer comprises the sequences CGCC (SEQ ID NO:175) and ATGTTC (SEQ ID NO:176) and GGATAGT (SEQ ID NO:177), or a variant thereof, and at least one operative sequence on either side (flanking) these three sequences. In certain non-limiting embodiments, a hydrocortisone-binding primary aptamer comprises the sequences CGCC (SEQ ID NO:175) and ATGTTC (SEQ ID NO:176) and GGATAGT (SEQ ID NO:177) or a variant thereof, and at least one operative sequence on either side (flanking) these four sequences, where two of said operative sequences contain mutually complementary portions and can form a duplex. For example, but not by way of limitation, a hydrocortisone-bidning aptamer may comprise the sequence: CTC TCG GGA CGA CGC CCG CAT GTT CCA TGG ATA GTC TTG ACT AGT CGT CCC (SEQ ID NO:5, FIG. 14A) or a short version thereof (FIG. 54A, SEQ ID NO:178).

In certain non-limiting embodiments, a hydrocortisone-binding primary aptamer comprises a core sequence as set forth in SEQ ID NO:91 (FIG. 14B; FIG. 54A). In certain non-limiting embodiments, a hydrocortisone-binding primary aptamer comprising a core sequence as set forth in SEQ ID NO:91 (FIG. 14B) has a binding affinity for hydrocortisone that is at least about 50 percent or at least about 75 percent the binding affinity of the primary aptamer having SEQ ID NO:178). In certain non-limiting embodiments, a hydrocortisone-binding primary aptamer comprising a core sequence as set forth in SEQ ID NO:91 (FIG. 14B) competes with primary aptamer having SEQ ID NO:178 for hydrocortisone binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a hydrocortisone-binding primary aptamer comprises a core sequence as set forth in SEQ ID NO:91 (FIG. 14B) and further comprises at least one operative sequence. In certain non-limiting embodiments, a hydrocortisone-binding primary aptamer comprises a core sequence as set forth in SEQ ID NO:91 (FIG. 14B) and further comprises at least one operative sequence, said operative sequence complementary to a sequence comprised in a sensor oligonucleotide. In certain non-limiting embodiments, a hydrocortisone-binding primary aptamer comprises a core sequence as set forth in SEQ ID NO:91 (FIG. 14B) and at least one operative sequence on either side (flanking) the core sequence. In certain non-limiting embodiments, a hydrocortisone-binding primary aptamer comprises a core sequence as set forth in SEQ ID NO:91 (FIG. 14B) and at least one operative sequence on either side (flanking) the core sequence, where two of said operative sequences contain mutually complementary portions and can form a duplex.

In certain non-limiting embodiments, a hydrocortisone-binding primary aptamer comprises the sequences CGCC (SEQ ID NO:175) and TACGA (SEQ ID NO:179) and GGATA (SEQ ID NO:180) or a variant of any of these sequences that differs in one or two bases by substitution, deletion, insertion or extension, where said primary aptamer binds to hydrocortisone in an aqueous solution at room temperature or 25° C. with a dissociation constant of less than 10−4 M and binds to hydrocortisone selectively versus steroids that do not have an oxygen at C.17 position (see FIG. 55B) In certain non-limiting embodiments, a hydrocortisone-binding primary aptamer comprises the sequences CGCC (SEQ ID NO:175) and TACGA (SEQ ID NO:179) and GGATA (SEQ ID NO:180) or a variant thereof and further comprises at least one operative sequence. In certain non-limiting embodiments, a hydrocortisone-binding primary aptamer comprises the sequences CGCC (SEQ ID NO:175) and TACGA (SEQ ID NO:179) and GGATA (SEQ ID NO:180), or a variant thereof, and further comprises at least one operative sequence, said operative sequence complementary to a sequence comprised in a sensor oligonucleotide. In certain non-limiting embodiments, a hydrocortisone-binding primary aptamer comprises the sequences CGCC (SEQ ID NO:175) and TACGA (SEQ ID NO:179) and GGATA (SEQ ID NO:180), or a variant thereof, and at least one operative sequence on either side (flanking) these three sequences. In certain non-limiting embodiments, a hydrocortisone-binding primary aptamer comprises the sequences CGCC (SEQ ID NO:175) and TACGA (SEQ ID NO:179) and GGATA (SEQ ID NO:180) or a variant thereof, and at least one operative sequence on either side (flanking) these four sequences, where two of said operative sequences contain mutually complementary portions and can form a duplex. For example, but not by way of limitation, a hydrocortisone-bidning aptamer may comprise the sequence: CTC TCG GGA CGA CTA GCG TAT GCG CCA GAA GTA TAC GAG GAT AGT CGT CCC (SEQ ID NO:6, FIG. 15A) or a short version thereof (FIG. 55A, SEQ ID NO:181).

In certain non-limiting embodiments, a hydrocortisone-binding primary aptamer comprises a core sequence as set forth in SEQ ID NO:93 (FIG. 15B; FIG. 55A). In certain non-limiting embodiments, a hydrocortisone-binding primary aptamer comprising a core sequence as set forth in SEQ ID NO:93 (FIG. 15B) has a binding affinity for hydrocortisone that is at least about 50 percent or at least about 75 percent the binding affinity of the primary aptamer having SEQ ID NO:181). In certain non-limiting embodiments, a hydrocortisone-binding primary aptamer comprising a core sequence as set forth in SEQ ID NO:93 (FIG. 15B) competes with primary aptamer having SEQ ID NO:181 for hydrocortisone binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a hydrocortisone-binding primary aptamer comprises a core sequence as set forth in SEQ ID NO:93 (FIG. 15B) and further comprises at least one operative sequence. In certain non-limiting embodiments, a hydrocortisone-binding primary aptamer comprises a core sequence as set forth in SEQ ID NO:93 (FIG. 15B) and further comprises at least one operative sequence, said operative sequence complementary to a sequence comprised in a sensor oligonucleotide. In certain non-limiting embodiments, a hydrocortisone-binding primary aptamer comprises a core sequence as set forth in SEQ ID NO:93 (FIG. 15B) and at least one operative sequence on either side (flanking) the core sequence. In certain non-limiting embodiments, a hydrocortisone-binding primary aptamer comprises a core sequence as set forth in SEQ ID NO:93 (FIG. 15B) and at least one operative sequence on either side (flanking) the core sequence, where two of said operative sequences contain mutually complementary portions and can form a duplex.

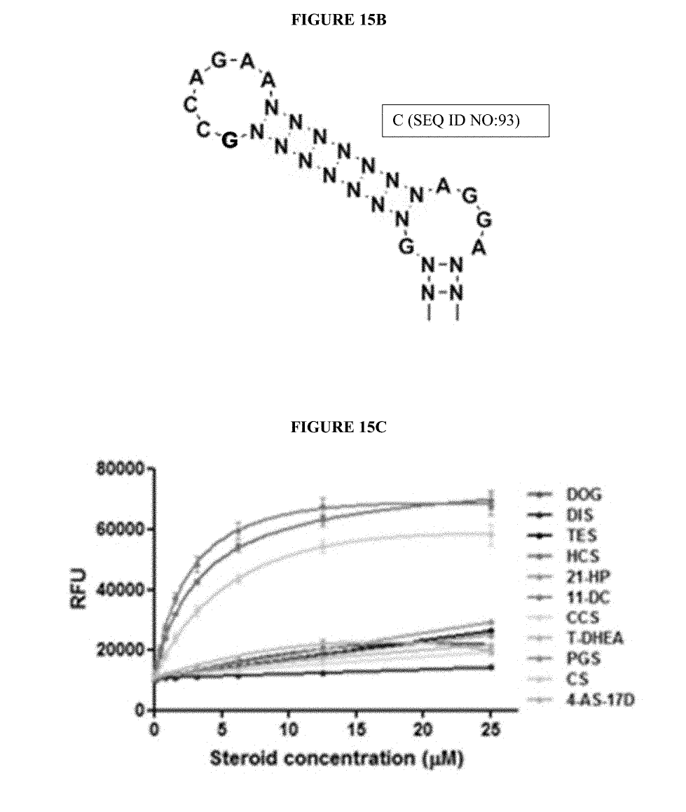

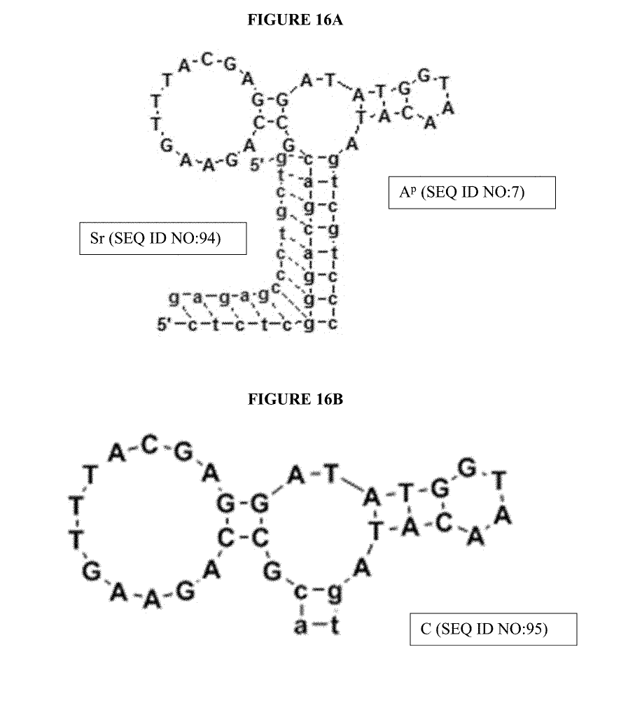

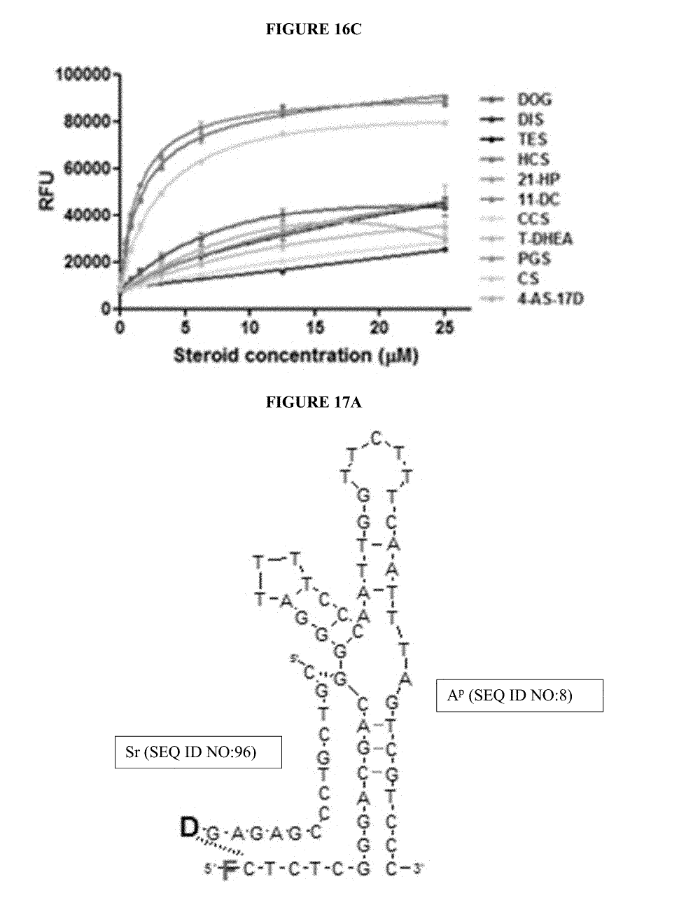

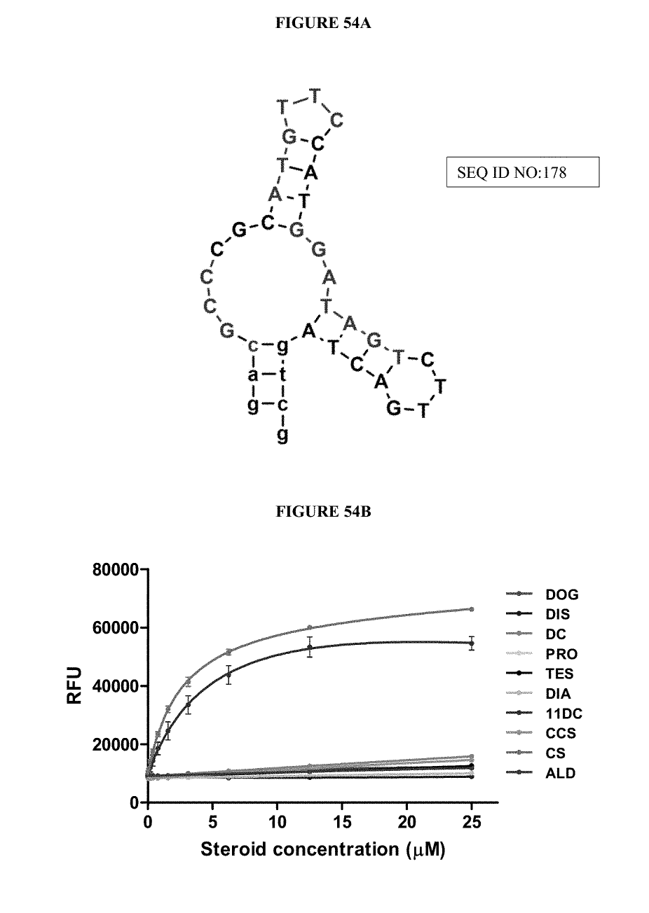

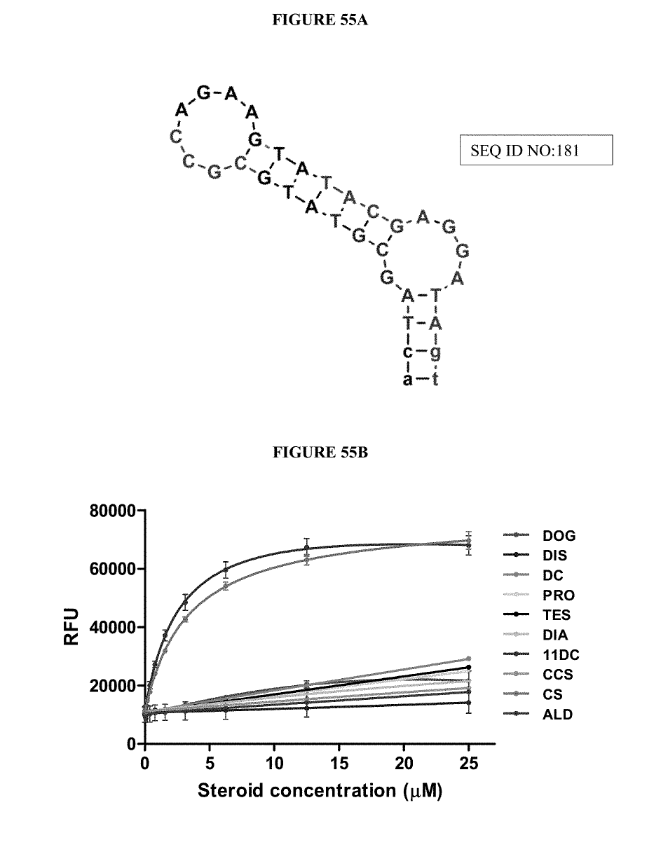

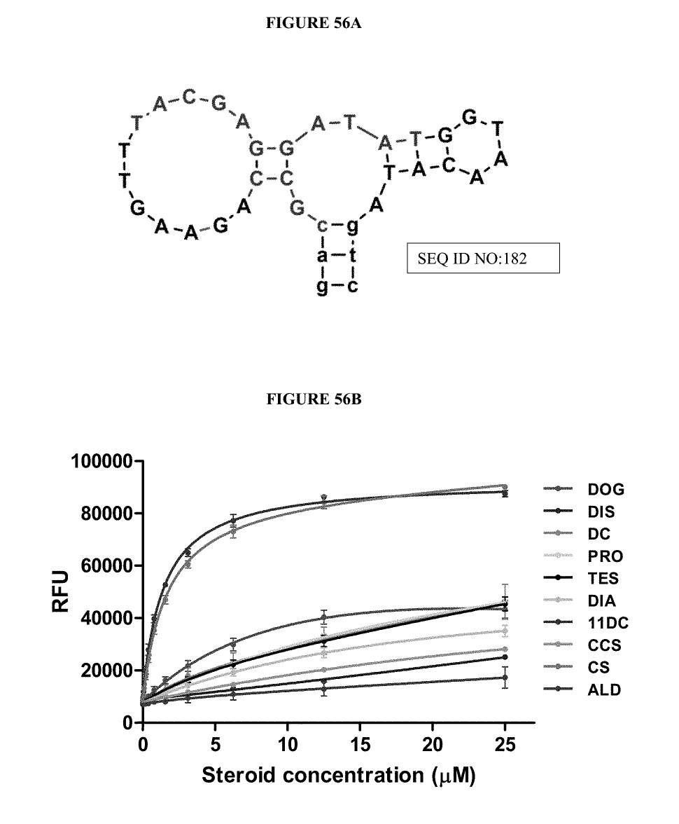

For example, but not by way of limitation, a hydrocortisone-bidning aptamer may comprise the sequence: CTC TCG GGA CGA CGC CAG AAG TTT ACG AGG ATA TGG TAA CAT AGT CGT CCC (SEQ ID NO:7, FIG. 16A) or a short version thereof (FIG. 56A, SEQ ID NO:182).

In certain non-limiting embodiments, a hydrocortisone-binding primary aptamer comprises a core sequence as set forth in SEQ ID NO:95 (FIG. 16B; FIG. 56A-B). In certain non-limiting embodiments, a hydrocortisone-binding primary aptamer comprising a core sequence as set forth in SEQ ID NO:95 (FIG. 16B) has a binding affinity for hydrocortisone that is at least about 50 percent or at least about 75 percent the binding affinity of the primary aptamer having SEQ ID NO:182). In certain non-limiting embodiments, a hydrocortisone-binding primary aptamer comprising a core sequence as set forth in SEQ ID NO:95 (FIG. 16B) competes with primary aptamer having SEQ ID NO:182 for hydrocortisone binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a hydrocortisone-binding primary aptamer comprises a core sequence as set forth in SEQ ID NO:95 (FIG. 16B) and further comprises at least one operative sequence. In certain non-limiting embodiments, a hydrocortisone-binding primary aptamer comprises a core sequence as set forth in SEQ ID NO:95 (FIG. 16B) and further comprises at least one operative sequence, said operative sequence complementary to a sequence comprised in a sensor oligonucleotide. In certain non-limiting embodiments, a hydrocortisone-binding primary aptamer comprises a core sequence as set forth in SEQ ID NO:95 (FIG. 16B) and at least one operative sequence on either side (flanking) the core sequence. In certain non-limiting embodiments, a hydrocortisone-binding primary aptamer comprises a core sequence as set forth in SEQ ID NO:95 (FIG. 16B) and at least one operative sequence on either side (flanking) the core sequence, where two of said operative sequences contain mutually complementary portions and can form a duplex.

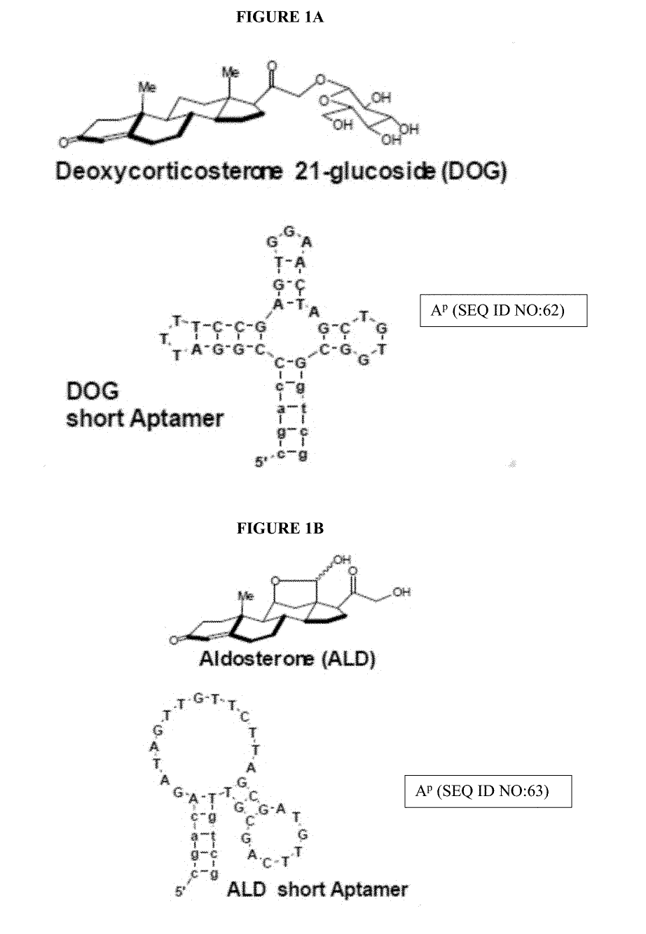

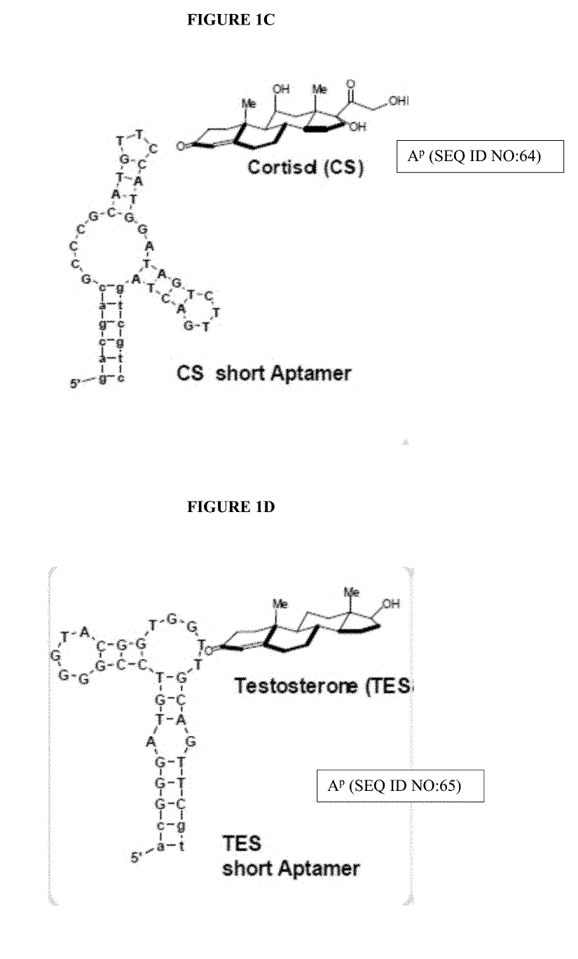

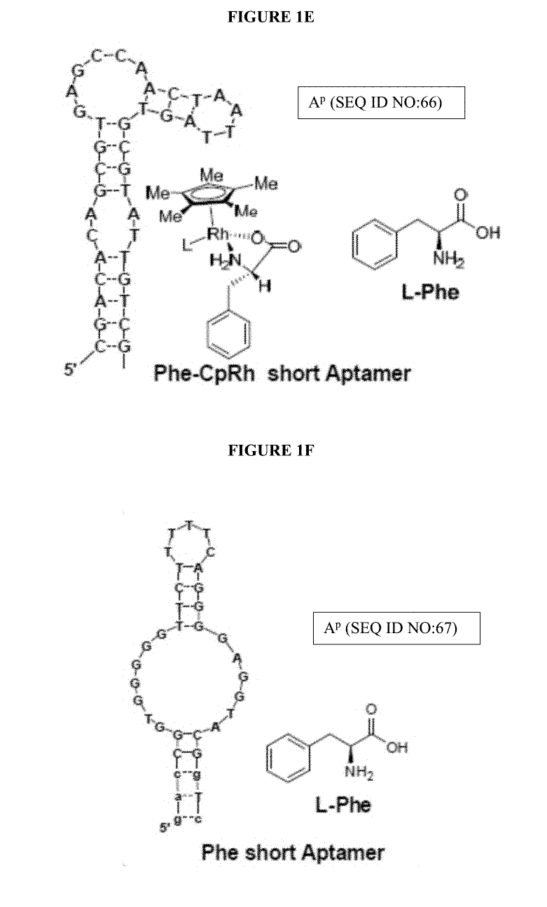

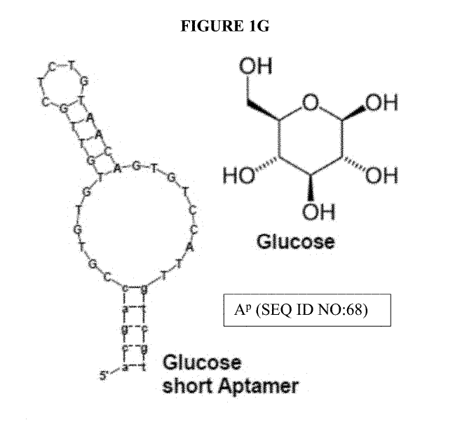

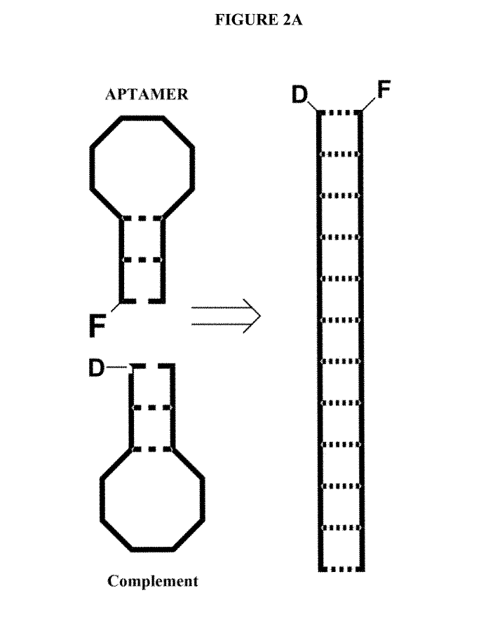

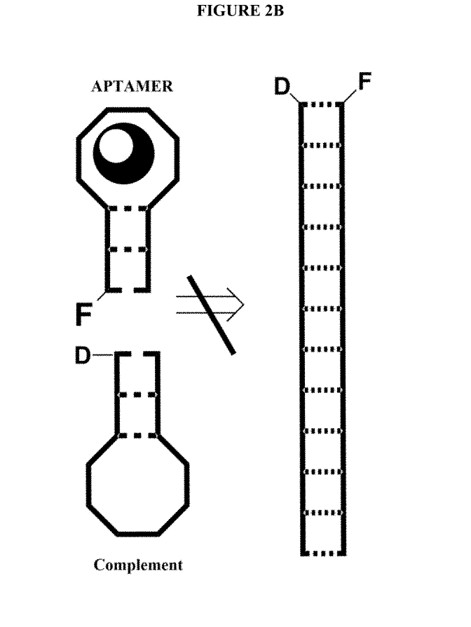

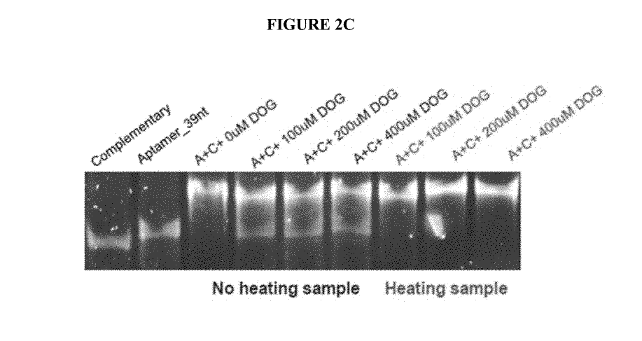

In certain non-limiting embodiments, isolated hydrocortisone-binding primary aptamers comprise the nucleotide sequence of SEQ ID NO:5, SEQ ID NO:6, SEQ ID NO:7, SEQ ID NO:64 (FIG. 1C), SEQ ID NO: 181, SEQ ID NO:148. or SEQ ID NO: 182, or variants of these sequences having at least about 80 percent, or at least about 85 percent, or at least about 90 percent, or at least about 95 percent, or at least about 98 percent homology to the original sequence, for example obtained by substitutions, deletions, and insertions of Watson-Crick base pairs or by mutations at non-conserved positions. Percent homology can be determined using standard software such as BLAST or FASTA. In certain non-limiting embodiments, isolated hydrocortisone-binding primary aptamers comprise the nucleotide sequence of SEQ ID NO:5, SEQ ID NO:6, SEQ ID NO:7, SEQ ID NO:64 (FIG. 1C), SEQ ID NO: 181, SEQ ID NO:148. or SEQ ID NO: 182. Said aptamers can bind to hydrocortisone and in their structure-switching formats (for example, in an anti-aptamer assay, a pseudosandwich assay, or a sandwich assay) they can respond by an increase in fluorescence.

5.1.5. Dehydroisoandrosterone-Binding Primary Aptamers

In certain non-limiting embodiments, a primary aptamer binds to dehydroisoandrosterone in an aqueous solution at room temperature or 25° C. with a dissociation constant of less than 10⁻⁴ M and binds selectively with dehydroisoandrosterone versus deoxycorticosterone.

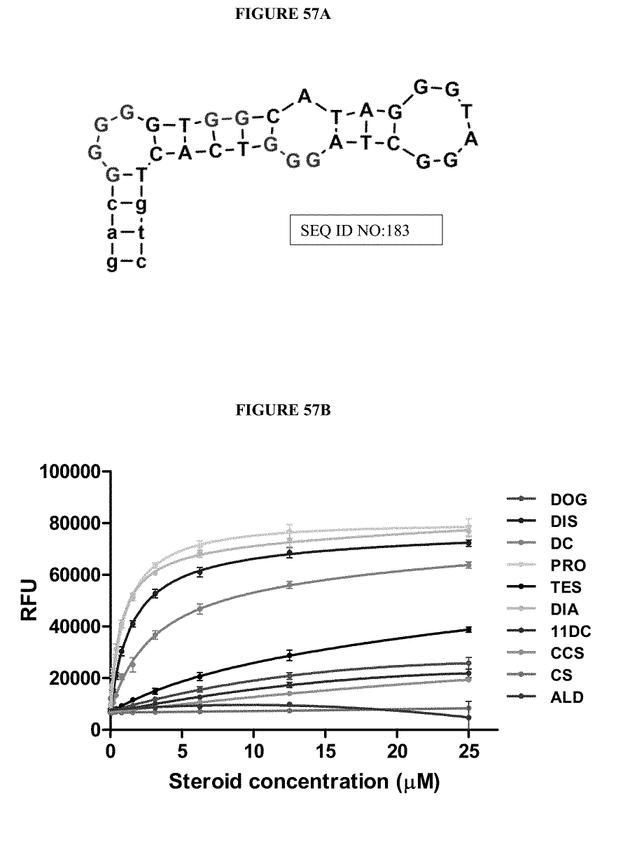

In certain non-limiting embodiments, a dehydroisoandrosterone-binding primary aptamer comprises the sequences GGG, GGGG (SEQ ID NO:169) and GG or a variant of any of these sequences that differs in one or two bases by substitution, deletion, insertion or extension, where said primary aptamer binds to dehydroisoandrosterone in an aqueous solution at room temperature or 25° C. with a dissociation constant of less than 10⁻⁴ M and binds to dehydroisoandrosterone selectively versus deoxycorticosterone and/or other steroids (see FIGS. 57B, 58B and 59B). In certain non-limiting embodiments, a dehydroisoandrosterone-binding primary aptamer comprises the sequences GGG, GGGG (SEQ ID NO:169) and GG or a variant thereof and further comprises at least one operative sequence. In certain non-limiting embodiments, a dehydroisoandrosterone-binding primary aptamer comprises the sequences GGG, GGGG (SEQ ID NO:169) and GG, or a variant thereof, and further comprises at least one operative sequence, said operative sequence complementary to a sequence comprised in a sensor oligonucleotide. In certain non-limiting embodiments, a dehydroisoandrosterone-binding primary aptamer comprises the sequences GGG, GGGG (SEQ ID NO:169) and GG, or a variant thereof, and at least one operative sequence on either side (flanking) these three sequences. In certain non-limiting embodiments, a dehydroisoandrosterone-binding primary aptamer comprises the sequences GGG, GGGG (SEQ ID NO:169) and GG, or a variant thereof, and at least one operative sequence on either side (flanking) these three sequences, where two of said operative sequences contain mutually complementary portions and can form a duplex.

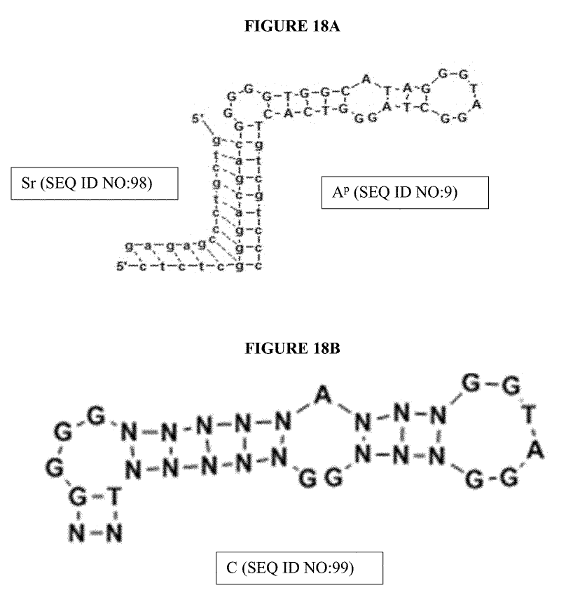

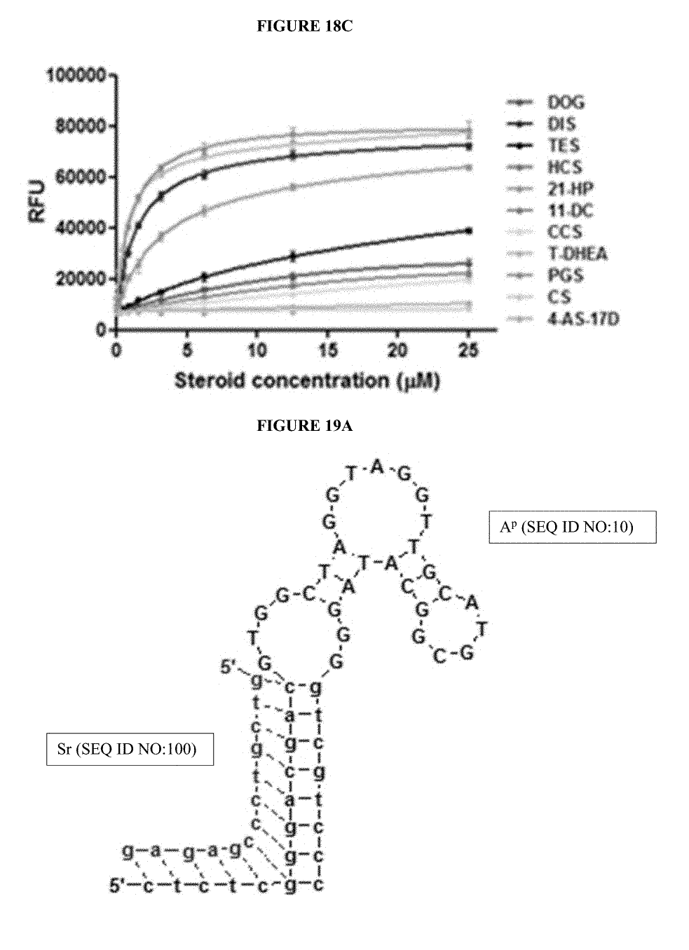

For example, but not by way of limitation, a dehydroisoandrosterone-bidning aptamer may comprise the sequence: CTC TCG GGA CGA CGG GGG TGG CAT AGG GTA GGC TAG GGT CAC TGT CGT CCC (SEQ ID NO:9) or related sequence (SEQ ID NO:183, FIG. 57A). In certain non-limiting embodiments, a dehydroisoandrosterone-binding primary aptamer comprises a core sequence as set forth in SEQ ID NO:99 (FIG. 18B; FIG. 57A). In certain non-limiting embodiments, a dehydroisoandrosterone-binding primary aptamer comprising a core sequence as set forth in SEQ ID NO:99 (FIG. 18B) has a binding affinity for dehydroisoandrosterone that is at least about 50 percent or at least about 75 percent the binding affinity of the primary aptamer having SEQ ID NO:183). In certain non-limiting embodiments, a dehydroisoandrosterone-binding primary aptamer comprising a core sequence as set forth in SEQ ID NO:99 (FIG. 18B) competes with primary aptamer having SEQ ID NO:183 for dehydroisoandrosterone binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a dehydroisoandrosterone-binding primary aptamer comprises a core sequence as set forth in SEQ ID NO:99 (FIG. 18B) and further comprises at least one operative sequence. In certain non-limiting embodiments, a dehydroisoandrosterone-binding primary aptamer comprises a core sequence as set forth in SEQ ID NO:99 (FIG. 18B) and further comprises at least one operative sequence, said operative sequence complementary to a sequence comprised in a sensor oligonucleotide. In certain non-limiting embodiments, a dehydroisoandrosterone-binding primary aptamer comprises a core sequence as set forth in SEQ ID NO:99 (FIG. 18B) and at least one operative sequence on either side (flanking) the core sequence. In certain non-limiting embodiments, a dehydroisoandrosterone-binding primary aptamer comprises a core sequence as set forth in SEQ ID NO:99 (FIG. 18B) and at least one operative sequence on either side (flanking) the core sequence, where two of said operative sequences contain mutually complementary portions and can form a duplex.

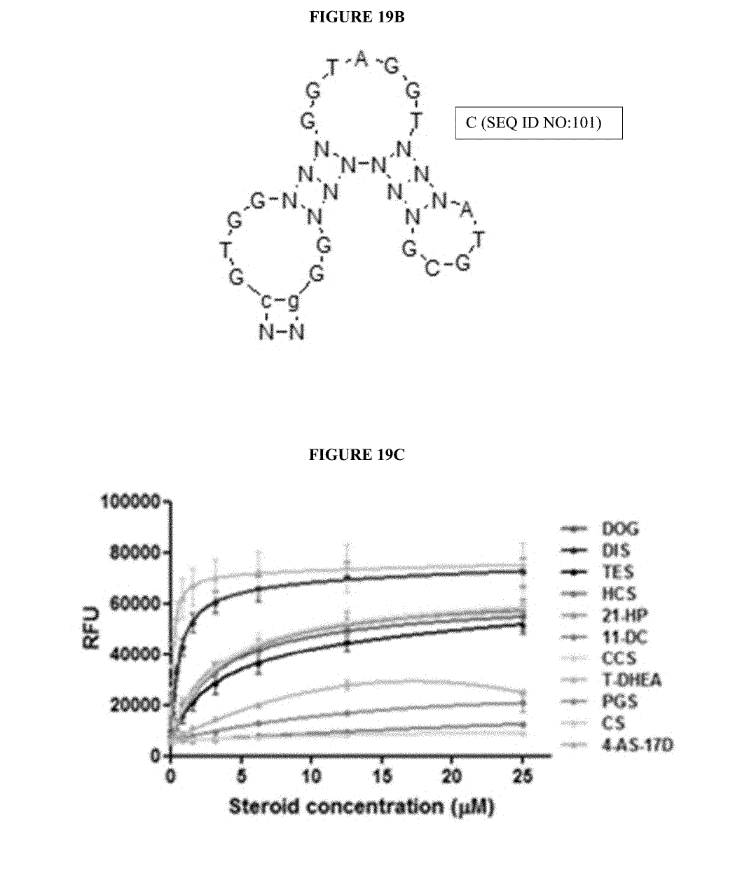

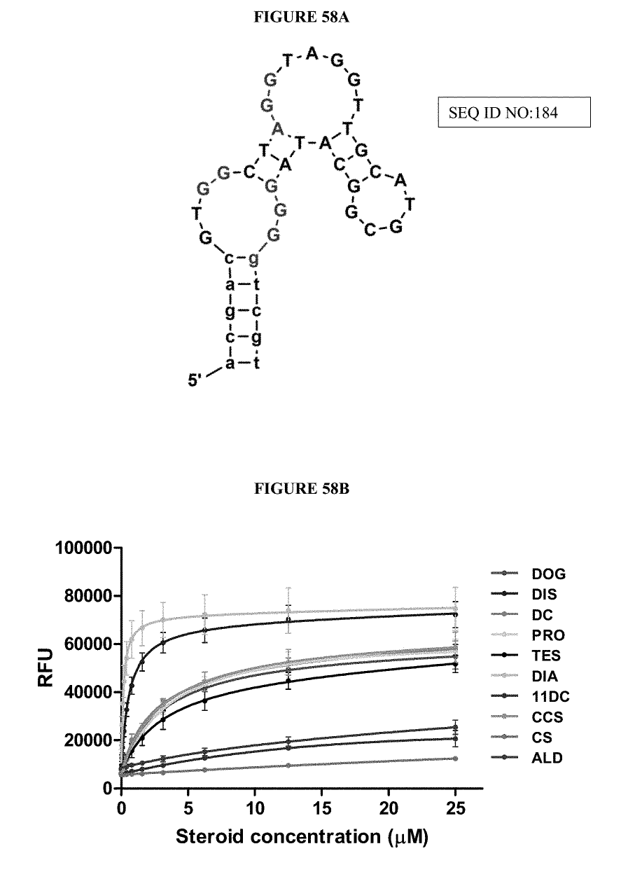

For example, but not by way of limitation, a dehydroisoandrosterone-bidning aptamer may comprise the sequence: CTC TCG GGA CGA CGT GGC TAG GTA GGT TGC ATG CGG CAT AGG GGT CGT CCC (SEQ ID NO:10) or related sequence (SEQ ID NO:184, FIG. 58A). In certain non-limiting embodiments, a dehydroisoandrosterone-binding primary aptamer comprises a core sequence as set forth in SEQ ID NO:101 (FIG. 19B; FIG. 58A). In certain non-limiting embodiments, a dehydroisoandrosterone-binding primary aptamer comprising a core sequence as set forth in SEQ ID NO:101 (FIG. 19B) has a binding affinity for dehydroisoandrosterone that is at least about 50 percent or at least about 75 percent the binding affinity of the primary aptamer having SEQ ID NO:184). In certain non-limiting embodiments, a dehydroisoandrosterone-binding primary aptamer comprising a core sequence as set forth in SEQ ID NO:101 (FIG. 19B) competes with primary aptamer having SEQ ID NO:184 for dehydroisoandrosterone binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a dehydroisoandrosterone-binding primary aptamer comprises a core sequence as set forth in SEQ ID NO:101 (FIG. 19B) and further comprises at least one operative sequence. In certain non-limiting embodiments, a dehydroisoandrosterone-binding primary aptamer comprises a core sequence as set forth in SEQ ID NO:101 (FIG. 19B) and further comprises at least one operative sequence, said operative sequence complementary to a sequence comprised in a sensor oligonucleotide. In certain non-limiting embodiments, a dehydroisoandrosterone-binding primary aptamer comprises a core sequence as set forth in SEQ ID NO:101 (FIG. 19B) and at least one operative sequence on either side (flanking) the core sequence. In certain non-limiting embodiments, a dehydroisoandrosterone-binding primary aptamer comprises a core sequence as set forth in SEQ ID NO:101 (FIG. 19B) and at least one operative sequence on either side (flanking) the core sequence, where two of said operative sequences contain mutually complementary portions and can form a duplex.

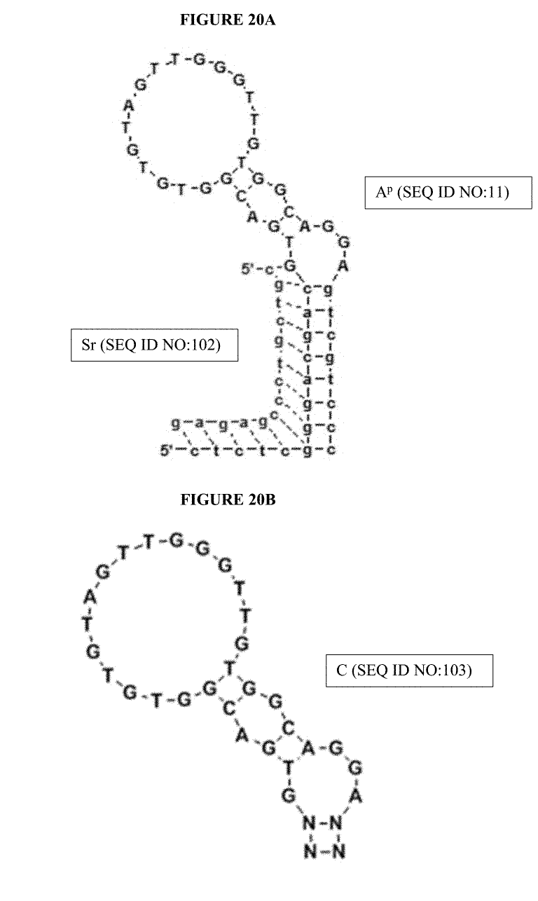

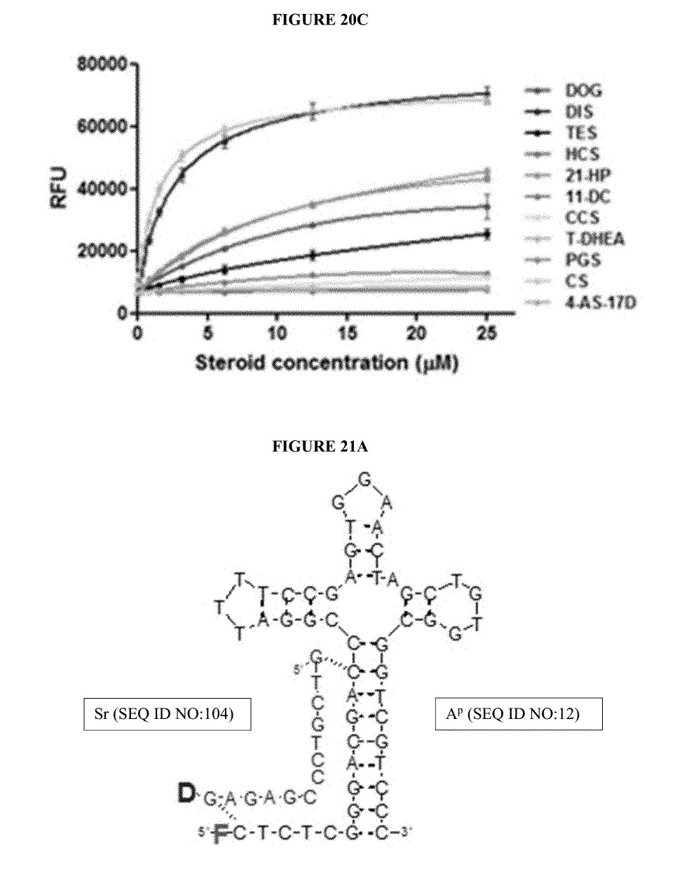

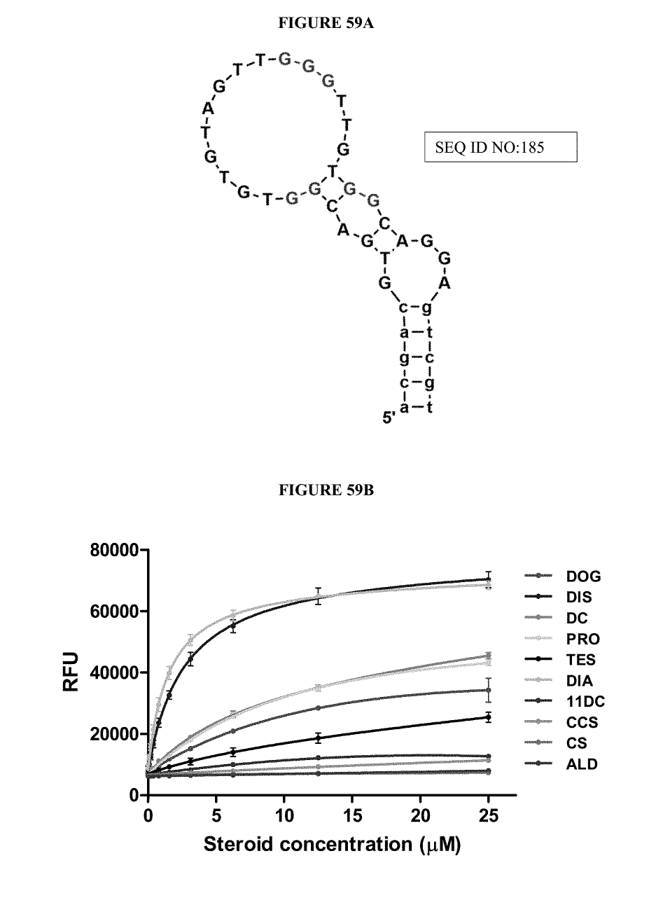

For example, but not by way of limitation, a dehydroisoandrosterone-bidning aptamer may comprise the sequence: CTC TCG GGA CGA CGT GAC GGT GTG TAG TTG GGT TGT GGC AGG AGT CGT CCC (SEQ ID NO:11) or related sequence (SEQ ID NO:185, FIG. 59A). In certain non-limiting embodiments, a dehydroisoandrosterone-binding primary aptamer comprises a core sequence as set forth in SEQ ID NO:103 (FIG. 20B; FIG. 59A). In certain non-limiting embodiments, a dehydroisoandrosterone-binding primary aptamer comprising a core sequence as set forth in SEQ ID NO:103 (FIG. 20B) has a binding affinity for dehydroisoandrosterone that is at least about 50 percent or at least about 75 percent the binding affinity of the primary aptamer having SEQ ID NO:185). In certain non-limiting embodiments, a dehydroisoandrosterone-binding primary aptamer comprising a core sequence as set forth in SEQ ID NO:103 (FIG. 20B) competes with primary aptamer having SEQ ID NO:185 for dehydroisoandrosterone binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a dehydroisoandrosterone-binding primary aptamer comprises a core sequence as set forth in SEQ ID NO:103 (FIG. 20B) and further comprises at least one operative sequence. In certain non-limiting embodiments, a dehydroisoandrosterone-binding primary aptamer comprises a core sequence as set forth in SEQ ID NO:103 (FIG. 20B) and further comprises at least one operative sequence, said operative sequence complementary to a sequence comprised in a sensor oligonucleotide. In certain non-limiting embodiments, a dehydroisoandrosterone-binding primary aptamer comprises a core sequence as set forth in SEQ ID NO:103 (FIG. 20B) and at least one operative sequence on either side (flanking) the core sequence. In certain non-limiting embodiments, a dehydroisoandrosterone-binding primary aptamer comprises a core sequence as set forth in SEQ ID NO:103 (FIG. 20B) and at least one operative sequence on either side (flanking) the core sequence, where two of said operative sequences contain mutually complementary portions and can form a duplex.

In certain non-limiting embodiments, isolated dehydroisoandrosterone-binding primary aptamers comprise the nucleotide sequence of SEQ ID NO:9, SEQ ID NO:10, SEQ ID NO:11, SEQ ID NO: 183, SEQ ID NO: 184, SEQ ID NO:185 or variants of these sequences having at least about 80 percent, or at least about 85 percent, or at least about 90 percent, or at least about 95 percent, or at least about 98 percent homology to the original sequence, for example obtained by substitutions, deletions, and insertions of Watson-Crick base pairs or by mutations at non-conserved positions. Percent homology can be determined using standard software such as BLAST or FASTA. In certain non-limiting embodiments, isolated dehydroisoandrosterone-binding primary aptamers comprise the nucleotide sequence of SEQ ID NO:9, SEQ ID NO:10, SEQ ID NO:11, SEQ ID NO: 183, SEQ ID NO: 184, or SEQ ID NO:185. Said aptamers can bind to dehydroisoandrosterone and in their structure-switching formats (for example, in an anti-aptamer assay, a pseudosandwich assay, or a sandwich assay) they can respond by an increase in fluorescence.

5.1.6 Deoxycorticosterone-Binding Primary Aptamers

In certain non-limiting embodiments, a primary aptamer binds to deoxycorticosterone in an aqueous solution at room temperature or 25° C. with a dissociation constant of less than 10⁻⁴ M and binds selectively with deoxycorticosterone versus dehydroisoandrosterone.

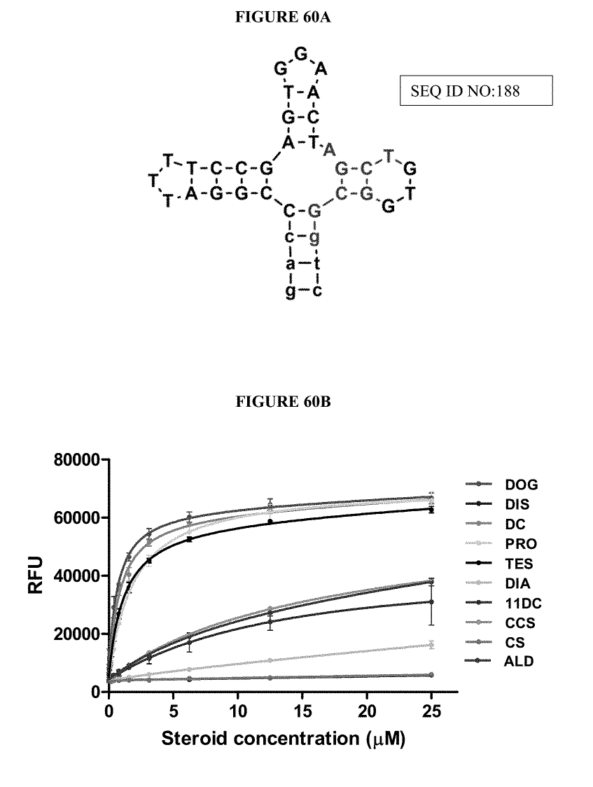

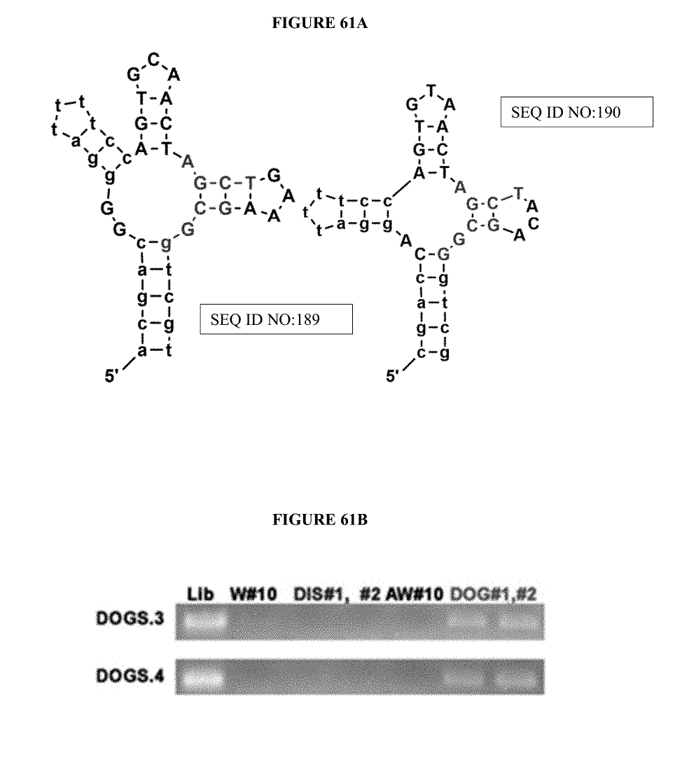

In certain non-limiting embodiments, a deoxycorticosterone-binding primary aptamer comprises the sequences AGCT (SEQ ID NO:186) and GCGG (SEQ ID NO:187) or a variant of any of these sequences that differs in one or two bases by substitution, deletion, insertion or extension, where said primary aptamer binds to deoxycorticosterone in an aqueous solution at room temperature or 25° C. with a dissociation constant of less than 10⁻⁴ M and binds to deoxycorticosterone selectively versus dehydroisoandrosterone and/or other steroids (see FIGS. 60B, 61B). In certain non-limiting embodiments, a deoxycorticosterone-binding primary aptamer comprises the sequences AGCT (SEQ ID NO:186) and GCGG (SEQ ID NO:187) or a variant thereof and further comprises at least one operative sequence. In certain non-limiting embodiments, a deoxycorticosterone-binding primary aptamer comprises the sequences AGCT (SEQ ID NO:186) and GCGG (SEQ ID NO:187), or a variant thereof, and further comprises at least one operative sequence, said operative sequence complementary to a sequence comprised in a sensor oligonucleotide. In certain non-limiting embodiments, a deoxycorticosterone-binding primary aptamer comprises the sequences AGCT (SEQ ID NO:186) and GCGG (SEQ ID NO:187), or a variant thereof, and at least one operative sequence on either side (flanking) these two sequences. In certain non-limiting embodiments, a deoxycorticosterone-binding primary aptamer comprises the sequences AGCT (SEQ ID NO:186) and GCGG (SEQ ID NO:187), or a variant thereof, and at least one operative sequence on either side (flanking) these two sequences, where two of said operative sequences contain mutually complementary portions and can form a duplex.

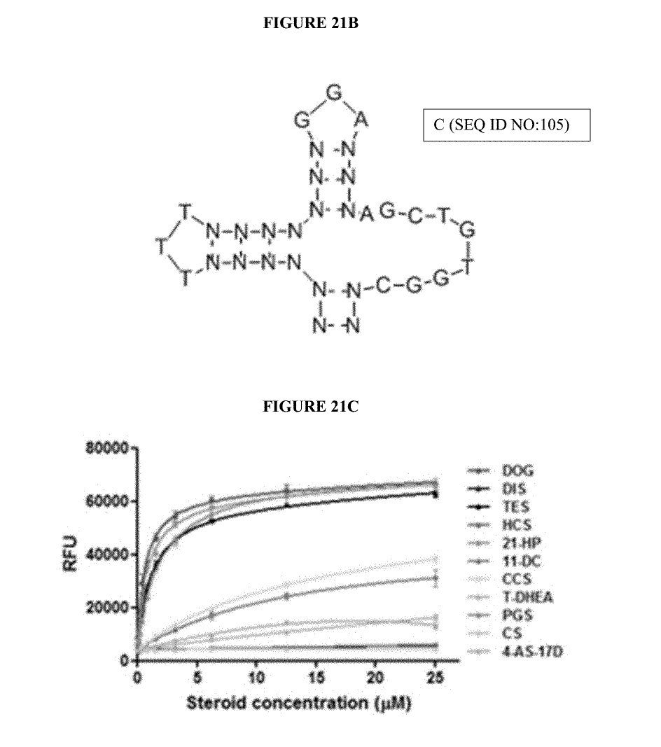

For example, but not by way of limitation, a deoxycorticosterone-bidning aptamer may comprise the sequence: CTC TCG GGA CGA CCC GGA TTT TCC GAG TGG AAC TAG CTG TGG CGG TCG TCC C (SEQ ID NO:12) or related sequence (e.g., SEQ ID NO:188, FIG. 60A; SEQ ID NO:62, FIG. 1A). In certain non-limiting embodiments, a deoxycorticosterone-binding primary aptamer comprises a core sequence as set forth in SEQ ID NO:105 (FIG. 21B; FIG. 60A). In certain non-limiting embodiments, a deoxycorticosterone-binding primary aptamer comprising a core sequence as set forth in SEQ ID NO:105 (FIG. 21B) has a binding affinity for deoxycorticosterone that is at least about 50 percent or at least about 75 percent the binding affinity of the primary aptamer having SEQ ID NO:188. In certain non-limiting embodiments, a deoxycorticosterone-binding primary aptamer comprising a core sequence as set forth in SEQ ID NO:105 (FIG. 21B) competes with primary aptamer having SEQ ID NO:188 for deoxycorticosterone binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a deoxycorticosterone-binding primary aptamer comprises a core sequence as set forth in SEQ ID NO:105 (FIG. 21B) and further comprises at least one operative sequence. In certain non-limiting embodiments, a deoxycorticosterone-binding primary aptamer comprises a core sequence as set forth in SEQ ID NO:105 (FIG. 21B) and further comprises at least one operative sequence, said operative sequence complementary to a sequence comprised in a sensor oligonucleotide. In certain non-limiting embodiments, a deoxycorticosterone-binding primary aptamer comprises a core sequence as set forth in SEQ ID NO:105 (FIG. 21B) and at least one operative sequence on either side (flanking) the core sequence. In certain non-limiting embodiments, a deoxycorticosterone-binding primary aptamer comprises a core sequence as set forth in SEQ ID NO:105 (FIG. 21B) and at least one operative sequence on either side (flanking) the core sequence, where two of said operative sequences contain mutually complementary portions and can form a duplex.

For example, but not by way of limitation, a deoxycorticosterone-bidning aptamer may comprise the sequence: CTC TCG GGA CGA CGG GGA TTT TCC AGT GCA ACT AGC TGA AAG CGG TCG TCC C (SEQ ID NO: 262).

For example, but not by way of limitation, a deoxycorticosterone-bidning aptamer may comprise the sequence: CTC TCG GGA CGA CCA GGA TTT TCC AGT GTA ACT AGC TAC AGC GGG TCG TCC C (SEQ ID NO: 263).

In certain non-limiting embodiments, isolated deoxycorticosterone-binding primary aptamers comprise the nucleotide sequence of SEQ ID NO:12, SEQ ID NO:62, SEQ ID NO:188, SEQ ID NO: 189, SEQ ID NO: 190, or variants of these sequences having at least about 80 percent, or at least about 85 percent, or at least about 90 percent, or at least about 95 percent, or at least about 98 percent homology to the original sequence, for example obtained by substitutions, deletions, and insertions of Watson-Crick base pairs or by mutations at non-conserved positions. Percent homology can be determined using standard software such as BLAST or FASTA. In certain non-limiting embodiments, isolated deoxycorticosterone-binding primary aptamers comprise the nucleotide sequence of SEQ ID NO:12, SEQ ID NO:62, SEQ ID NO:188, SEQ ID NO: 189, or SEQ ID NO: 190. Said aptamers can bind to deoxycorticosterone and in their structure-switching formats (for example, in an anti-aptamer assay, a pseudosandwich assay, or a sandwich assay) they can respond by an increase in fluorescence.

5.1.7 Testosterone-Binding Primary Aptamers

In certain non-limiting embodiments, a primary aptamer binds to testosterone in an aqueous solution at room temperature or 25° C. with a dissociation constant of less than 10⁻⁴ M and binds selectively with testosterone versus 11-deoxycorticosterone.

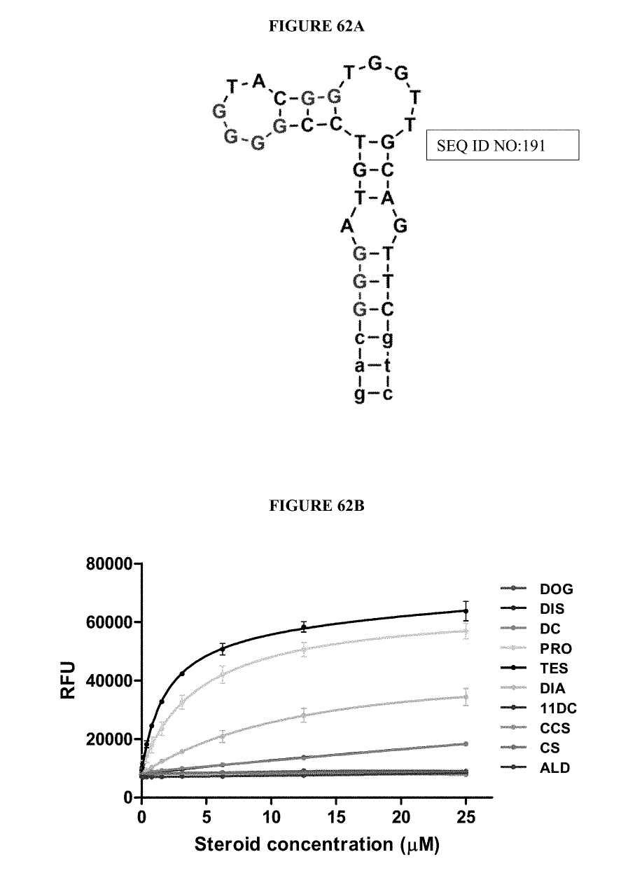

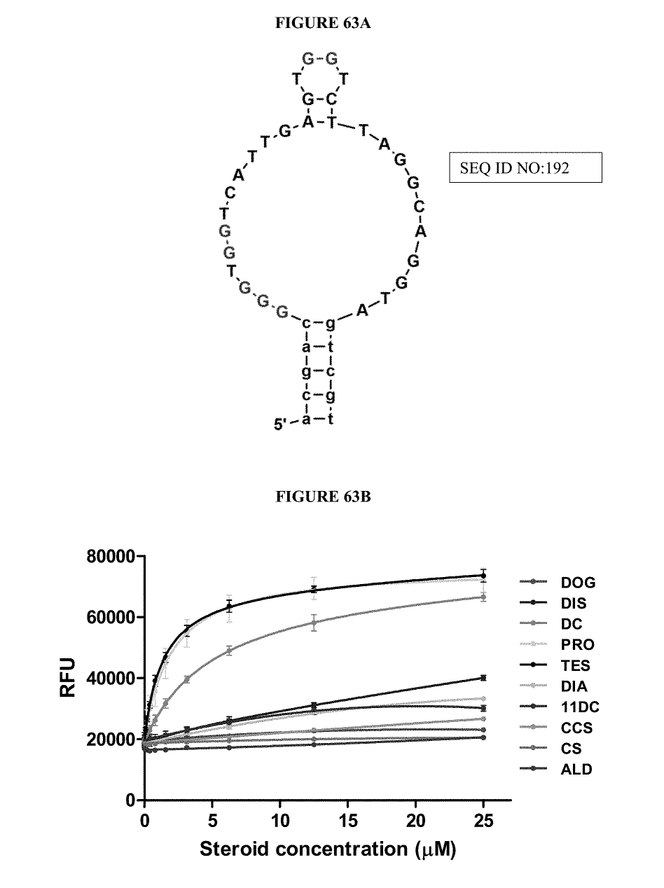

In certain non-limiting embodiments, a testosterone-binding primary aptamer comprises the sequences GGG and GGGG (SEQ ID NO:169) and GG, or a variant of any of these sequences that differs in one or two bases by substitution, deletion, insertion or extension, including addition or deletion of a G residue, where said primary aptamer binds to testosterone in an aqueous solution at room temperature or 25° C. with a dissociation constant of less than 10⁻⁴ M and binds to testosterone (see FIGS. 62B and 63B) selectively versus 11-deoxycorticosterone. In certain non-limiting embodiments, a testosterone-binding primary aptamer comprises the sequences GGG and GGGG (SEQ ID NO:169) and GG or a variant thereof and further comprises at least one operative sequence. In certain non-limiting embodiments, a testosterone-binding primary aptamer comprises the sequences GGG and GGGG (SEQ ID NO:169) and GG, or a variant thereof, and further comprises at least one operative sequence, said operative sequence complementary to a sequence comprised in a sensor oligonucleotide. In certain non-limiting embodiments, a testosterone-binding primary aptamer comprises the sequences GGG and GGGG (SEQ ID NO:169) and GG, or a variant thereof, and at least one operative sequence on either side (flanking) these three sequences. In certain non-limiting embodiments, a testosterone-binding primary aptamer comprises the sequences GGG and GGGG (SEQ ID NO:169) and GG, or a variant thereof, and at least one operative sequence on either side (flanking) these three sequences, where two of said operative sequences contain mutually complementary portions and can form a duplex.

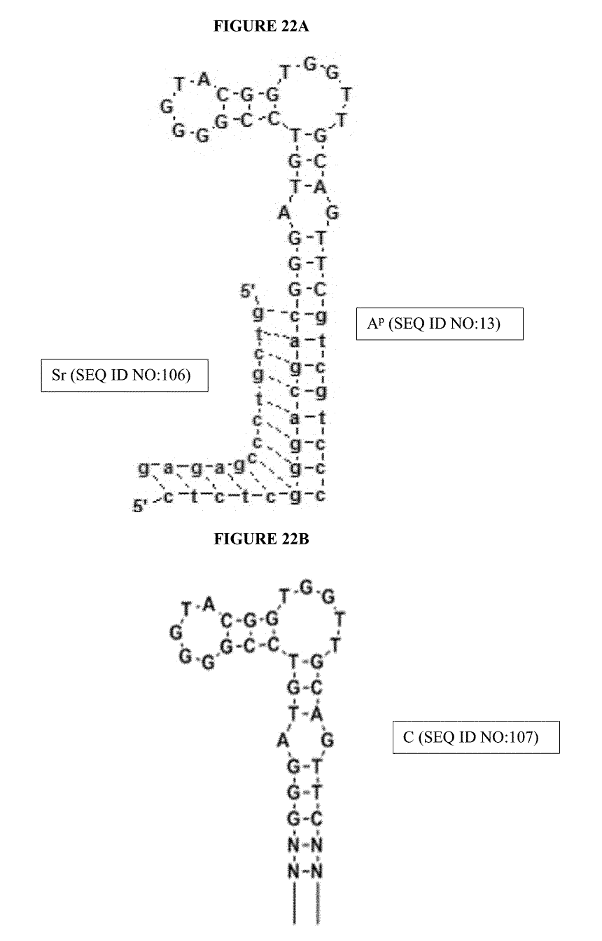

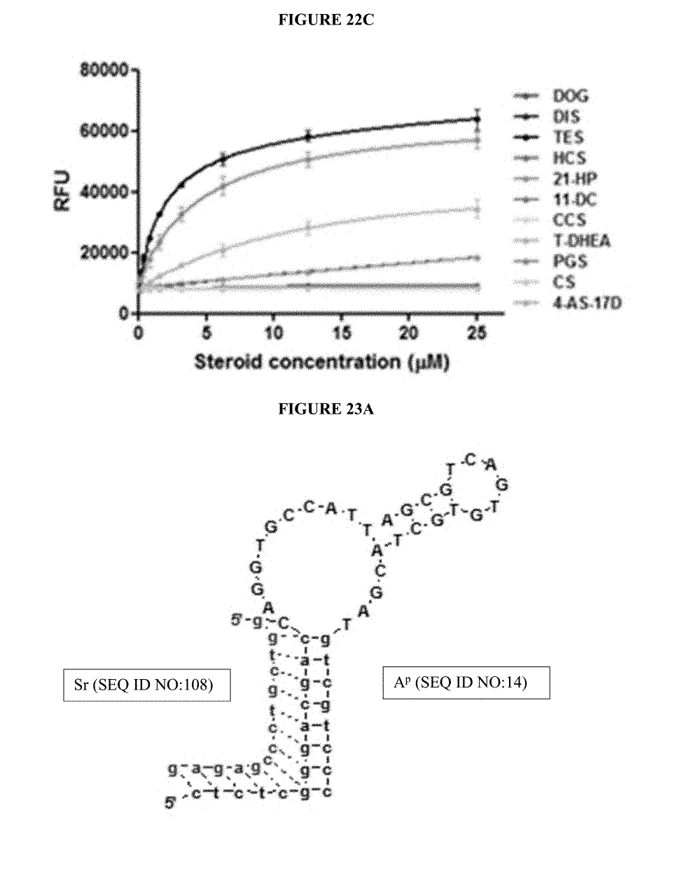

For example, but not by way of limitation, a testosterone-binding aptamer may comprise the sequence: CTC TCG GGA CGA CGG GAT GTC CGG GGT ACG GTG GTT GCA GTT CGT CGT CCC (SEQ ID NO:13) or a related sequence (e.g., SEQ ID NO:65, FIG. 1D; SEQ ID NO:191, FIG. 62A). In certain non-limiting embodiments, a testosterone-binding primary aptamer comprises a core sequence as set forth in SEQ ID NO:107 (FIG. 22B; FIG. 62A). In certain non-limiting embodiments, a testosterone-binding primary aptamer comprising a core sequence as set forth in SEQ ID NO:107 (FIG. 22B) has a binding affinity for testosterone that is at least about 50 percent or at least about 75 percent the binding affinity of the primary aptamer having SEQ ID NO:191). In certain non-limiting embodiments, a testosterone-binding primary aptamer comprising a core sequence as set forth in SEQ ID NO:107 (FIG. 22B) competes with primary aptamer having SEQ ID NO:191 for testosterone binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a testosterone-binding primary aptamer comprises a core sequence as set forth in SEQ ID NO:107 (FIG. 22B) and further comprises at least one operative sequence. In certain non-limiting embodiments, a testosterone-binding primary aptamer comprises a core sequence as set forth in SEQ ID NO:107 (FIG. 22B) and further comprises at least one operative sequence, said operative sequence complementary to a sequence comprised in a sensor oligonucleotide. In certain non-limiting embodiments, a testosterone-binding primary aptamer comprises a core sequence as set forth in SEQ ID NO:107 (FIG. 22B) and at least one operative sequence on either side (flanking) the core sequence. In certain non-limiting embodiments, a testosterone-binding primary aptamer comprises a core sequence as set forth in SEQ ID NO:107 (FIG. 22B) and at least one operative sequence on either side (flanking) the core sequence, where two of said operative sequences contain mutually complementary portions and can form a duplex.

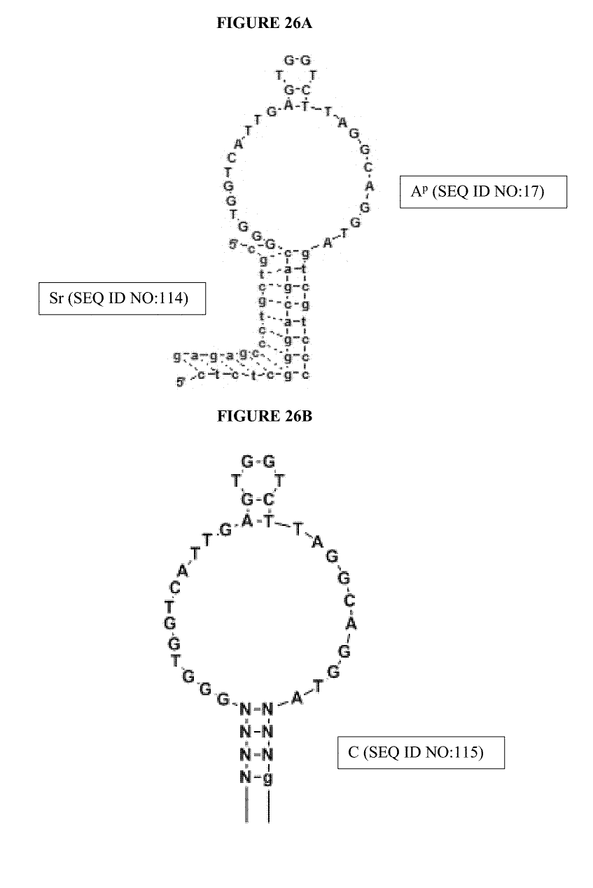

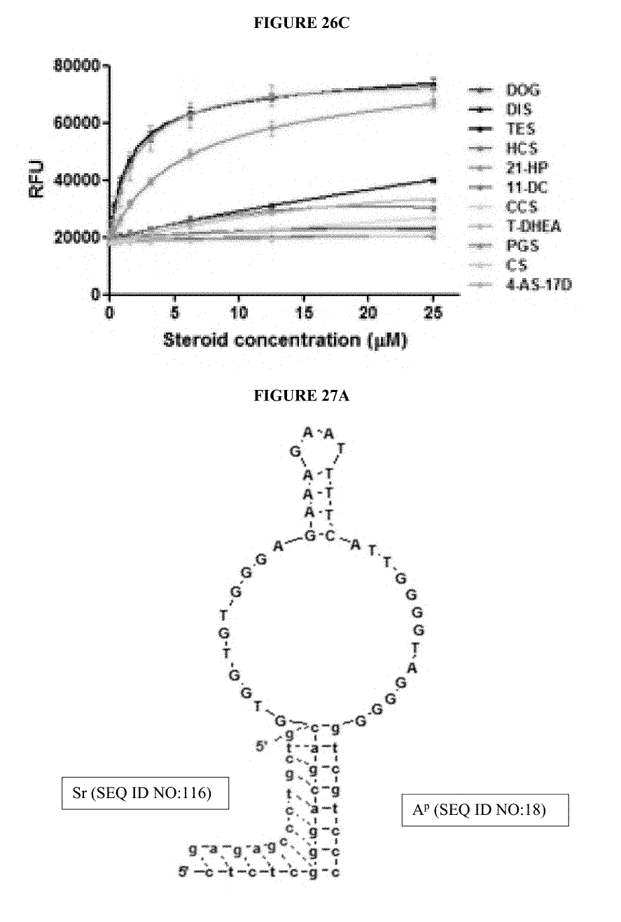

For example, but not by way of limitation, a testosterone-binding aptamer may comprise the sequence: CTC TCG GGA CGA CGG GTG GTC ATT GAG TGG TCT TAG GCA GGT AGT CGT CCC (SEQ ID NO:17) or related sequence (SEQ ID NO:192, FIG. 63A). In certain non-limiting embodiments, a testosterone-binding primary aptamer comprises a core sequence as set forth in SEQ ID NO:115 (FIG. 26B; FIG. 63A). In certain non-limiting embodiments, a testosterone-binding primary aptamer comprising a core sequence as set forth in SEQ ID NO:115 (FIG. 26B) has a binding affinity for testosterone that is at least about 50 percent or at least about 75 percent the binding affinity of the primary aptamer having SEQ ID NO:192). In certain non-limiting embodiments, a testosterone-binding primary aptamer comprising a core sequence as set forth in SEQ ID NO:115 (FIG. 26B) competes with primary aptamer having SEQ ID NO:192 for testosterone binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a testosterone-binding primary aptamer comprises a core sequence as set forth in SEQ ID NO:115 (FIG. 26B) and further comprises at least one operative sequence. In certain non-limiting embodiments, a testosterone-binding primary aptamer comprises a core sequence as set forth in SEQ ID NO:115 (FIG. 26B) and further comprises at least one operative sequence, said operative sequence complementary to a sequence comprised in a sensor oligonucleotide. In certain non-limiting embodiments, a testosterone-binding primary aptamer comprises a core sequence as set forth in SEQ ID NO:115 (FIG. 26B) and at least one operative sequence on either side (flanking) the core sequence. In certain non-limiting embodiments, a testosterone-binding primary aptamer comprises a core sequence as set forth in SEQ ID NO:115 (FIG. 26B) and at least one operative sequence on either side (flanking) the core sequence, where two of said operative sequences contain mutually complementary portions and can form a duplex.

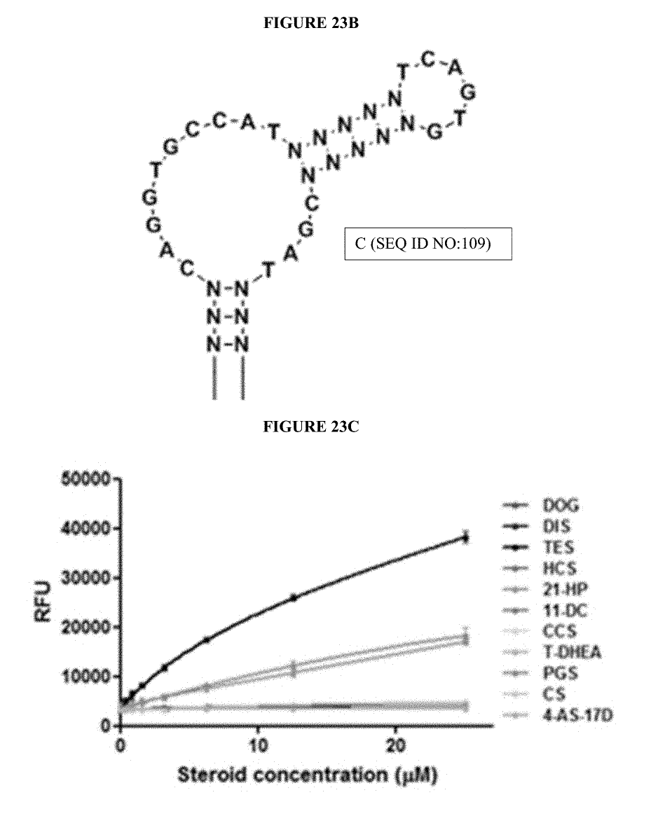

In certain non-limiting embodiments, a testosterone-binding primary aptamer comprises a core sequence as set forth in SEQ ID NO:109 (FIG. 23B). In certain non-limiting embodiments, a testosterone-binding primary aptamer comprising a core sequence as set forth in SEQ ID NO:109 (FIG. 23B) has a binding affinity for testosterone that is at least about 50 percent or at least about 75 percent the binding affinity of the primary aptamer having SEQ ID NO:14). In certain non-limiting embodiments, a testosterone-binding primary aptamer comprising a core sequence as set forth in SEQ ID NO:109 (FIG. 23B) competes with primary aptamer having SEQ ID NO:14 for testosterone binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a testosterone-binding primary aptamer comprises a core sequence as set forth in SEQ ID NO:109 (FIG. 23B) and further comprises at least one operative sequence. In certain non-limiting embodiments, a testosterone-binding primary aptamer comprises a core sequence as set forth in SEQ ID NO:109 (FIG. 23B) and further comprises at least one operative sequence, said operative sequence complementary to a sequence comprised in a sensor oligonucleotide.

In certain non-limiting embodiments, a testosterone-binding primary aptamer comprises a core sequence as set forth in SEQ ID NO:109 (FIG. 23B) and at least one operative sequence on either side (flanking) the core sequence. In certain non-limiting embodiments, a testosterone-binding primary aptamer comprises a core sequence as set forth in SEQ ID NO:109 (FIG. 23B) and at least one operative sequence on either side (flanking) the core sequence, where two of said operative sequences contain mutually complementary portions and can form a duplex.

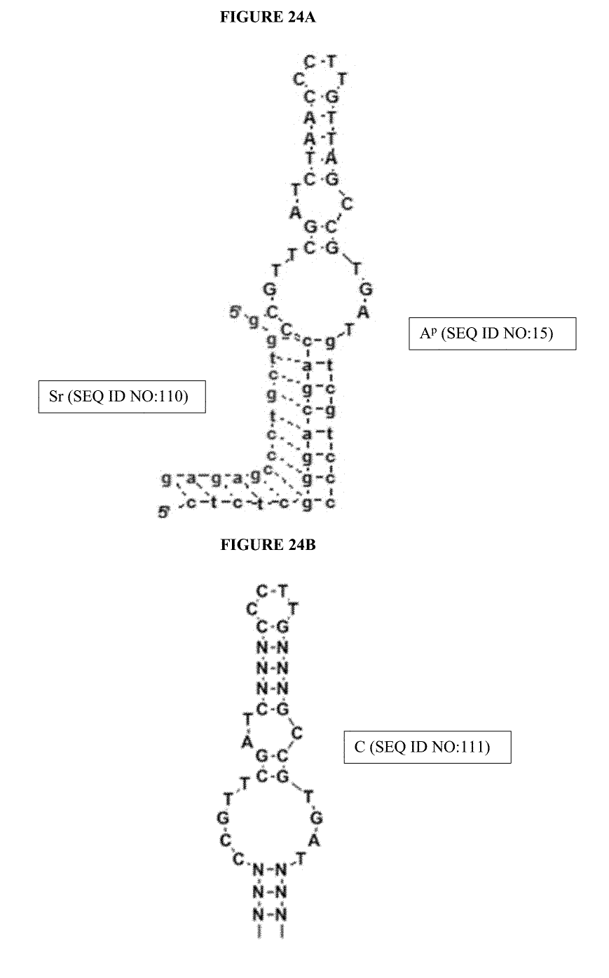

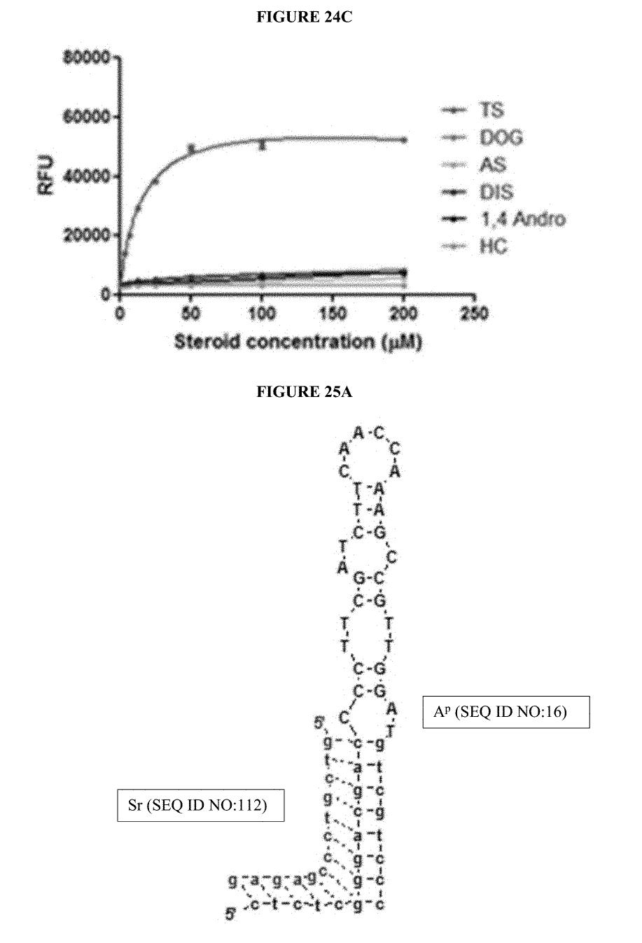

In certain non-limiting embodiments, a testosterone-binding primary aptamer comprises a core sequence as set forth in SEQ ID NO:111 (FIG. 24B). In certain non-limiting embodiments, a testosterone-binding primary aptamer comprising a core sequence as set forth in SEQ ID NO:111 (FIG. 24B) has a binding affinity for testosterone that is at least about 50 percent or at least about 75 percent the binding affinity of the primary aptamer having SEQ ID NO:15). In certain non-limiting embodiments, a testosterone-binding primary aptamer comprising a core sequence as set forth in SEQ ID NO:111 (FIG. 24B) competes with primary aptamer having SEQ ID NO:15 for testosterone binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a testosterone-binding primary aptamer comprises a core sequence as set forth in SEQ ID NO:111 (FIG. 24B) and further comprises at least one operative sequence. In certain non-limiting embodiments, a testosterone-binding primary aptamer comprises a core sequence as set forth in SEQ ID NO:111 (FIG. 24B) and further comprises at least one operative sequence, said operative sequence complementary to a sequence comprised in a sensor oligonucleotide. In certain non-limiting embodiments, a testosterone-binding primary aptamer comprises a core sequence as set forth in SEQ ID NO:111 (FIG. 24B) and at least one operative sequence on either side (flanking) the core sequence. In certain non-limiting embodiments, a testosterone-binding primary aptamer comprises a core sequence as set forth in SEQ ID NO:111 (FIG. 24B) and at least one operative sequence on either side (flanking) the core sequence, where two of said operative sequences contain mutually complementary portions and can form a duplex.

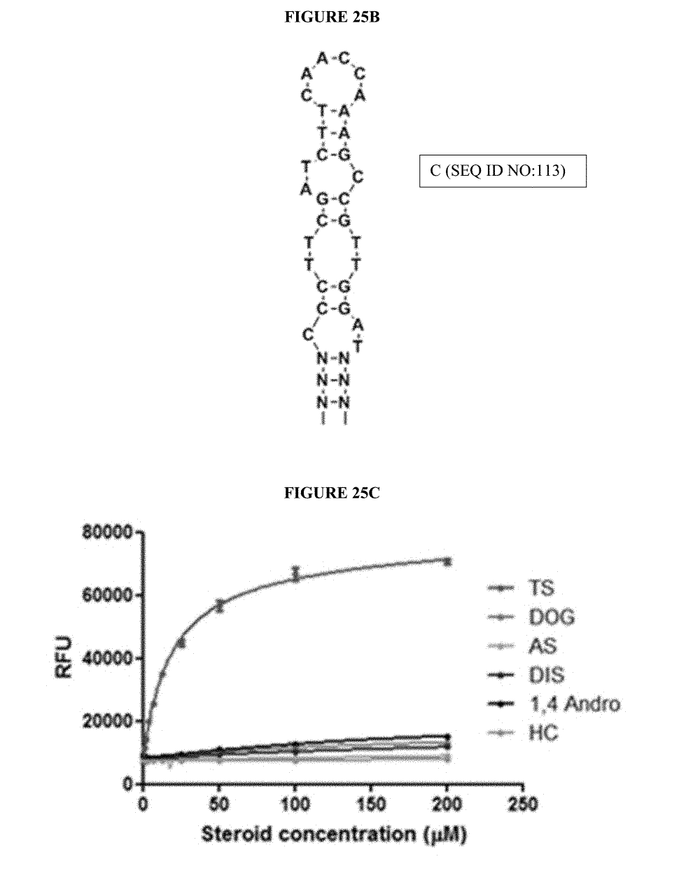

In certain non-limiting embodiments, a testosterone-binding primary aptamer comprises a core sequence as set forth in SEQ ID NO:113 (FIG. 25B). In certain non-limiting embodiments, a testosterone-binding primary aptamer comprising a core sequence as set forth in SEQ ID NO:111 (FIG. 25B) has a binding affinity for testosterone that is at least about 50 percent or at least about 75 percent the binding affinity of the primary aptamer having SEQ ID NO:16). In certain non-limiting embodiments, a testosterone-binding primary aptamer comprising a core sequence as set forth in SEQ ID NO:113 (FIG. 25B) competes with primary aptamer having SEQ ID NO:16 for testosterone binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a testosterone-binding primary aptamer comprises a core sequence as set forth in SEQ ID NO:113 (FIG. 25B) and further comprises at least one operative sequence. In certain non-limiting embodiments, a testosterone-binding primary aptamer comprises a core sequence as set forth in SEQ ID NO:113 (FIG. 25B) and further comprises at least one operative sequence, said operative sequence complementary to a sequence comprised in a sensor oligonucleotide. In certain non-limiting embodiments, a testosterone-binding primary aptamer comprises a core sequence as set forth in SEQ ID NO:113 (FIG. 25B) and at least one operative sequence on either side (flanking) the core sequence. In certain non-limiting embodiments, a testosterone-binding primary aptamer comprises a core sequence as set forth in SEQ ID NO:113 (FIG. 25B) and at least one operative sequence on either side (flanking) the core sequence, where two of said operative sequences contain mutually complementary portions and can form a duplex.

In certain non-limiting embodiments, isolated testosterone-binding primary aptamers comprise the nucleotide sequence of SEQ ID NO:13, SEQ ID NO:14, SEQ ID NO:15, SEQ ID NO: 16, SEQ ID NO: 17, SEQ ID NO:65, SEQ ID NO:191, or SEQ ID NO:192, or variants of these sequences having at least about 80 percent, or at least about 85 percent, or at least about 90 percent, or at least about 95 percent, or at least about 98 percent homology to the original sequence, for example obtained by substitutions, deletions, and insertions of Watson-Crick base pairs or by mutations at non-conserved positions. Percent homology can be determined using standard software such as BLAST or FASTA. In certain non-limiting embodiments, isolated testosterone-binding primary aptamers comprise the nucleotide sequence of SEQ ID NO:13, SEQ ID NO:14, SEQ ID NO:15, SEQ ID NO: 16, SEQ ID NO: 17, SEQ ID NO:65, SEQ ID NO:191, or SEQ ID NO:192. Said aptamers can bind to testosterone and in their structure-switching formats (for example, in an anti-aptamer assay, a pseudosandwich assay, or a sandwich assay) they can respond by an increase in fluorescence.

5.1.8 Sphingosine-1-Phosphate-Binding Primary Aptamers

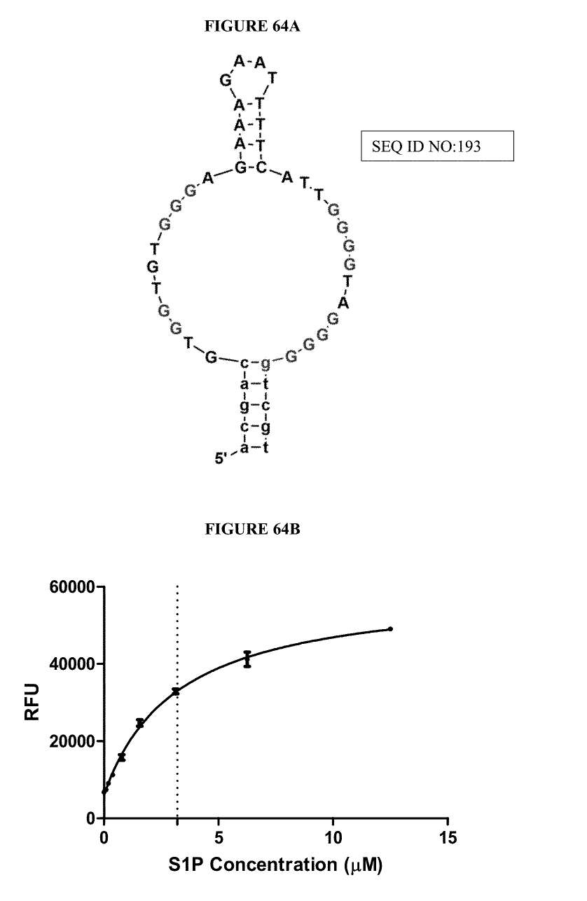

In certain non-limiting embodiments, a primary aptamer binds to sphingosine-1-phosphate in an aqueous solution at room temperature or 25° C. with a dissociation constant of less than 10⁻⁴ M (see FIG. 64B).

In certain non-limiting embodiments, a sphingosine-1-phosphate-binding primary aptamer comprises the sequences GG and GGGG (SEQ ID NO:169) and GGGGG (SEQ ID NO:168) or a variant of any of these sequences that differs in one or two bases by substitution, deletion, insertion or extension, where said primary aptamer binds to sphingosine-1-phosphate in an aqueous solution at room temperature or 25° C. with a dissociation constant of less than 10⁻⁴ M and binds selectively to sphingosine-1-phosphate. In certain non-limiting embodiments, a sphingosine-1-phosphate-binding primary aptamer comprises the sequences GG and GGGG (SEQ ID NO:169) and GGGGG (SEQ ID NO:168) or a variant thereof and further comprises at least one operative sequence. In certain non-limiting embodiments, a sphingosine-1-phosphate-binding primary aptamer comprises the sequences GG and GGGG (SEQ ID NO:169) and GGGGG (SEQ ID NO:168), or a variant thereof, and further comprises at least one operative sequence, said operative sequence complementary to a sequence comprised in a sensor oligonucleotide. In certain non-limiting embodiments, a sphingosine-1-phosphate-binding primary aptamer comprises the sequences GG and GGGG (SEQ ID NO:169) and GGGGG (SEQ ID NO:168), or a variant thereof, and at least one operative sequence on either side (flanking) these three sequences. In certain non-limiting embodiments, a sphingosine-1-phosphate-binding primary aptamer comprises the sequences GG and GGGG (SEQ ID NO:169) and GGGGG (SEQ ID NO:168) or a variant thereof, and at least one operative sequence on either side (flanking) these three sequences, where two of said operative sequences contain mutually complementary portions and can form a duplex.

For example, but not by way of limitation, a sphingosine-1-phosphate-binding aptamer may comprise the sequence: CTC TCG GGA CGA CGT GGT GTG GGA GAA AGA ATT TTC ATT GGG GTA GGG GGT CGT CCC (SEQ ID NO:18) or related sequence (SEQ ID NO:193, FIG. 64A).

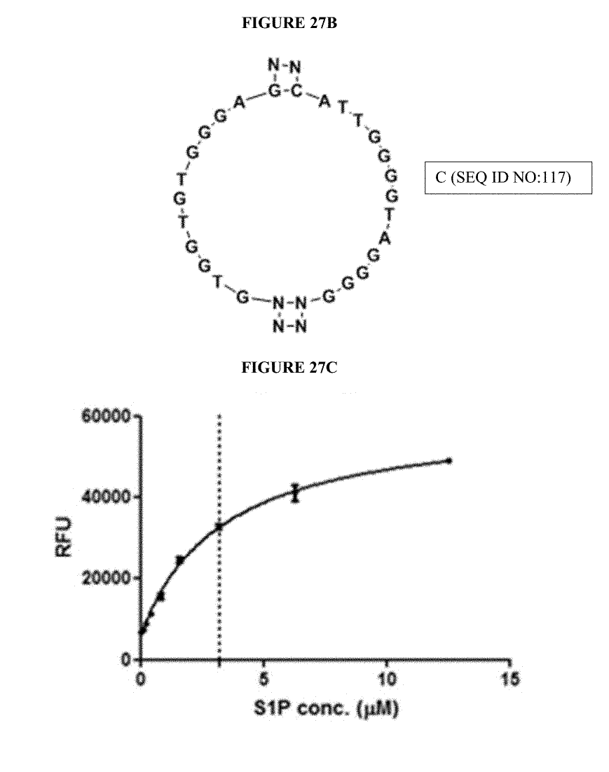

In certain non-limiting embodiments, a sphingosine-1-phosphate-binding primary aptamer comprises a core sequence as set forth in SEQ ID NO:117 (FIG. 27B; FIG. 64A). In certain non-limiting embodiments, a sphingosine-1-phosphate-binding primary aptamer comprising a core sequence as set forth in SEQ ID NO:117 (FIG. 27B) has a binding affinity for sphingosine-1-phosphate that is at least about 50 percent or at least about 75 percent the binding affinity of the primary aptamer having SEQ ID NO:193). In certain non-limiting embodiments, a sphingosine-1-phosphate-binding primary aptamer comprising a core sequence as set forth in SEQ ID NO:117 (FIG. 27B) competes with primary aptamer having SEQ ID NO:193 for sphingosine-1-phosphate binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a sphingosine-1-phosphate-binding primary aptamer comprises a core sequence as set forth in SEQ ID NO:117 (FIG. 27B) and further comprises at least one operative sequence. In certain non-limiting embodiments, a sphingosine-1-phosphate-binding primary aptamer comprises a core sequence as set forth in SEQ ID NO:117 (FIG. 27B) and further comprises at least one operative sequence, said operative sequence complementary to a sequence comprised in a sensor oligonucleotide. In certain non-limiting embodiments, a sphingosine-1-phosphate-binding primary aptamer comprises a core sequence as set forth in SEQ ID NO:117 (FIG. 27B) and at least one operative sequence on either side (flanking) the core sequence. In certain non-limiting embodiments, a sphingosine-1-phosphate-binding primary aptamer comprises a core sequence as set forth in SEQ ID NO:117 (FIG. 27B) and at least one operative sequence on either side (flanking) the core sequence, where two of said operative sequences contain mutually complementary portions and can form a duplex.

In certain non-limiting embodiments, isolated sphingosine-1-phosphate-binding primary aptamers comprise the nucleotide sequence of SEQ ID NO:18, SEQ ID NO:193, or variants of these sequences having at least about 80 percent, or at least about 85 percent, or at least about 90 percent, or at least about 95 percent, or at least about 98 percent homology to the original sequence, for example obtained by substitutions, deletions, and insertions of Watson-Crick base pairs or by mutations at non-conserved positions. Percent homology can be determined using standard software such as BLAST or FASTA. In certain non-limiting embodiments, isolated sphingosine-1-phosphate-binding primary aptamers comprise the nucleotide sequence of SEQ ID NO:18 or SEQ ID NO:193. Said aptamers can bind to sphingosine-1-phosphate and in their structure-switching formats (for example, in an anti-aptamer assay, a pseudosandwich assay, or a sandwich assay) they can respond by an increase in fluorescence.

5.1.9 Dopamine-Binding Primary Aptamers

In certain non-limiting embodiments, a primary aptamer binds to dopamine in an aqueous solution at room temperature or 25° C. with a dissociation constant of less than 10⁻⁴ M and binds selectively with dopamine.

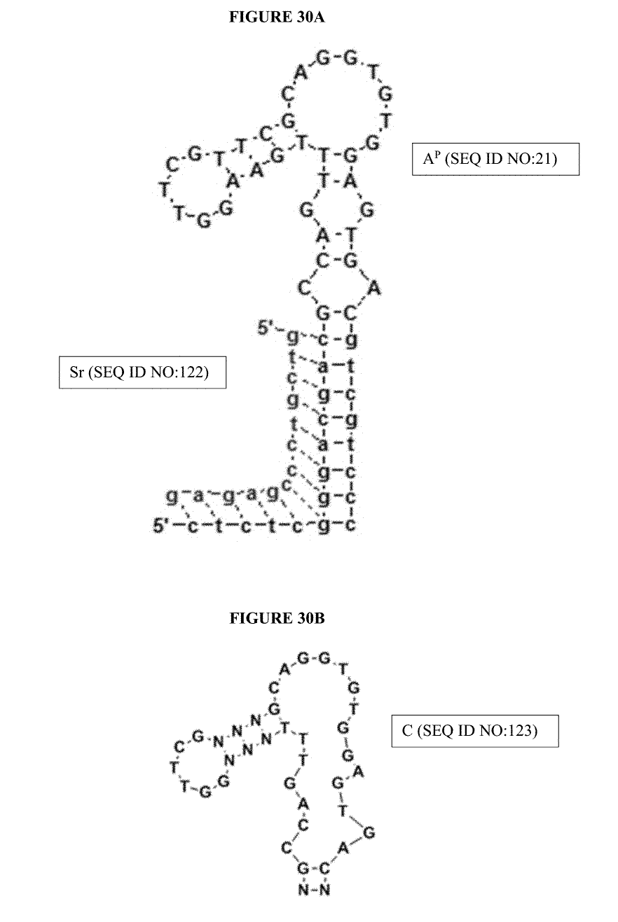

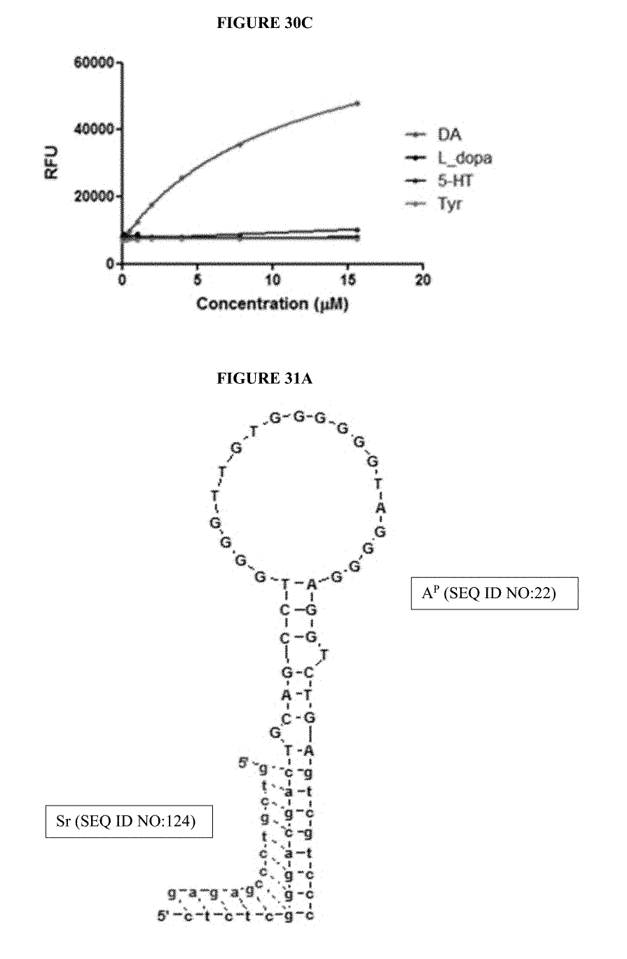

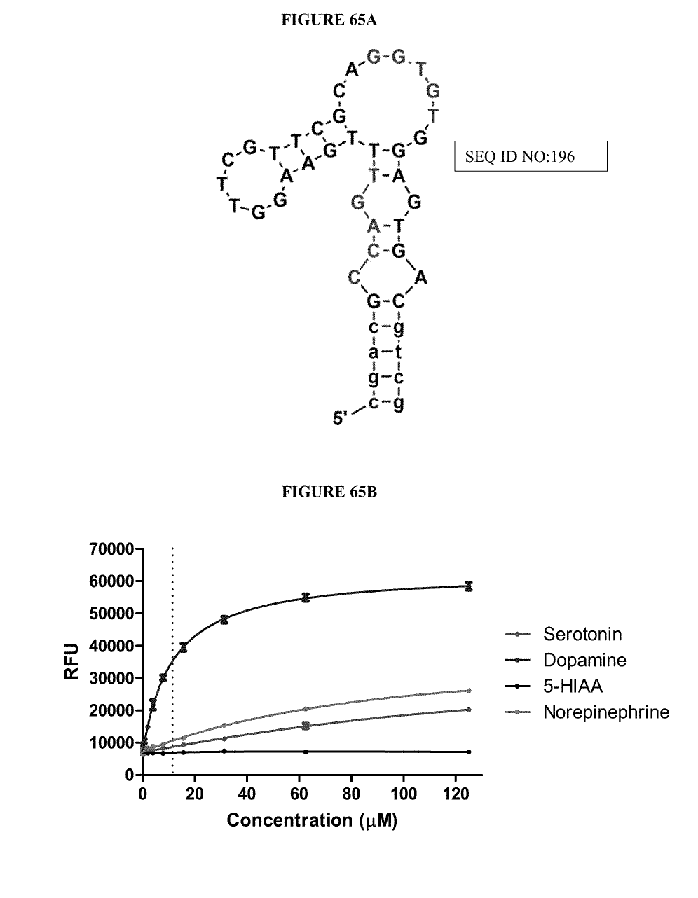

In certain non-limiting embodiments, a dopamine-binding primary aptamer comprises the sequences CCGAT (SEQ ID NO:194) and GGTGT (SEQ ID NO:195) or a variant of any of these sequences that differs in one or two bases by substitution, deletion, insertion or extension, where said primary aptamer binds to dopamine in an aqueous solution at room temperature or 25° C. with a dissociation constant of less than 10⁻⁴ M and binds to dopamine selectively versus serotonin or norepinephrine (FIG. 65B). In certain non-limiting embodiments, a dopamine-binding primary aptamer comprises the sequences CCGAT (SEQ ID NO:194) and GGTGT (SEQ ID NO:195) or a variant thereof and further comprises at least one operative sequence. In certain non-limiting embodiments, a dopamine-binding primary aptamer comprises the sequences CCGAT (SEQ ID NO:194) and GGTGT (SEQ ID NO:195), or a variant thereof, and further comprises at least one operative sequence, said operative sequence complementary to a sequence comprised in a sensor oligonucleotide. In certain non-limiting embodiments, a dopamine-binding primary aptamer comprises the sequences CCGAT (SEQ ID NO:194) and GGTGT (SEQ ID NO:195), or a variant thereof, and at least one operative sequence on either side (flanking) these two sequences. In certain non-limiting embodiments, a dopamine-binding primary aptamer comprises the sequences CCGAT (SEQ ID NO:194) and GGTGT (SEQ ID NO:195) or a variant thereof, and at least one operative sequence on either side (flanking) these two sequences, where two of said operative sequences contain mutually complementary portions and can form a duplex. For example, but not by way of limitation, a dopamine-binding aptamer may comprise the sequence: CTC TCG GGA CGA CGC CAG TTT GAA GGT TCG TTC GCA GGT GTG GAG TGA CGT CGT CCC (SEQ ID NO:21) or related sequence (SEQ ID NO:196, FIG. 65A). In certain non-limiting embodiments, a dopamine-binding primary aptamer comprises a core sequence as set forth in SEQ ID NO:123 (FIG. 30B; FIG. 65A). In certain non-limiting embodiments, a dopamine-binding primary aptamer comprising a core sequence as set forth in SEQ ID NO:123 (FIG. 30B) has a binding affinity for dopamine that is at least about 50 percent or at least about 75 percent the binding affinity of the primary aptamer having SEQ ID NO:196). In certain non-limiting embodiments, a dopamine-binding primary aptamer comprising a core sequence as set forth in SEQ ID NO:123 (FIG. 30B) competes with primary aptamer having SEQ ID NO:196 for dopamine binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a dopamine-binding primary aptamer comprises a core sequence as set forth in SEQ ID NO:123 (FIG. 30B) and further comprises at least one operative sequence. In certain non-limiting embodiments, a dopamine-binding primary aptamer comprises a core sequence as set forth in SEQ ID NO:123 (FIG. 30B) and further comprises at least one operative sequence, said operative sequence complementary to a sequence comprised in a sensor oligonucleotide. In certain non-limiting embodiments, a dopamine-binding primary aptamer comprises a core sequence as set forth in SEQ ID NO:123 (FIG. 30B) and at least one operative sequence on either side (flanking) the core sequence. In certain non-limiting embodiments, a dopamine-binding primary aptamer comprises a core sequence as set forth in SEQ ID NO:123 (FIG. 30B) and at least one operative sequence on either side (flanking) the core sequence, where two of said operative sequences contain mutually complementary portions and can form a duplex.

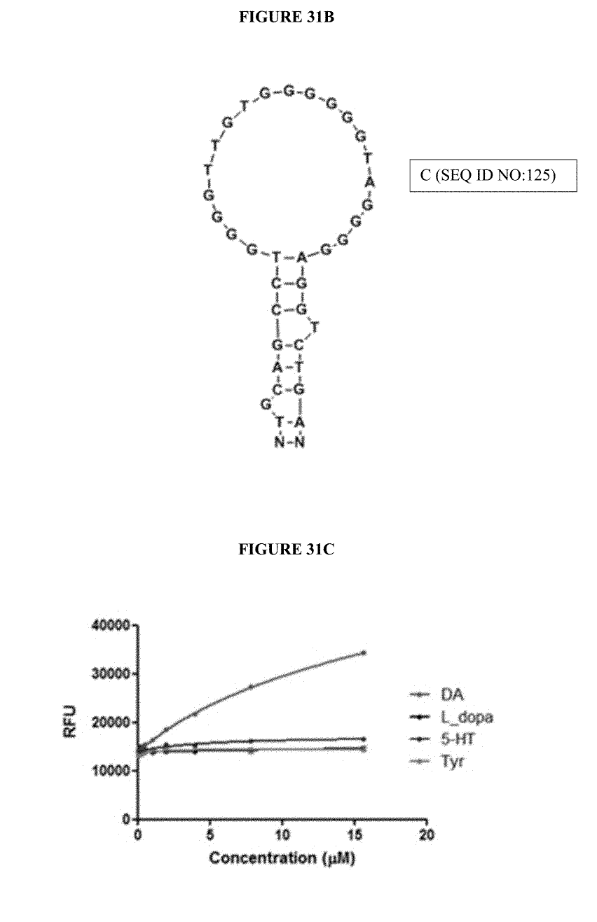

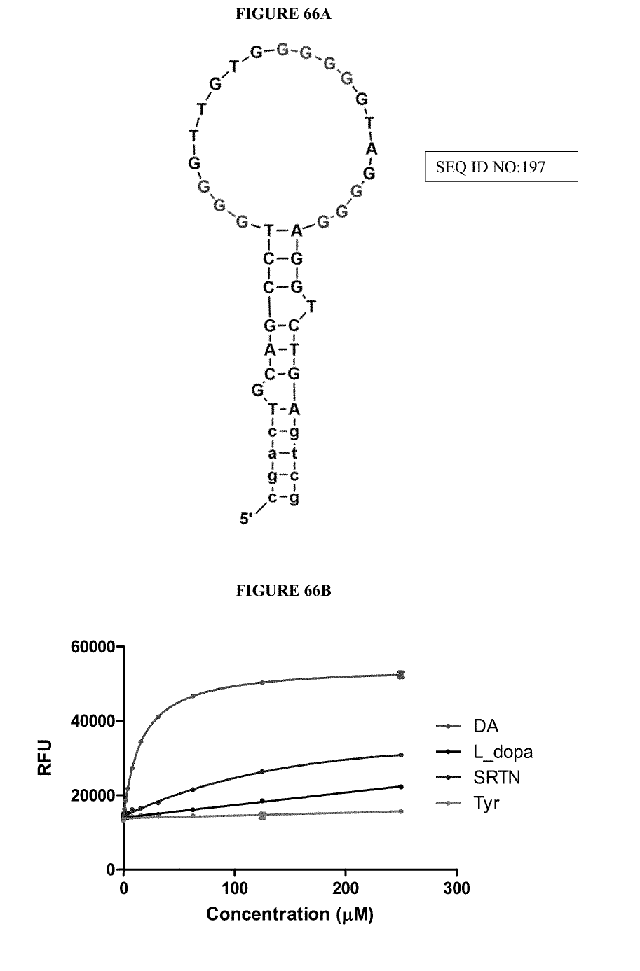

In certain non-limiting embodiments, a dopamine-binding primary aptamer comprises the sequences GGG and GGGG (SEQ ID NO:169) or a variant of any of these sequences that differs in one or two bases by substitution, deletion, insertion or extension, where said primary aptamer binds to dopamine in an aqueous solution at room temperature or 25° C. with a dissociation constant of less than 10⁻⁴ M and binds to dopamine selectively versus serotonin or tyrosine (FIG. 66B). In certain non-limiting embodiments, a dopamine-binding primary aptamer comprises the sequences GGG and GGGG (SEQ ID NO:169) or a variant thereof and further comprises at least one operative sequence. In certain non-limiting embodiments, a dopamine-binding primary aptamer comprises the sequences GGG and GGGG (SEQ ID NO:169), or a variant thereof, and further comprises at least one operative sequence, said operative sequence complementary to a sequence comprised in a sensor oligonucleotide. In certain non-limiting embodiments, a dopamine-binding primary aptamer comprises the sequences GGG and GGGG (SEQ ID NO:169), or a variant thereof, and at least one operative sequence on either side (flanking) these two sequences. In certain non-limiting embodiments, a dopamine-binding primary aptamer comprises the sequences GGG and GGGG (SEQ ID NO:169) or a variant thereof, and at least one operative sequence on either side (flanking) these two sequences, where two of said operative sequences contain mutually complementary portions and can form a duplex. For example, but not by way of limitation, a dopamine-binding aptamer may comprise the sequence: CTC TCG GGA CGA CTG CAG CCT GGG GTT GTG GGG GGT AGG GGA GGT CTG AGT CGT CCC (SEQ ID NO:22; FIG. 31A) or related sequence (SEQ ID NO:197, FIG. 66A). In certain non-limiting embodiments, a dopamine-binding primary aptamer comprises a core sequence as set forth in SEQ ID NO:125 (FIG. 31B; FIG. 66A). In certain non-limiting embodiments, a dopamine-binding primary aptamer comprising a core sequence as set forth in SEQ ID NO:125 (FIG. 31B) has a binding affinity for dopamine that is at least about 50 percent or at least about 75 percent the binding affinity of the primary aptamer having SEQ ID NO:197). In certain non-limiting embodiments, a dopamine-binding primary aptamer comprising a core sequence as set forth in SEQ ID NO:125 (FIG. 31B) competes with primary aptamer having SEQ ID NO:197 for dopamine binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a dopamine-binding primary aptamer comprises a core sequence as set forth in SEQ ID NO:125 (FIG. 31B) and further comprises at least one operative sequence. In certain non-limiting embodiments, a dopamine-binding primary aptamer comprises a core sequence as set forth in SEQ ID NO:125 (FIG. 31B) and further comprises at least one operative sequence, said operative sequence complementary to a sequence comprised in a sensor oligonucleotide. In certain non-limiting embodiments, a dopamine-binding primary aptamer comprises a core sequence as set forth in SEQ ID NO:125 (FIG. 31B) and at least one operative sequence on either side (flanking) the core sequence. In certain non-limiting embodiments, a dopamine-binding primary aptamer comprises a core sequence as set forth in SEQ ID NO:125 (FIG. 31B) and at least one operative sequence on either side (flanking) the core sequence, where two of said operative sequences contain mutually complementary portions and can form a duplex.

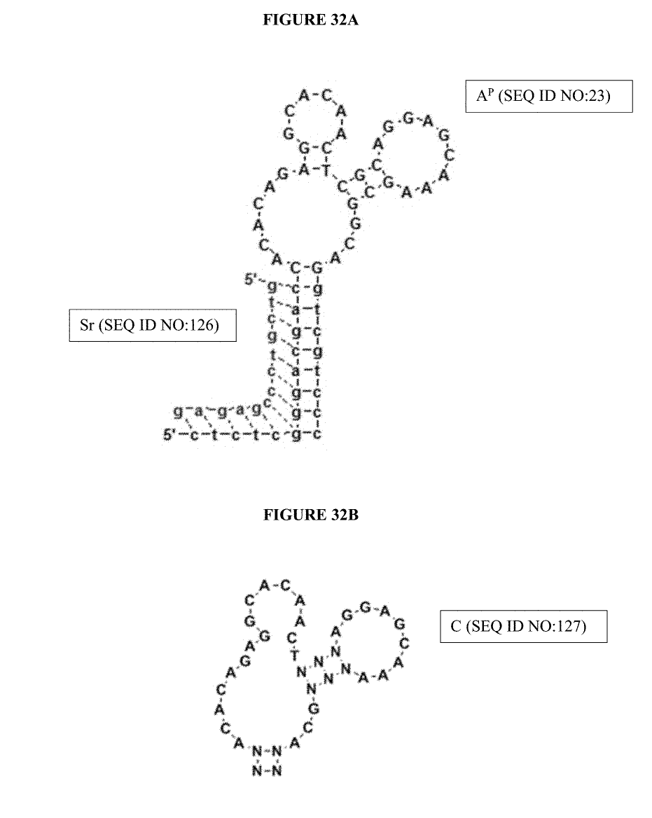

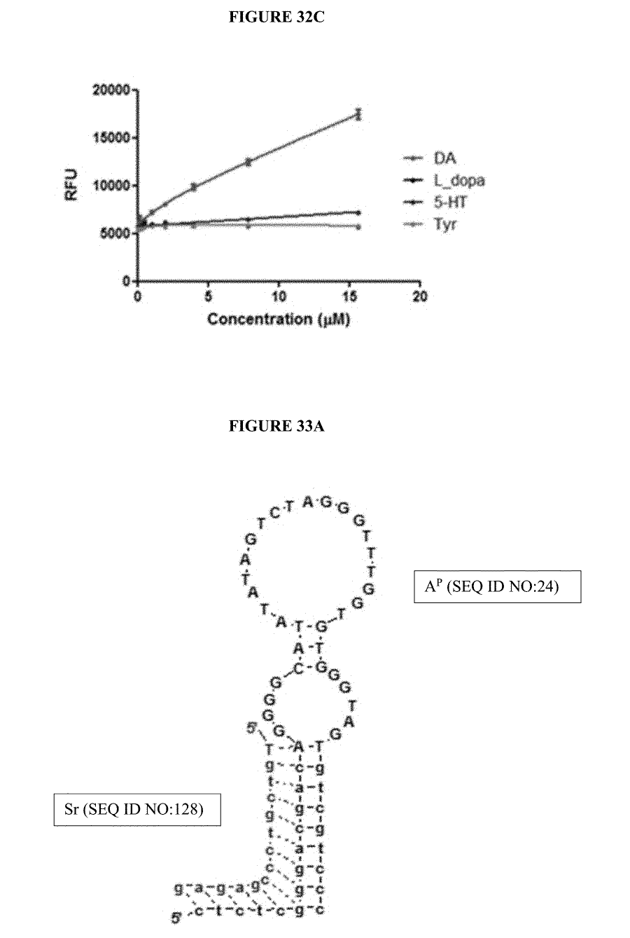

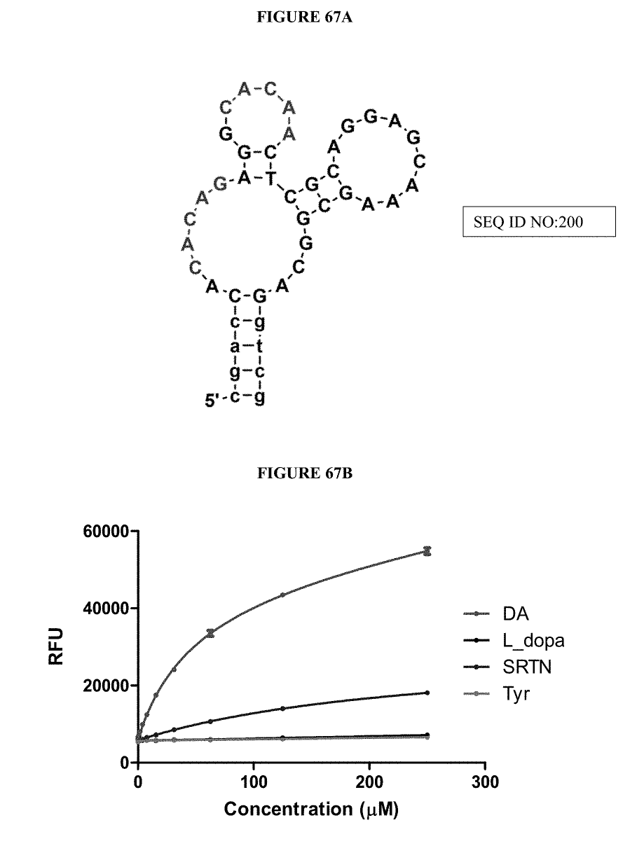

In certain non-limiting embodiments, a dopamine-binding primary aptamer comprises the sequences CACAG (SEQ ID NO:198) and CACAA (SEQ ID NO:199) or a variant of any of these sequences that differs in one or two bases by substitution, deletion, insertion or extension, where said primary aptamer binds to dopamine in an aqueous solution at room temperature or 25° C. with a dissociation constant of less than 10⁻⁴ M and binds to dopamine selectively versus serotonin, melatonin or tyrosine (FIG. 67B). In certain non-limiting embodiments, a dopamine-binding primary aptamer comprises the sequences CACAG (SEQ ID NO:198) and CACAA (SEQ ID NO:199) or a variant thereof and further comprises at least one operative sequence. In certain non-limiting embodiments, a dopamine-binding primary aptamer comprises the sequences CACAG (SEQ ID NO:198) and CACAA (SEQ ID NO:199) or a variant thereof, and further comprises at least one operative sequence, said operative sequence complementary to a sequence comprised in a sensor oligonucleotide. In certain non-limiting embodiments, a dopamine-binding primary aptamer comprises the sequences CACAG (SEQ ID NO:198) and CACAA (SEQ ID NO:199), or a variant thereof, and at least one operative sequence on either side (flanking) these two sequences. In certain non-limiting embodiments, a dopamine-binding primary aptamer comprises the sequences CACAG (SEQ ID NO:198) and CACAA (SEQ ID NO:199) or a variant thereof, and at least one operative sequence on either side (flanking) these two sequences, where two of said operative sequences contain mutually complementary portions and can form a duplex. For example, but not by way of limitation, a dopamine-binding aptamer may comprise the sequence: CTC TCG GGA CGA CCA CAC AGA GGC ACA ACT CGC AGG AGC AAA GCG GCA GGT CGT CCC (SEQ ID NO:23; FIG. 32A) or related sequence (SEQ ID NO:200, FIG. 67A). In certain non-limiting embodiments, a dopamine-binding primary aptamer comprises a core sequence as set forth in SEQ ID NO:127 (FIG. 32B; FIG. 67A). In certain non-limiting embodiments, a dopamine-binding primary aptamer comprising a core sequence as set forth in SEQ ID NO:127 (FIG. 32B) has a binding affinity for dopamine that is at least about 50 percent or at least about 75 percent the binding affinity of the primary aptamer having SEQ ID NO:200). In certain non-limiting embodiments, a dopamine-binding primary aptamer comprising a core sequence as set forth in SEQ ID NO:125 (FIG. 31B) competes with primary aptamer having SEQ ID NO:200 for dopamine binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a dopamine-binding primary aptamer comprises a core sequence as set forth in SEQ ID NO:127 (FIG. 32B) and further comprises at least one operative sequence. In certain non-limiting embodiments, a dopamine-binding primary aptamer comprises a core sequence as set forth in SEQ ID NO:127 (FIG. 32B) and further comprises at least one operative sequence, said operative sequence complementary to a sequence comprised in a sensor oligonucleotide. In certain non-limiting embodiments, a dopamine-binding primary aptamer comprises a core sequence as set forth in SEQ ID NO:127 (FIG. 32B) and at least one operative sequence on either side (flanking) the core sequence. In certain non-limiting embodiments, a dopamine-binding primary aptamer comprises a core sequence as set forth in SEQ ID NO:127 (FIG. 32B) and at least one operative sequence on either side (flanking) the core sequence, where two of said operative sequences contain mutually complementary portions and can form a duplex.

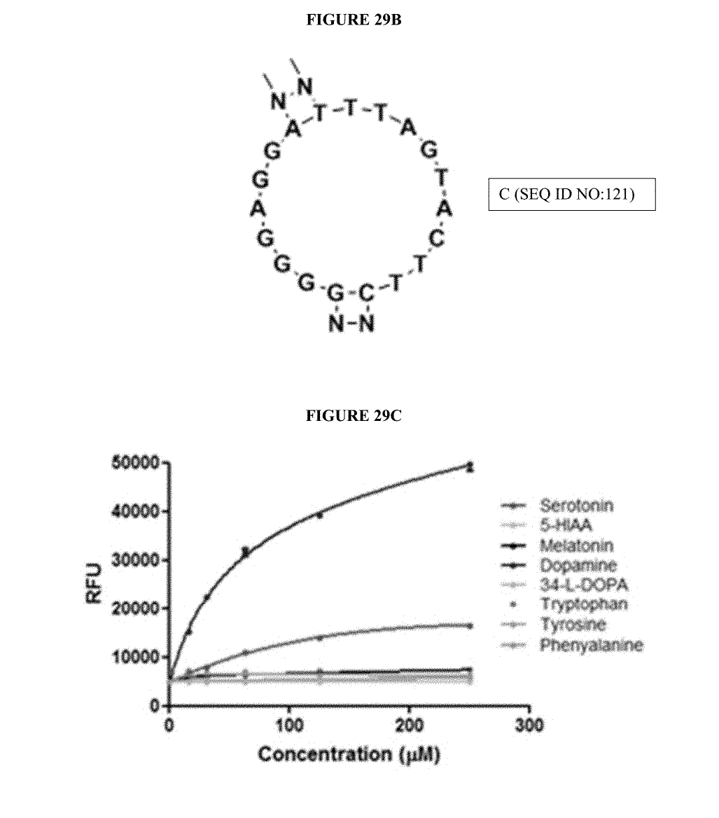

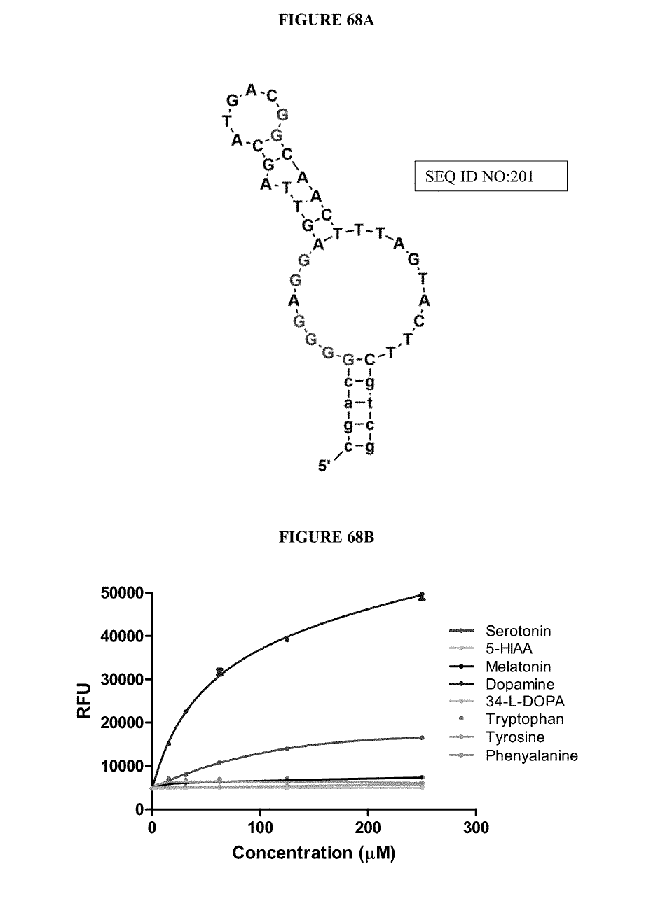

In certain non-limiting embodiments, a dopamine-binding primary aptamer comprises the sequences GGGG (SEQ ID NO:169) and GG or a variant of any of these sequences that differs in one or two bases by substitution, deletion, insertion or extension, where said primary aptamer binds to dopamine in an aqueous solution at room temperature or 25° C. with a dissociation constant of less than 10⁻⁴ M and binds to dopamine selectively versus serotonin, melatonin or tyrosine (FIG. 68B). In certain non-limiting embodiments, a dopamine-binding primary aptamer comprises the sequences GGGG (SEQ ID NO:169) and GG or a variant thereof and further comprises at least one operative sequence. In certain non-limiting embodiments, a dopamine-binding primary aptamer comprises the sequences GGGG (SEQ ID NO:169) and GG or a variant thereof, and further comprises at least one operative sequence, said operative sequence complementary to a sequence comprised in a sensor oligonucleotide. In certain non-limiting embodiments, a dopamine-binding primary aptamer comprises the sequences GGGG (SEQ ID NO:169) and GG, or a variant thereof, and at least one operative sequence on either side (flanking) these two sequences. In certain non-limiting embodiments, a dopamine-binding primary aptamer comprises the sequences GGGG (SEQ ID NO:169) and GG or a variant thereof, and at least one operative sequence on either side (flanking) these two sequences, where two of said operative sequences contain mutually complementary portions and can form a duplex. For example, but not by way of limitation, a dopamine-binding aptamer may comprise the sequence: CTC TCG GGA CGA CGG GGA GGA GTT AGC ATG ACG GCA ACT TTA GTA CTT CGT CGT CCC (SEQ ID NO:20; FIG. 29A) or related sequence (SEQ ID NO:201, FIG. 68A). In certain non-limiting embodiments, a dopamine-binding primary aptamer comprises a core sequence as set forth in SEQ ID NO:121 (FIG. 29B; FIG. 68A). In certain non-limiting embodiments, a dopamine-binding primary aptamer comprising a core sequence as set forth in SEQ ID NO:121 (FIG. 29B) has a binding affinity for dopamine that is at least about 50 percent or at least about 75 percent the binding affinity of the primary aptamer having SEQ ID NO:201). In certain non-limiting embodiments, a dopamine-binding primary aptamer comprising a core sequence as set forth in SEQ ID NO:121 (FIG. 29B) competes with primary aptamer having SEQ ID NO:201 for dopamine binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a dopamine-binding primary aptamer comprises a core sequence as set forth in SEQ ID NO:121 (FIG. 29B) and further comprises at least one operative sequence. In certain non-limiting embodiments, a dopamine-binding primary aptamer comprises a core sequence as set forth in SEQ ID NO:121 (FIG. 29B) and further comprises at least one operative sequence, said operative sequence complementary to a sequence comprised in a sensor oligonucleotide. In certain non-limiting embodiments, a dopamine-binding primary aptamer comprises a core sequence as set forth in SEQ ID NO:121 (FIG. 29B) and at least one operative sequence on either side (flanking) the core sequence. In certain non-limiting embodiments, a dopamine-binding primary aptamer comprises a core sequence as set forth in SEQ ID NO:121 (FIG. 29B) and at least one operative sequence on either side (flanking) the core sequence, where two of said operative sequences contain mutually complementary portions and can form a duplex.

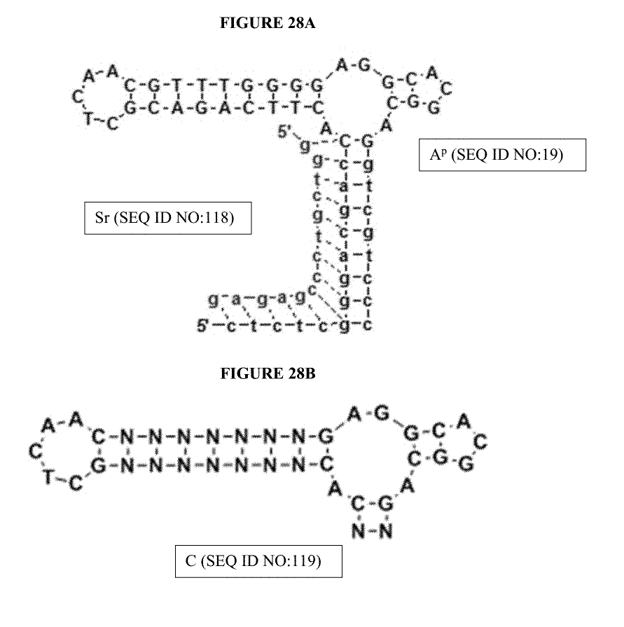

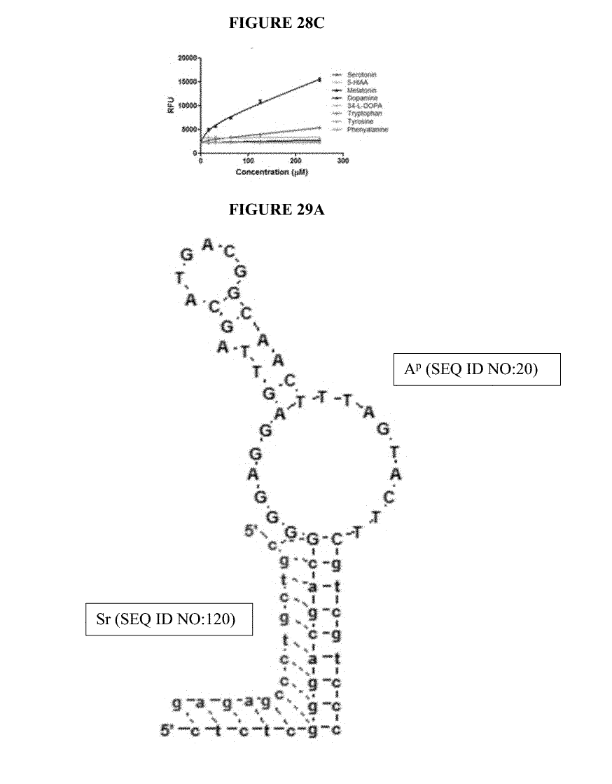

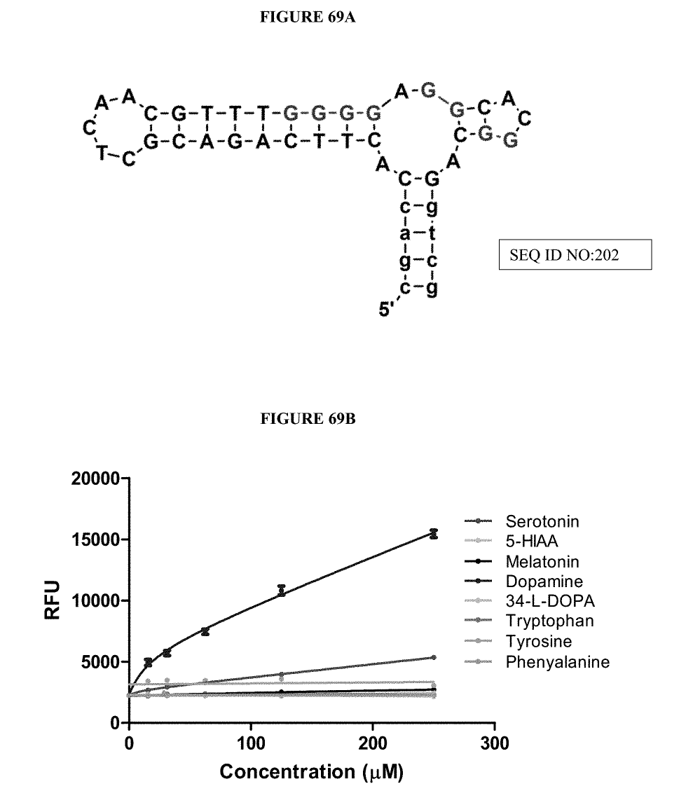

In certain non-limiting embodiments, a dopamine-binding primary aptamer comprises the sequences GGGG (SEQ ID NO:169) and GG or a variant of any of these sequences that differs in one or two bases by substitution, deletion, insertion or extension, where said primary aptamer binds to dopamine in an aqueous solution at room temperature or 25° C. with a dissociation constant of less than 10⁻⁴ M and binds to dopamine selectively versus serotonin, melatonin or tyrosine (FIG. 69B). In certain non-limiting embodiments, a dopamine-binding primary aptamer comprises the sequences GGGG (SEQ ID NO:169) and GG or a variant thereof and further comprises at least one operative sequence. In certain non-limiting embodiments, a dopamine-binding primary aptamer comprises the sequences GGGG (SEQ ID NO:169) and GG or a variant thereof, and further comprises at least one operative sequence, said operative sequence complementary to a sequence comprised in a sensor oligonucleotide. In certain non-limiting embodiments, a dopamine-binding primary aptamer comprises the sequences GGGG (SEQ ID NO:169) and GG, or a variant thereof, and at least one operative sequence on either side (flanking) these two sequences. In certain non-limiting embodiments, a dopamine-binding primary aptamer comprises the sequences GGGG (SEQ ID NO:169) and GG or a variant thereof, and at least one operative sequence on either side (flanking) these two sequences, where two of said operative sequences contain mutually complementary portions and can form a duplex. For example, but not by way of limitation, a dopamine-binding aptamer may comprise the sequence: CTC TCG GGA CGA CCA CTT CAG ACG CTC AAC GTT TGG GGA GGC ACG GCA GGT CGT CCC (SEQ ID NO:19; FIG. 28A) or related sequence (SEQ ID NO:202, FIG. 69A). In certain non-limiting embodiments, a dopamine-binding primary aptamer comprises a core sequence as set forth in SEQ ID NO:119 (FIG. 28B; FIG. 69A). In certain non-limiting embodiments, a dopamine-binding primary aptamer comprising a core sequence as set forth in SEQ ID NO:119 (FIG. 28B) has a binding affinity for dopamine that is at least about 50 percent or at least about 75 percent the binding affinity of the primary aptamer having SEQ ID NO:202). In certain non-limiting embodiments, a dopamine-binding primary aptamer comprising a core sequence as set forth in SEQ ID NO:119 (FIG. 28B) competes with primary aptamer having SEQ ID NO:202 for dopamine binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a dopamine-binding primary aptamer comprises a core sequence as set forth in SEQ ID NO:119 (FIG. 28B) and further comprises at least one operative sequence. In certain non-limiting embodiments, a dopamine-binding primary aptamer comprises a core sequence as set forth in SEQ ID NO:119 (FIG. 28B) and further comprises at least one operative sequence, said operative sequence complementary to a sequence comprised in a sensor oligonucleotide. In certain non-limiting embodiments, a dopamine-binding primary aptamer comprises a core sequence as set forth in SEQ ID NO:119 (FIG. 28B) and at least one operative sequence on either side (flanking) the core sequence. In certain non-limiting embodiments, a dopamine-binding primary aptamer comprises a core sequence as set forth in SEQ ID NO:119 (FIG. 28B) and at least one operative sequence on either side (flanking) the core sequence, where two of said operative sequences contain mutually complementary portions and can form a duplex.

In certain non-limiting embodiments, isolated dopamine-binding primary aptamers comprise the nucleotide sequence of SEQ ID NO:21, SEQ ID NO:196, SEQ ID NO:22, SEQ ID NO: 197, SEQ ID NO:23, SEQ ID NO:200, SEQ ID NO:20, SEQ ID NO:201, SEQ ID NO:19, SEQ ID NO:202 or variants of these sequences having at least about 80 percent, or at least about 85 percent, or at least about 90 percent, or at least about 95 percent, or at least about 98 percent homology to the original sequence, for example obtained by substitutions, deletions, and insertions of Watson-Crick base pairs or by mutations at non-conserved positions. Percent homology can be determined using standard software such as BLAST or FASTA. In certain non-limiting embodiments, isolated dopamine-binding primary aptamers comprise the nucleotide sequence of SEQ ID NO:21, SEQ ID NO:196, SEQ ID NO:22, SEQ ID NO: 197, SEQ ID NO:23, SEQ ID NO:200, SEQ ID NO:20, SEQ ID NO:201, SEQ ID NO:19, or SEQ ID NO:202. Said aptamers can bind to dopamine and in their structure-switching formats (for example, in an anti-aptamer assay, a pseudosandwich assay, or a sandwich assay) they can respond by an increase in fluorescence.

5.1.10 Serotonin-Binding Primary Aptamers

In certain non-limiting embodiments, a primary aptamer binds to serotonin in an aqueous solution at room temperature or 25° C. with a dissociation constant of less than 10⁻⁴ M and binds selectively with serotonin versus dopamine, melatonin, and 5-hydroxytryptophan.

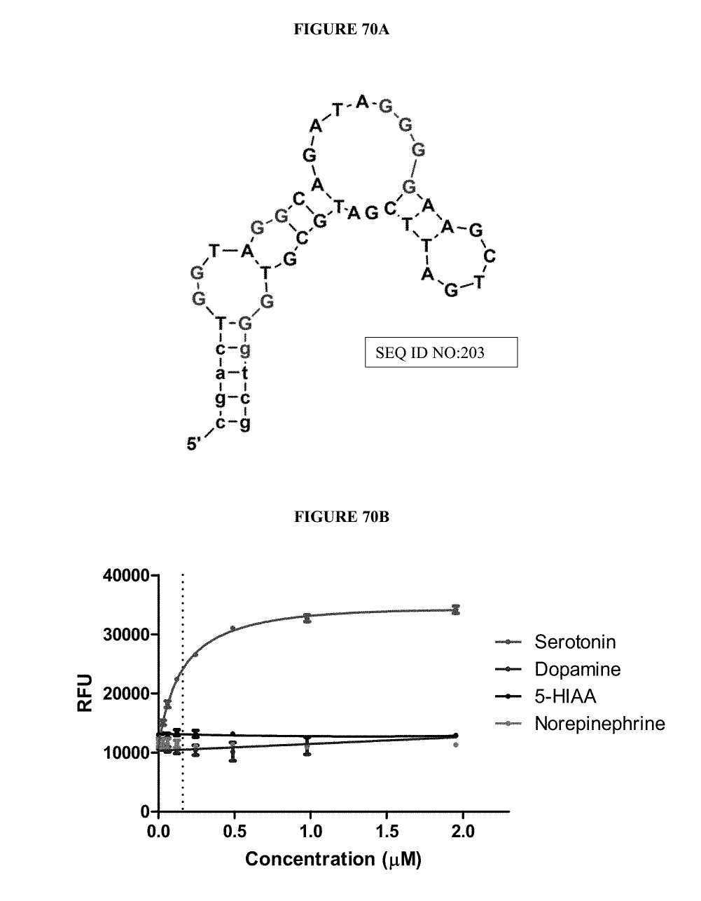

In certain non-limiting embodiments, a serotonin-binding primary aptamer comprises the sequences GG and GGGG (SEQ ID NO:169) and GGG or a variant of any of these sequences that differs in one or two bases by substitution, deletion, insertion or extension, where said primary aptamer binds to serotonin in an aqueous solution at room temperature or 25° C. with a dissociation constant of less than 10⁻⁴ M and binds to serotonin selectively versus dopamine, melatonin, or 5-hydroxytryptophan (FIGS. 70B, 71B, 72B, 73B, 74B, 75B, 76B). In certain non-limiting embodiments, a serotonin-binding primary aptamer comprises the sequences GG and GGGG (SEQ ID NO:169) and GGG or a variant thereof and further comprises at least one operative sequence. In certain non-limiting embodiments, a serotonin-binding primary aptamer comprises the sequences GG and GGGG (SEQ ID NO:169) and GGG or a variant thereof, and further comprises at least one operative sequence, said operative sequence complementary to a sequence comprised in a sensor oligonucleotide. In certain non-limiting embodiments, a serotonin-binding primary aptamer comprises the sequences GG and GGGG (SEQ ID NO:169) and GGG, or a variant thereof, and at least one operative sequence on either side (flanking) these three sequences. In certain non-limiting embodiments, a serotonin-binding primary aptamer comprises the sequences GG and GGGG (SEQ ID NO:169) and GGG or a variant thereof, and at least one operative sequence on either side (flanking) these three sequences, where two of said operative sequences contain mutually complementary portions and can form a duplex.

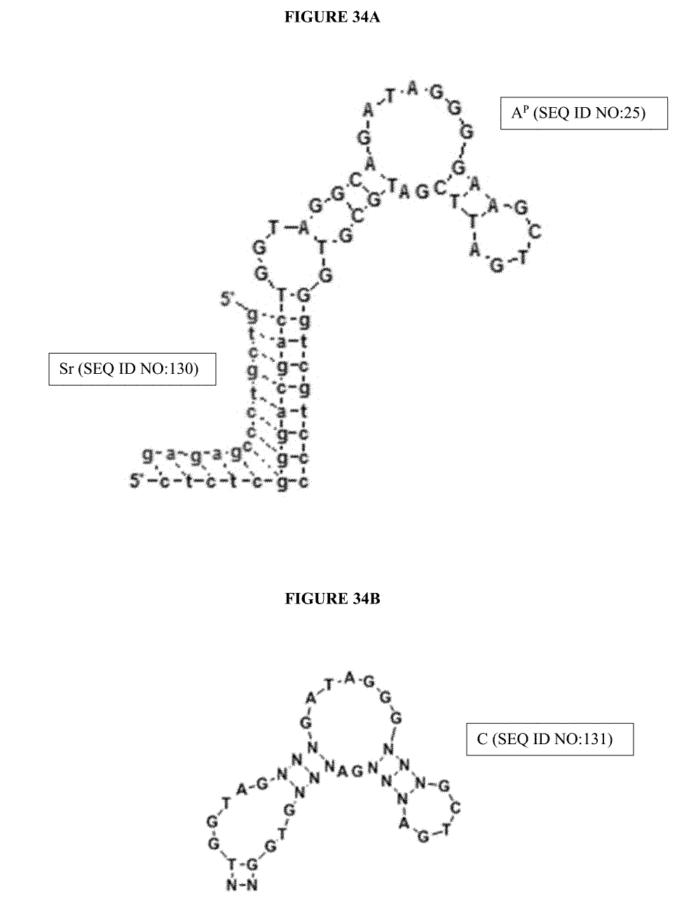

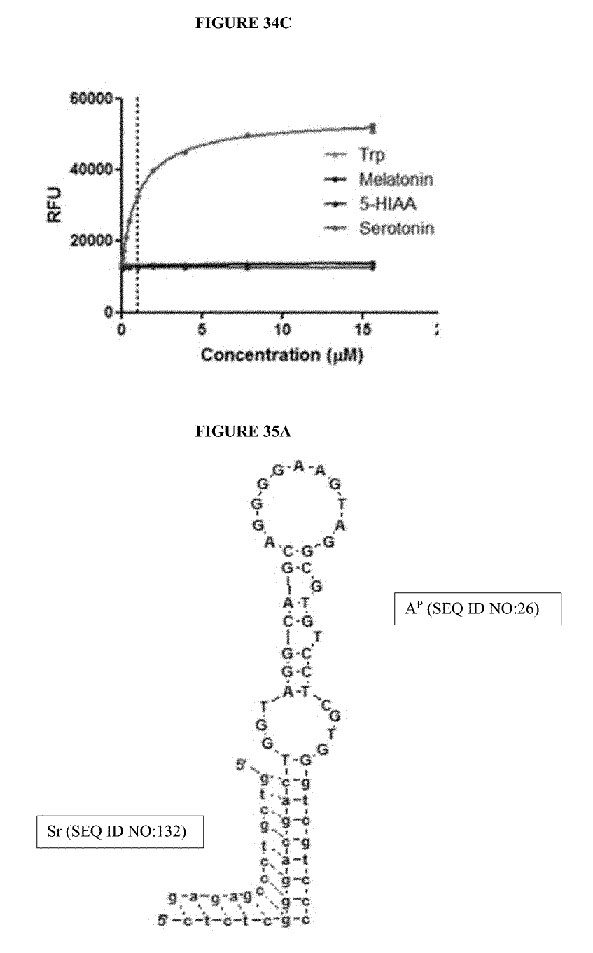

For example, but not by way of limitation, a serotonin-binding aptamer may comprise the sequence: CTC TCG GGA CGA CTG GTA GGC AGA TAG GGG AAG CTG ATT CGA TGC GTG GGT CGT CCC (SEQ ID NO:25; FIG. 34A) or related sequence (SEQ ID NO:203, FIG. 70A). In certain non-limiting embodiments, a serotonin-binding primary aptamer comprises a core sequence as set forth in SEQ ID NO:131 (FIG. 34B; FIG. 70A). In certain non-limiting embodiments, a serotonin-binding primary aptamer comprising a core sequence as set forth in SEQ ID NO:131 (FIG. 34B) has a binding affinity for serotonin that is at least about 50 percent or at least about 75 percent the binding affinity of the primary aptamer having SEQ ID NO:203). In certain non-limiting embodiments, a serotonin-binding primary aptamer comprising a core sequence as set forth in SEQ ID NO: 131 (FIG. 34B) competes with primary aptamer having SEQ ID NO:203 for serotonin binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a serotonin-binding primary aptamer comprises a core sequence as set forth in SEQ ID NO: 131 (FIG. 34B) and further comprises at least one operative sequence. In certain non-limiting embodiments, a serotonin-binding primary aptamer comprises a core sequence as set forth in SEQ ID NO: 131 (FIG. 34B) and further comprises at least one operative sequence, said operative sequence complementary to a sequence comprised in a sensor oligonucleotide. In certain non-limiting embodiments, a serotonin-binding primary aptamer comprises a core sequence as set forth in SEQ ID NO: 131 (FIG. 34B) and at least one operative sequence on either side (flanking) the core sequence. In certain non-limiting embodiments, a serotonin-binding primary aptamer comprises a core sequence as set forth in SEQ ID NO: 131 (FIG. 34B) and at least one operative sequence on either side (flanking) the core sequence, where two of said operative sequences contain mutually complementary portions and can form a duplex.

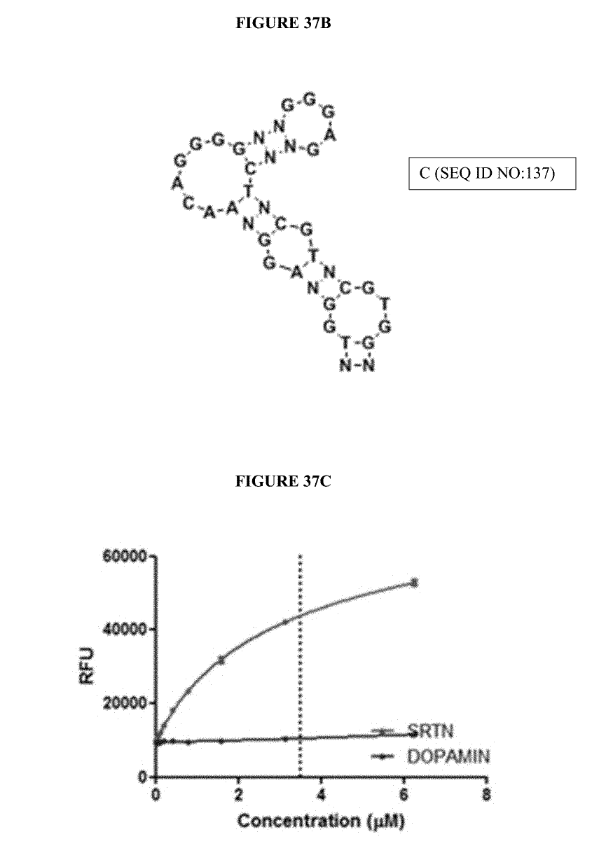

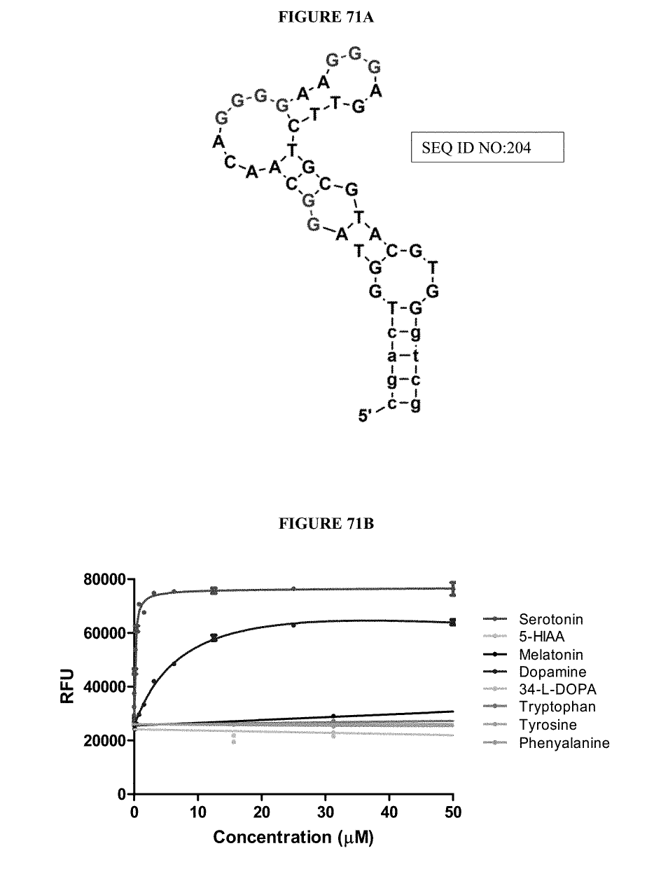

For example, but not by way of limitation, a serotonin-binding aptamer may comprise the sequence: CTC TCG GGA CGA CTG GTA GGC AAC AGG GGA AGG GAG TTC TGC GTA CGT GGG TCG TCC C (SEQ ID NO:28; FIG. 37A) or related sequence (SEQ ID NO:204, FIG. 71A). In certain non-limiting embodiments, a serotonin-binding primary aptamer comprises a core sequence as set forth in SEQ ID NO:137 (FIG. 37B; FIG. 71A). In certain non-limiting embodiments, a serotonin-binding primary aptamer comprising a core sequence as set forth in SEQ ID NO: 137 (FIG. 37B) has a binding affinity for serotonin that is at least about 50 percent or at least about 75 percent the binding affinity of the primary aptamer having SEQ ID NO:204). In certain non-limiting embodiments, a serotonin-binding primary aptamer comprising a core sequence as set forth in SEQ ID NO: 137 (FIG. 37B) competes with primary aptamer having SEQ ID NO:204 for serotonin binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a serotonin-binding primary aptamer comprises a core sequence as set forth in SEQ ID NO: 137 (FIG. 37B) and further comprises at least one operative sequence. In certain non-limiting embodiments, a serotonin-binding primary aptamer comprises a core sequence as set forth in SEQ ID NO: 137 (FIG. 37B) and further comprises at least one operative sequence, said operative sequence complementary to a sequence comprised in a sensor oligonucleotide. In certain non-limiting embodiments, a serotonin-binding primary aptamer comprises a core sequence as set forth in SEQ ID NO: 137 (FIG. 37 B) and at least one operative sequence on either side (flanking) the core sequence. In certain non-limiting embodiments, a serotonin-binding primary aptamer comprises a core sequence as set forth in SEQ ID NO: 137 (FIG. 37B) and at least one operative sequence on either side (flanking) the core sequence, where two of said operative sequences contain mutually complementary portions and can form a duplex.

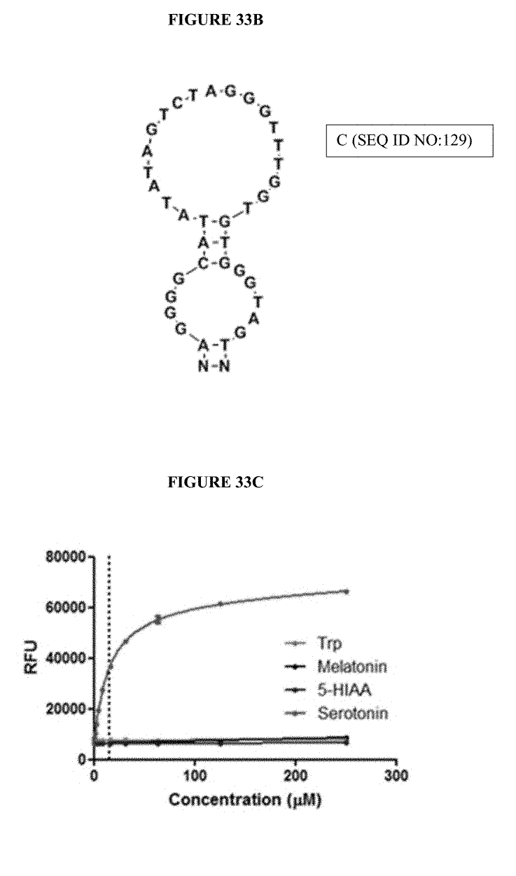

For example, but not by way of limitation, a serotonin-binding aptamer may comprise the sequence: CTC TCG GGA CGA CAG GGG CAT ATA TAG TCT AGG GTT TGG TGT GGG TAG TGT CGT CCC (SEQ ID NO:24; FIG. 33A) or related sequence (SEQ ID NO:205, FIG. 72A). In certain non-limiting embodiments, a serotonin-binding primary aptamer comprises a core sequence as set forth in SEQ ID NO:129 (FIG. 33B; FIG. 72A). In certain non-limiting embodiments, a serotonin-binding primary aptamer comprising a core sequence as set forth in SEQ ID NO: 129 (FIG. 33B) has a binding affinity for serotonin that is at least about 50 percent or at least about 75 percent the binding affinity of the primary aptamer having SEQ ID NO:205). In certain non-limiting embodiments, a serotonin-binding primary aptamer comprising a core sequence as set forth in SEQ ID NO: 129 (FIG. 33B) competes with primary aptamer having SEQ ID NO:205 for serotonin binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a serotonin-binding primary aptamer comprises a core sequence as set forth in SEQ ID NO: 129 (FIG. 33B) and further comprises at least one operative sequence. In certain non-limiting embodiments, a serotonin-binding primary aptamer comprises a core sequence as set forth in SEQ ID NO: 129 (FIG. 33B and further comprises at least one operative sequence, said operative sequence complementary to a sequence comprised in a sensor oligonucleotide. In certain non-limiting embodiments, a serotonin-binding primary aptamer comprises a core sequence as set forth in SEQ ID NO: 129 (FIG. 33B) and at least one operative sequence on either side (flanking) the core sequence. In certain non-limiting embodiments, a serotonin-binding primary aptamer comprises a core sequence as set forth in SEQ ID NO: 129 (FIG. 33B) and at least one operative sequence on either side (flanking) the core sequence, where two of said operative sequences contain mutually complementary portions and can form a duplex.

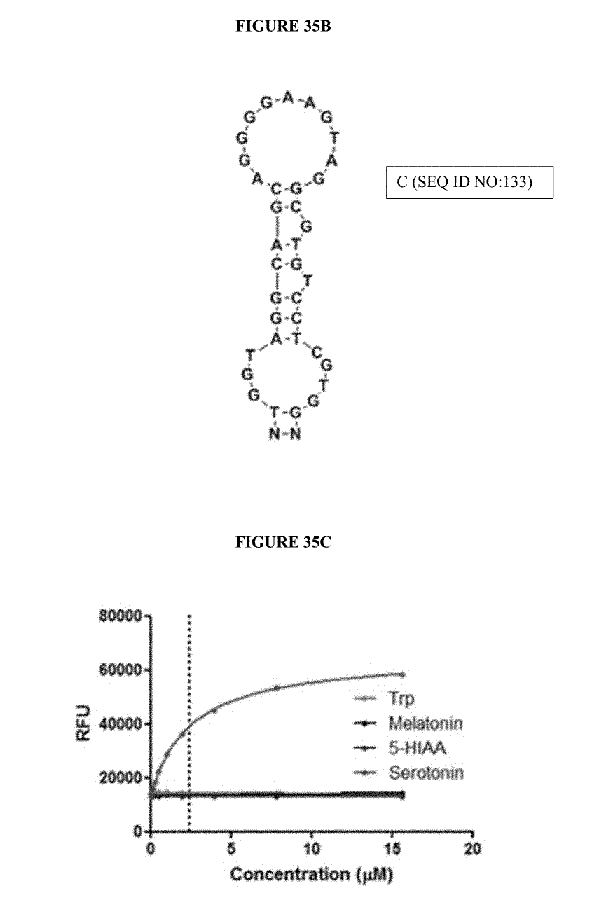

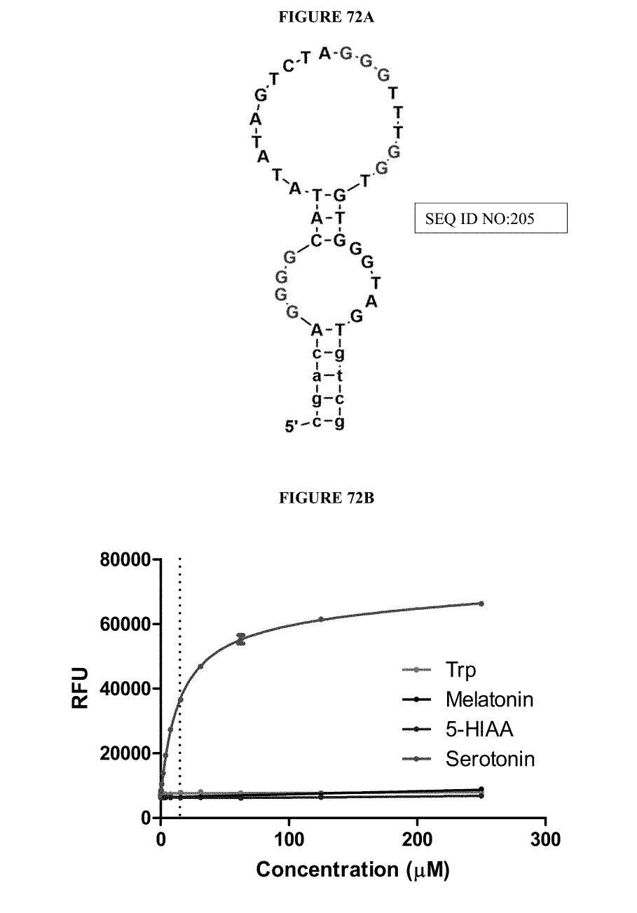

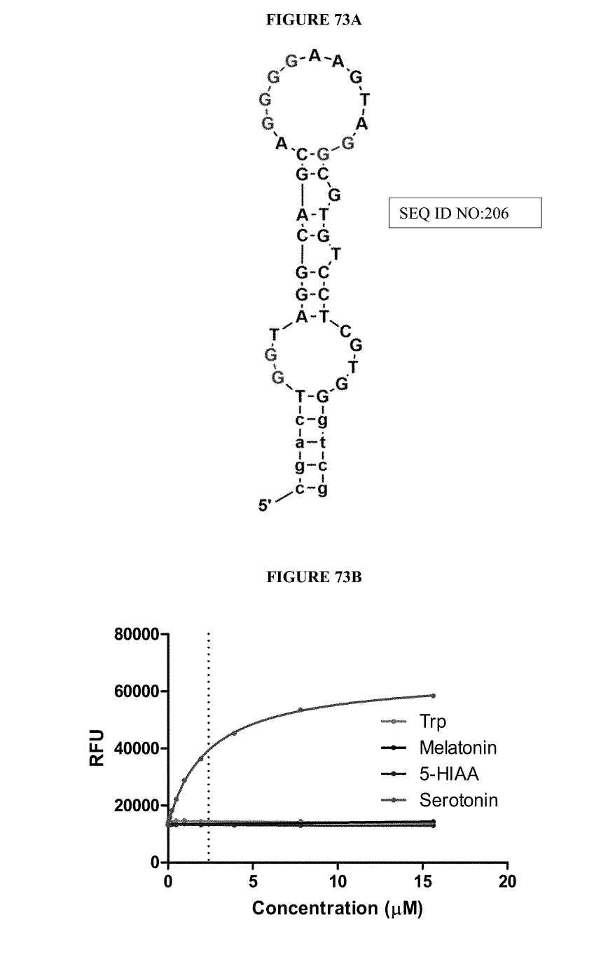

For example, but not by way of limitation, a serotonin-binding aptamer may comprise the sequence: CTC TCG GGA CGA CTG GTA GGC AGC AGG GGA AGT AGG CGT GTC CTC GTG GGT CGT CCC (SEQ ID NO:26; FIG. 35A) or related sequence (SEQ ID NO:206, FIG. 73A). In certain non-limiting embodiments, a serotonin-binding primary aptamer comprises a core sequence as set forth in SEQ ID NO:133 (FIG. 35B; FIG. 73A). In certain non-limiting embodiments, a serotonin-binding primary aptamer comprising a core sequence as set forth in SEQ ID NO: 133 (FIG. 35B) has a binding affinity for serotonin that is at least about 50 percent or at least about 75 percent the binding affinity of the primary aptamer having SEQ ID NO:206). In certain non-limiting embodiments, a serotonin-binding primary aptamer comprising a core sequence as set forth in SEQ ID NO: 133 (FIG. 35B) competes with primary aptamer having SEQ ID NO:206 for serotonin binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a serotonin-binding primary aptamer comprises a core sequence as set forth in SEQ ID NO: 133 (FIG. 35B) and further comprises at least one operative sequence. In certain non-limiting embodiments, a serotonin-binding primary aptamer comprises a core sequence as set forth in SEQ ID NO: 133 (FIG. 35B and further comprises at least one operative sequence, said operative sequence complementary to a sequence comprised in a sensor oligonucleotide. In certain non-limiting embodiments, a serotonin-binding primary aptamer comprises a core sequence as set forth in SEQ ID NO: 133 (FIG. 35B) and at least one operative sequence on either side (flanking) the core sequence. In certain non-limiting embodiments, a serotonin-binding primary aptamer comprises a core sequence as set forth in SEQ ID NO: 133 (FIG. 35 B) and at least one operative sequence on either side (flanking) the core sequence, where two of said operative sequences contain mutually complementary portions and can form a duplex.

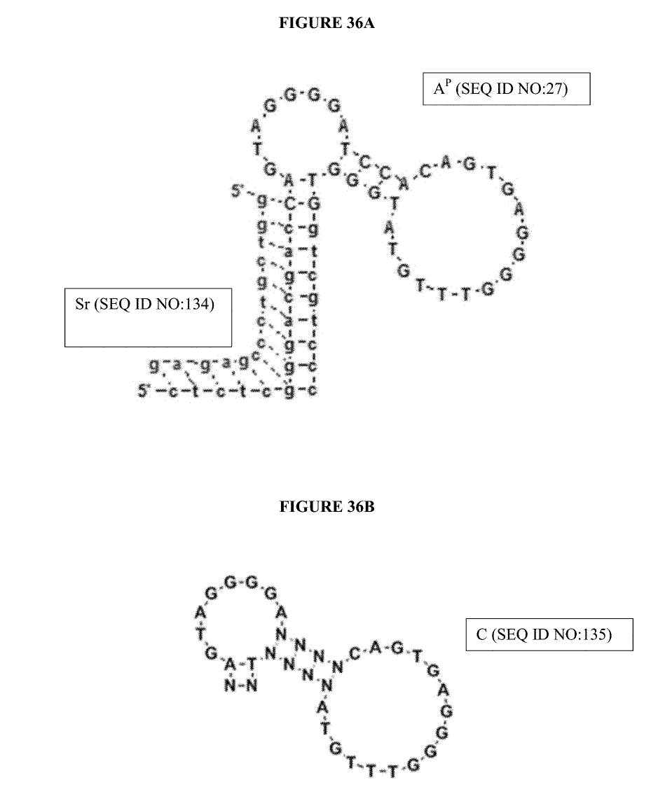

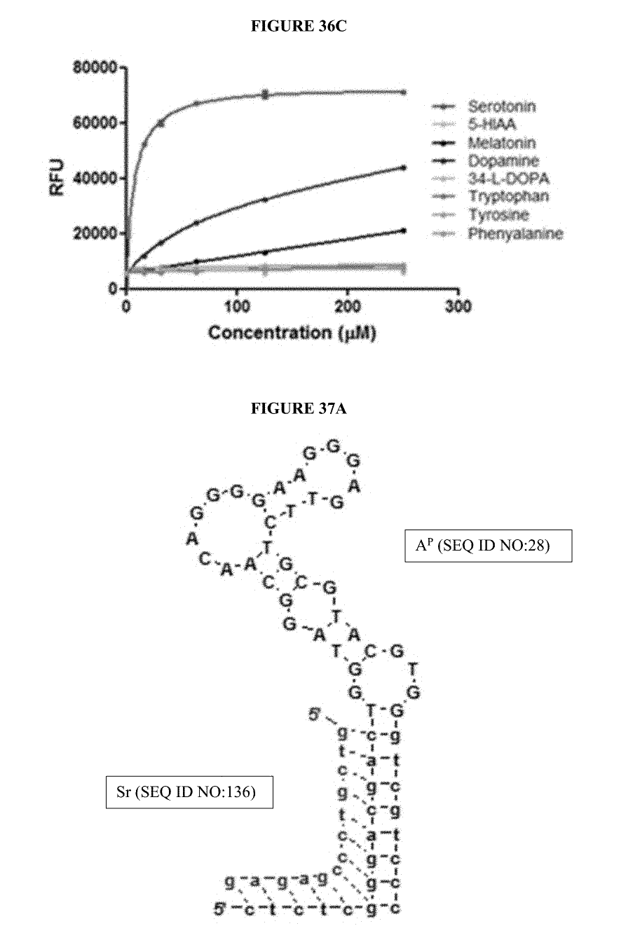

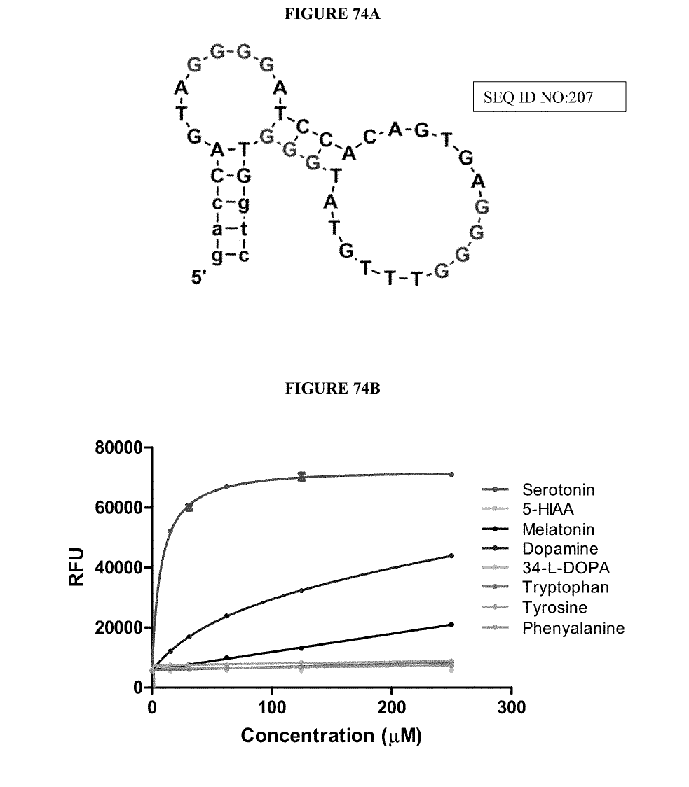

For example, but not by way of limitation, a serotonin-binding aptamer may comprise the sequence: CTC TCG GGA CGA CCA GTA GGG GAT CCA CAG TGA GGG GTT TGT ATG GGT GGT CGT CCC (SEQ ID NO:27; FIG. 36A) or related sequence (SEQ ID NO:207, FIG. 74A). In certain non-limiting embodiments, a serotonin-binding primary aptamer comprises a core sequence as set forth in SEQ ID NO:135 (FIG. 36B; FIG. 74A). In certain non-limiting embodiments, a serotonin-binding primary aptamer comprising a core sequence as set forth in SEQ ID NO: 135 (FIG. 36B) has a binding affinity for serotonin that is at least about 50 percent or at least about 75 percent the binding affinity of the primary aptamer having SEQ ID NO:207). In certain non-limiting embodiments, a serotonin-binding primary aptamer comprising a core sequence as set forth in SEQ ID NO: 135 (FIG. 36B) competes with primary aptamer having SEQ ID NO:207 for serotonin binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a serotonin-binding primary aptamer comprises a core sequence as set forth in SEQ ID NO: 135 (FIG. 36B) and further comprises at least one operative sequence. In certain non-limiting embodiments, a serotonin-binding primary aptamer comprises a core sequence as set forth in SEQ ID NO: 135 (FIG. 36B and further comprises at least one operative sequence, said operative sequence complementary to a sequence comprised in a sensor oligonucleotide. In certain non-limiting embodiments, a serotonin-binding primary aptamer comprises a core sequence as set forth in SEQ ID NO: 135 (FIG. 36B) and at least one operative sequence on either side (flanking) the core sequence. In certain non-limiting embodiments, a serotonin-binding primary aptamer comprises a core sequence as set forth in SEQ ID NO: 135 (FIG. 36 B) and at least one operative sequence on either side (flanking) the core sequence, where two of said operative sequences contain mutually complementary portions and can form a duplex.

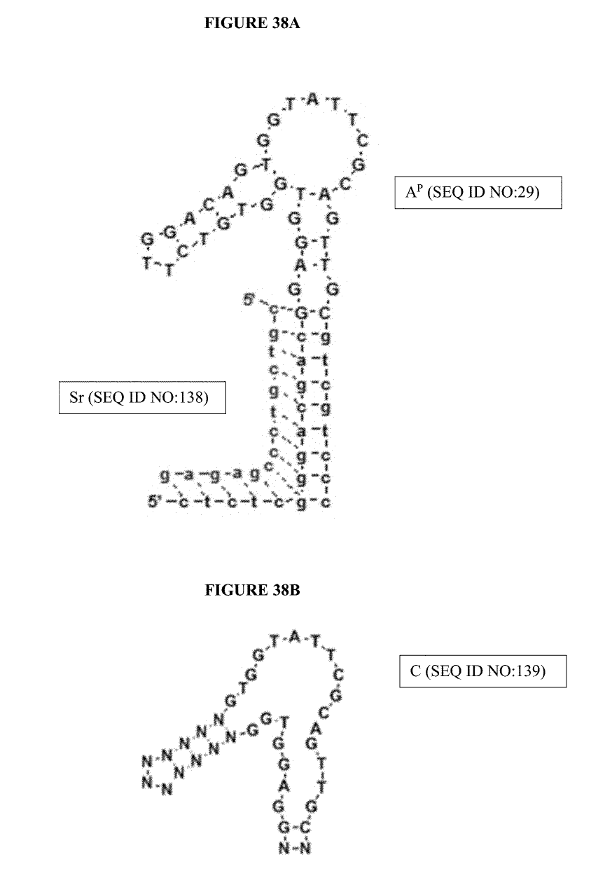

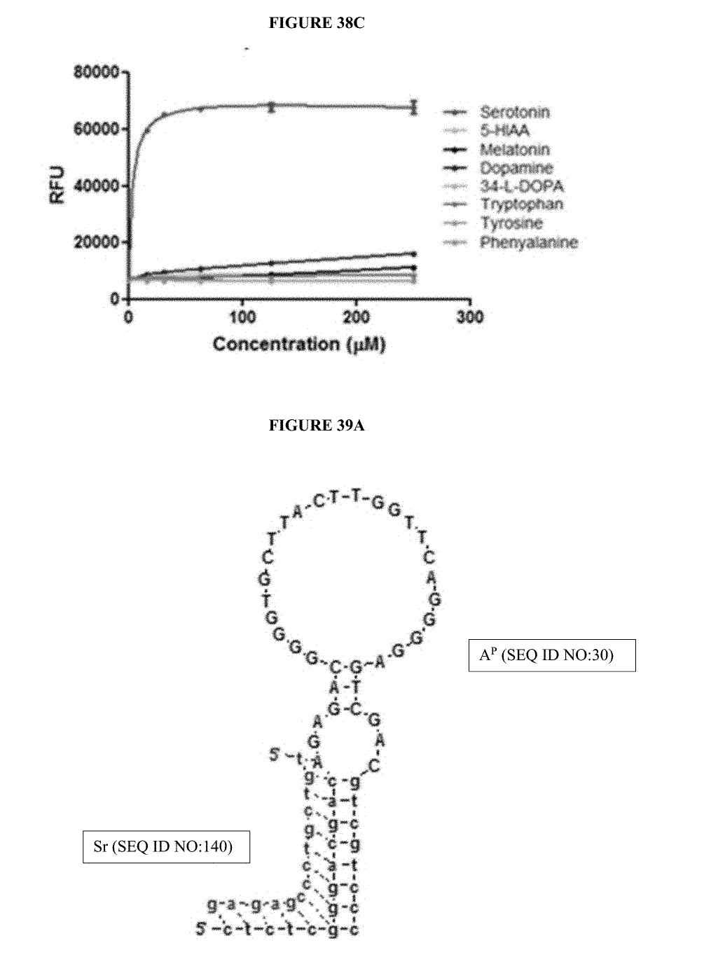

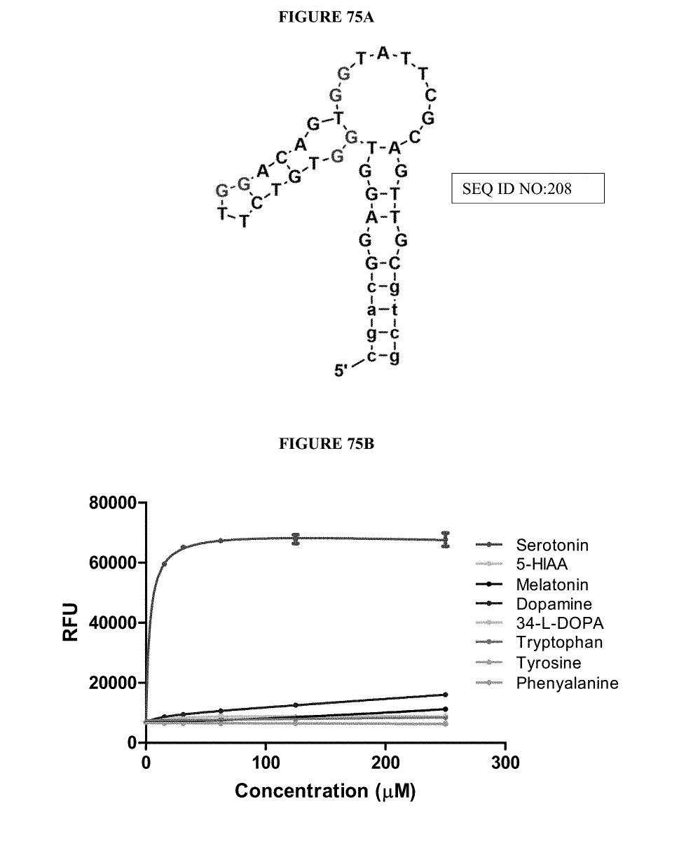

For example, but not by way of limitation, a serotonin-binding aptamer may comprise the sequence: CTC TCG GGA CGA CGG AGG TGG TGT CTT GGA CAG TGG TAT TCG CAG TTG CGT CGT CCC (SEQ ID NO:29; FIG. 38A) or related sequence (SEQ ID NO:208, FIG. 75A). In certain non-limiting embodiments, a serotonin-binding primary aptamer comprises a core sequence as set forth in SEQ ID NO:139 (FIG. 38B; FIG. 75A). In certain non-limiting embodiments, a serotonin-binding primary aptamer comprising a core sequence as set forth in SEQ ID NO: 139 (FIG. 38B) has a binding affinity for serotonin that is at least about 50 percent or at least about 75 percent the binding affinity of the primary aptamer having SEQ ID NO:208). In certain non-limiting embodiments, a serotonin-binding primary aptamer comprising a core sequence as set forth in SEQ ID NO: 139 (FIG. 38B) competes with primary aptamer having SEQ ID NO:208 for serotonin binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a serotonin-binding primary aptamer comprises a core sequence as set forth in SEQ ID NO: 139 (FIG. 38B) and further comprises at least one operative sequence. In certain non-limiting embodiments, a serotonin-binding primary aptamer comprises a core sequence as set forth in SEQ ID NO: 139 (FIG. 38B and further comprises at least one operative sequence, said operative sequence complementary to a sequence comprised in a sensor oligonucleotide. In certain non-limiting embodiments, a serotonin-binding primary aptamer comprises a core sequence as set forth in SEQ ID NO: 139 (FIG. 38B) and at least one operative sequence on either side (flanking) the core sequence. In certain non-limiting embodiments, a serotonin-binding primary aptamer comprises a core sequence as set forth in SEQ ID NO: 139 (FIG. 38 B) and at least one operative sequence on either side (flanking) the core sequence, where two of said operative sequences contain mutually complementary portions and can form a duplex.

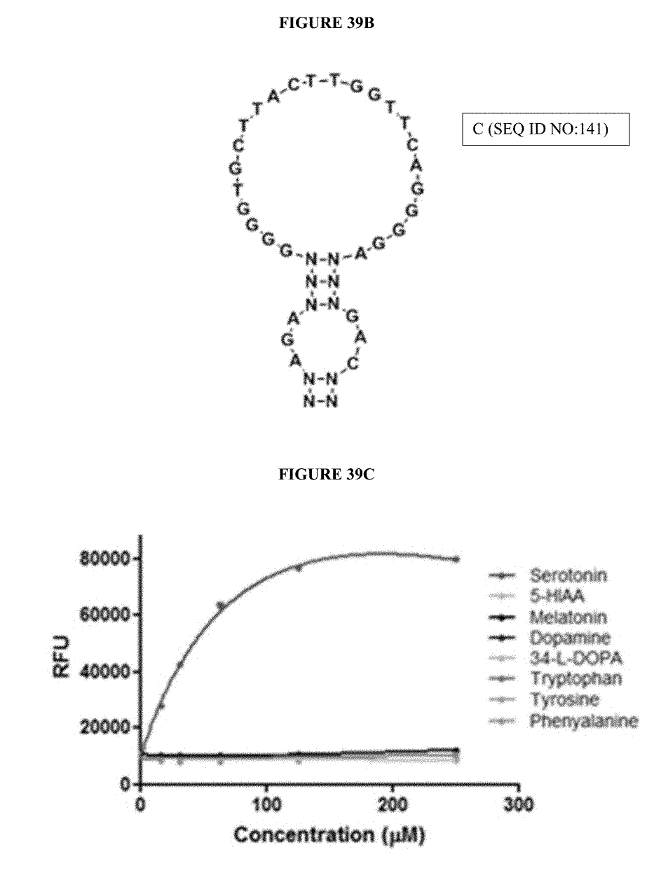

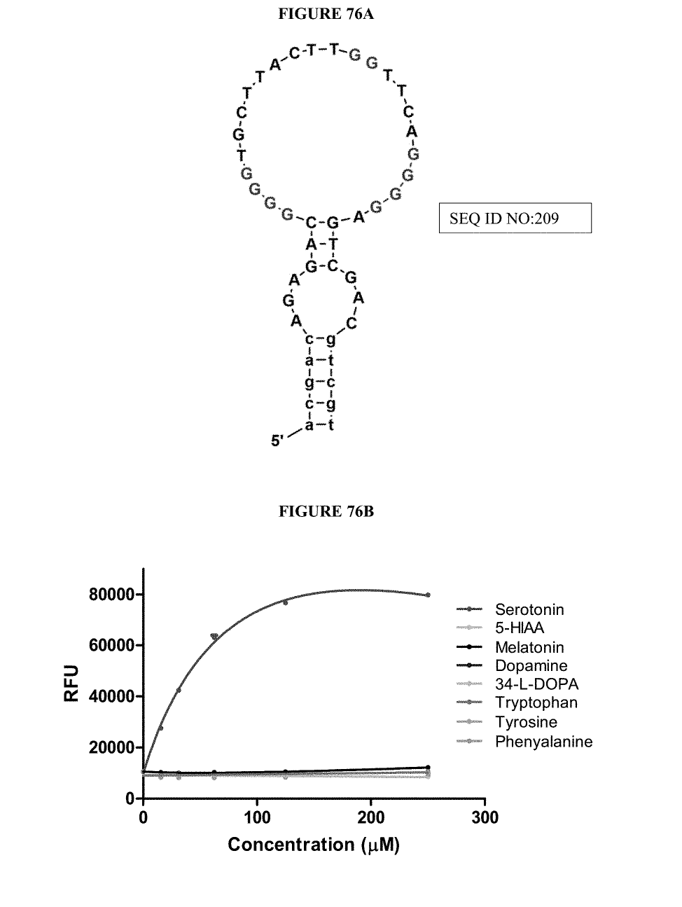

For example, but not by way of limitation, a serotonin-binding aptamer may comprise the sequence: CTC TCG GGA CGA CAG AGA CGG GGT GCT TAC TTG GTT CAG GGG AGT CGA CGT CGT CCC (SEQ ID NO:30; FIG. 39A) or related sequence (SEQ ID NO:209, FIG. 76A). In certain non-limiting embodiments, a serotonin-binding primary aptamer comprises a core sequence as set forth in SEQ ID NO:141 (FIG. 39B; FIG. 76A). In certain non-limiting embodiments, a serotonin-binding primary aptamer comprising a core sequence as set forth in SEQ ID NO: 141 (FIG. 39B) has a binding affinity for serotonin that is at least about 50 percent or at least about 75 percent the binding affinity of the primary aptamer having SEQ ID NO:209). In certain non-limiting embodiments, a serotonin-binding primary aptamer comprising a core sequence as set forth in SEQ ID NO: 141 (FIG. 39B) competes with primary aptamer having SEQ ID NO:209 for serotonin binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a serotonin-binding primary aptamer comprises a core sequence as set forth in SEQ ID NO: 141 (FIG. 39B) and further comprises at least one operative sequence. In certain non-limiting embodiments, a serotonin-binding primary aptamer comprises a core sequence as set forth in SEQ ID NO: 141 (FIG. 39B and further comprises at least one operative sequence, said operative sequence complementary to a sequence comprised in a sensor oligonucleotide. In certain non-limiting embodiments, a serotonin-binding primary aptamer comprises a core sequence as set forth in SEQ ID NO: 141 (FIG. 39B) and at least one operative sequence on either side (flanking) the core sequence. In certain non-limiting embodiments, a serotonin-binding primary aptamer comprises a core sequence as set forth in SEQ ID NO: 139 (FIG. 38 B) and at least one operative sequence on either side (flanking) the core sequence, where two of said operative sequences contain mutually complementary portions and can form a duplex.

In certain non-limiting embodiments, isolated serotonin-binding primary aptamers comprise the nucleotide sequence of SEQ ID NO:25, SEQ ID NO:203, SEQ ID NO:28, SEQ ID NO:204, SEQ ID NO:24, SEQ ID NO:205, SEQ ID NO:26, SEQ ID NO:206, SEQ ID NO:27, SEQ ID NO:207, SEQ ID NO:29, SEQ ID NO: 208, SEQ ID NO:30, SEQ ID NO:209, or variants of these sequences having at least about 80 percent, or at least about 85 percent, or at least about 90 percent, or at least about 95 percent, or at least about 98 percent homology to the original sequence, for example obtained by substitutions, deletions, and insertions of Watson-Crick base pairs or by mutations at non-conserved positions. Percent homology can be determined using standard software such as BLAST or FASTA. In certain non-limiting embodiments, isolated serotonin-binding primary aptamers comprise the nucleotide sequence of SEQ ID NO:25, SEQ ID NO:203, SEQ ID NO:28, SEQ ID NO:204, SEQ ID NO:24, SEQ ID NO:205, SEQ ID NO:26, SEQ ID NO:206, SEQ ID NO:27, SEQ ID NO:207, SEQ ID NO:29, SEQ ID NO: 208, SEQ ID NO:30, or SEQ ID NO:209. Said aptamers can bind to serotonin and in their structure-switching formats (for example, in an anti-aptamer assay, a pseudosandwich assay, or a sandwich assay) they can respond by an increase in fluorescence.

5.1.11 Melatonin-Binding Primary Aptamers

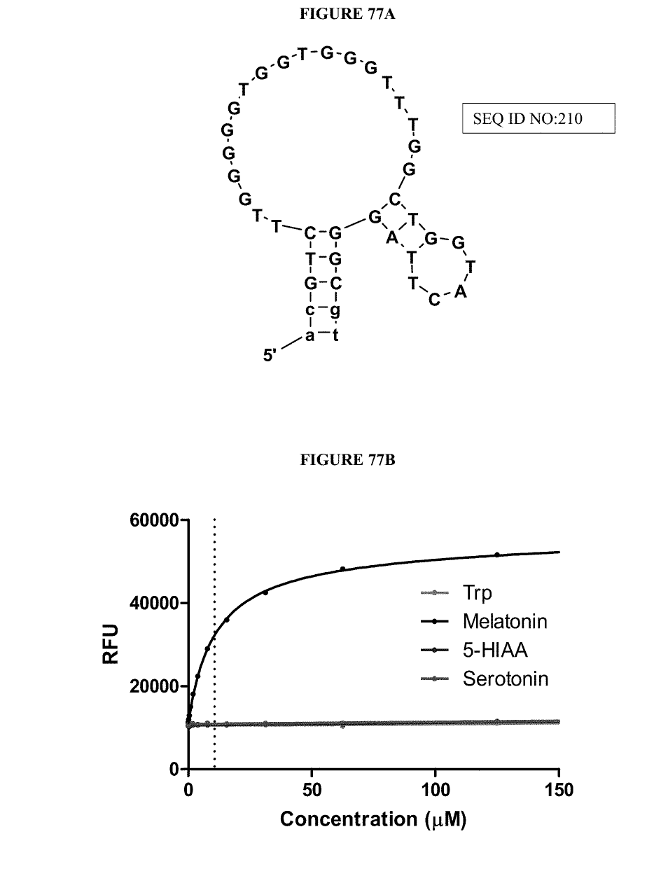

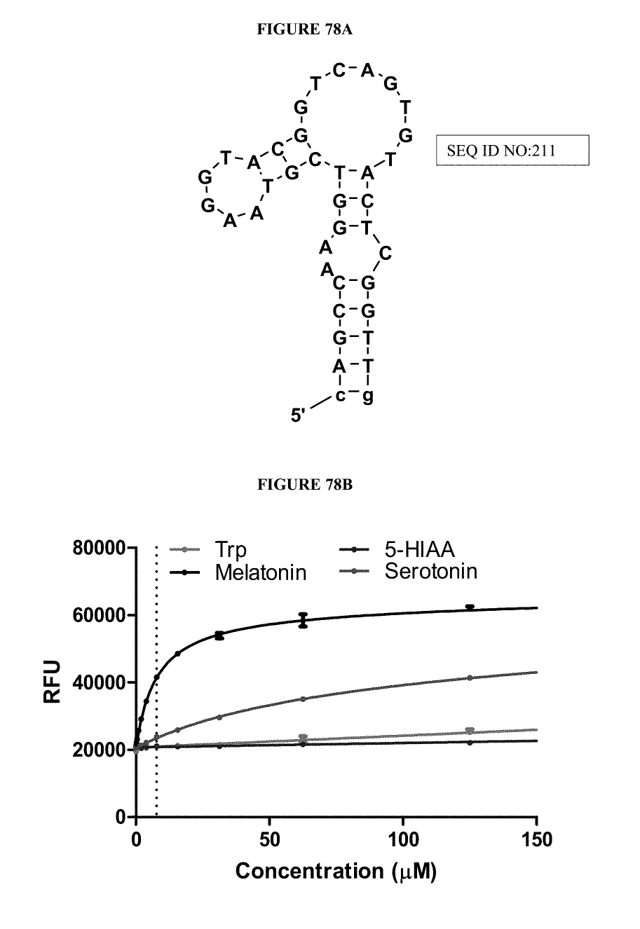

In certain non-limiting embodiments, a primary aptamer binds to melatonin in an aqueous solution at room temperature or 25° C. with a dissociation constant of less than 10⁻⁴ M and binds selectively with melatonin versus serotonin or tryptophan (see FIGS. 77B and 78B).

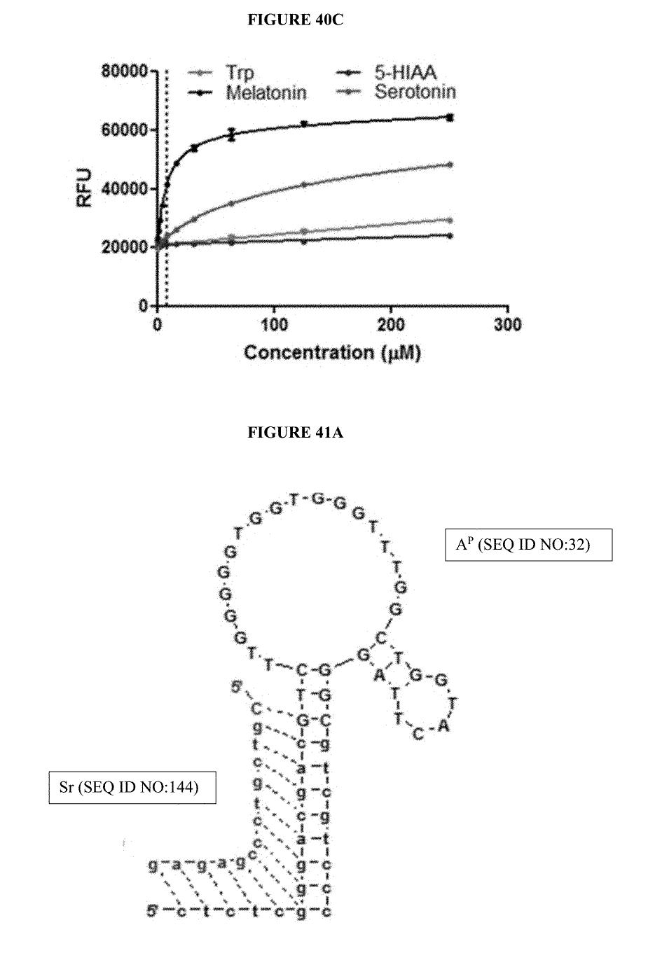

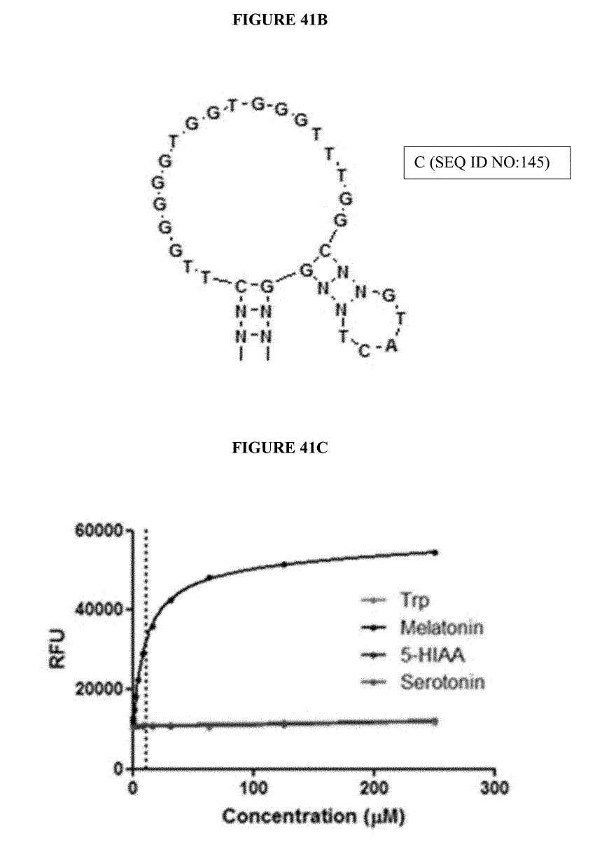

For example, but not by way of limitation, a melatonin-binding aptamer may comprise the sequence: CTC TCG GGA CGA CGT CTT GGG GGT GGT GGG TTT GGC TGG TAC TTA GGG CGT CGT CCC (SEQ ID NO:32; FIG. 41A) or related sequence (SEQ ID NO:210, FIG. 77A). In certain non-limiting embodiments, a melatonin-binding primary aptamer comprises a core sequence as set forth in SEQ ID NO:145 (FIG. 41B; FIG. 77A). In certain non-limiting embodiments, a melatonin-binding primary aptamer comprising a core sequence as set forth in SEQ ID NO: 145 (FIG. 41B) has a binding affinity for melatonin that is at least about 50 percent or at least about 75 percent the binding affinity of the primary aptamer having SEQ ID NO:210). In certain non-limiting embodiments, a melatonin-binding primary aptamer comprising a core sequence as set forth in SEQ ID NO: 145 (FIG. 41B) competes with primary aptamer having SEQ ID NO:210 for melatonin binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a melatonin-binding primary aptamer comprises a core sequence as set forth in SEQ ID NO: 145 (FIG. 41B) and further comprises at least one operative sequence. In certain non-limiting embodiments, a melatonin-binding primary aptamer comprises a core sequence as set forth in SEQ ID NO: 145 (FIG. 41B and further comprises at least one operative sequence, said operative sequence complementary to a sequence comprised in a sensor oligonucleotide. In certain non-limiting embodiments, a melatonin-binding primary aptamer comprises a core sequence as set forth in SEQ ID NO: 145 (FIG. 41B) and at least one operative sequence on either side (flanking) the core sequence. In certain non-limiting embodiments, a melatonin-binding primary aptamer comprises a core sequence as set forth in SEQ ID NO: 145 (FIG. 41 B) and at least one operative sequence on either side (flanking) the core sequence, where two of said operative sequences contain mutually complementary portions and can form a duplex.

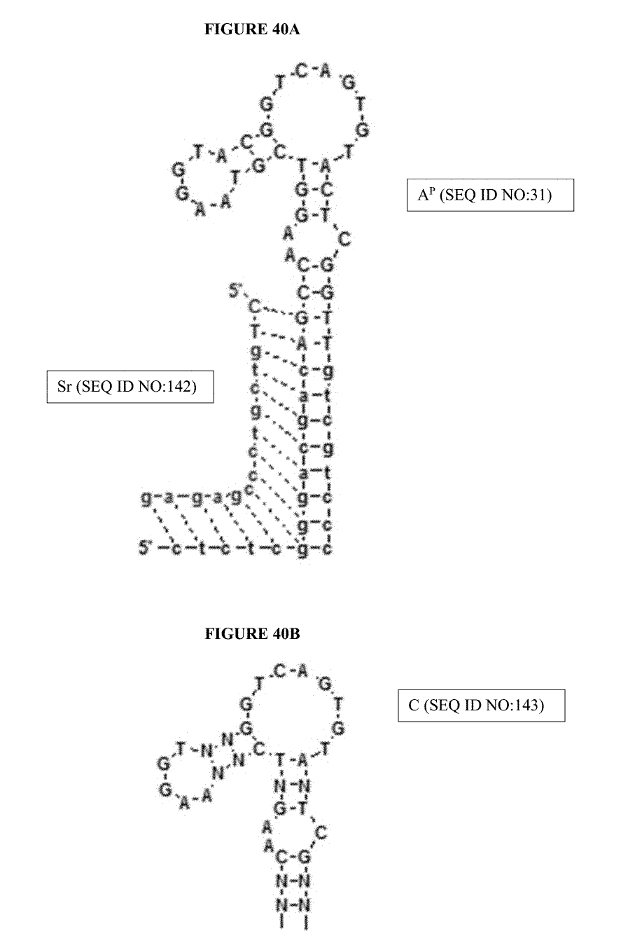

For example, but not by way of limitation, a melatonin-binding aptamer may comprise the sequence: CTC TCG GGA CGA CAG CCA AGG TCG TAA GGT ACG GTC AGT GTA CTC GGT TGT CGT CCC (SEQ ID NO:31; FIG. 40A) or related sequence (SEQ ID NO:211, FIG. 78A). In certain non-limiting embodiments, a melatonin-binding primary aptamer comprises a core sequence as set forth in SEQ ID NO:143 (FIG. 40B; FIG. 78A). In certain non-limiting embodiments, a melatonin-binding primary aptamer comprising a core sequence as set forth in SEQ ID NO: 143 (FIG. 40B) has a binding affinity for melatonin that is at least about 50 percent or at least about 75 percent the binding affinity of the primary aptamer having SEQ ID NO:211). In certain non-limiting embodiments, a melatonin-binding primary aptamer comprising a core sequence as set forth in SEQ ID NO: 143 (FIG. 40B) competes with primary aptamer having SEQ ID NO:211 for melatonin binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a melatonin-binding primary aptamer comprises a core sequence as set forth in SEQ ID NO: 143 (FIG. 40B) and further comprises at least one operative sequence. In certain non-limiting embodiments, a melatonin-binding primary aptamer comprises a core sequence as set forth in SEQ ID NO: 143 (FIG. 40B and further comprises at least one operative sequence, said operative sequence complementary to a sequence comprised in a sensor oligonucleotide. In certain non-limiting embodiments, a melatonin-binding primary aptamer comprises a core sequence as set forth in SEQ ID NO: 143 (FIG. 40B) and at least one operative sequence on either side (flanking) the core sequence. In certain non-limiting embodiments, a melatonin-binding primary aptamer comprises a core sequence as set forth in SEQ ID NO: 143 (FIG. 40 B) and at least one operative sequence on either side (flanking) the core sequence, where two of said operative sequences contain mutually complementary portions and can form a duplex.

In certain non-limiting embodiments, isolated melatonin-binding primary aptamers comprise the nucleotide sequence of SEQ ID NO:31, SEQ ID NO:211, SEQ ID NO:32, SEQ ID NO:210, or variants of these sequences having at least about 80 percent, or at least about 85 percent, or at least about 90 percent, or at least about 95 percent, or at least about 98 percent homology to the original sequence, for example obtained by substitutions, deletions, and insertions of Watson-Crick base pairs or by mutations at non-conserved positions. Percent homology can be determined using standard software such as BLAST or FASTA. In certain non-limiting embodiments, isolated melatonin-binding primary aptamers comprise the nucleotide sequence of SEQ ID NO:31, SEQ ID NO:211, SEQ ID NO:32, or SEQ ID NO:210. Said aptamers can bind to melatonin and in their structure-switching formats (for example, in an anti-aptamer assay, a pseudosandwich assay, or a sandwich assay) they can respond by an increase in fluorescence.

5.1.12 Tyrosine-Binding Primary Aptamers

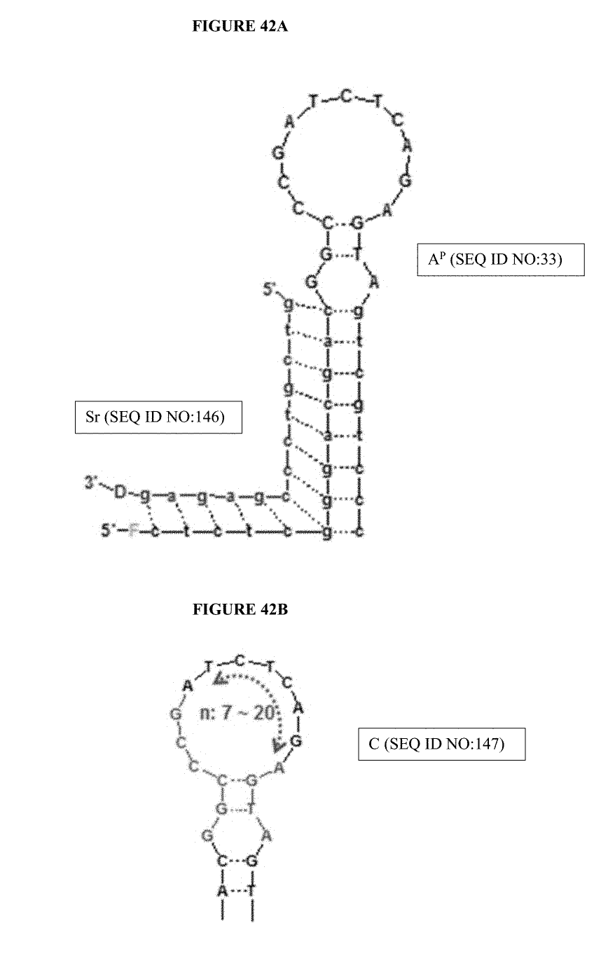

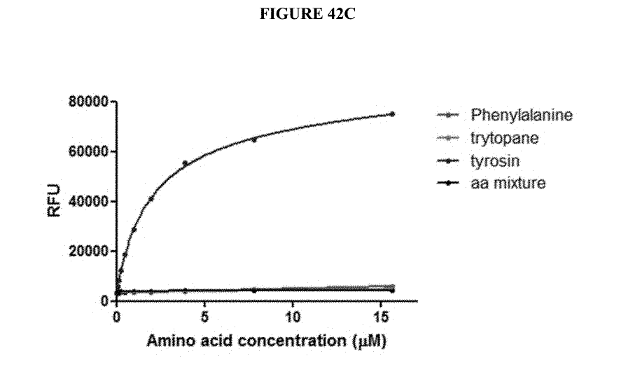

In certain non-limiting embodiments, a tyrosine-binding primary aptamer comprising a core sequence as set forth in SEQ ID NO: 147 (FIG. 42B) has a binding affinity for tyrosine that is at least about 50 percent or at least about 75 percent the binding affinity of the primary aptamer having SEQ ID NO:33). In certain non-limiting embodiments, a tyrosine-binding primary aptamer comprising a core sequence as set forth in SEQ ID NO: 147 (FIG. 42B) competes with primary aptamer having SEQ ID NO:33 for tyrosine binding. In non-limiting embodiments, said primary aptamer has a length of between about 30 and about 100 nucleotides, or between about 30 and 80 nucleotides, or between about 30 and 70 nucleotides, or between about 30 and 60 nucleotides. In certain non-limiting embodiments, a tyrosine-binding primary aptamer comprises a core sequence as set forth in SEQ ID NO: 147 (FIG. 42B) and further comprises at least one operative sequence. In certain non-limiting embodiments, a tyrosine-binding primary aptamer comprises a core sequence as set forth in SEQ ID NO: 147 (FIG. 42B) and further comprises at least one operative sequence, said operative sequence complementary to a sequence comprised in a sensor oligonucleotide. In certain non-limiting embodiments, a tyrosine-binding primary aptamer comprises a core sequence as set forth in SEQ ID NO: 147 (FIG. 42B) and at least one operative sequence on either side (flanking) the core sequence. In certain non-limiting embodiments, a tyrosine-binding primary aptamer comprises a core sequence as set forth in SEQ ID NO: 147 (FIG. 42 B) and at least one operative sequence on either side (flanking) the core sequence, where two of said operative sequences contain mutually complementary portions and can form a duplex.

In certain non-limiting embodiments, isolated tyrosine-binding primary aptamers comprise the nucleotide sequence of SEQ ID NO:33, SEQ ID NO:147, or variants of these sequences having at least about 80 percent, or at least about 85 percent, or at least about 90 percent, or at least about 95 percent, or at least about 98 percent homology to the original sequence, for example obtained by substitutions, deletions, and insertions of Watson-Crick base pairs or by mutations at non-conserved positions. Percent homology can be determined using standard software such as BLAST or FASTA. In certain non-limiting embodiments, isolated tyrosine-binding primary aptamers comprise the nucleotide sequence of SEQ ID NO:33 or SEQ ID NO:147. Said aptamers can bind to tyrosine and in their structure-switching formats (for example, in an anti-aptamer assay, a pseudosandwich assay, or a sandwich assay) they can respond by an increase in fluorescence.

5.1.13 Aldosterone-Binding Primary Aptamers

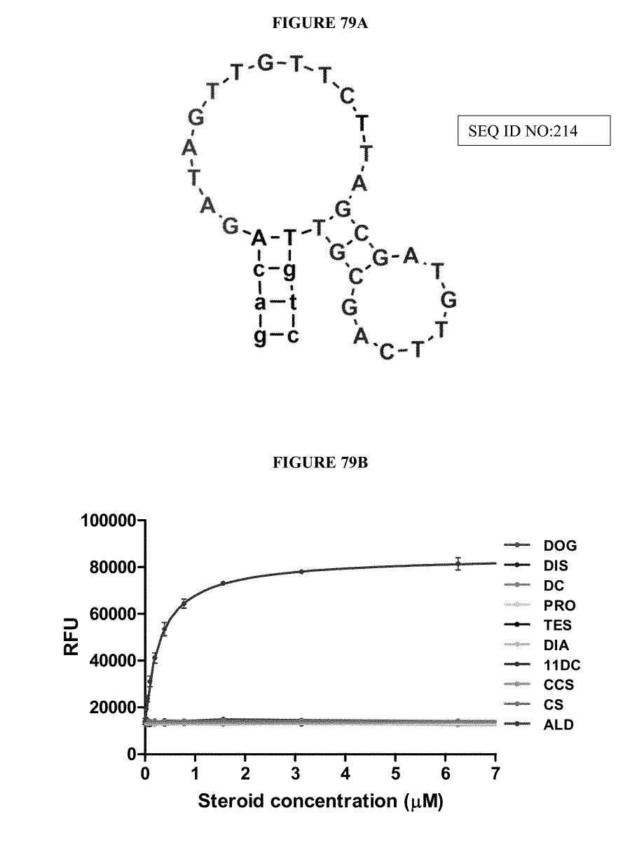

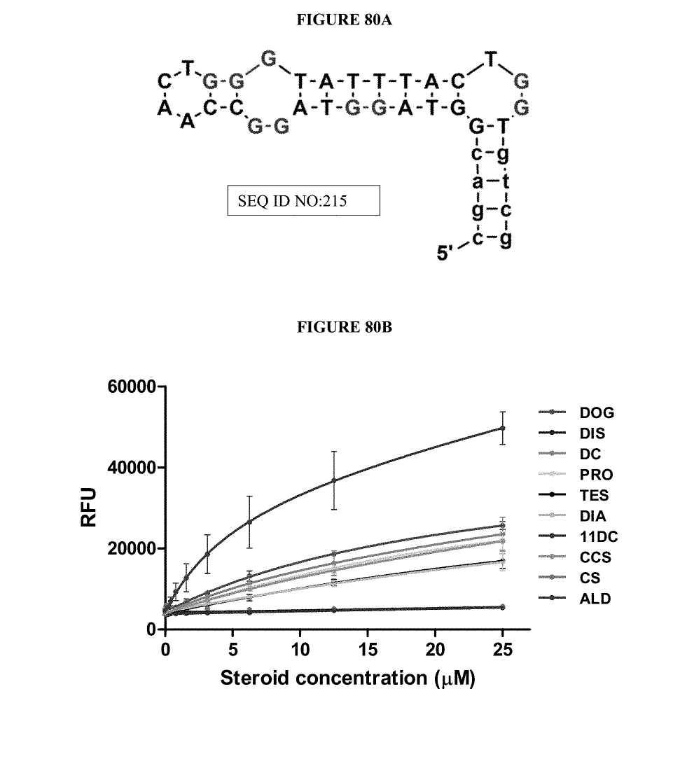

In certain non-limiting embodiments, a primary aptamer binds to aldosterone in an aqueous solution at room temperature or 25° C. with a dissociation constant of less than 10⁻⁴ M and binds selectively with aldosterone (see FIGS. 79B and 80B) versus cortisone, cortisol or dexycortisone, or binds selectively to a steroid having C18 bound to one or two oxygen atoms, versus a steroid having a methyl linked to the C18 position.

In certain non-limiting embodiments, an aldosterone-binding primary aptamer comprises the sequences GATAGT (SEQ ID NO:212) and ATGTTC (SEQ ID NO:213) or a variant of any of these sequences that differs in one or two bases by substitution, deletion, insertion or extension, where said primary aptamer binds to aldosterone in an aqueous solution at room temperature or 25° C. with a dissociation constant of less than 10⁻⁴ M and binds to aldosterone selectively versus cortisone, cortisol or deoxycortisone. In certain non-limiting embodiments, an aldosterone-binding primary aptamer comprises the sequences GATAGT (SEQ ID NO:212) and ATGTTC (SEQ ID NO:213) or a variant thereof and further comprises at least one operative sequence. In certain non-limiting embodiments, a aldosterone-binding primary aptamer comprises the sequences GATAGT (SEQ ID NO:212) and ATGTTC (SEQ ID NO:213) or a variant thereof, and further comprises at least one operative sequence, said operative sequence complementary to a sequence comprised in a sensor oligonucleotide. In certain non-limiting embodiments, a aldosterone-binding primary aptamer comprises the sequences GATAGT (SEQ ID NO:212) and ATGTTC (SEQ ID NO:213), or a variant thereof, and at least one operative sequence on either side (flanking) these two sequences. In certain non-limiting embodiments, a aldosterone-binding primary aptamer comprises the sequences GATAGT (SEQ ID NO:212) and ATGTTC (SEQ ID NO:213), or a variant thereof, and at least one operative sequence on either side (flanking) these two sequences, where two of said operative sequences contain mutually complementary portions and can form a duplex.

For example, but not by way of limitation, a aldosterone-binding aptamer may comprise the sequence: CTC TCG GGA CGA CAG ATA GTT GTT CTT AGC GAT GTT CAG CGT TGT CGT CCC (SEQ ID NO: 264) or related sequence (SEQ ID NO:63, FIG. 1B).

For example, but not by way of limitation, a aldosterone-binding aptamer may comprise the sequence: CTC TCG GGA CGA CGG TAG GTA GGC CAA CTG GGT ATT TAC TGG TGT CGT CCC (SEQ ID NO: 265).

In certain non-limiting embodiments, isolated aldosterone-binding primary aptamers comprise the nucleotide sequence of SEQ ID NO:214, SEQ ID NO:215, SEQ ID NO:63, or variants of these sequences having at least about 80 percent, or at least about 85 percent, or at least about 90 percent, or at least about 95 percent, or at least about 98 percent homology to the original sequence, for example obtained by substitutions, deletions, and insertions of Watson-Crick base pairs or by mutations at non-conserved positions. Percent homology can be determined using standard software such as BLAST or FASTA. In certain non-limiting embodiments, isolated aldosterone-binding primary aptamers comprise the nucleotide sequence of SEQ ID NO:214, SEQ ID NO:215, or SEQ ID NO:63. Said aptamers can bind to aldosterone and in their structure-switching formats (for example, in an anti-aptamer assay, a pseudosandwich assay, or a sandwich assay) they can respond by an increase in fluorescence.

5.1.14 Tobramycin-Binding Primary Aptamers

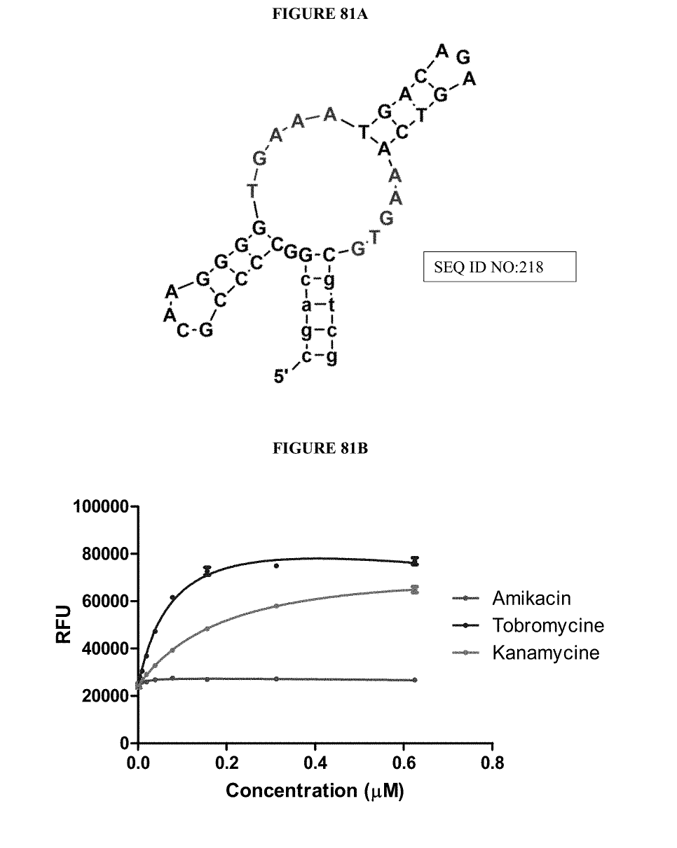

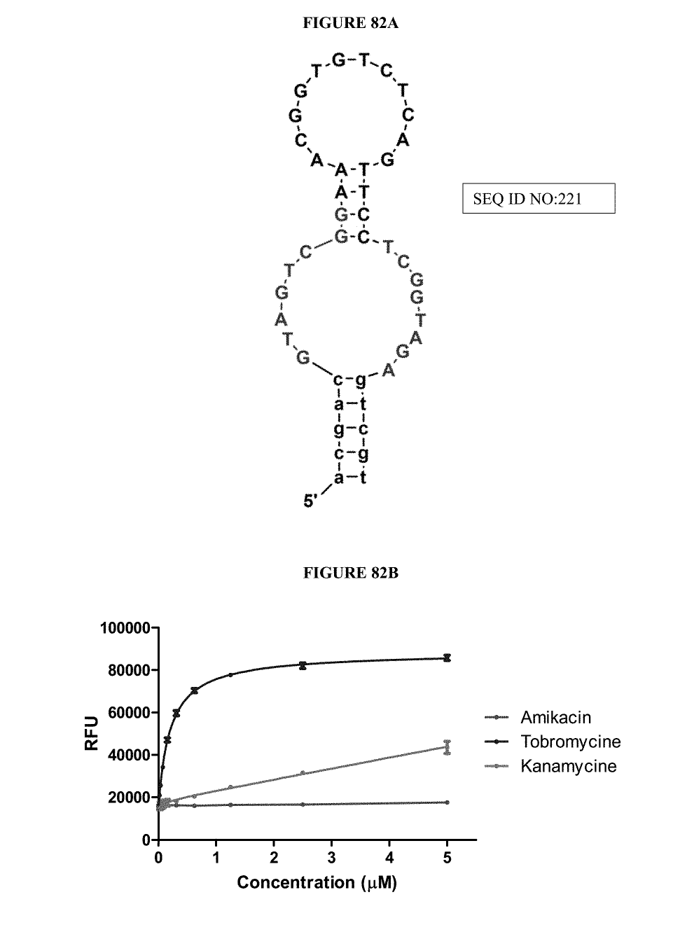

In certain non-limiting embodiments, a primary aptamer binds to tobramycin in an aqueous solution at room temperature or 25° C. with a dissociation constant of less than 10⁻³ M and binds selectively with tobramycin (see FIGS. 81B and 82B) versus amikacin or kanamycin.

In certain non-limiting embodiments, an tobramycin-binding primary aptamer comprises the sequence(s) TGAAA (SEQ ID NO:216) and/or AAGTG (SEQ ID NO:217) or a variant of any of these sequences that differs in one or two bases by substitution, deletion, insertion or extension, where said primary aptamer binds to tobramycin in an aqueous solution at room temperature or 25° C. with a dissociation constant of less than 10⁻³ M and binds to tobramycin selectively versusamikacin or kanamycin. In certain non-limiting embodiments, an tobramycin-binding primary aptamer comprises the sequence(s) TGAAA (SEQ ID NO:216) and/or AAGTG (SEQ ID NO:217) or a variant thereof and further comprises at least one operative sequence. In certain non-limiting embodiments, a tobramycin-binding primary aptamer comprises the sequence(s) TGAAA (SEQ ID NO:216) and/or AAGTG (SEQ ID NO:217) or a variant thereof, and further comprises at least one operative sequence, said operative sequence complementary to a sequence comprised in a sensor oligonucleotide. In certain non-limiting embodiments, a tobramycin-binding primary aptamer comprises the sequence(s) TGAAA (SEQ ID NO:216) and/or AAGTG (SEQ ID NO:217), or a variant thereof, and at least one operative sequence on either side (flanking) this sequence or sequences. In certain non-limiting embodiments, a tobramycin-binding primary aptamer comprises the sequence(s) TGAAA (SEQ ID NO:216) and/or AAGTG (SEQ ID NO:217), or a variant thereof, and at least one operative sequence on either side (flanking) this sequence or sequences, where two of said operative sequences contain mutually complementary portions and can form a duplex.

For example, but not by way of limitation, a tobramycin-binding aptamer may comprise the sequence: CTC TCG GGA CGA CGG CCC CGC AAG GGG TGA AAT GAC AGA GTC AAA GTG CGT CGT CCC (SEQ ID NO: 266).

In certain non-limiting embodiments, an tobramycin-binding primary aptamer comprises the sequence(s) GTAGTC (SEQ ID NO:219) and/or TCGGTAG (SEQ ID NO:220) or a variant of any of these sequences that differs in one or two bases by substitution, deletion, insertion or extension, where said primary aptamer binds to tobramycin in an aqueous solution at room temperature or 25° C. with a dissociation constant of less than 10⁻⁴ M and binds to tobramycin selectively versusamikacin or kanamycin. In certain non-limiting embodiments, an tobramycin-binding primary aptamer comprises the sequence(s) GTAGTC (SEQ ID NO:219) and/or TCGGTAG (SEQ ID NO:220) or a variant thereof and further comprises at least one operative sequence. In certain non-limiting embodiments, a tobramycin-binding primary aptamer comprises the sequence(s) GTAGTC (SEQ ID NO:219) and/or TCGGTAG (SEQ ID NO:220) or a variant thereof, and further comprises at least one operative sequence, said operative sequence complementary to a sequence comprised in a sensor oligonucleotide. In certain non-limiting embodiments, a tobramycin-binding primary aptamer comprises the sequence(s) GTAGTC (SEQ ID NO:219) and/or TCGGTAG (SEQ ID NO:220), or a variant thereof, and at least one operative sequence on either side (flanking) this sequence or sequences. In certain non-limiting embodiments, a tobramycin-binding primary aptamer comprises the sequence(s) GTAGTC (SEQ ID NO:219) and/or TCGGTAG (SEQ ID NO:220), or a variant thereof, and at least one operative sequence on either side (flanking) this sequence or sequences, where two of said operative sequences contain mutually complementary portions and can form a duplex.

For example, but not by way of limitation, a tobramycin-binding aptamer may comprise the sequence: CTC TCG GGA CGA CGT AGT CGG AAA CGG TGT CTC AGT TCC TCG GTA GAG TCG TCC C (SEQ ID NO: 267).

In certain non-limiting embodiments, isolated tobramycin-binding primary aptamers comprise the nucleotide sequence of SEQ ID NO:218, SEQ ID NO:221, or variants of these sequences having at least about 80 percent, or at least about 85 percent, or at least about 90 percent, or at least about 95 percent, or at least about 98 percent homology to the original sequence, for example obtained by substitutions, deletions, and insertions of Watson-Crick base pairs or by mutations at non-conserved positions. Percent homology can be determined using standard software such as BLAST or FASTA. In certain non-limiting embodiments, isolated tobramycin-binding primary aptamers comprise the nucleotide sequence of SEQ ID NO:218 or SEQ ID NO:221. Said aptamers can bind to tobramycin and in their structure-switching formats (for example, in an anti-aptamer assay, a pseudosandwich assay, or a sandwich assay) they can respond by an increase in fluorescence.

5.1.15 Amikacin-Binding Primary Aptamers

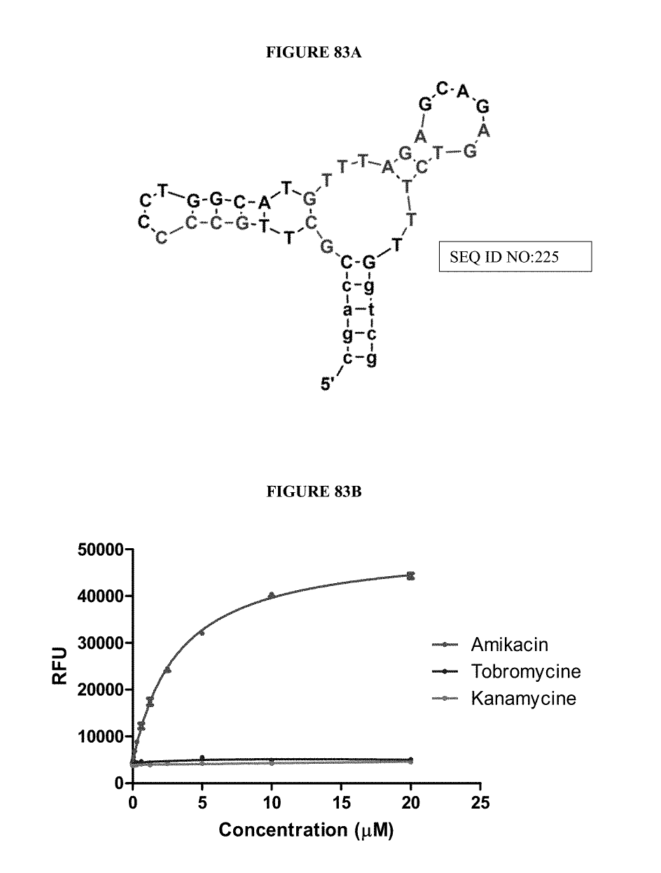

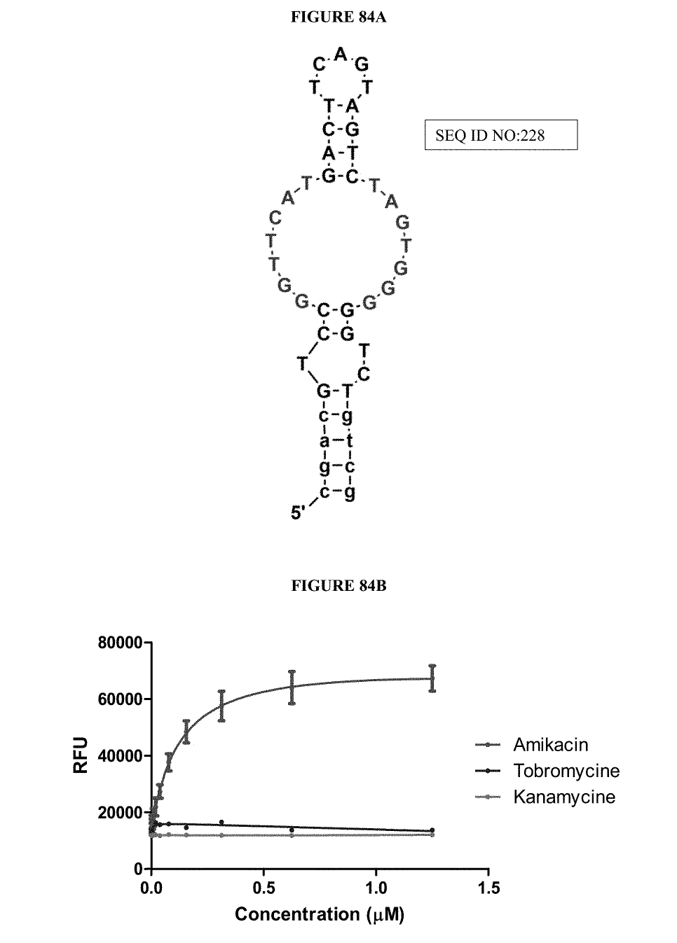

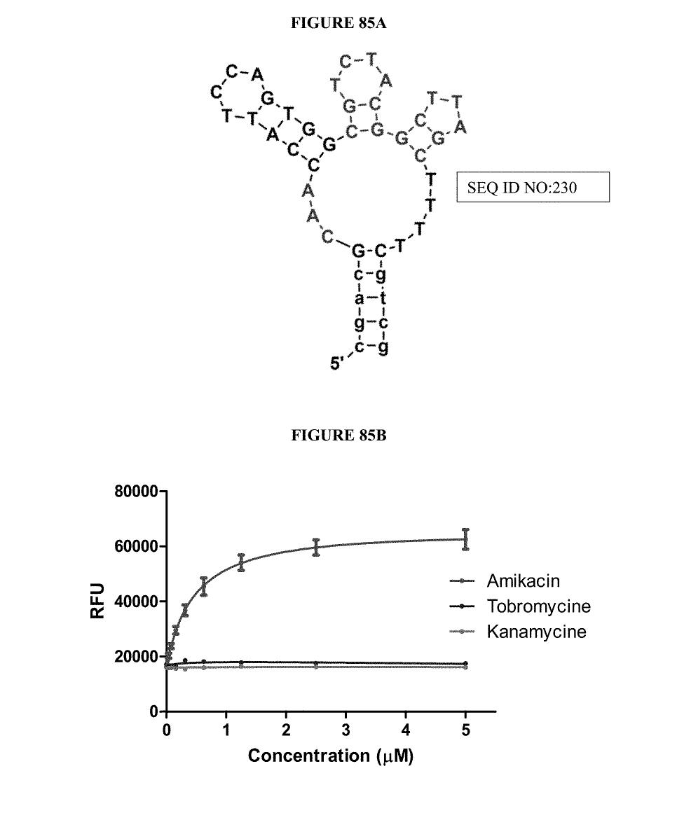

In certain non-limiting embodiments, a primary aptamer binds to amikacin in an aqueous solution at room temperature or 25° C. with a dissociation constant of less than 10⁻³ M and binds selectively with amikacin versus tobramycin or kanamycin (see FIGS. 83B, 84B and 85B).

In certain non-limiting embodiments, an amikacin-binding primary aptamer comprises the sequences GC and GCCC(SEQ ID NO:222) and GTTTAGA (SEQ ID NO:223) and AGTCTT (SEQ ID NO:224) or a variant of any of these sequences that differs in one or two bases by substitution, deletion, insertion or extension, where said primary aptamer binds to amikacin in an aqueous solution at room temperature or 25° C. with a dissociation constant of less than 10⁻³ M and binds to amikacin selectively versustobramycin and kanamycin. In certain non-limiting embodiments, a amikacin-binding primary aptamer comprises the sequences GC and GCCC(SEQ ID NO:222) and GTTTAGA (SEQ ID NO:223) and AGTCTT (SEQ ID NO:224) or a variant thereof and further comprises at least one operative sequence. In certain non-limiting embodiments, a amikacin-binding primary aptamer comprises the sequences GC and GCCC(SEQ ID NO:222) and GTTTAGA (SEQ ID NO:223) and AGTCTT (SEQ ID NO:224), or a variant thereof, and further comprises at least one operative sequence, said operative sequence complementary to a sequence comprised in a sensor oligonucleotide. In certain non-limiting embodiments, a amikacin-binding primary aptamer comprises the sequences GC and GCCC(SEQ ID NO:222) and GTTTAGA (SEQ ID NO:223) and AGTCTT (SEQ ID NO:224), or a variant thereof, and at least one operative sequence on either side (flanking) these four sequences. In certain non-limiting embodiments, a amikacin-binding primary aptamer comprises the sequences GC and GCCC(SEQ ID NO:222) and GTTTAGA (SEQ ID NO:223) and AGTCTT (SEQ ID NO:224), or a variant thereof, and at least one operative sequence on either side (flanking) these four sequences, where two of said operative sequences contain mutually complementary portions and can form a duplex. For example, but not by way of limitation, a amikacin-binding aptamer may comprise the sequence: CTC TCG GGA CGA CCG CTT GCC CCC TGG CAT GTT TAG AGC AGA GTC TTT GGT CGT CCC (SEQ ID NO: 268).

In certain non-limiting embodiments, an amikacin-binding primary aptamer comprises the sequences GGTTCAT (SEQ ID NO:226) and ATGTGGG (SEQ ID NO:227) or a variant of any of these sequences that differs in one or two bases by substitution, deletion, insertion or extension, where said primary aptamer binds to amikacin in an aqueous solution at room temperature or 25° C. with a dissociation constant of less than 10⁻⁴ M and binds to amikacin selectively versustobramycin and kanamycin. In certain non-limiting embodiments, a amikacin-binding primary aptamer comprises the sequences GGTTCAT (SEQ ID NO:226) and ATGTGGG (SEQ ID NO:227) or a variant thereof and further comprises at least one operative sequence. In certain non-limiting embodiments, a amikacin-binding primary aptamer comprises the sequences GGTTCAT (SEQ ID NO:226) and ATGTGGG (SEQ ID NO:227), or a variant thereof, and further comprises at least one operative sequence, said operative sequence complementary to a sequence comprised in a sensor oligonucleotide. In certain non-limiting embodiments, a amikacin-binding primary aptamer comprises the sequences GGTTCAT (SEQ ID NO:226) and ATGTGGG (SEQ ID NO:227), or a variant thereof, and at least one operative sequence on either side (flanking) these two sequences. In certain non-limiting embodiments, a amikacin-binding primary aptamer comprises the sequences GGTTCAT (SEQ ID NO:226) and ATGTGGG (SEQ ID NO:227), or a variant thereof, and at least one operative sequence on either side (flanking) these two sequences, where two of said operative sequences contain mutually complementary portions and can form a duplex. For example, but not by way of limitation, a amikacin-binding aptamer may comprise the sequence: CTC TCG GGA CGA CGT CCG GTT CAT GAC TTC AGT AGT CTA GTG GGG GTC TGT CGT CCC (SEQ ID NO: 269).

In certain non-limiting embodiments, an amikacin-binding primary aptamer comprises the sequences CAA and CGTCTACGGCTTAGC (SEQ ID NO:229) or a variant of any of these sequences that differs in one or two bases by substitution, deletion, insertion or extension, where said primary aptamer binds to amikacin in an aqueous solution at room temperature or 25° C. with a dissociation constant of less than 10⁻⁴ M and binds to amikacin selectively versustobramycin and kanamycin. In certain non-limiting embodiments, a amikacin-binding primary aptamer comprises the sequences CAA and CGTCTACGGCTTAGC (SEQ ID NO:229) or a variant thereof and further comprises at least one operative sequence. In certain non-limiting embodiments, a amikacin-binding primary aptamer comprises the sequences CAA and CGTCTACGGCTTAGC (SEQ ID NO:229), or a variant thereof, and further comprises at least one operative sequence, said operative sequence complementary to a sequence comprised in a sensor oligonucleotide. In certain non-limiting embodiments, a amikacin-binding primary aptamer comprises the sequences CAA and CGTCTACGGCTTAGC (SEQ ID NO:229), or a variant thereof, and at least one operative sequence on either side (flanking) these two sequences. In certain non-limiting embodiments, a amikacin-binding primary aptamer comprises the sequences CAA and CGTCTACGGCTTAGC (SEQ ID NO:229), or a variant thereof, and at least one operative sequence on either side (flanking) these two sequences, where two of said operative sequences contain mutually complementary portions and can form a duplex. For example, but not by way of limitation, a amikacin-binding aptamer may comprise the sequence: CTC TCG GGA CGA CGC AAC CAT TCC AGT GGC GTC TAC GGC TTA GCT TTT CGT CGT CCC (SEQ ID NO: 270).

In certain non-limiting embodiments, isolated amikacin-binding primary aptamers comprise the nucleotide sequence of SEQ ID NO:225, SEQ ID NO:228, SEQ ID NO:230, or variants of these sequences having at least about 80 percent, or at least about 85 percent, or at least about 90 percent, or at least about 95 percent, or at least about 98 percent homology to the original sequence, for example obtained by substitutions, deletions, and insertions of Watson-Crick base pairs or by mutations at non-conserved positions. Percent homology can be determined using standard software such as BLAST or FASTA. In certain non-limiting embodiments, isolated amikacin-binding primary aptamers comprise the nucleotide sequence of SEQ ID NO:225, SEQ ID NO:228, or SEQ ID NO:230. Said aptamers can bind to amikacin and in their structure-switching formats (for example, in an anti-aptamer assay, a pseudosandwich assay, or a sandwich assay) they can respond by an increase in fluorescence.

5.1.16 Methylene Blue-Binding Primary Aptamers

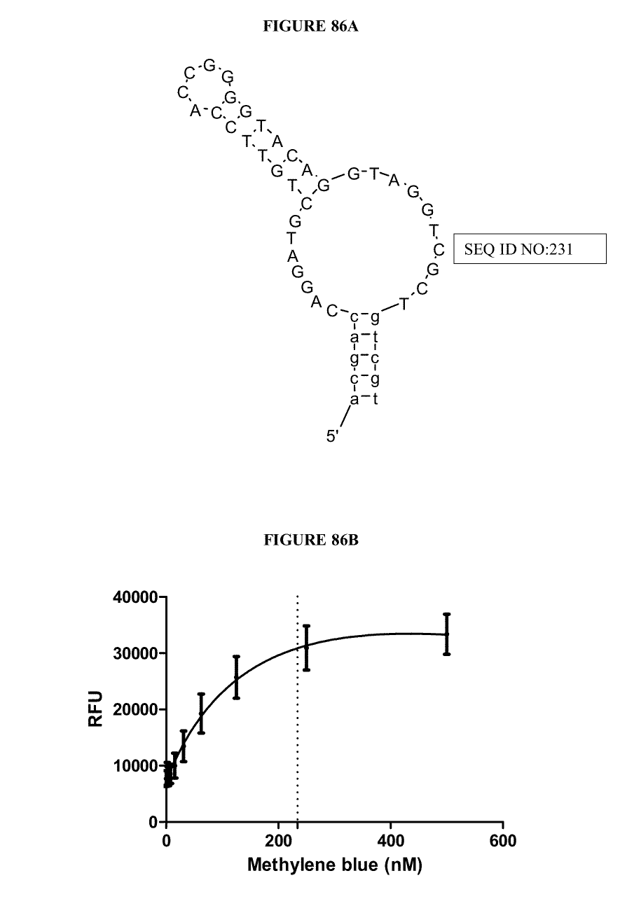

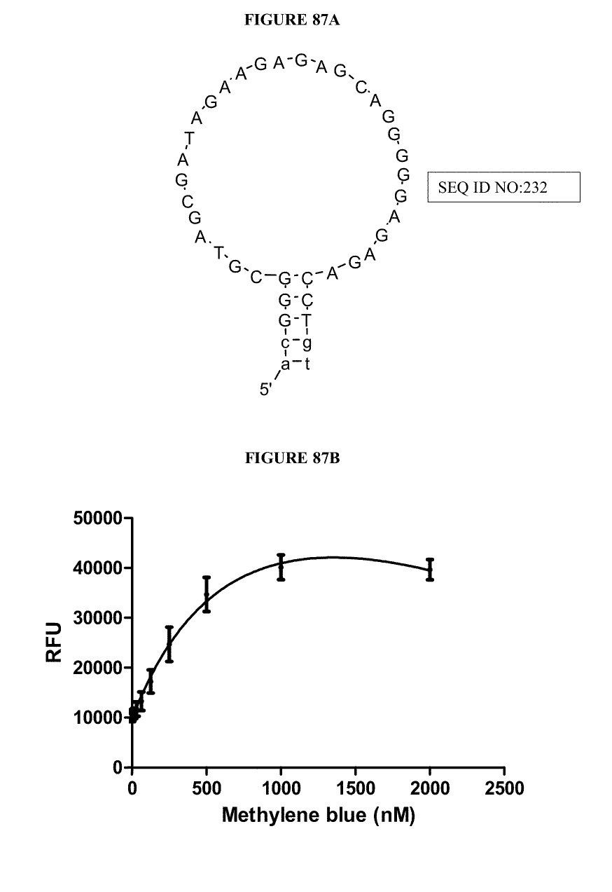

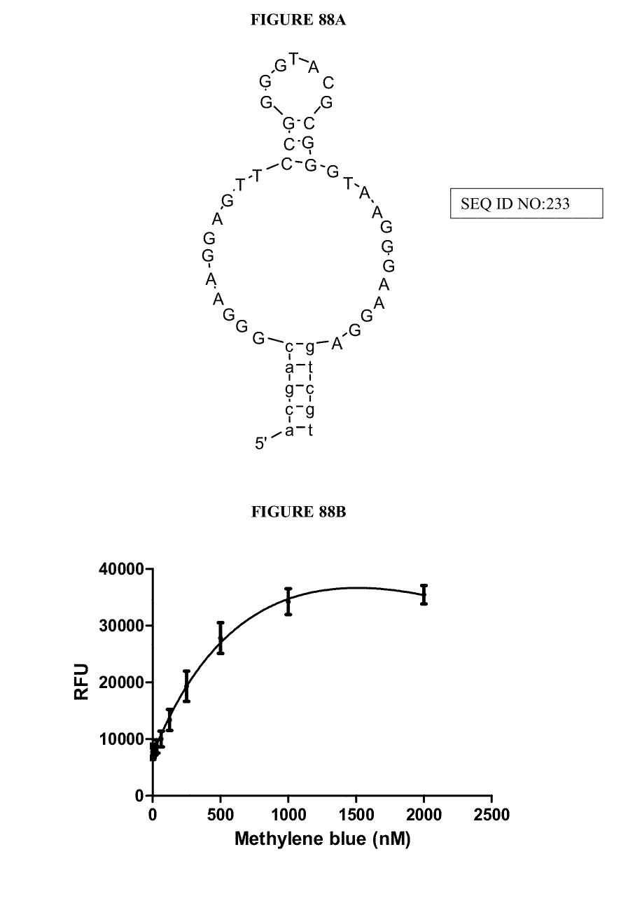

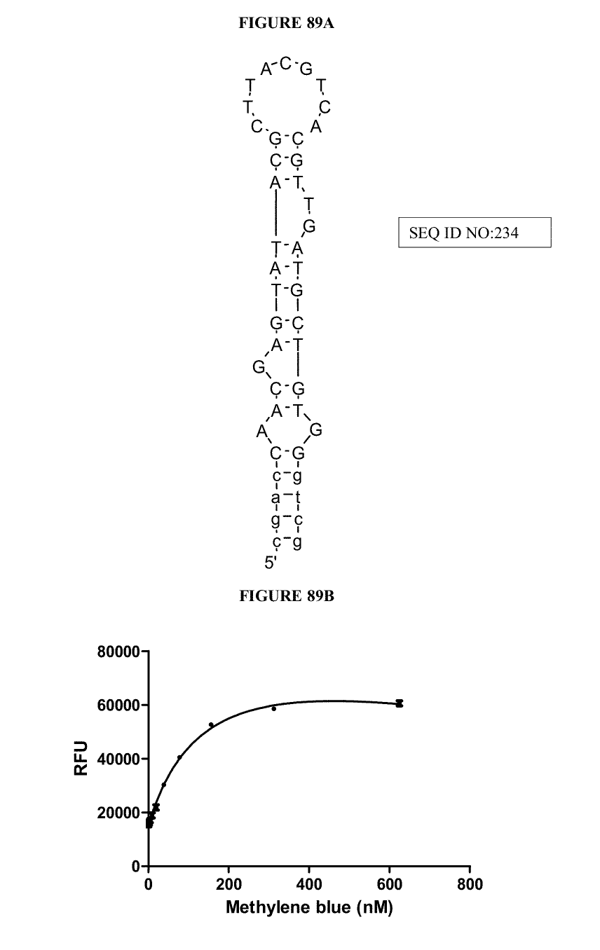

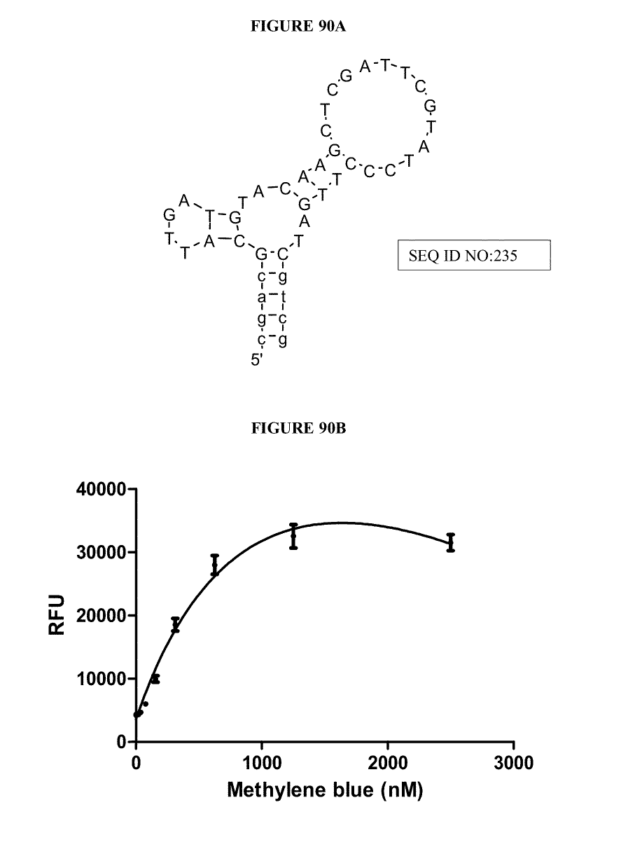

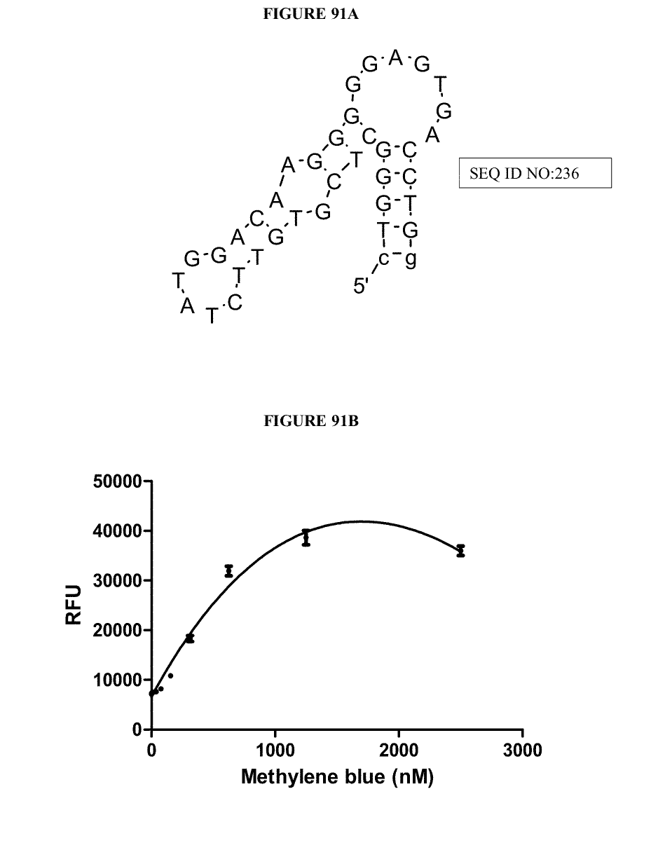

In certain non-limiting embodiments, a primary aptamer binds to methylene blue in an aqueous solution at room temperature or 25° C. with a dissociation constant of less than 10⁻⁶ M and binds selectively with methylene blue (see FIGS. 86B, 87B, 88B, 89B, 90B and 91B).

For example, but not by way of limitation, a methylene blue-binding aptamer may comprise the sequence: CTC TCG GGA CGA CCA GGA TGC TGT TCC ACC GGG GTA CAG GTA GGT CGC TGT CGT CCC (SEQ ID NO: 271).

For example, but not by way of limitation, a methylene blue-binding aptamer may comprise the sequence: CTC TCG GGA CGA CGG GCG TAG CGA TAG AAG AGA GCA GGG GGA GAG ACC TGT CGT CCC (SEQ ID NO: 272).

For example, but not by way of limitation, a methylene blue-binding aptamer may comprise the sequence: CTC TCG GGA CGA CGG GAA GGA GTT CCG GGG TAC GCG GGT AAG GGA AGG AGT CGT CCC (SEQ ID NO: 273).

For example, but not by way of limitation, a methylene blue-binding aptamer may comprise the sequence: CTC TCG GGA CGA CCA ACG AGT ATA CGC TTA CGT CAC GTT GAT GCT GTG GGT CGT CCC (SEQ ID NO: 274).

For example, but not by way of limitation, a methylene blue-binding aptamer may comprise the sequence: CTC TCG GGA CGA CGC ATT GAT GTA CAA GCT CGA TTC GTA TCC CTT GAT CGT CGT CCC (SEQ ID NO: 275).

For example, but not by way of limitation, a methylene blue-binding aptamer may comprise the sequence: CTC TCG GGA CGA CTG GGC TCG TGT TCT ATG GAC AAG GGG GAG TGA CCT GGT CGT CCC (SEQ ID NO: 276).

In certain non-limiting embodiments, isolated methylene blue-binding primary aptamers comprise the nucleotide sequence of SEQ ID NO:231, SEQ ID NO:232, SEQ ID NO:233, SEQ ID NO:234, SEQ ID NO:235, SEQ ID NO:236 or variants of these sequences having at least about 80 percent, or at least about 85 percent, or at least about 90 percent, or at least about 95 percent, or at least about 98 percent homology to the original sequence, for example obtained by substitutions, deletions, and insertions of Watson-Crick base pairs or by mutations at non-conserved positions. Percent homology can be determined using standard software such as BLAST or FASTA. In certain non-limiting embodiments, isolated methylene blue-binding primary aptamers comprise the nucleotide sequence of SEQ ID NO:231, SEQ ID NO:232, SEQ ID NO:233, SEQ ID NO:234, SEQ ID NO:235, or SEQ ID NO:236. Said aptamers can bind to methylene blue and in their structure-switching formats (for example, in an anti-aptamer assay, a pseudosandwich assay, or a sandwich assay) they can respond by an increase in fluorescence.

5.1.17 Ammonium-Binding Primary Aptamers

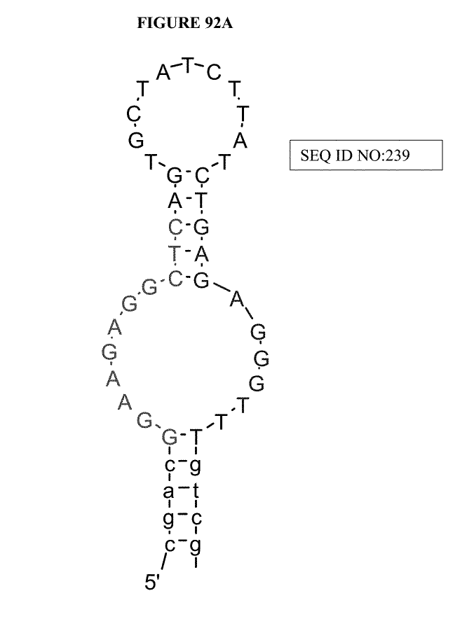

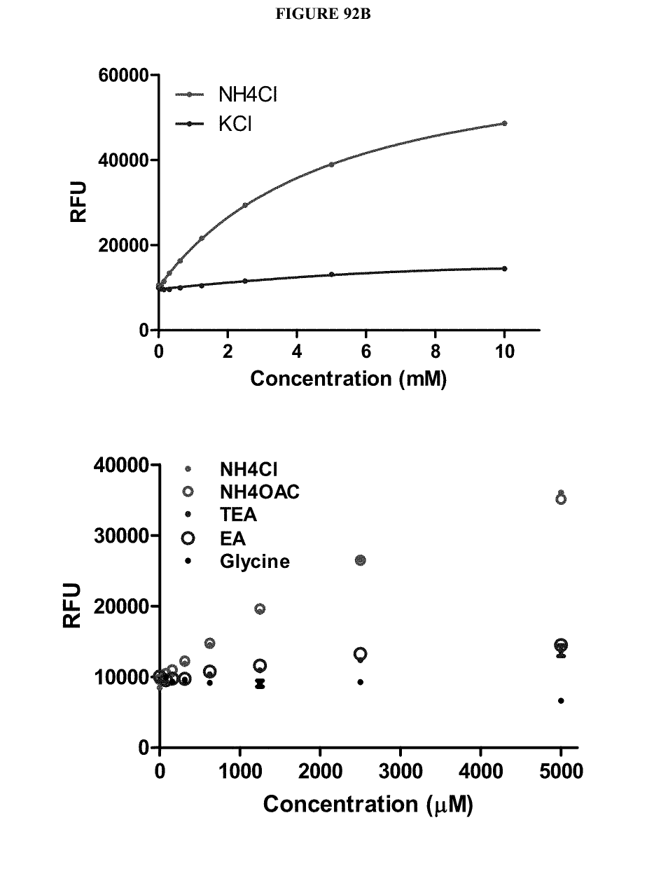

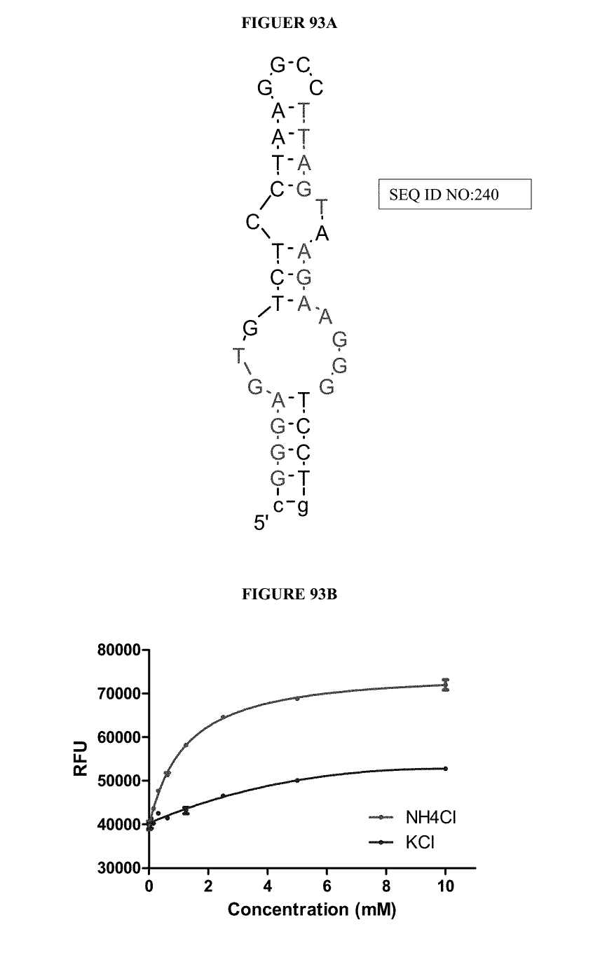

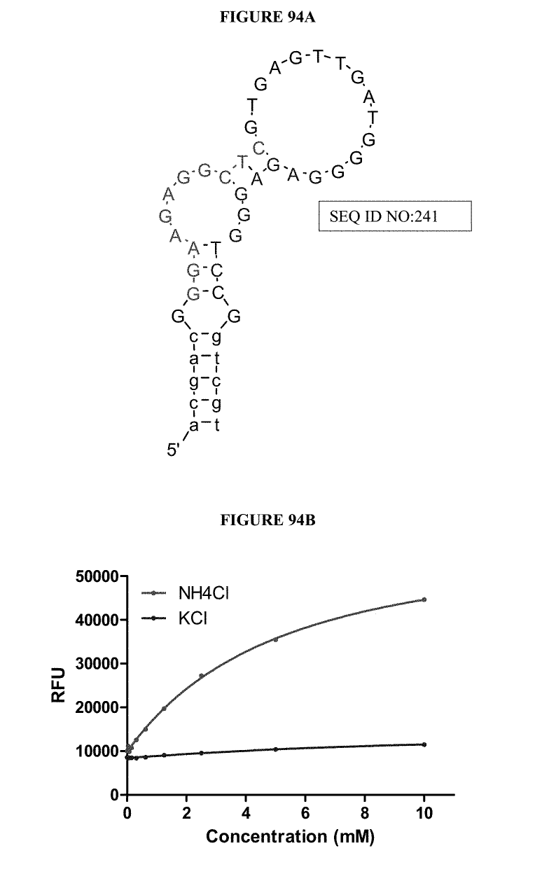

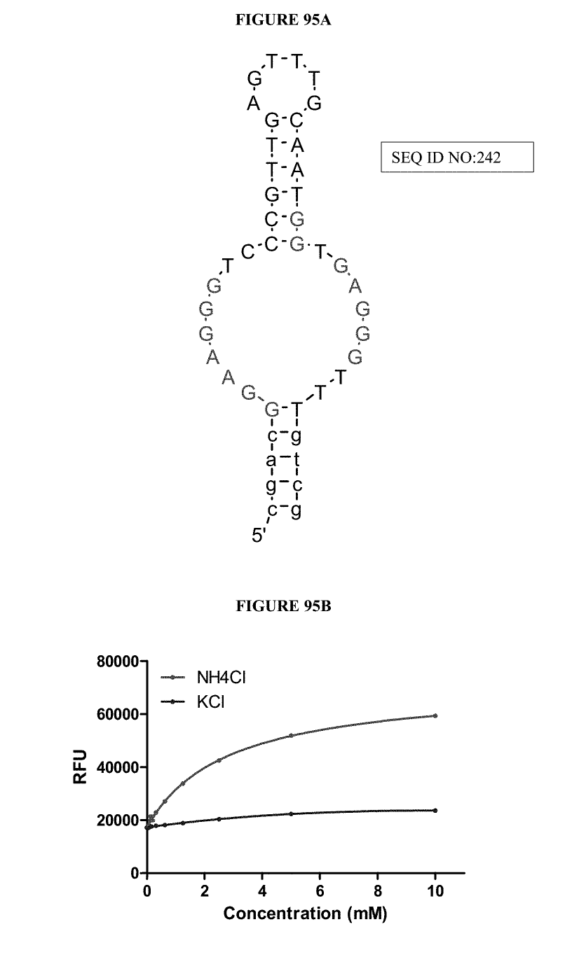

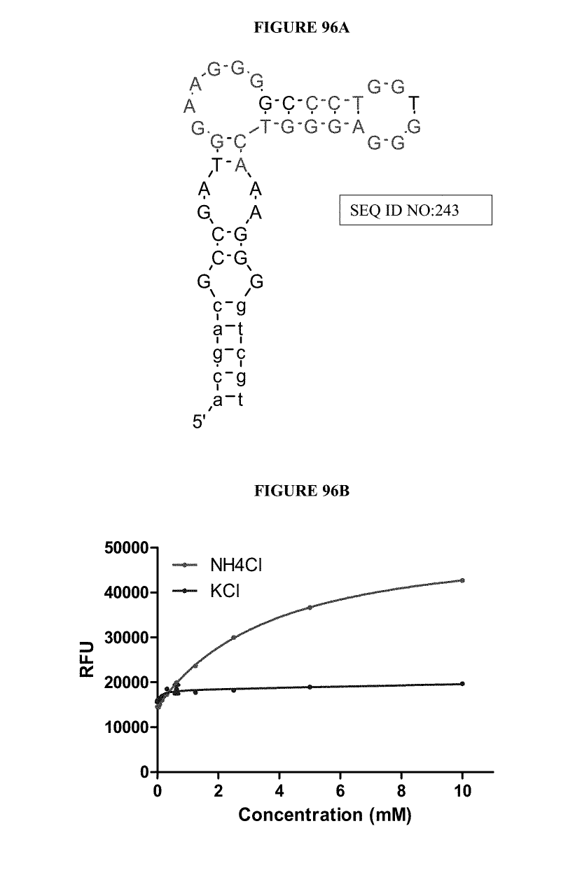

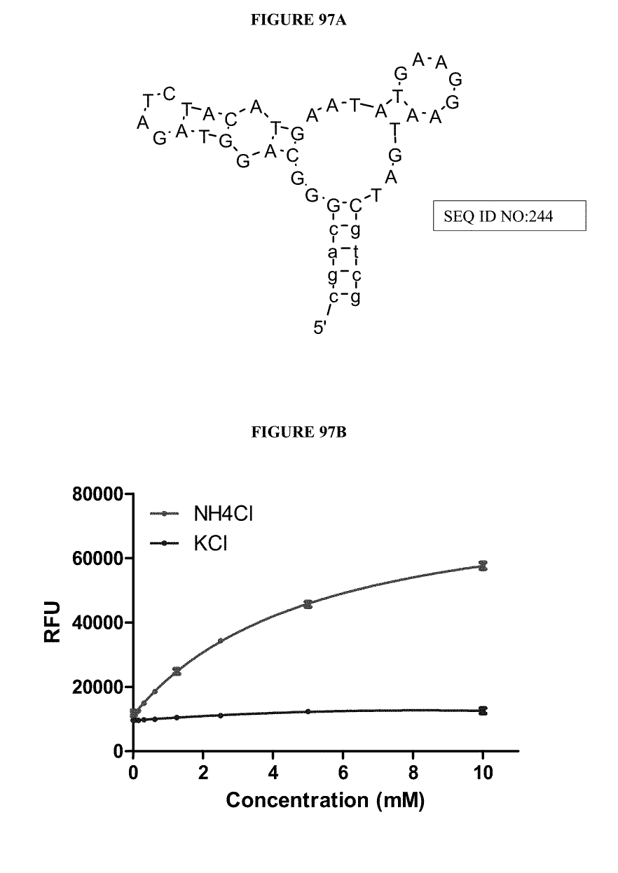

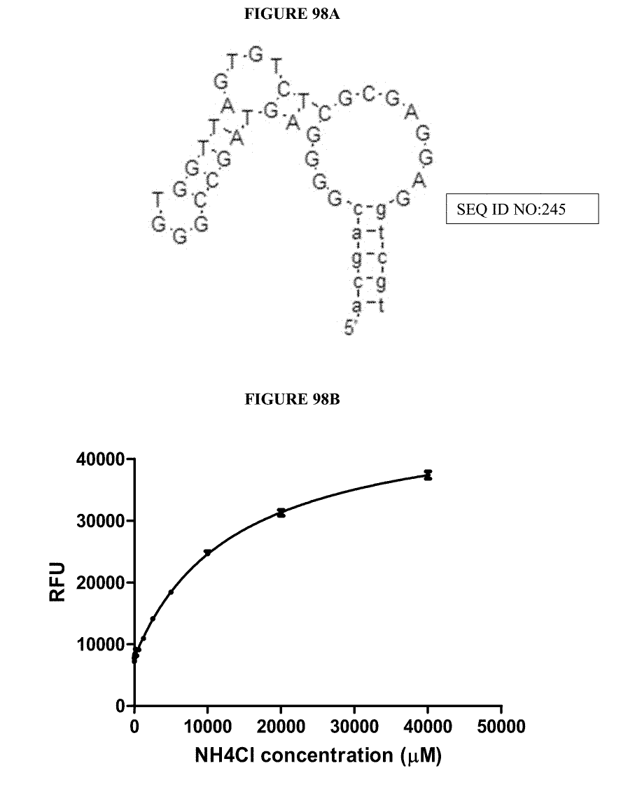

In certain non-limiting embodiments, a primary aptamer binds to ammonium ion in an aqueous solution at room temperature or 25° C. with a dissociation constant of less than 10⁻² M and binds selectively with ammonium versus glycine or ethanolamine or potassium ion (see FIGS. 92B, 93B, 94B, 95B, 96B, 97B, and 98B).

For example, but not by way of limitation, an ammonium-binding aptamer may comprise the sequence: CTC TCG GGA CGA CGG AAG AGG CTC AGT GCT ATC TTA TCT GAG AGG GTT TGT CGT CCC (SEQ ID NO: 277).

For example, but not by way of limitation, an ammonium-binding aptamer may comprise the sequence: CTC TCG GGA CGA CGG GAG TGT CTC CTA AGG CCT TAG TAA GAA GGG TCC TGT CGT CCC (SEQ ID NO: 278).

For example, but not by way of limitation, an ammonium-binding aptamer may comprise the sequence: CTC TCG GGA CGA CGG GAA GAG GCT CGT GAG TTG ATG GGG AGA GGG TCC GGT CGT CCC (SEQ ID NO: 279).

For example, but not by way of limitation, an ammonium-binding aptamer may comprise the sequence: CTC TCG GGA CGA CGG AAG GGT CCC GTT GAG TTT GCA ATG GTG AGG GTT TGT CGT CCC (SEQ ID NO: 280).

For example, but not by way of limitation, an ammonium-binding aptamer may comprise the sequence: CTC TCG GGA CGA CGC CGA TGG AAG GGG CCC TGG TGG GAG GGT CAA AGG GGT CGT CCC (SEQ ID NO: 281).

For example, but not by way of limitation, an ammonium-binding aptamer may comprise the sequence: CTC TCG GGA CGA CGG GCA GGT AGA TCT ACA TGA ATA TGA AGG AAT GAT CGT CGT CCC (SEQ ID NO: 282).

For example, but not by way of limitation, an ammonium-binding aptamer may comprise the sequence: CTC TCG GGA CGA CGG GGA GTA GCC GGG TGG TTA GTG TCT CGC GAG GAA GTC GTC CC (SEQ ID NO: 283).

In certain non-limiting embodiments, isolated ammonium-binding primary aptamers comprise the nucleotide sequence of SEQ ID NO:239, SEQ ID NO:240, SEQ ID NO:241, SEQ ID NO:242, SEQ ID NO:243, SEQ ID NO:244, SEQ ID NO:245 or variants of these sequences having at least about 80 percent, or at least about 85 percent, or at least about 90 percent, or at least about 95 percent, or at least about 98 percent homology to the original sequence, for example obtained by substitutions, deletions, and insertions of Watson-Crick base pairs or by mutations at non-conserved positions. Percent homology can be determined using standard software such as BLAST or FASTA. In certain non-limiting embodiments, isolated ammonium-binding primary aptamers comprise the nucleotide sequence of, SEQ ID NO:239, SEQ ID NO:240, SEQ ID NO:241, SEQ ID NO:242, SEQ ID NO:243, SEQ ID NO:244, SEQ ID NO:245. Said aptamers can bind to ammonium and in their structure-switching formats (for example, in an anti-aptamer assay, a pseudosandwich assay, or a sandwich assay) they can respond by an increase in fluorescence.

5.1.18 Boronic Acid-Binding Primary Aptamers

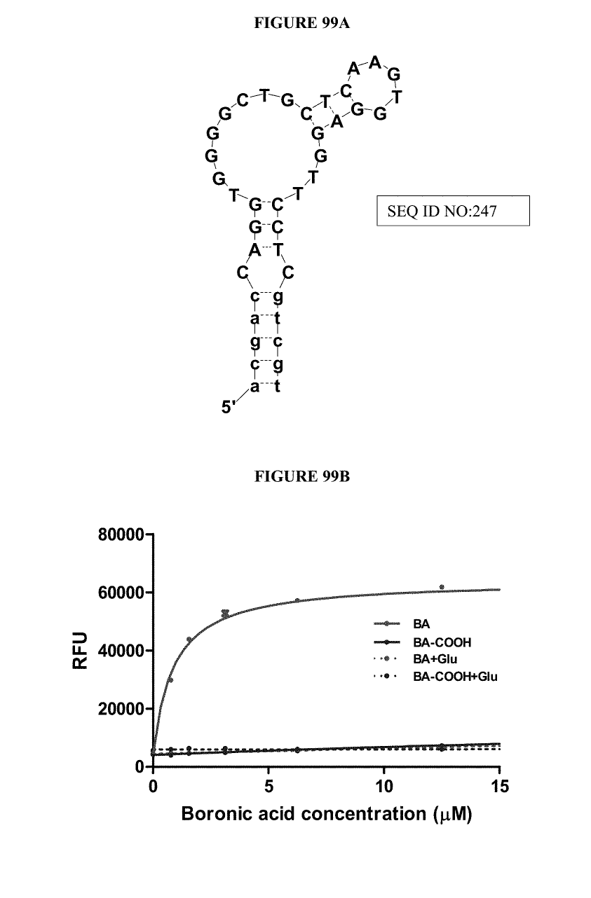

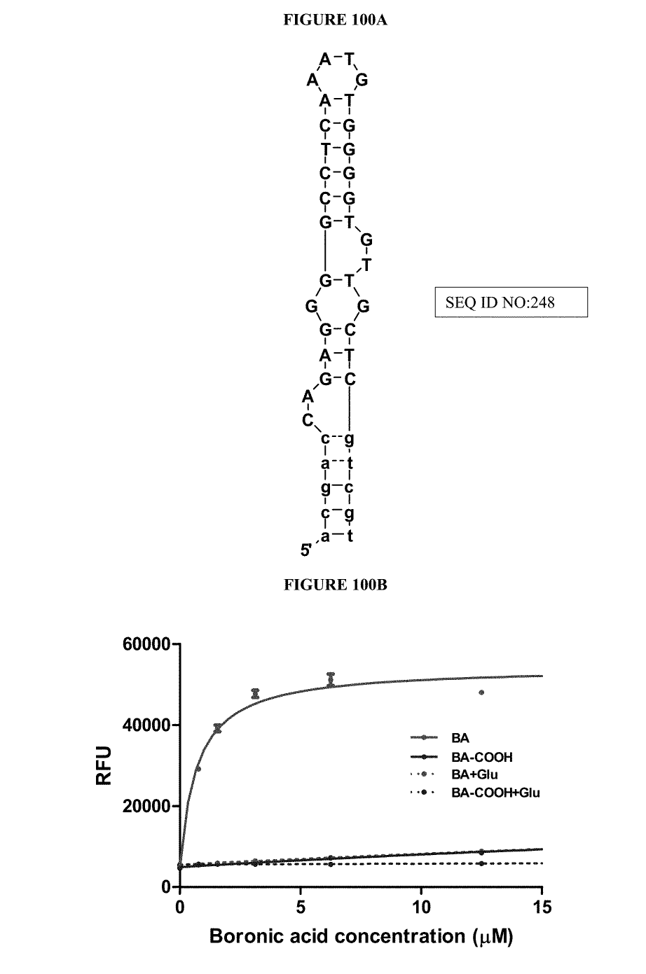

In certain non-limiting embodiments, a primary aptamer binds to boronic acid in an aqueous solution at room temperature or 25° C. with a dissociation constant of less than 10⁻² M and binds selectively with boronic acid versus bisboronic acid, e.g., complexed with glucose (FIG. 99B, 100B).

For example, but not by way of limitation, a boronic acid-binding aptamer may comprise the sequence: CTC TCG GGA CGA CCA GGT GGG GCT GCT CAA GTG GAG GTT CCT CGT CGT CCC (SEQ ID NO: 284).

For example, but not by way of limitation, a boronic acid-binding aptamer may comprise the sequence: CTC TCG GGA CGA CCA GAG GGG CCT CAA ATG TGG GGT GTT GCT CGT CGT CCC (SEQ ID NO: 285).

In certain non-limiting embodiments, isolated boronic acid-binding primary aptamers comprise the nucleotide sequence of SEQ ID NO:247, SEQ ID NO:248, or variants of these sequences having at least about 80 percent, or at least about 85 percent, or at least about 90 percent, or at least about 95 percent, or at least about 98 percent homology to the original sequence, for example obtained by substitutions, deletions, and insertions of Watson-Crick base pairs or by mutations at non-conserved positions. Percent homology can be determined using standard software such as BLAST or FASTA. In certain non-limiting embodiments, isolated boronic acid-binding primary aptamers comprise the nucleotide sequence of, SEQ ID NO:247 or SEQ ID NO:248. Said aptamers can bind to boronic acid and in their structure-switching formats (for example, in an anti-aptamer assay, a pseudosandwich assay, or a sandwich assay) they can respond by an increase in fluorescence.

5.1.19 Epinephrine-Binding Primary Aptamers

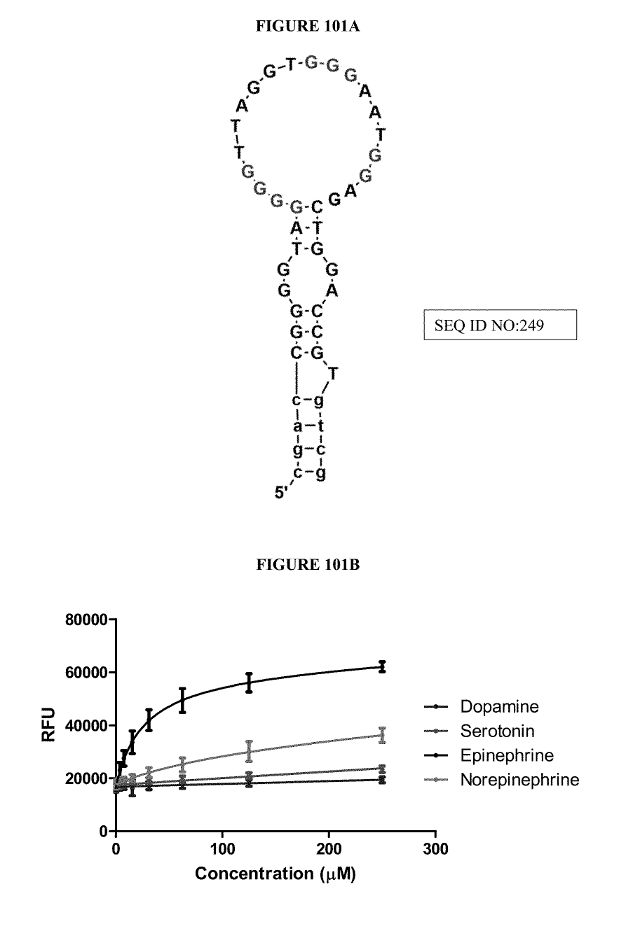

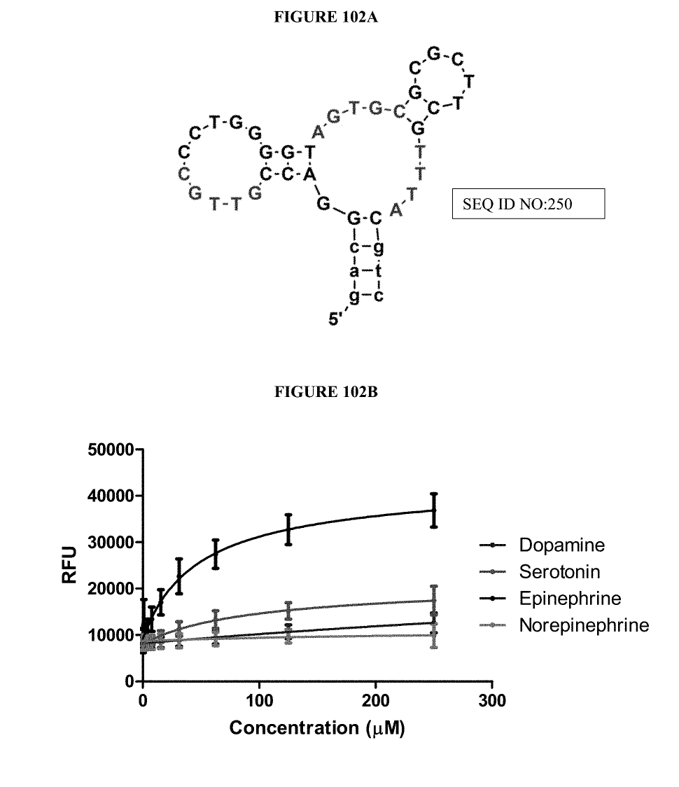

In certain non-limiting embodiments, a primary aptamer binds to epinephrine in an aqueous solution at room temperature or 25° C. with a dissociation constant of less than 10⁻³ M and binds selectively with epinephrine versus serotonin, norepinephrine or dpoamine (FIG. 101B, 102B).

For example, but not by way of limitation, an epinephrine-binding aptamer may comprise the sequence: CTC TCG GGA CGA CCG GGG TAG GGG TTA GGT GGG AAT GGA GCT GGA CCG TGT CGT CCC (SEQ ID NO: 286).

For example, but not by way of limitation, an epinephrine-binding aptamer may comprise the sequence: CTC TCG GGA CGA CGG ACC GTT GCC CTG GGG TAG TGC GCG CTT CGT TTA CGT CGT CCC (SEQ ID NO: 287).

In certain non-limiting embodiments, isolated epinephrine-binding primary aptamers comprise the nucleotide sequence of SEQ ID NO:249, SEQ ID NO:250, or variants of these sequences having at least about 80 percent, or at least about 85 percent, or at least about 90 percent, or at least about 95 percent, or at least about 98 percent homology to the original sequence, for example obtained by substitutions, deletions, and insertions of Watson-Crick base pairs or by mutations at non-conserved positions. Percent homology can be determined using standard software such as BLAST or FASTA. In certain non-limiting embodiments, isolated epinephrine-binding primary aptamers comprise the nucleotide sequence of, SEQ ID NO:249 or SEQ ID NO:250. Said aptamers can bind to epinephrine and in their structure-switching formats (for example, in an anti-aptamer assay, a pseudosandwich assay, or a sandwich assay) they can respond by an increase in fluorescence.

5.1.20 Creatinine-Binding Primary Aptamers

In certain non-limiting embodiments, a primary aptamer binds to creatinine in an aqueous solution at room temperature or 25° C. with a dissociation constant of less than 10⁻² M and binds selectively with creatinine versus creatine or urea.

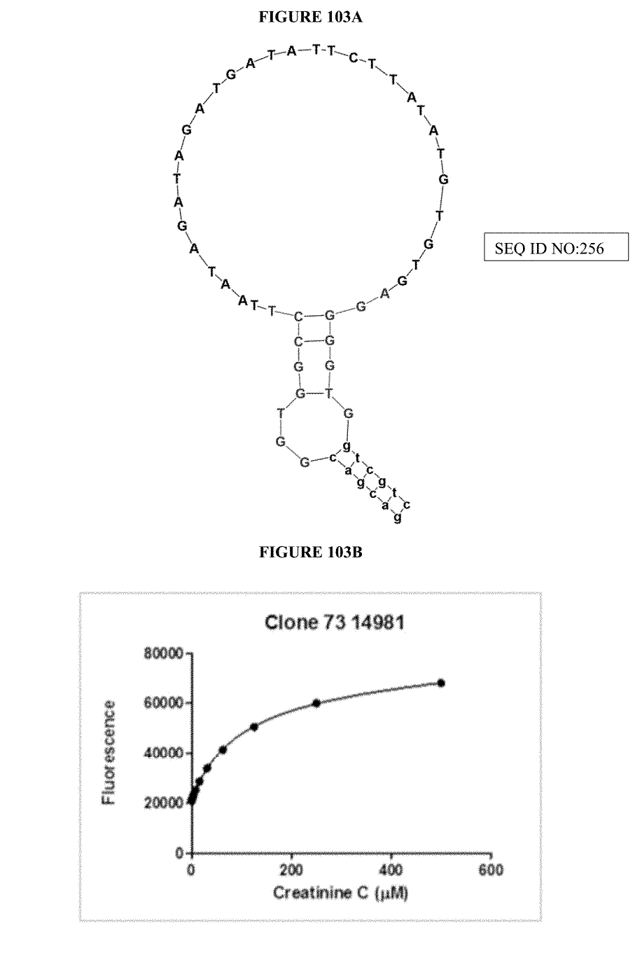

In certain non-limiting embodiments, a creatinine-binding primary aptamer comprises the sequences GGTGGCCT (SEQ ID NO:254) and AGGGGTG (SEQ ID NO:255) or a variant of any of these sequences that differs in one or two bases by substitution, deletion, insertion or extension, where said primary aptamer binds to creatinine (see FIGS. 103B, 104B. 105B, 106B) in an aqueous solution at room temperature or 25° C. with a dissociation constant of less than 10⁻² M and binds to creatinine selectively versus creatine or urea. In certain non-limiting embodiments, a creatinine-binding primary aptamer comprises the sequences GGTGGCCT (SEQ ID NO:254) and AGGGGTG (SEQ ID NO:255) or a variant thereof and further comprises at least one operative sequence. In certain non-limiting embodiments, a creatinine-binding primary aptamer comprises the sequences GGTGGCCT (SEQ ID NO:254) and AGGGGTG (SEQ ID NO:255) or a variant thereof, and further comprises at least one operative sequence, said operative sequence complementary to a sequence comprised in a sensor oligonucleotide. In certain non-limiting embodiments, a creatinine-binding primary aptamer comprises the sequences GGTGGCCT (SEQ ID NO:254) and AGGGGTG (SEQ ID NO:255), or a variant thereof, and at least one operative sequence on either side (flanking) these two sequences. In certain non-limiting embodiments, a creatinine-binding primary aptamer comprises the sequences GGTGGCCT (SEQ ID NO:254) and AGGGGTG (SEQ ID NO:255) or a variant thereof, and at least one operative sequence on either side (flanking) these two sequences, where two of said operative sequences contain mutually complementary portions and can form a duplex.

For example, but not by way of limitation, a creatinine-binding aptamer may comprise the sequence: GA CGA CGGTGGCCTTAATAGATAGATGATATTCTTAT ATGTG TGAGGGGTG GT CGT C (SEQ ID NO:256; FIG. 103A).

For example, but not by way of limitation, a creatinine-binding aptamer may comprise the sequence: GA CGA C GGTGGCCTATATTGGTATGTATGAA GAATAGAACTATTAGGGGGT GT C (SEQ ID NO: 288).

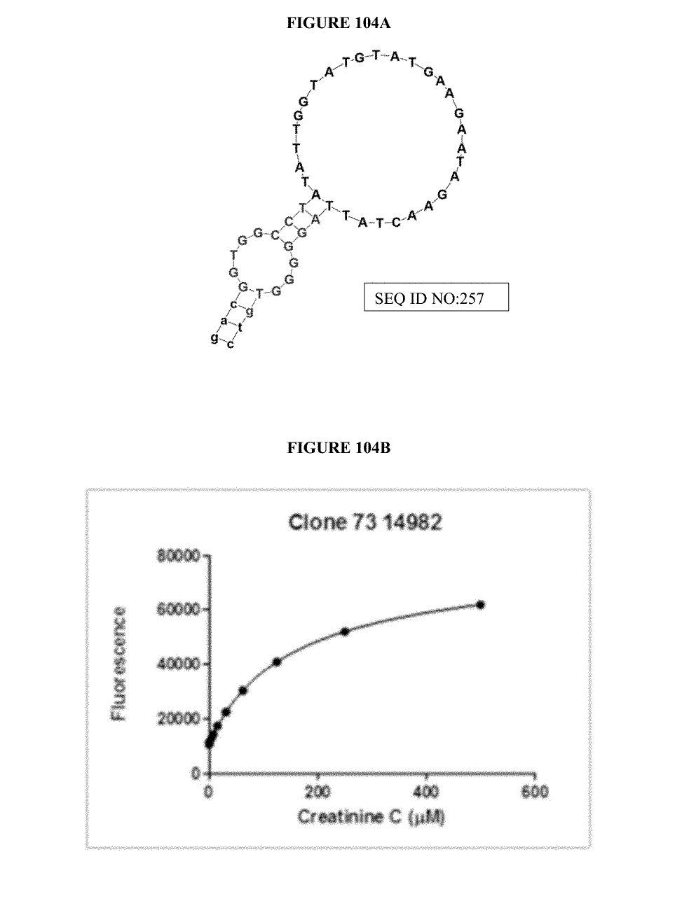

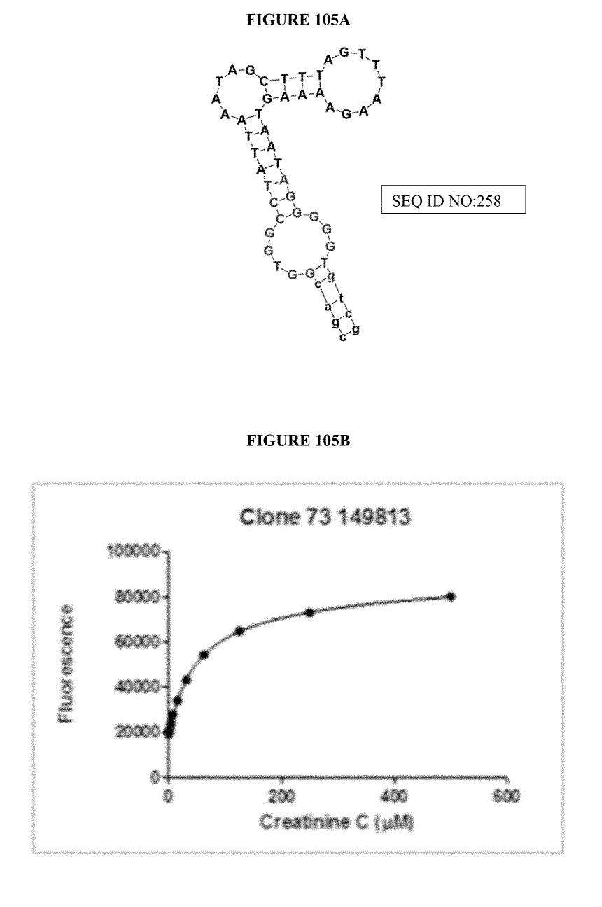

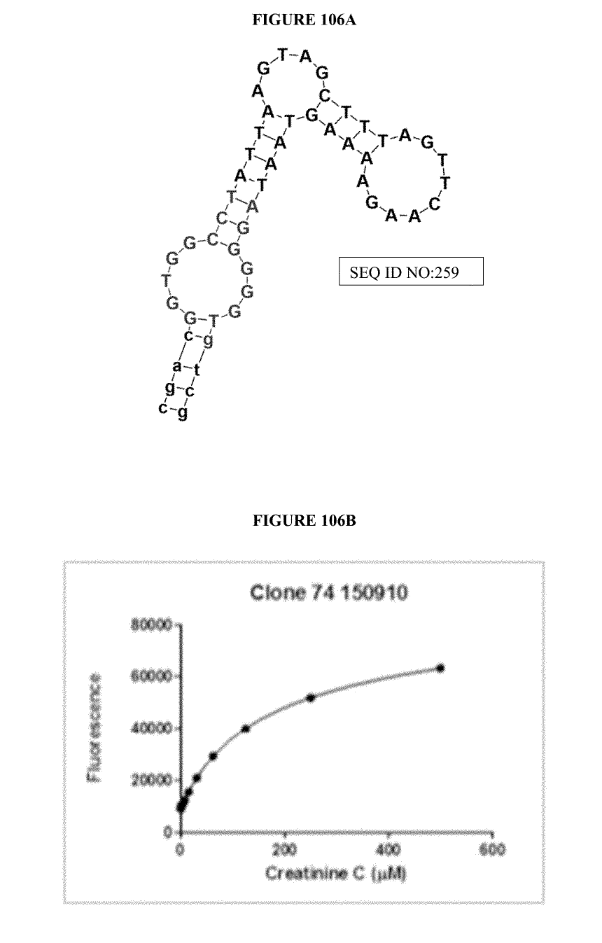

For example, but not by way of limitation, a creatinine-binding aptamer may comprise the sequence: CGA C GGTGGCCTATTAAATAGCTTTAGTT TAAGAAAAGTAATAGGGGGT GT CG (SEQ ID NO:258; FIG. 105A).

For example, but not by way of limitation, a creatinine-binding aptamer may comprise the sequence: CTC TCG GGA CGA C GGTGGCCTATTAAGTAGCTTTA GTTCAAGAAAAGTAATAGGGGGT GT CGT CCC (SEQ ID NO: 289).

In certain non-limiting embodiments, isolated creatinine-binding primary aptamers comprise the nucleotide sequence of SEQ ID NO:256, SEQ ID NO:257, SEQ ID NO:258, SEQ ID NO:259 or variants of these sequences having at least about 80 percent, or at least about 85 percent, or at least about 90 percent, or at least about 95 percent, or at least about 98 percent homology to the original sequence, for example obtained by substitutions, deletions, and insertions of Watson-Crick base pairs or by mutations at non-conserved positions. Percent homology can be determined using standard software such as BLAST or FASTA. In certain non-limiting embodiments, isolated creatinine-binding primary aptamers comprise the nucleotide sequence of SEQ ID NO:256, SEQ ID NO:257, SEQ ID NO:258, or SEQ ID NO:259. Said aptamers can bind to creatinine and in their structure-switching formats (for example, in an anti-aptamer assay, a pseudosandwich assay, or a sandwich assay) they can respond by an increase in fluorescence.

5.1.21. Vasopressin-Binding Primary Aptamers

In certain non-limiting embodiments, a primary aptamer binds to vasopressin in an aqueous solution at room temperature or 25° C. with a dissociation constant of less than 10⁻⁴ M and binds selectively with vasopressin versus oxytocin or pressinoic acid.

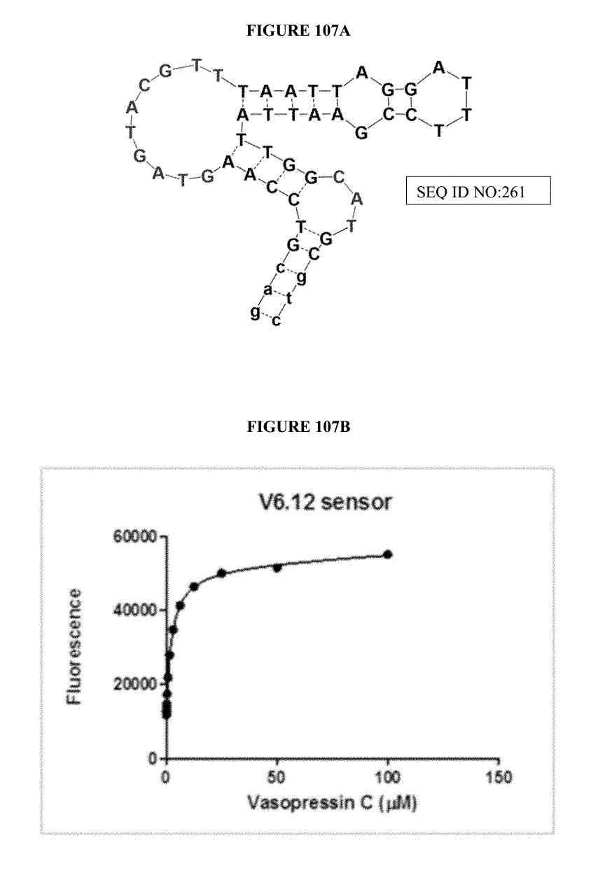

In certain non-limiting embodiments, a vasopressin-binding primary aptamer comprises the sequences GTAGTACGTT (SEQ ID NO:260) and CAT or a variant of any of these sequences that differs in one or two bases by substitution, deletion, insertion or extension, where said primary aptamer binds to vasopressin (see FIG. 107B) in an aqueous solution at room temperature or 25° C. with a dissociation constant of less than 10⁻⁴ M and binds to vasopressin selectively versus oxytocin or pressinoic acid. In certain non-limiting embodiments, a vasopressin-binding primary aptamer comprises the sequences GTAGTACGTT (SEQ ID NO:260) and CAT or a variant thereof and further comprises at least one operative sequence. In certain non-limiting embodiments, a vasopressin-binding primary aptamer comprises the sequences GTAGTACGTT (SEQ ID NO:260) and CAT or a variant thereof, and further comprises at least one operative sequence, said operative sequence complementary to a sequence comprised in a sensor oligonucleotide. In certain non-limiting embodiments, a vasopressin-binding primary aptamer comprises the sequences GTAGTACGTT (SEQ ID NO:260) and CAT, or a variant thereof, and at least one operative sequence on either side (flanking) these two sequences. In certain non-limiting embodiments, a vasopressin-binding primary aptamer comprises the sequences GTAGTACGTT (SEQ ID NO:260) and CAT or a variant thereof, and at least one operative sequence on either side (flanking) these two sequences, where two of said operative sequences contain mutually complementary portions and can form a duplex.

For example, but not by way of limitation, a vasopressin-binding aptamer may comprise the sequence: GA C GTCCAAGTAGTACGTTTAATTAGG ATTTCCGAATTATTGGCATGC GT C (SEQ ID NO:261; FIG. 107A)

In certain non-limiting embodiments, isolated vasopressin-binding primary aptamers comprise the nucleotide sequence of SEQ ID NO:261 or variants of these sequences having at least about 80 percent, or at least about 85 percent, or at least about 90 percent, or at least about 95 percent, or at least about 98 percent homology to the original sequence, for example obtained by substitutions, deletions, and insertions of Watson-Crick base pairs or by mutations at non-conserved positions. Percent homology can be determined using standard software such as BLAST or FASTA. In certain non-limiting embodiments, isolated vasopressin-binding primary aptamers comprise the nucleotide sequence of SEQ ID NO:261. Said aptamers can bind to vasopressin and in their structure-switching formats (for example, in an anti-aptamer assay, a pseudosandwich assay, or a sandwich assay) they can respond by an increase in fluorescence.

5.2 Anti-Aptamer Assays

In certain embodiments, an “anti-aptamer assay” is provided, in which a sample to be tested for the presence and/or amount of an analyte of interest is contacted with effective amounts of (1) a primary aptamer comprising a core sequence that binds to the analyte and (2) an “anti-aptamer” which is complementary to at least a portion of the primary aptamer, wherein the primary aptamer and/or anti-aptamer comprise a detectable moiety(ies) which detect whether the primary aptamer and anti-aptamer are bound to each other or unbound; and wherein a primary aptamer bound to the analyte does not bind to its anti-aptamer.

Certain embodiments provide for a method of detecting or measuring the presence or amount of analyte of interest in a sample, comprising (i) contacting at least a portion of a sample with effective amounts of a primary aptamer and an anti-aptamer that is complementary to at least a portion of the primary aptamer, said primary aptamer and/or anti-aptamer comprising a moiety which allows the amount of primary aptamer bound to analyte to be detected and/or measured, under conditions that would permit duplex formation between the primary aptamer and anti-aptamer if target analyte were not present; and (ii) detecting and optionally quantifying the amount of primary aptamer that is not bound to anti-aptamer.

A sample may be, for example and not limitation, a blood sample, a plasma sample, a serum sample, a urine sample, a tissue sample, a cerebrospinal fluid sample, a sputum sample, a fecal sample, a water sample, an industrial sample, etc.

As an illustrative example and not by way of limitation, the presence or amount of analyte of interest in a sample may be determined by (i) contacting at least a portion of the sample with effective amounts of (a) a primary aptamer comprising a fluorescent label and (b) an anti-aptamer, complementary to at least 80 percent or at least 90 percent or at least 95 percent or at least 98 percent of the primary aptamer, comprising a moiety that quenches fluorescence of said fluorescent label if primary aptamer and anti-aptamer are bound together in a duplex (for example, as a double-stranded molecule), under conditions that would permit duplex formation between primary aptamer and anti-aptamer to occur if analyte were not present; and (ii) detecting and optionally quantifying the amount of fluorescence. The amount of fluorescence may further be compared to the amount of fluorescence that results from a control mixture of primary aptamer and anti-aptamer in the absence of sample or in the presence of a known amount of analyte (e.g., a standard curve). This example may be modified as would be known to one skilled in the art. For example, different moieties may be used to detect duplex formation, such as a colorimetric label, an enzymatic label, a radiolabel, etc., and the detectable label may be carried on the anti-aptamer, depending on assay design.

In certain embodiments, a primary aptamer comprises one or more operative sequence which is complementary to anti-aptamer and facilitates duplex formation. For example, an operative sequence with a nucleotide composition which favors duplex formation may be used (a.k.a. a “toe hold” sequence).

The anti-aptamer assay and method of using it may utilize any primary aptamer that binds an analyte of interest, including but not limited to those set forth herein.

An anti-aptamer has a sequence which is complementary to at least 80 percent or at least 90 percent or at least 95 percent or at least 98 percent of its corresponding primary aptamer.

A primary aptamer or anti-aptamer for use in the anti-aptamer assay may be between about 30 and about 200 nucleotides, or between about 30 and 100, or between about 30 and 80, nucleotides in length.

In certain non-limiting embodiments, the primary aptamer and anti-aptamer may be present, in the assay, in a ratio of about 1:1. Depending on the strength of binding between analyte and primary aptamer and/or between primary aptamer and anti-aptamer, it may be desirable to alter this ratio to produce a dose-response curve having the desired features (for example, being able to measure/detect analyte at a particular concentration). As an illustrative example, and not by way of limitation, if binding between primary aptamer and analyte is very strong relative to the affinity between primary aptamer and anti-aptamer, it may be desirable to increase the relative amount of anti-aptamer, so that the ratio is 1:greater than 1. Non-limiting examples are about 1:1;

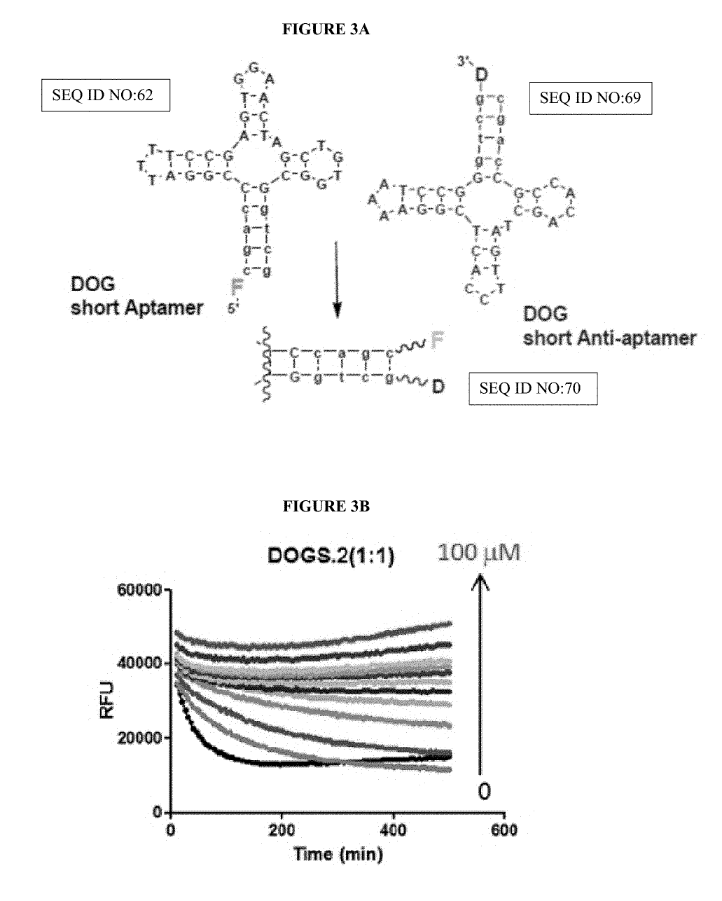

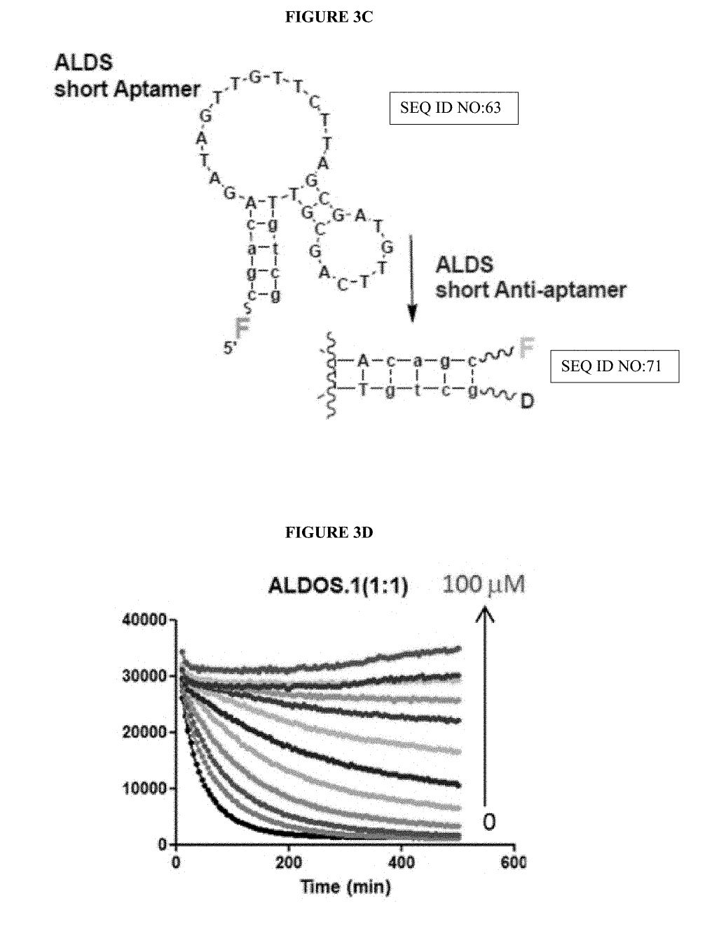

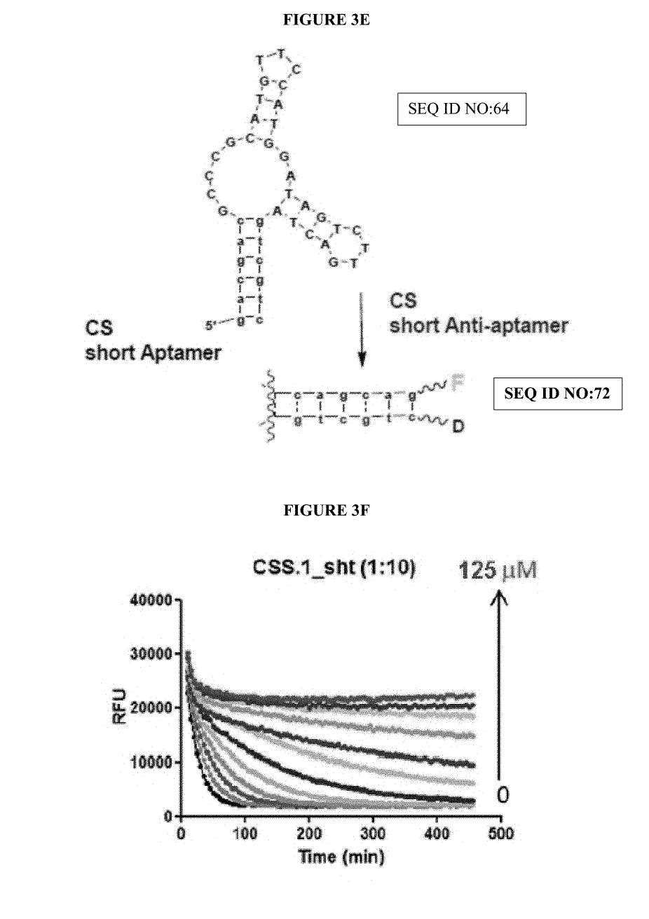

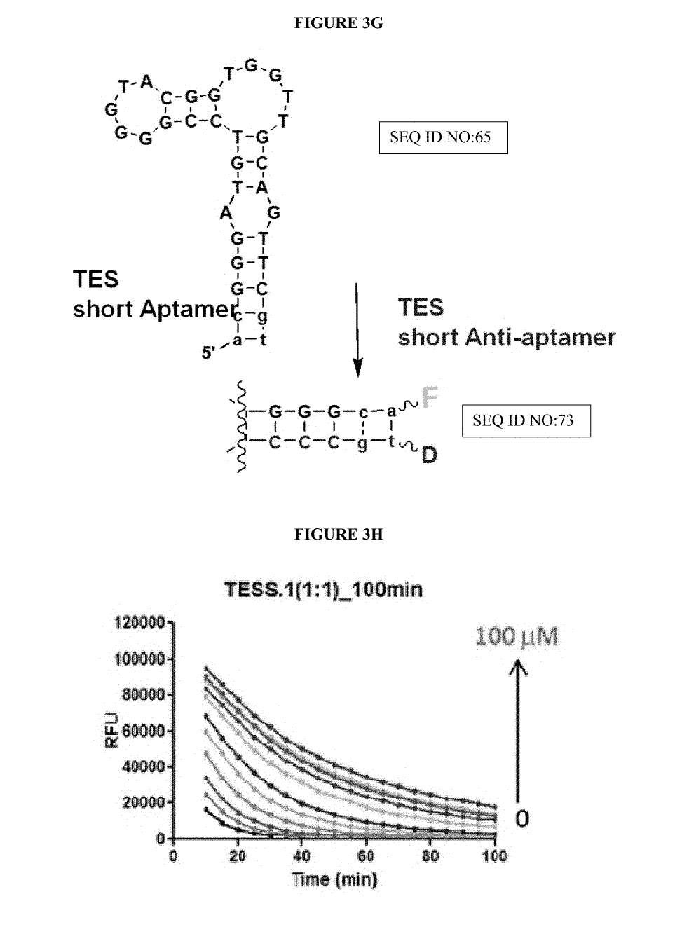

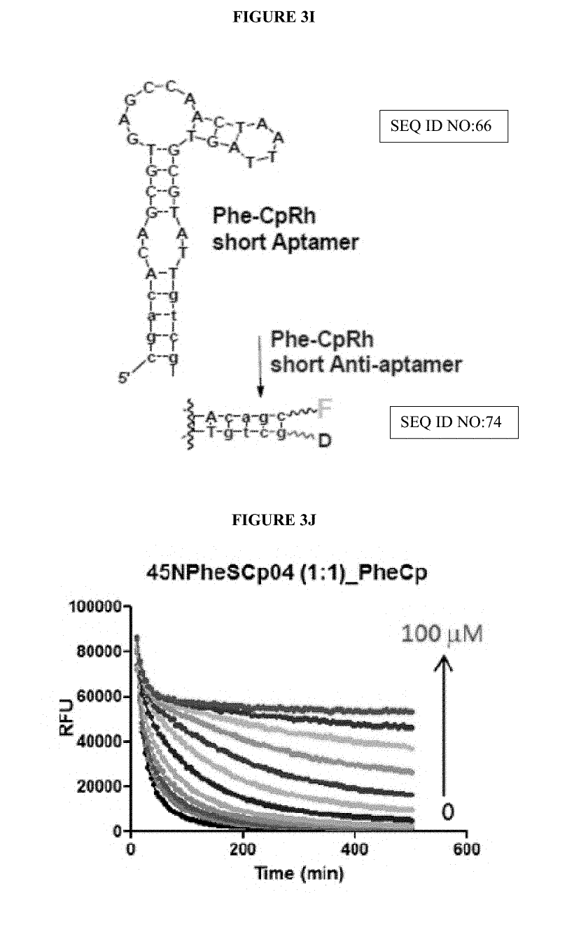

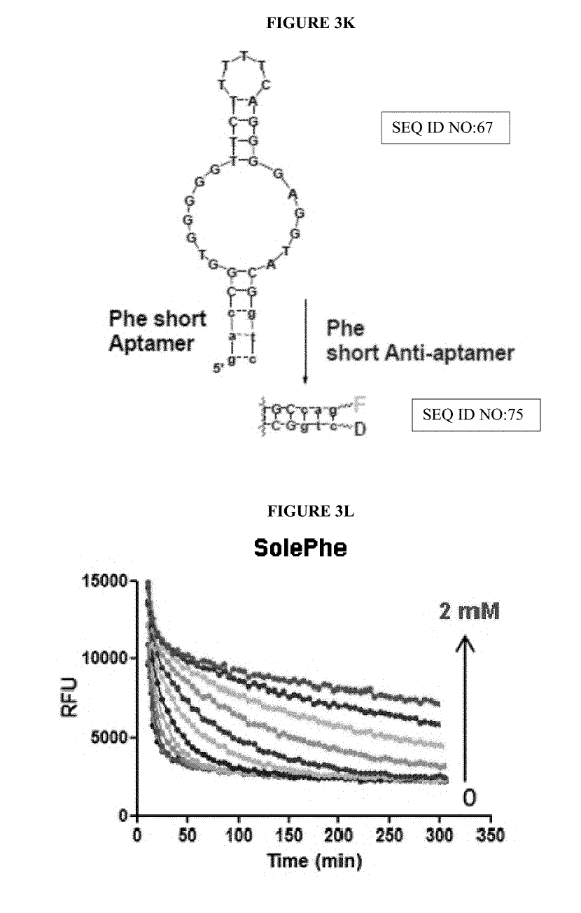

In certain non-limiting embodiments, an anti-aptamer assay may be performed in solution. Non-limiting examples of the use of anti-aptamer assay, in solution, for detecting various analytes are shown in FIG. 3A-L, showing results for measuring deoxycortisoen (FIG. 3A-B); aldosterone (FIG. 3C-D); cortison (FIG. 3E-F); testosterone (FIG. 3G-H); Phen-CpRh (FIG. 3I-J), and phenylalanine (FIG. 3K-L). In a specific non-limiting example, provided as illustration, 400 nM FAM-aptamer solution and four-times concentrated Iowa Black-anti-aptamer (“complement strand”) solution are prepared separately in buffer (20 mM HEPES, 1 M NaCl, 10 mM MgCl2, 5 mM KCl, pH 7.5). These solutions are annealed separately by incubating in boiling water for 5 min, and cooled down at room temperature for ˜30 min. Meanwhile, a two-times concentrate of each aptamer's target solutions are prepared. After the 30 min incubation period of the aptamer and anti-aptamer solutions, 18.75 ul of the aptamer solution is added first to the 384-well plate, then 37.5 ul of the target solution is added, and then immediately added is 18.75 ul of anti-aptamer solution. Without incubation, the fluorescent measurement is started and read every 5 min at room temperature for >8 hours by using Fluorescence plate reader (Victor II microplate reader, PerkinElmer). The final concentration of aptamer is 100 nM, the concentration of complementary strand is 100 nM for all except CS aptamer (1000 nM) in 75 μL reaction volume.

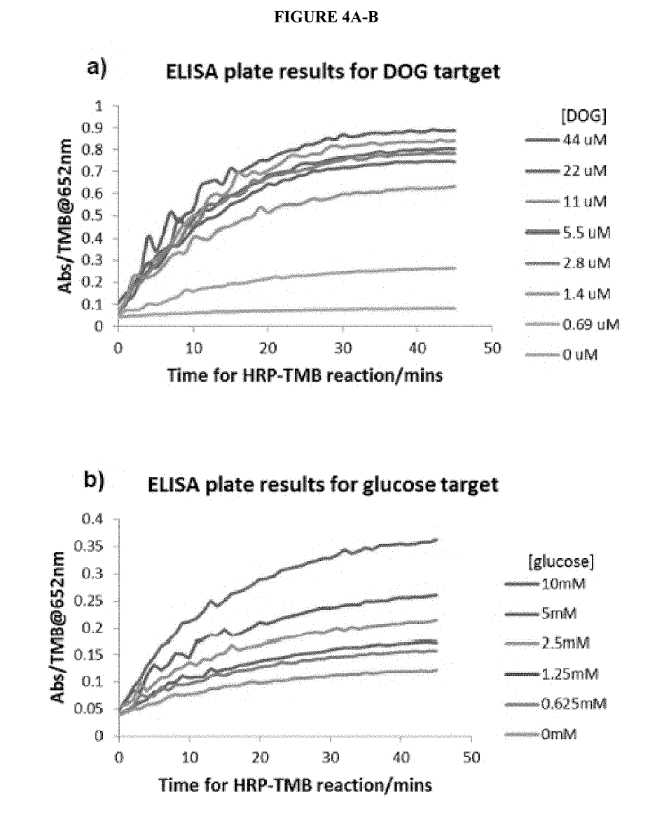

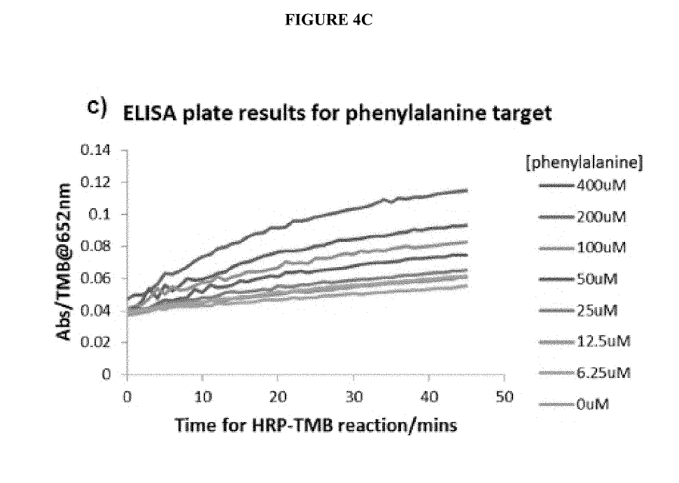

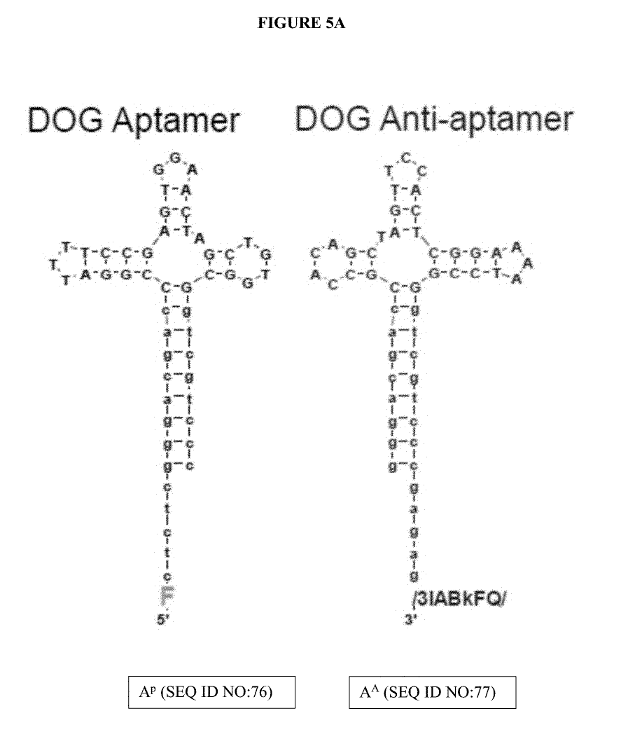

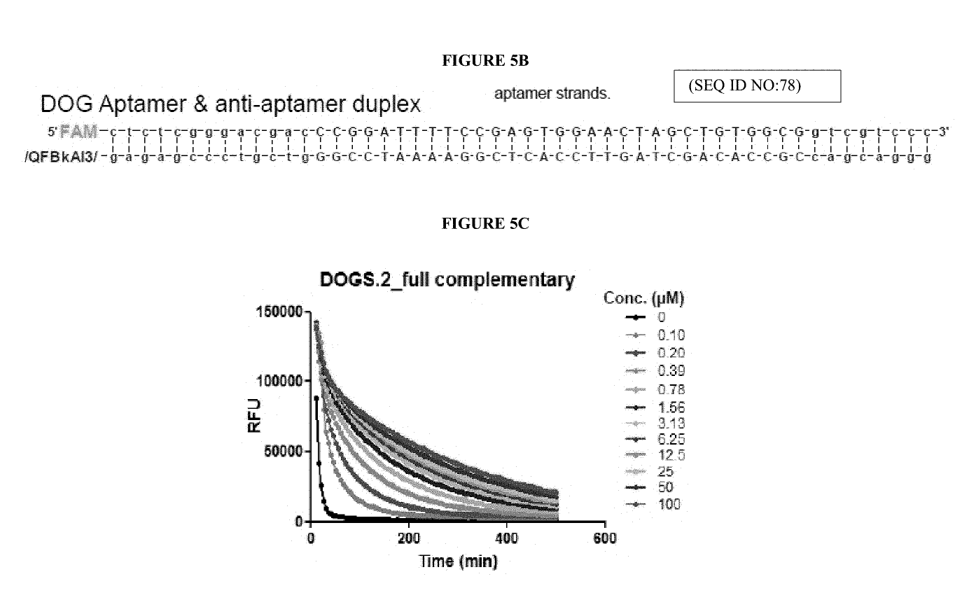

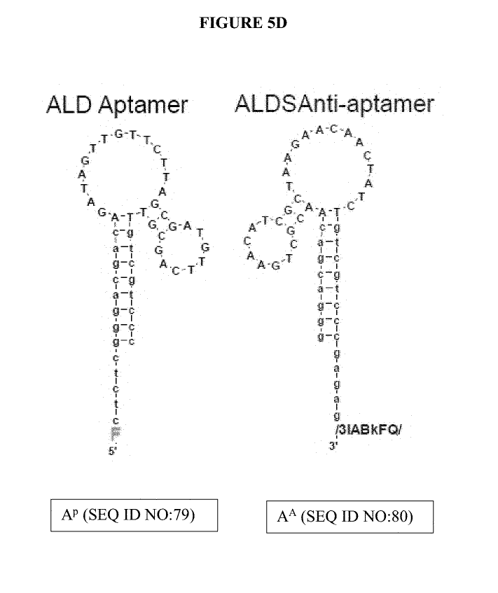

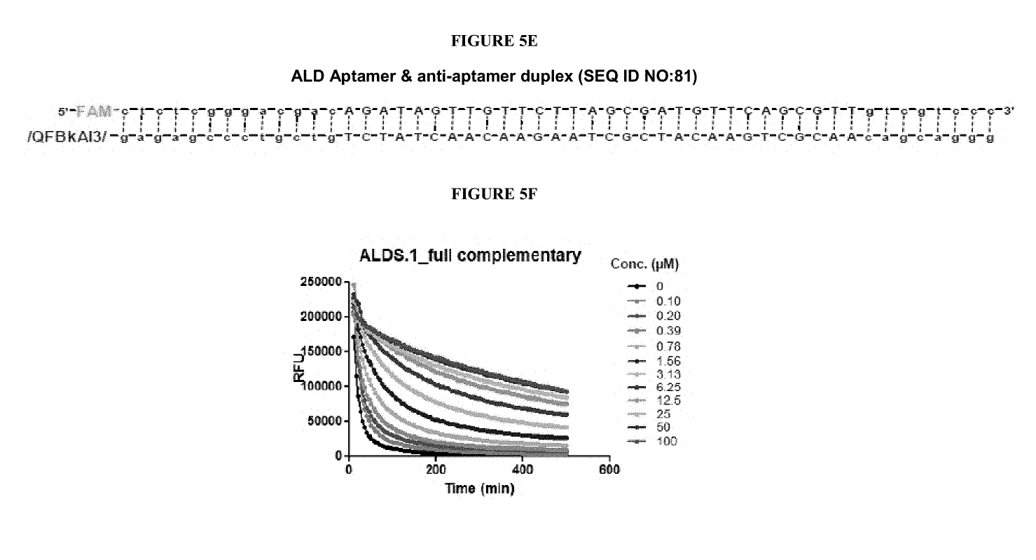

In certain non-limiting embodiments, an anti-aptamer assay may be performed in as a solid phase assay, with primary aptamer or anti-aptamer bound a solid phase (e.g., an ELISA-like format). Non-limiting examples of the use of anti-aptamer assay as a solid-phase assay, for detecting various analytes are shown in FIG. 4A-C for deoxycortisone (FIG. 4A), glucose (FIG. 4B) and phenylalanine (FIG. 4C).

In certain non-limiting embodiments, a kit is provided for practicing an anti-aptamer assay as described herein. For example, said kit comprises a primary aptamer directed toward an analyte of interest and a corresponding anti-aptamer. Non-limiting examples of primary aptamers that may be comprised in such a kit include the primary aptamers set forth herein, for example as described in Section 5.1 and FIGS. 50A-B to 102AB, and a corresponding anti-aptamer as described herein. Said kit may optionally further comprise target analyte, for example a control solution comprising target analyte, and/or a standard curve.

5.3 Pseudosandwich Assay

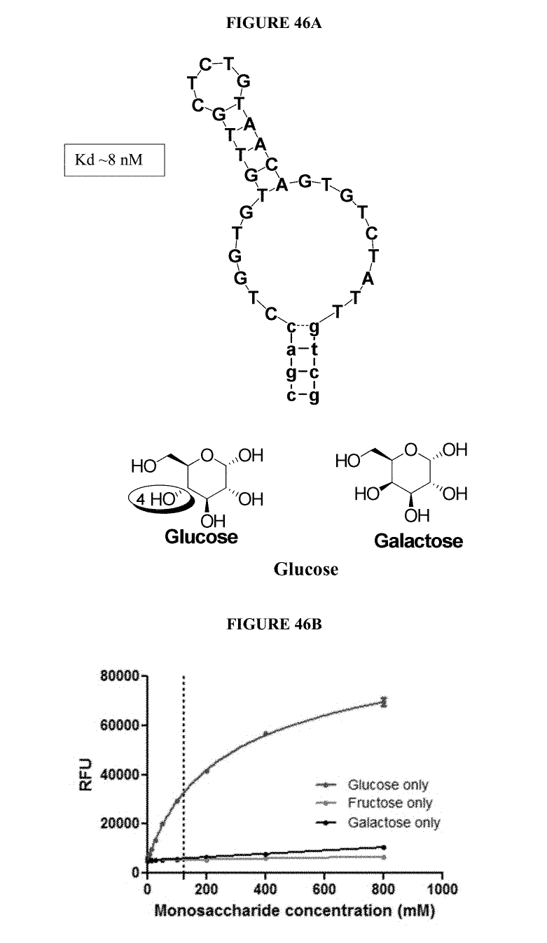

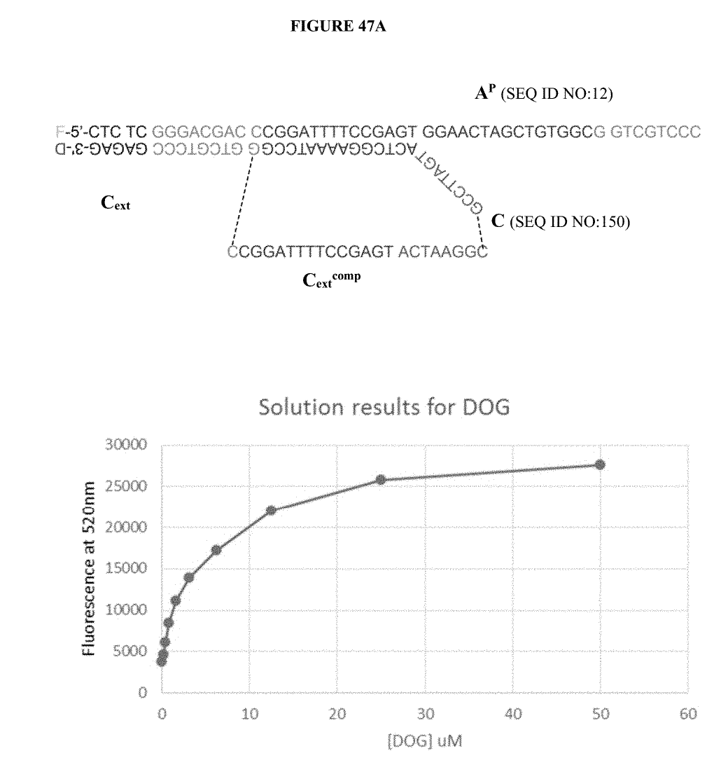

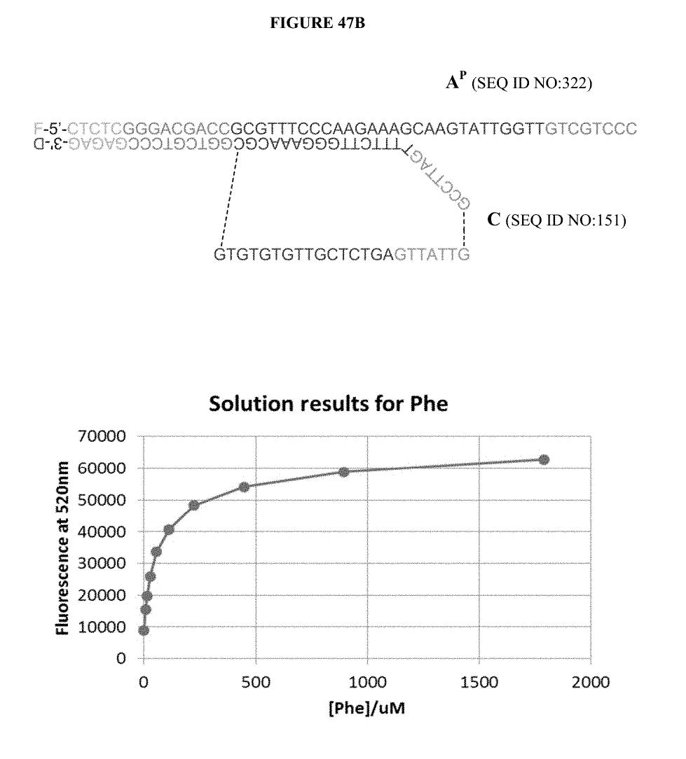

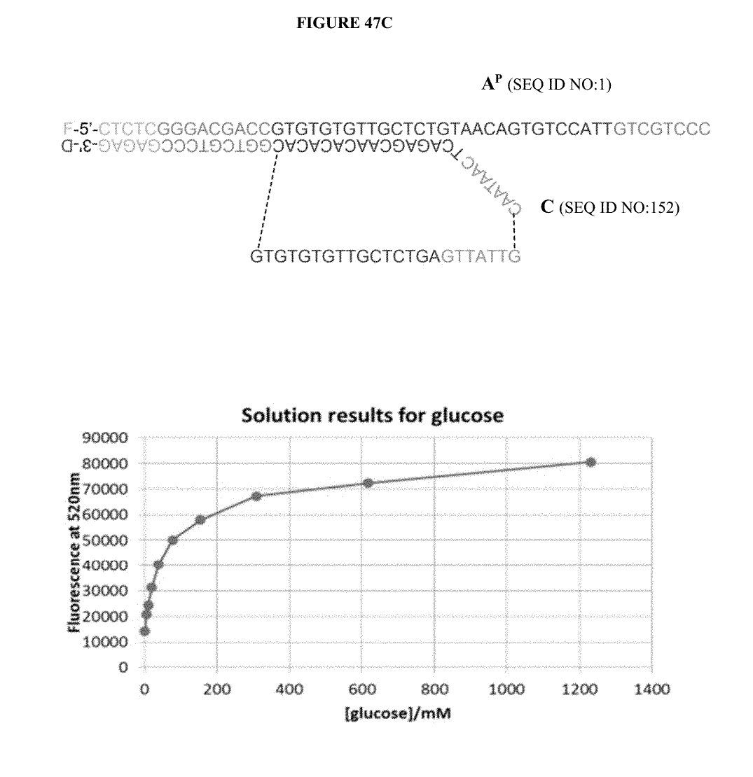

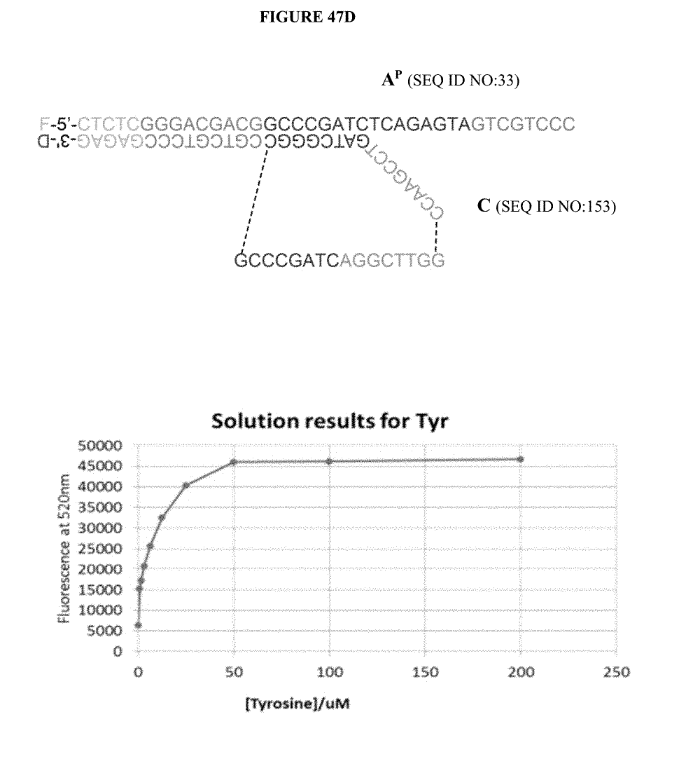

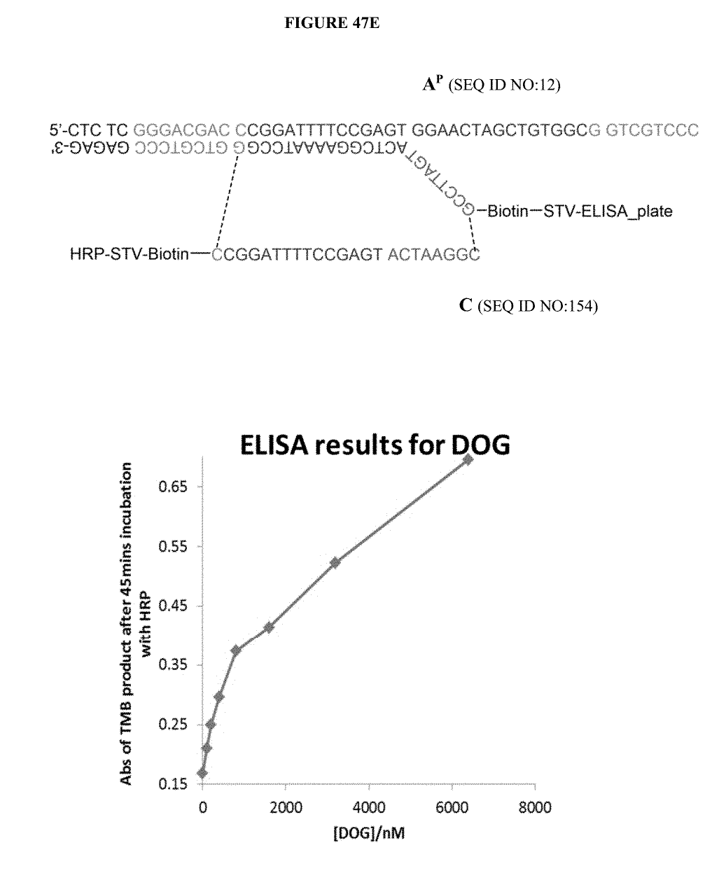

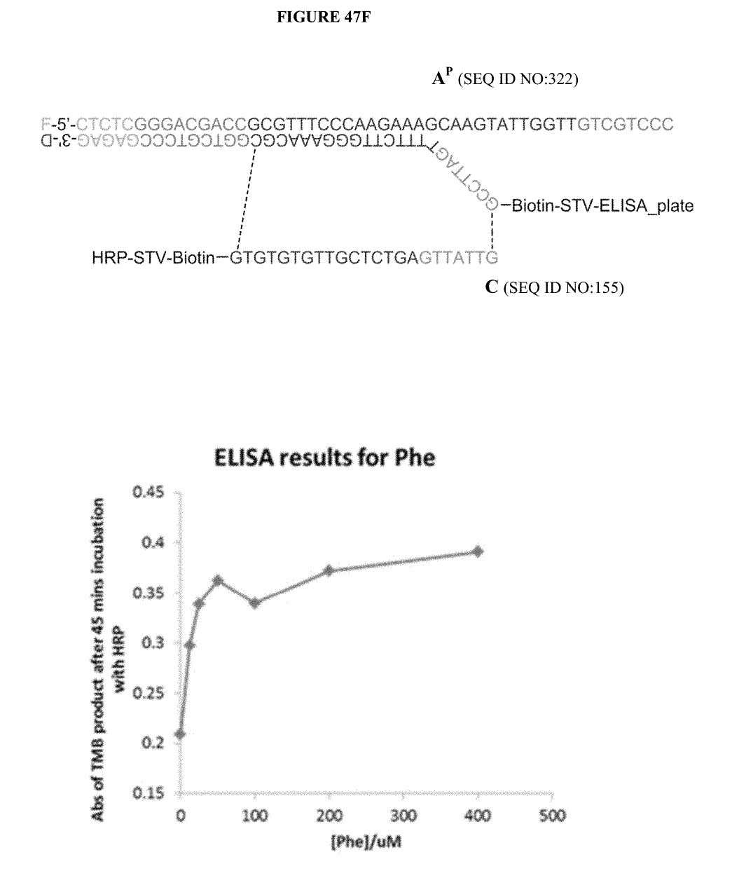

The present application discloses pseudosandwich assays in solution (FIG. 47A-D) and on plates (FIG. 47E-F). In certain non-limiting embodiments, the pseudosandwich assays employ a primary aptamer.

Certain embodiments provide for a “pseudosandwich assay”, or method of detecting or measuring the presence or amount of an analyte of interest in a sample, comprising (i) contacting at least a portion of a sample with effective amounts of (a) a primary aptamer comprising a core sequence that binds to the analyte and a portion complementary to a sensor oligonucleotide; (b) a sensor oligonucleotide; and optionally (c) a comp oligonucleotide; one or more of which is bound to a detectable moiety(ies) which can detect whether the primary aptamer and sensor oligonucleotide are bound to each other or whether primary aptamer is bound to analyte.

In certain non-limiting embodiments, the method comprises:

(1) Isolating primary aptamers (APs) by solution-phase (FIG. 7) or solid-phase selection, unless they are already available; use of enantiomeric aptamer (spiegelmers), if desired to minimize Watson-Crick base pairing (e.g., fusing aptamers or to minimize background interactions without analyte);

(2) Testing of the primary aptamer in its structure-switching form and modifying its structure switching form, which is then turned into pseudo-sandwich assay format (FIG. 6C);

(3) Isolating secondary aptamers (A^{s}s) by either solution-phase or solid phase selections (FIG. 9), using primary aptamers or spiegelmers in their complexes with targets; and

(4) Implementing sandwich assays for targets (FIG. 8).

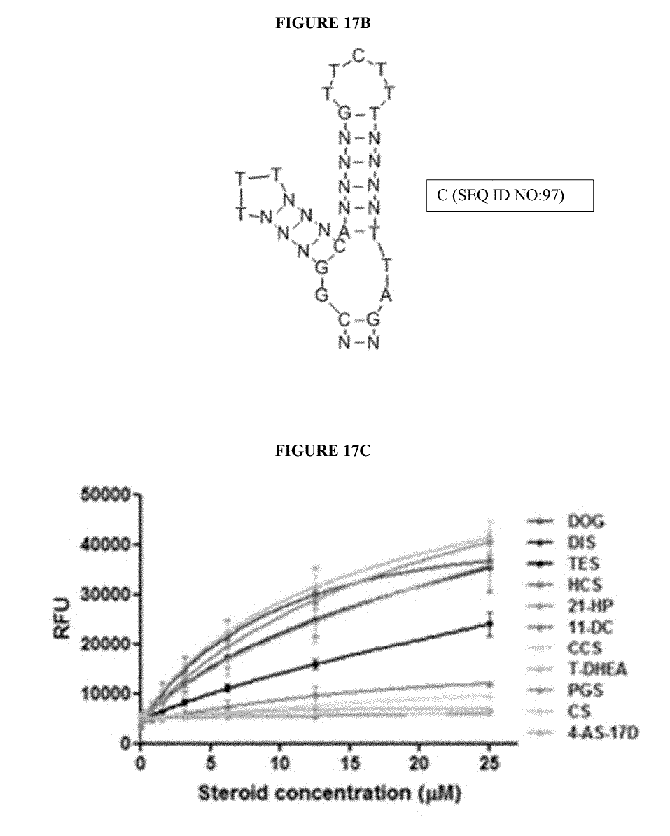

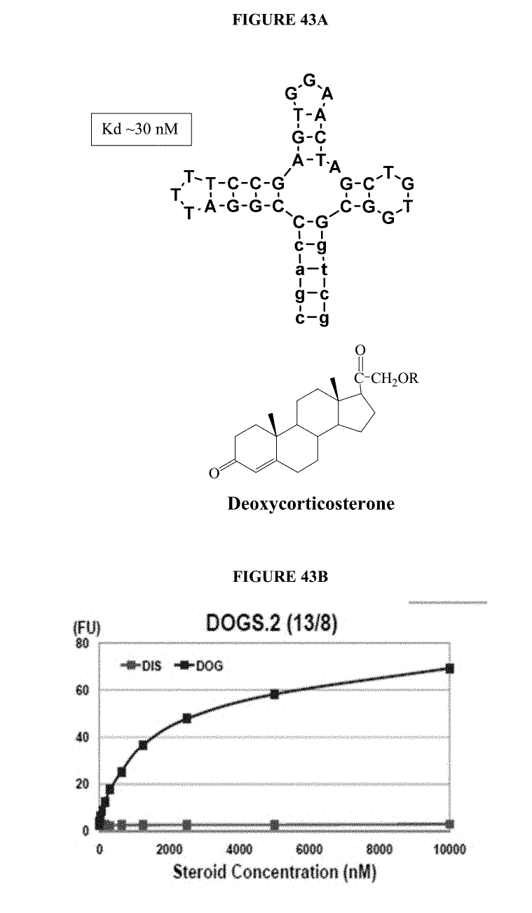

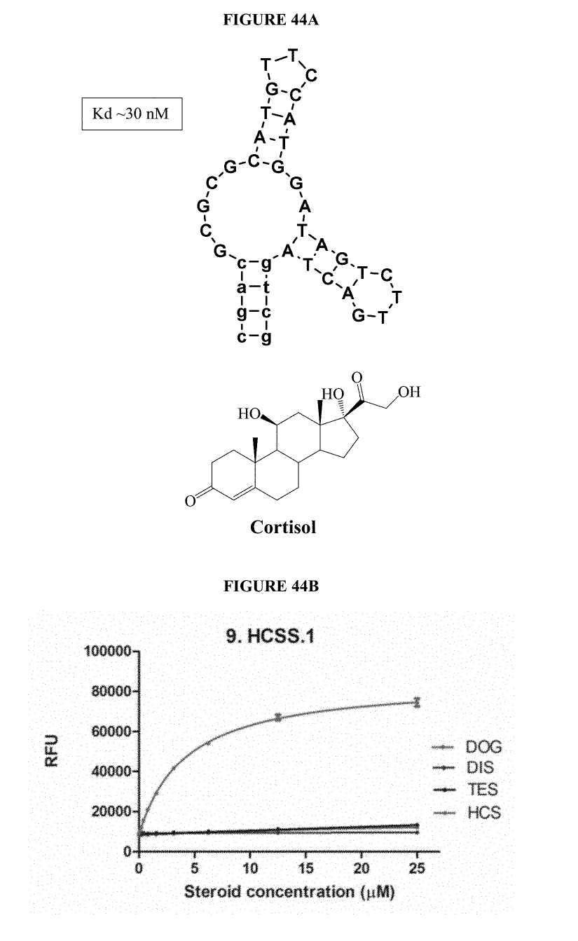

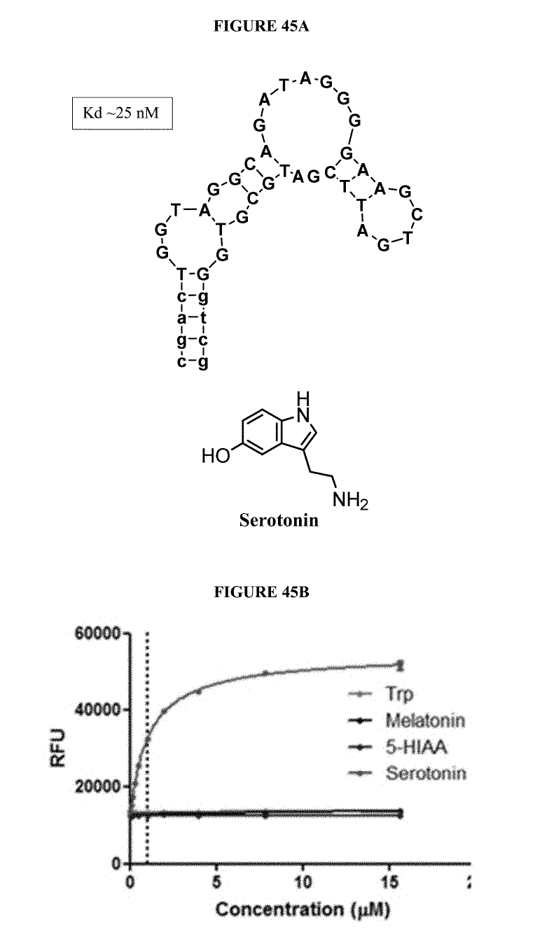

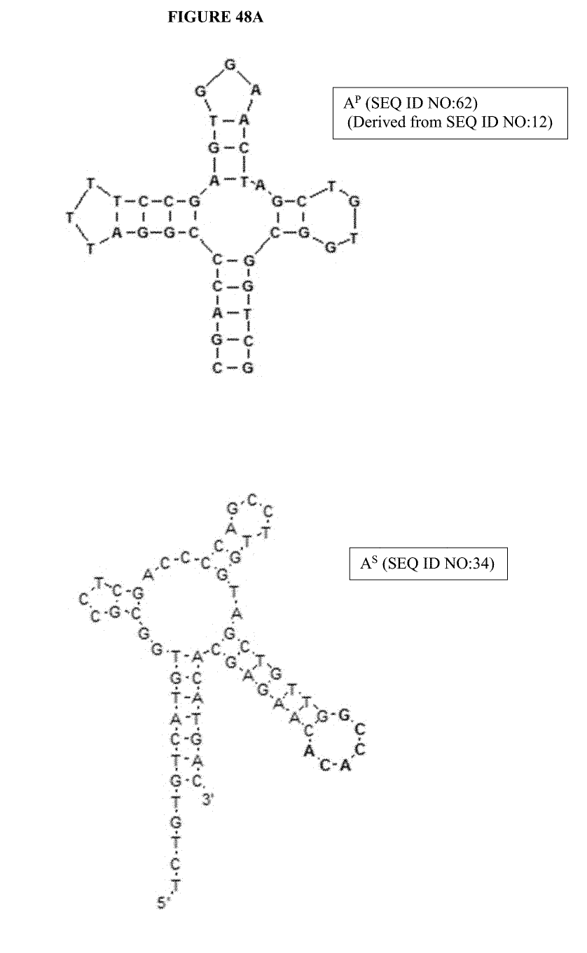

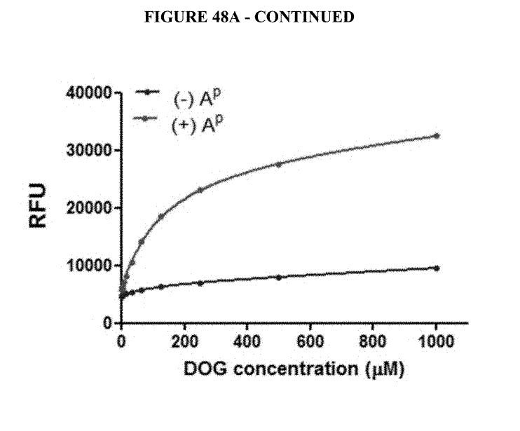

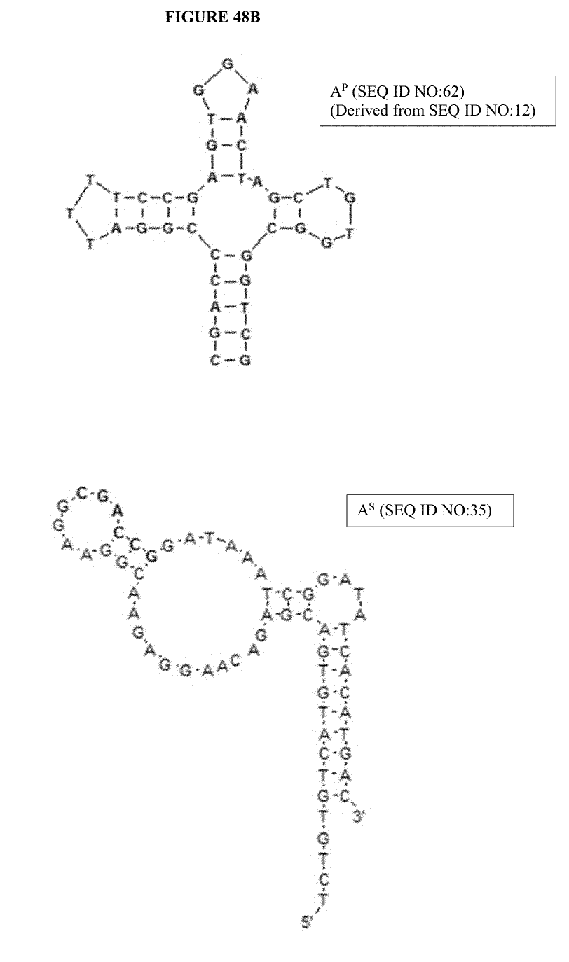

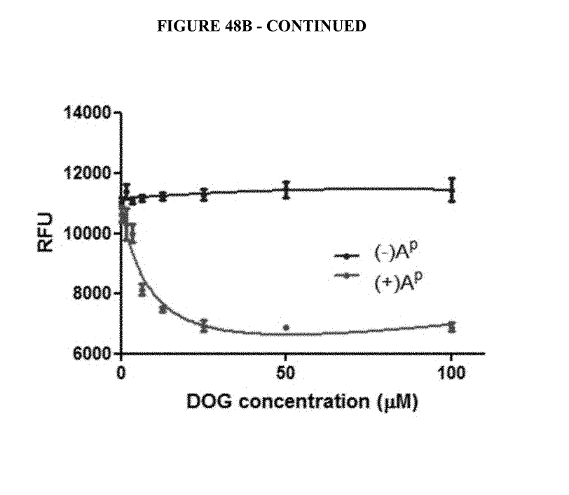

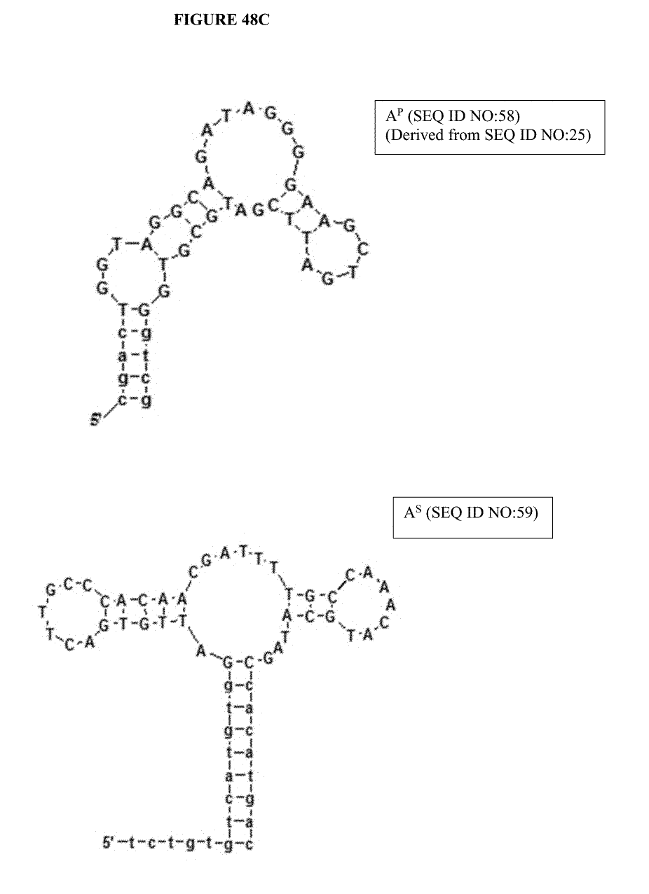

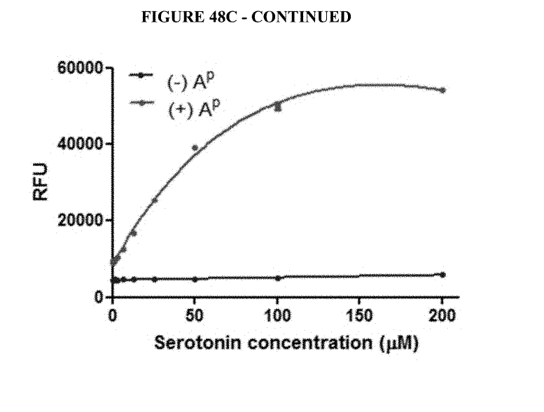

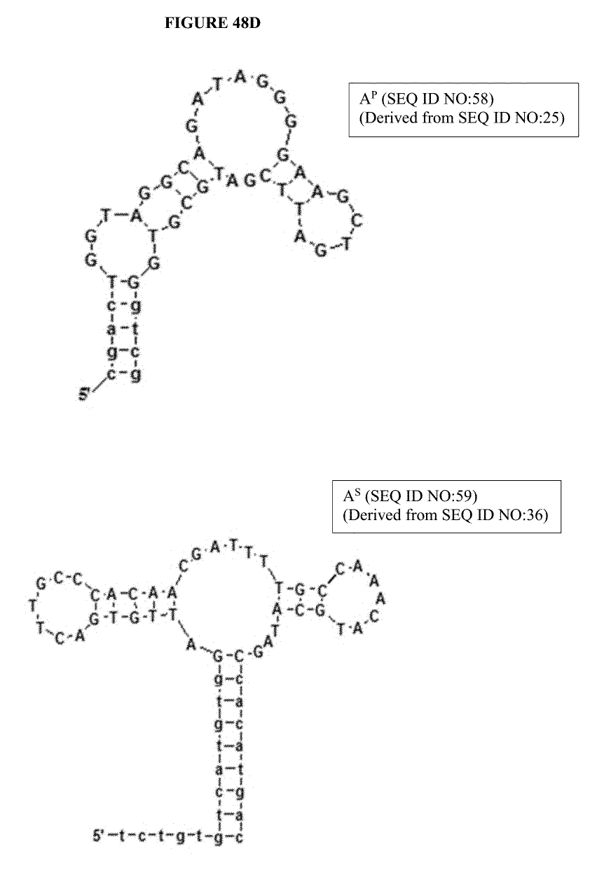

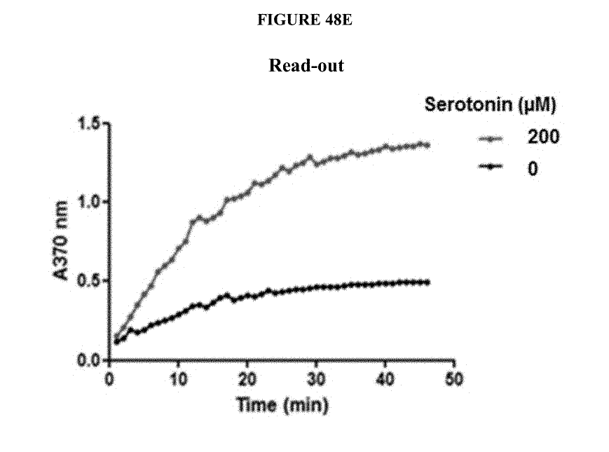

An exemplary list of the disclosed primary aptamers are provided in FIG. 10-46 in their sensor (structure-switching) forms¹⁶⁻²¹, together with their associated sensor oligonucleotides. The disclosed primary aptamers were isolated using solution-phase selection and can be used in aptamer-based assays, not limited to sandwich and pseudosandwich assays. In certain non-limiting embodiments, primary aptamers (FIG. 10-46) can be spiegelmers or unnatural enantiomers of nucleic acids²²⁻²⁵. In certain non-limiting embodiments, primary aptamers can be directly engineered to be used in “pseudosandwich” assays (see FIG. 6, FIG. 47 and FIG. 48).

A “pseudo-sandwich” assay transforms reversible interactions of analyte-binding aptamers (primary aptamers or A′) into a more stable partially double helical complex, which can be used in solution-phase assays (FIG. 6 and FIG. 47A-D), or in solution-state assays (plates, beads or components of lateral flow devices), in which case it may allow extensive washing (FIG. 6 and FIG. 47E-F).

The stable double helical product released once a primary aptamer is bound to its analyte can be also captured to a solid state surface (e.g., plate well or beads or lateral flow pad) and subjected to extensive washing, therefore eliminating many sources of high background in other aptamer-based assays using structure switching principles. The oligonucleotide in complex with capture oligonucleotide can be then used to generate amplified readout indicative of presence and quantity initially bound analyte, like in a typical sandwich ELISA.

For example, a complementary oligonucleotide (C_{D}) can be extended (C_{Dext}) into an aptamer. While C_{D} on its own is in equilibrium and 3-5 equivalents are used to achieve quenching of aptamers with fluorescein, C_{Dext} requires only one equivalent and it binds nearly irreversibly to the aptamers. This helps with the half-response point.

C_{Dext} can then be further extended by adding a “toehold” region that allows this kinetically protected stable complex to interact with an oligonucleotide complementary to C_{Dext} (C_{Dext}^{comp}). The presence of an excess of C_{Dext}^{comp}) in solution allows small amounts of aptamer to form binding pockets and interact with the L. This triggers establishment of equilibrium leading to a certain amount of double helix formations which is substantially increase with the release of double helix into solution. This process can be monitored by an increase in fluorescence in solution.

Pseudosandwich Assays—in Solution:

In certain non-limiting embodiments pseudosandwich assays in solution can be performed, wherein a double helix formation is triggered by deoxycorticosterone. In certain non-limiting embodiments, primary aptamers comprising the nucleotide sequence of SEQ ID NO: 12 or analogs can form a complex with C_{Dext} and in the presence of C_{Dext}^{comp} can produce an increase in fluorescence in the presence of deoxycorticosterone and analogs.

In certain non-limiting embodiments pseudosandwich assays in solution can be performed, wherein a double helix formation is triggered by tyrosine. In certain non-limiting embodiments, primary aptamers comprising the nucleotide sequence of SEQ ID NO: 33 or analogs can form a complex with C_{Dext} and in the presence of C_{Dext}^{comp} can produce an increase in fluorescence in the presence of tyrosine and analogs.

In certain non-limiting embodiments pseudosandwich assays in solution can be performed, wherein a double helix formation is triggered by glucose. In certain non-limiting embodiments, primary aptamers comprising the nucleotide sequence of SEQ ID NO: 1 or analogs can form a complex with C_{Dext} and in the presence of C_{Dext}^{comp} can produce an increase in fluorescence in the presence of glucose and analogs.

In certain non-limiting embodiments pseudosandwich assays in solution can be performed, wherein a double helix formation is triggered by Phenylalanine. In certain non-limiting embodiments, primary aptamers comprising the nucleotide sequence of SEQ ID NO: 2 or analogs can form a complex with C_{Dext} and in the presence of C_{Dext}^{comp} can produce an increase in fluorescence in the presence of Phenylalanine and analogs.

In certain non-limiting embodiments, if the C_{Dext} is deposited on plates, the double helix remains attached to the plate when the rest of solution is removed and it can now be extensively washed. This double helix can be used in numerous ways for the signal development.

Pseudosandwich Assays on Plate:

In certain non-limiting embodiments solution-evolved aptameric sensors are adapted for use in a solid surface-format application, for example, ELISA-type assays. Specifically, the solution sensor—composed of an analyte and a specific aptamer comprising the nucleotide sequence of SEQ ID NO: 12, which is partially hybridized to C_{Dext}—is modified with attachment chemistry to enable its anchoring to a solid surface.

In certain non-limiting embodiments the attachment chemistry comprises use of biotin or covalent bonding between maleimide and sulfhydryl functional groups. In addition, the complementary strand to the competitor oligonucleotide C_{Dext} (C_{Dext}^{comp}) (optimized to bind to the C_{Dext} in the presence of analyte) is modified with a biotin-tag, which is used to capture a “read-out” producing molecule e.g. HRP conjugate. In certain non-limiting embodiments, the “read-out” is performed using 3,3′,5,5′-Tetramethylbenzidine (TMB), the substrate for HRP, for visualization.

In certain non-limiting embodiments, optimization of response of the surface-bound sensor, relative to +/−analyte, can be carried out by adjustment of several parameters: Amount of sensor on surface; attachment chemistry, concentration of complementary competitor oligonucleotide; buffer in each step; washing steps; HRP concentration; HRP substrate.

In certain embodiments, a primary aptamer comprises at least one operative sequence which is complementary to at least a portion of a sensor oligonucleotide.

Non-limiting illustrative examples showing pairs of primary aptamer and sensor oligonucleotide are shown in FIGS. 10A-42A. For example, but not by way of limitation, glucose-binding primary aptamer (SEQ ID NO:1) comprises an operative sequence complementary to sensor oligonucleotide (SEQ ID NO:82); phenylalanine-binding primary aptamer SEQ ID NO:2 comprises an operative sequence complementary to sensor oligonucleotide (SEQ ID NO:84); phenylalanine-binding primary aptamer SEQ ID NO:3 comprises an operative sequence complementary to sensor oligonucleotide (SEQ ID NO:86; hydrocortisone-binding primary aptamer (SEQ ID NO5) comprises an operative sequence complementary to sensor oligonucleotide (SEQ ID NO:90), etc. In certain non-limiting embodiments, a pseudosandwich assay on plate can be performed to detect deoxycorticosterone.

A sample may be, for example and not limitation, a blood sample, a plasma sample, a serum sample, a urine sample, a tissue sample, a cerebrospinal fluid sample, a sputum sample, a fecal sample, a water sample, an industrial sample, etc.

In certain non-limiting embodiments, the foregoing assays can be used to detect and/or quantitate any analyte of interest, and can be particularly advantageous over existing methods in detecting and/or quantitating analytes that have a molecular weight less than about 1000 Daltons, or less than about 500 Daltons, or less than about 200 Daltons, including but not limited to steroid compounds, such as but not limited to cortisol, aldosterone, dehydroisoandrosterone, progesterone, testosterone; glucose; amino acids such as but not limited to phenylalanine, leucine, isoleucine, valine, citrulline, tyrosine, alanine; pharmaceutical compounds, vitamins, toxins, neurotransmitters (e.g., catecholamines, serotonin), peptides (vasopressin, oxytocin, angiotensin, natriuretic peptides, glucagon, insulin and others), antibiotics and antifungal compounds (e.g., aminoglucosides), macrocyclic immunosuppresants, or lipids or lipid complexes.

In certain non-limiting embodiments, the assays can also be applied to large molecules, for example, above 1000 D.

In certain non-limiting embodiments, a kit is provided for practicing a pseudo-sandwich assay as described herein. In certain embodiments, said kit comprises a primary aptamer directed toward an analyte of interest and a corresponding sensor oligonucleotide. Non-limiting examples of primary aptamers that may be comprised in such a kit include the primary aptamers set forth herein, for example as described in Section 6.1 and FIGS. 50A-B to 102AB, and a corresponding sensor oligonucleotide as described herein. Non-limiting examples of primary aptamer/sensor oligonucleotide pairs are set forth in FIGS. 10A-42A. Said kit may further comprise one or more comp oligonucleotide, as described herein. Said kit may optionally further comprise target analyte, for example a control solution comprising target analyte, and/or a standard curve.

5.4 Sandwich Assay

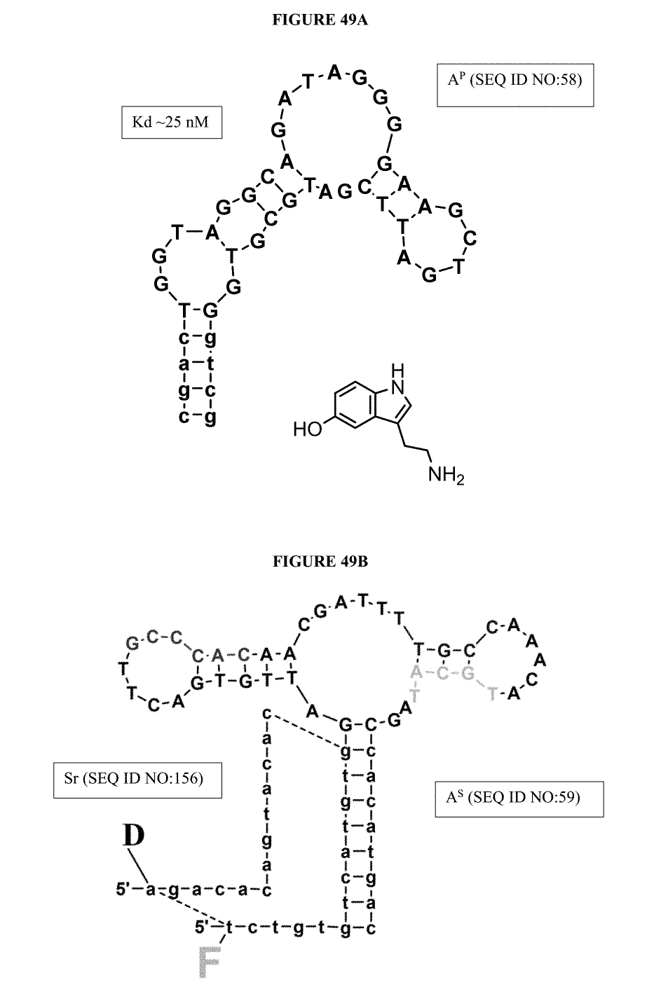

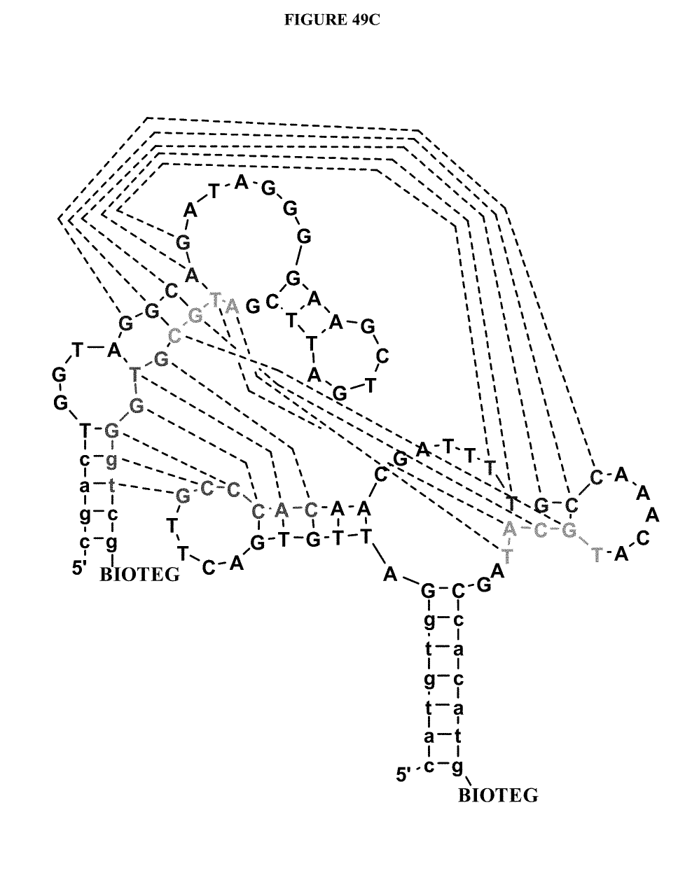

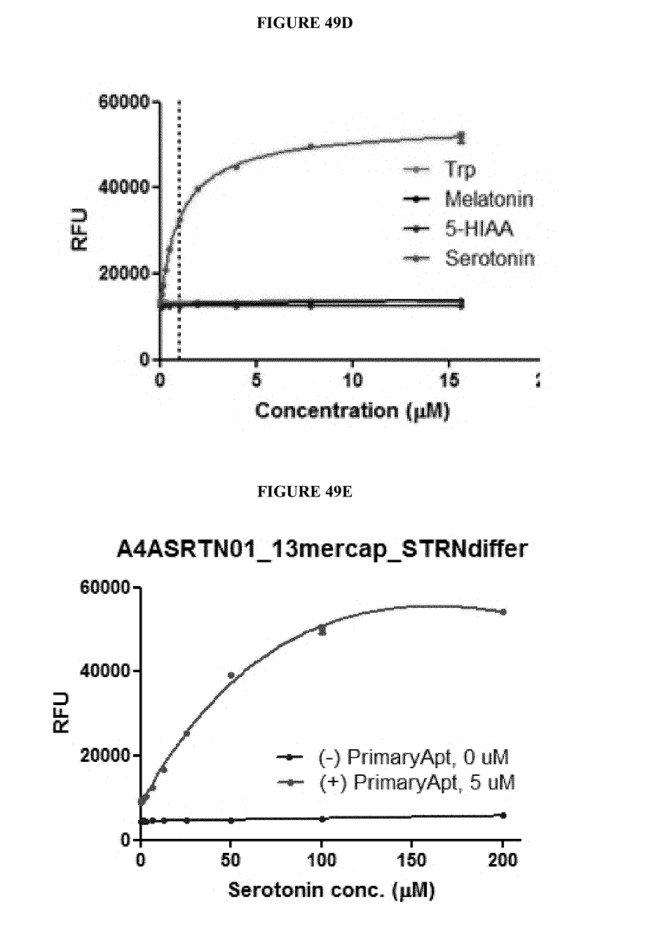

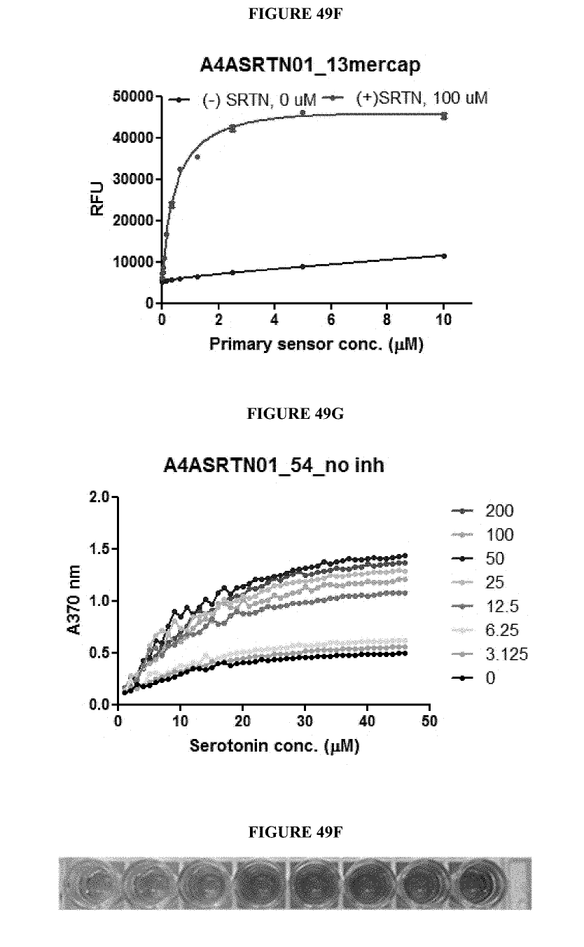

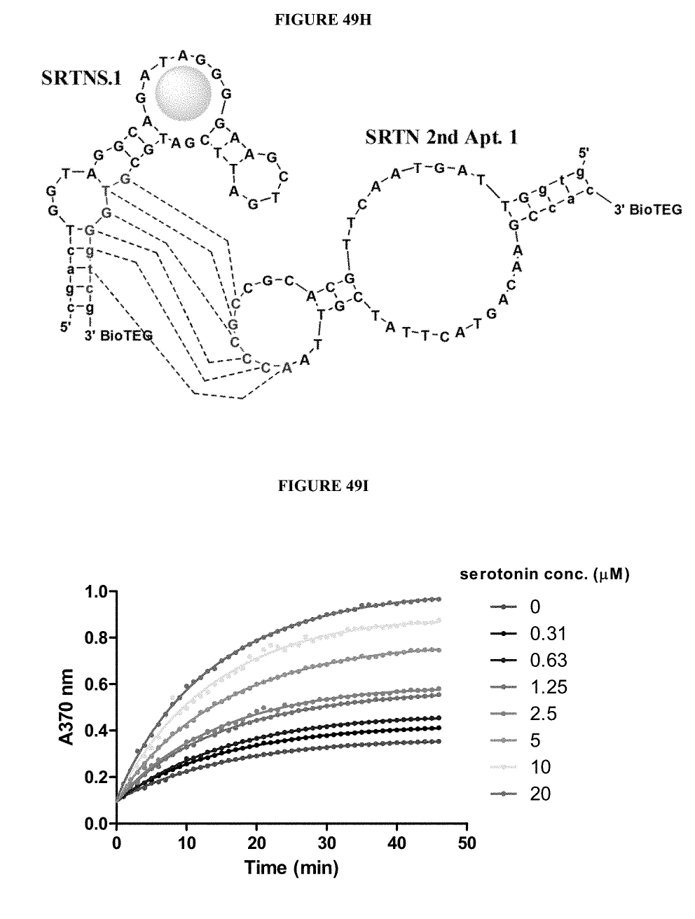

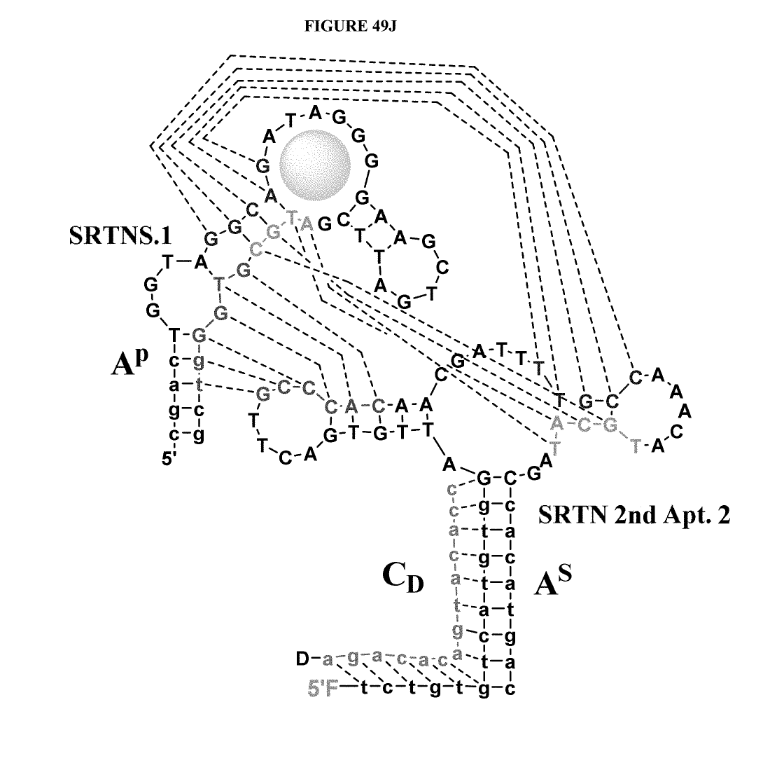

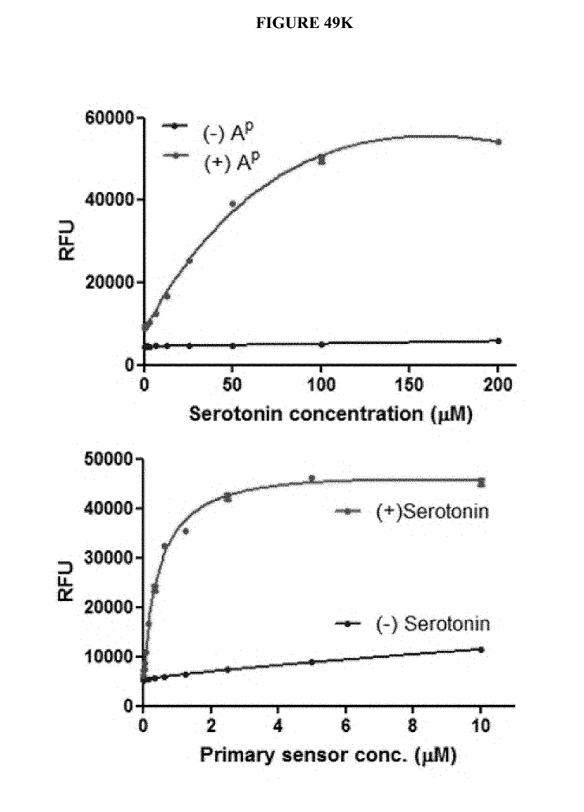

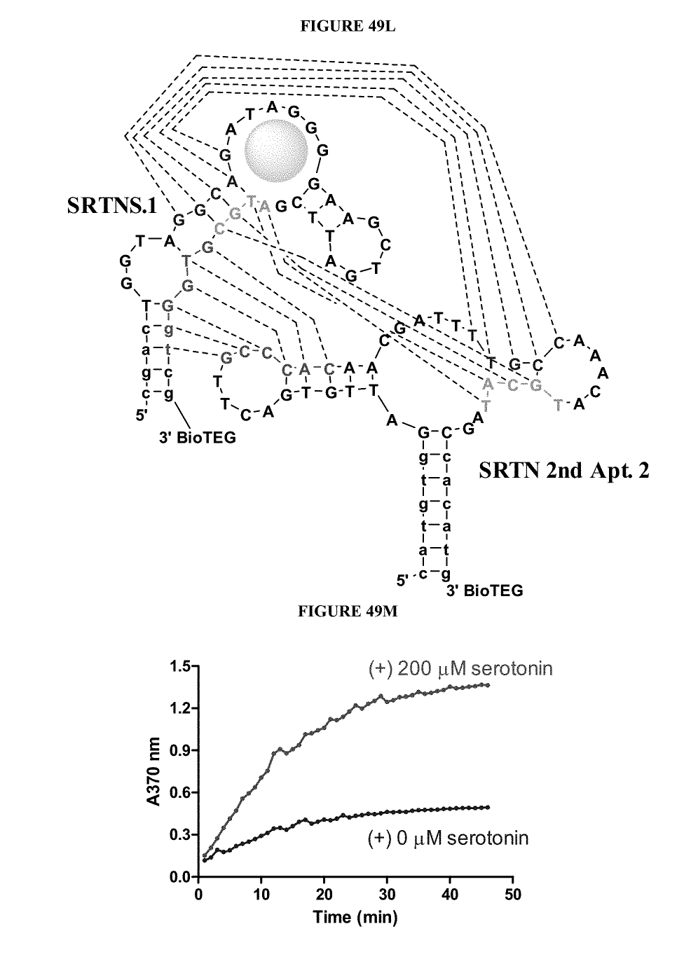

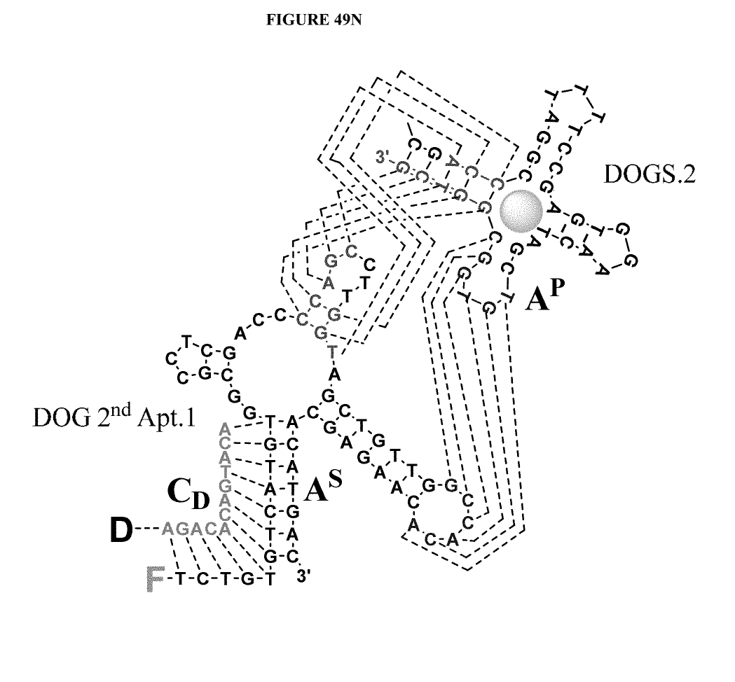

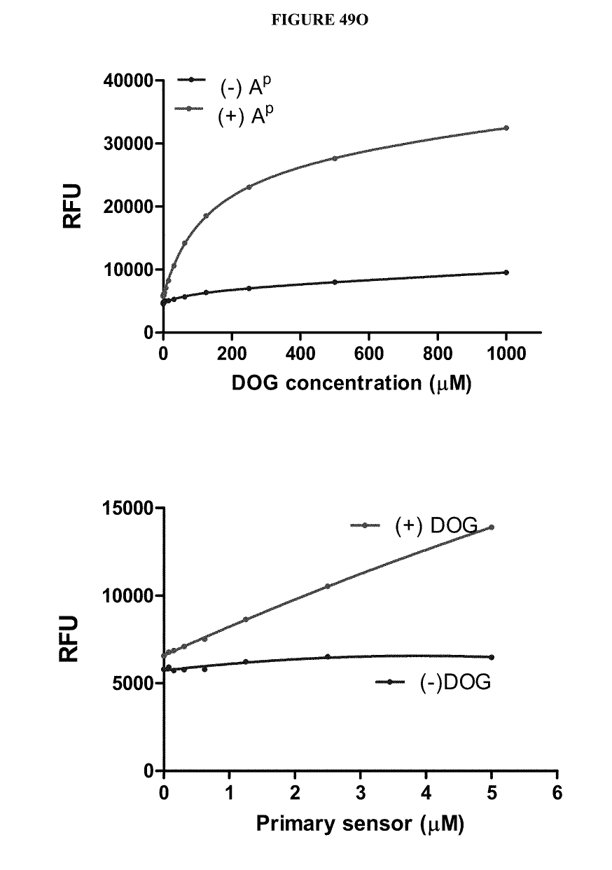

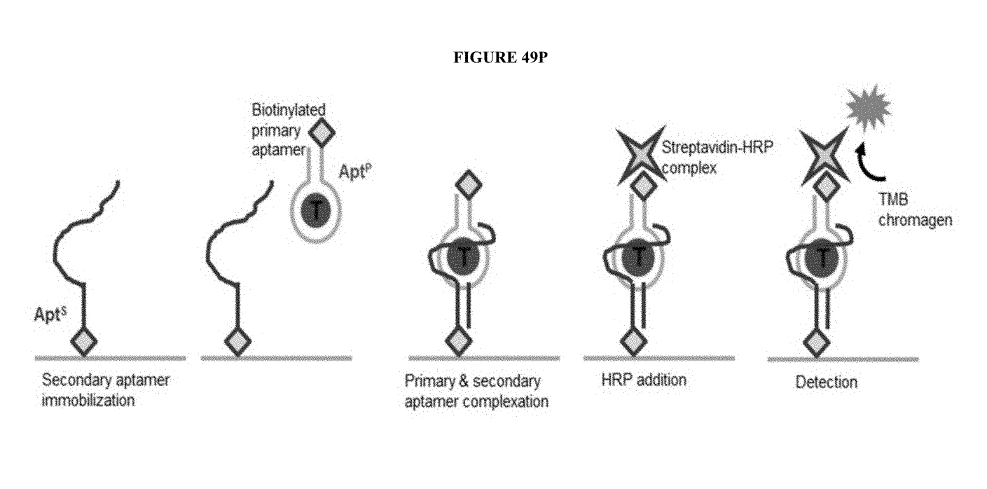

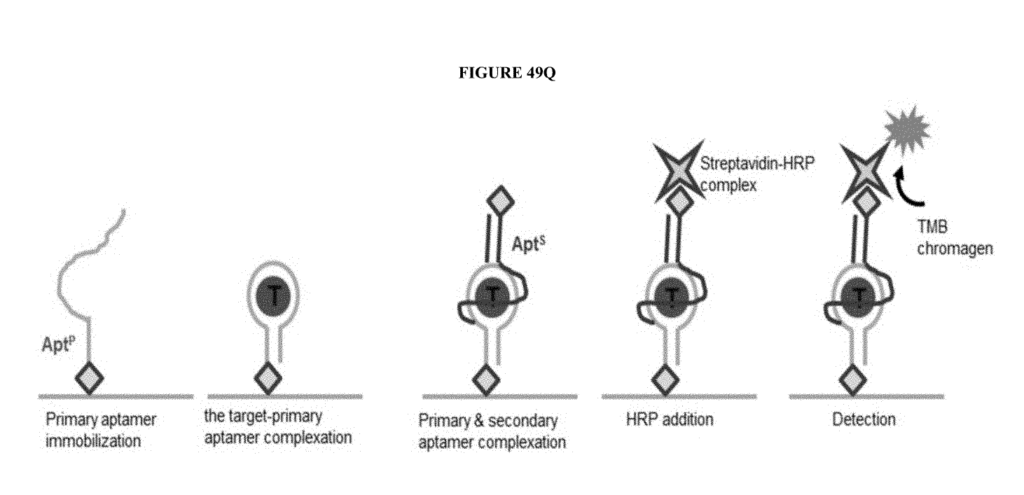

A “secondary aptamer” (A^{S}) binds a complex (A^{P}*L) of a primary aptamer (A^{P}) to its target analyte (ligand, L) selectively over free primary aptamers, forming a ternary complex (A^{p}*L*A^{S}). The secondary aptamers are isolated by solution-phase or solid-phase selections. An exemplary non-limiting list of the disclosed secondary aptamers is provided in FIGS. 48 and 49. The disclosed secondary aptamers were isolated by either of the aforementioned selections.

In certain embodiments, a sandwich assay is provided, which comprises a method of detecting or measuring the presence or amount of an analyte of interest in a sample, comprising (i) contacting at least a portion of a sample with effective amounts of (a) a primary aptamer comprising a core sequence that binds to the analyte and a portion that, when primary aptamer is bound to analyte, binds to a secondary “sandwich” aptamer; (b) a secondary “sandwich” aptamer; one or more of which is bound to a detectable moiety(ies) which can detect whether the primary aptamer and secondary “sandwich” aptamer are bound to each other or unbound.

In certain embodiments, a primary aptamer comprises at least one operative sequence which binds to a sandwich aptamer (a.k.a. secondary aptamer).

In certain, non-limiting embodiments, a secondary aptamer comprises an aptamer (‘secondary’) that binds to an aptamer (primary) with former binding to the latter preferentially when the latter is bound to its ligand (an analyte of interest).

In certain, non-limiting embodiments, a secondary aptamer comprises a sequence (‘secondary’) that binds to another sequence (primary) with former binding to the latter preferentially when the latter bound to a molecule.

In certain, non-limiting embodiments, the present invention discloses an assay for a molecule (fluorescence assay and ELISA-like) that uses two oligonucleotides with one oligonucleotide that preferentially binds to the other, when the latter is bound to that molecule.

In certain, non-limiting embodiments, one aptamer (primary) is sufficient to form a receptor.

A “sandwich” assay, without availability of the second binding site (epitope) in a molecule, is enabled by a secondary aptamer (A^{S}; a.k.a. sandwich aptamer or secondary sandwich aptamer) forming a ternary complex (A^{P}*L*A^{S}) (FIG. 8). In the disclosed sandwich assay, the primary and secondary aptamers are “sandwiching” components, with L being a target. In certain non-limiting embodiments, the sandwich assay can be implemented in solution (FIGS. 48 and 49) or on solid surface (FIG. 49) in non-limiting examples, plates, beads, or any component of a lateral flow device).

In certain non-limiting embodiments, a kit is provided for practicing a sandwich assay as described herein. For example, said kit comprises a primary aptamer directed toward an analyte of interest and a corresponding sandwich aptamer (a.k.a., secondary aptamer. Non-limiting examples of primary aptamers that may be comprised in such a kit include the primary aptamers set forth herein, for example as described in Section 6.1 and FIGS. 50A-B to 102AB, and a corresponding sandwich/secondary aptamer as described herein. Said kit may optionally further comprise target analyte, for example a control solution comprising target analyte, and/or a standard curve.

A sample may be, for example and not limitation, a blood sample, a plasma sample, a serum sample, a urine sample, a tissue sample, a cerebrospinal fluid sample, a sputum sample, a fecal sample, a water sample, an industrial sample, etc.

5.4.1. Isolation of Secondary Aptamers

The present application discloses the isolation of secondary aptamers using the SELEX process.

In certain non-limiting embodiments, methods for selecting secondary aptamers (A^{s}) can be performed, which can also be combined in some cases (FIG. 9) comprise a solid-state selection and a solution-phase selection.

Solid-State Election

During the solid-state selection a target aptamer (A^{p}) is attached to a matrix (e.g., beads), incubated with a library (e.g., but not limited to, pre-structured or unstructured N₂₀₋₁₀₀) in the presence of target analyte L to isolate aptamer candidates with affinity for A^{p}*L complex (FIG. 9A). These oligonucleotides are PCR-amplified, single-stranded species regenerated, and then used in the next selection cycle. The process is repeated until convergence is reached, pools cloned and sequenced, leading to A^{s} candidates. The counter-selection is performed by elimination of binders to A^{p} in the absence of L, ensuring binding to the complex.

Solution-Phase Selection

The process in solution-phase uses pre-structured library to enable formation of stem between primers attached to matrix via complementary oligonucleotides, and A^{s} candidates are selected because their loop is closed through binding to A^{p} and they get released from the solid surface (FIG. 9B). Counter-selection in this case is against A^{p} without ligand, and ligand itself.

In certain non-limiting embodiments, primary aptamers used in selection can be made from DNA, RNA, modified nucleotides, or spiegelmers. Spiegelmers are particularly suitable to minimize background, increase affinity, and improve properties of secondary aptamers, because these are mirror-image nucleic acids²⁰⁻²³ with L-deoxyribose or ribose, that do not firm contiguous Watson Crick base pairing with natural DNA. In certain non-limiting embodiments, such spiegelmers can be obtained by inverting aptamers for planar molecules or molecules with planes of symmetry, such as serotonin or dopamine (aptamers depicted in FIG. 28-39 can be inverted) or other neurotransmitters.

5.4.2. Secondary Aptamers to Glucose-Binding Primary Aptamers

More specifically, in certain non-limiting embodiments, secondary aptamers can be isolated according to the aforementioned method for glucose. In certain non-limiting embodiments, secondary aptamers or their analogs obtained by substitutions, deletions, and insertions of Watson-Crick base pairs or by mutations at non-conserved positions, bind to a primary aptamer, when the latter is in the complex with glucose. In certain non-limiting embodiments, secondary aptamers or their analogs obtained by substitutions, deletions, and insertions of Watson-Crick base pairs or by mutations at non-conserved positions, bind to a primary aptamer, when the latter is in the complex with a mono- or oligo-saccharide. In certain non-limiting embodiments, secondary aptamers or their analogs bind to a primary aptamer, when the latter is in the complex with glucose, wherein the analog comprises a nucleotide sequence at least 80% identical to the nucleotide sequence of the secondary aptamer; a nucleotide sequence at least 95% identical to the nucleotide sequence of the secondary aptamer; and a nucleotide sequence at least 99% identical to the nucleotide sequence of the secondary aptamer.

5.4.3. Secondary Aptamers to Phenylalanine-Binding Primary Aptamers

In certain non-limiting embodiments, secondary aptamers can be isolated according to the aforementioned method for phenylalanine. In certain non-limiting embodiments, secondary aptamers or their analogs obtained by substitutions, deletions, and insertions of Watson-Crick base pairs or by mutations at non-conserved positions, bind to a primary aptamer, when the latter is in the complex with Phenylalanine. In certain non-limiting embodiments, secondary aptamers or their analogs obtained by substitutions, deletions, and insertions of Watson-Crick base pairs or by mutations at non-conserved positions, bind to a primary aptamer, when the latter is in the complex with an amino acid or a peptide. In certain non-limiting embodiments, secondary aptamers or their analogs bind to a primary aptamer, when the latter is in the complex with Phenylalanine, wherein the analog comprises a nucleotide sequence at least 80% identical to the nucleotide sequence of the secondary aptamer; a nucleotide sequence at least 95% identical to the nucleotide sequence of the secondary aptamer; and a nucleotide sequence at least 99% identical to the nucleotide sequence of the secondary aptamer.

5.4.4. Secondary Aptamers to Hydrocortisone-Binding Primary Aptamers

In certain non-limiting embodiments, secondary aptamers can be isolated according to the aforementioned method for hydrocortisone. In certain non-limiting embodiments, secondary aptamers or their analogs obtained by substitutions, deletions, and insertions of Watson-Crick base pairs or by mutations at non-conserved positions, bind to a primary aptamer, when the latter is in the complex with hydrocortisone. In certain non-limiting embodiments, secondary aptamers or their analogs bind to a primary aptamer, when the latter is in the complex with hydrocortisone, wherein the analog comprises a nucleotide sequence at least 80% identical to the nucleotide sequence of the secondary aptamer; a nucleotide sequence at least 95% identical to the nucleotide sequence of the secondary aptamer; and a nucleotide sequence at least 99% identical to the nucleotide sequence of the secondary aptamer.

5.4.5. Secondary Aptamers to Dehydroisoandrosterone-Binding Primary Aptamers

In certain non-limiting embodiments, secondary aptamers can be isolated according to the aforementioned method for dehydroisoandrosterone. In certain non-limiting embodiments, secondary aptamers or their analogs obtained by substitutions, deletions, and insertions of Watson-Crick base pairs or by mutations at non-conserved positions, bind to a primary aptamer, when the latter is in the complex with dehydroisoandrosterone. In certain non-limiting embodiments, secondary aptamers or their analogs bind to a primary aptamer, when the latter is in the complex with dehydroisoandrosterone, wherein the analog comprises a nucleotide sequence at least 80% identical to the nucleotide sequence of the secondary aptamer; a nucleotide sequence at least 95% identical to the nucleotide sequence of the secondary aptamer; and a nucleotide sequence at least 99% identical to the nucleotide sequence of the secondary aptamer.

5.4.6. Secondary Aptamers to Deoxycorticosterone-Binding Primary Aptamers

In certain non-limiting embodiments, secondary aptamers can be isolated using the aforementioned method for deoxycorticosterone. In certain non-limiting embodiments, secondary aptamers comprising the nucleotide sequence of any one of SEQ ID NO: 34-35 (FIG. 48) and their analogs obtained by substitutions, deletions, and insertions of Watson-Crick base pairs or by mutations at non-conserved positions, bind to a primary aptamer comprising the nucleotide sequence of SEQ ID NO: 12 (FIG. 21), when the latter is in the complex with deoxycorticosterone. In certain non-limiting embodiments, secondary aptamers or their analogs bind to a primary aptamer, when the latter is in the complex with deoxycorticosterone, wherein the analog comprises a nucleotide sequence at least 80% identical to the nucleotide sequence of the secondary aptamer; a nucleotide sequence at least 95% identical to the nucleotide sequence of the secondary aptamer; and a nucleotide sequence at least 99% identical to the nucleotide sequence of the secondary aptamer.

5.4.7. Secondary Aptamers to Testosterone-Binding Primary Aptamers

In certain non-limiting embodiments, secondary aptamers can be isolated according to the aforementioned method for testosterone. In certain non-limiting embodiments, secondary aptamers or their analogs obtained by substitutions, deletions, and insertions of Watson-Crick base pairs or by mutations at non-conserved positions, bind to a primary aptamer, when the latter is in the complex with testosterone. In certain non-limiting embodiments, secondary aptamers or their analogs bind to a primary aptamer, when the latter is in the complex with testosterone, wherein the analog comprises a nucleotide sequence at least 80% identical to the nucleotide sequence of the secondary aptamer; a nucleotide sequence at least 95% identical to the nucleotide sequence of the secondary aptamer; and a nucleotide sequence at least 99% identical to the nucleotide sequence of the secondary aptamer.

5.4.8. Secondary Aptamers to Sphingosine-1-Phosphate-Binding Primary Aptamers

In certain non-limiting embodiments, secondary aptamers can be isolated according to the aforementioned method for sphingosine-1-phosphate. In certain non-limiting embodiments, secondary aptamers or their analogs obtained by substitutions, deletions, and insertions of Watson-Crick base pairs or by mutations at non-conserved positions, bind to a primary aptamer, when the latter is in the complex with sphingosine-1-phosphate. In certain non-limiting embodiments, secondary aptamers or their analogs bind to a primary aptamer, when the latter is in the complex with sphingosine-1-phosphate, wherein the analog comprises a nucleotide sequence at least 80% identical to the nucleotide sequence of the secondary aptamer; a nucleotide sequence at least 95% identical to the nucleotide sequence of the secondary aptamer; and a nucleotide sequence at least 99% identical to the nucleotide sequence of the secondary aptamer.

5.4.9. Secondary Aptamers to Dopamine-Binding Primary Aptamers

In certain non-limiting embodiments, secondary aptamers can be isolated according to the aforementioned method for dopamine. In certain non-limiting embodiments, secondary aptamers or their analogs obtained by substitutions, deletions, and insertions of Watson-Crick base pairs or by mutations at non-conserved positions, bind to a primary aptamer, when the latter is in the complex with dopamine. In certain non-limiting embodiments, secondary aptamers or their analogs bind to a primary aptamer, when the latter is in the complex with dopamine, wherein the analog comprises a nucleotide sequence at least 80% identical to the nucleotide sequence of the secondary aptamer; a nucleotide sequence at least 95% identical to the nucleotide sequence of the secondary aptamer; and a nucleotide sequence at least 99% identical to the nucleotide sequence of the secondary aptamer.

5.4.10. Secondary Aptamers to Serotonin-Binding Primary Aptamers

In certain non-limiting embodiments, secondary aptamers were isolated using the aforementioned method for serotonin. In certain non-limiting embodiments, secondary aptamers comprising any one of the nucleotide sequences of SEQ ID NO: 36, SEQ ID NO: 59, and SEQ ID NO: 60 (FIG. 48) and any one of their analogs obtained by substitutions, deletions, and insertions of Watson-Crick base pairs or by mutations at non-conserved positions, bind to a primary aptamer comprising any one of the nucleotide sequences of SEQ ID NO: 25 and SEQ ID NO: 58 (FIG. 34 and FIGS. 48C and D), when the latter is in the complex with serotonin. In certain non-limiting embodiments, secondary aptamers or their analogs bind to a primary aptamer, when the latter is in the complex with serotonin, wherein the analog comprises a nucleotide sequence at least 80% identical to the nucleotide sequence of the secondary aptamer; a nucleotide sequence at least 95% identical to the nucleotide sequence of the secondary aptamer; and a nucleotide sequence at least 99% identical to the nucleotide sequence of the secondary aptamer.

5.4.11. Secondary Aptamers to Tyrosine-Binding Primary Aptamers

In certain non-limiting embodiments, secondary aptamers can be isolated according to the aforementioned method for tyrosine. In certain non-limiting embodiments, secondary aptamers or their analogs obtained by substitutions, deletions, and insertions of Watson-Crick base pairs or by mutations at non-conserved positions, bind to a primary aptamer, when the latter is in the complex with tyrosine. In certain non-limiting embodiments, secondary aptamers or their analogs bind to a primary aptamer, when the latter is in the complex with tyrosine, wherein the analog comprises a nucleotide sequence at least 80% identical to the nucleotide sequence of the secondary aptamer; a nucleotide sequence at least 95% identical to the nucleotide sequence of the secondary aptamer; and a nucleotide sequence at least 99% identical to the nucleotide sequence of the secondary aptamer.

5.4.12. Secondary Aptamers to L-Tyrosine-Binding Primary Aptamers

In certain non-limiting embodiments, secondary aptamers can be isolated according to the aforementioned method for L-tyrosine. In certain non-limiting embodiments, secondary aptamers or their analogs obtained by substitutions, deletions, and insertions of Watson-Crick base pairs or by mutations at non-conserved positions, bind to a primary aptamer, when the latter is in the complex with L-tyrosine. In certain non-limiting embodiments, secondary aptamers or their analogs bind to a primary aptamer, when the latter is in the complex with L-tyrosine, wherein the analog comprises a nucleotide sequence at least 80% identical to the nucleotide sequence of the secondary aptamer; a nucleotide sequence at least 95% identical to the nucleotide sequence of the secondary aptamer; and a nucleotide sequence at least 99% identical to the nucleotide sequence of the secondary aptamer.

In certain non-limiting embodiments, this protocol can be suitably modified and optimized to isolate secondary aptamers to all non-limiting examples of primary aptamers and their analogs (including spiegelmers) as depicted in FIG. 49. It can also be applied to primary aptamers for any molecule that has molecular mass below 100000 Daltons or below 2000 Daltons or below 1000 Daltons or below 500 Daltons or below 200 Daltons, and it cannot have two epitopes that do not compete against each other.

5.4.13 Additional Sandwich Assay Embodiments

In certain non-limiting embodiments, the present application provides sandwich assays, with fluorescent detections and ELISA-like formats.

In the pseudosandwich assay described above, there were no true sandwich interactions (thus, ‘pseudo-sandwich’) and ligand*aptamer interactions were “translated” into a presence of a double helix allowing a format similar to a non-competitive sandwich ELISA. However, in certain embodiments a sandwich assay comprises two aptamers binding to one molecule at the same time without competing with each other.

In certain non-limiting embodiments of the present application aptamers against aptamer*ligand complexes or secondary aptamer bind primary aptamers when in complexes with ligands have been generated (FIG. 8 and FIG. 48). In certain embodiments a sandwich assay leads to high sensitivity. In certain non-limiting embodiments, aptamers against aptamer*ligand complexes can act as sophisticated switches with finely tuned complementarity; upon sensing ligand by one of them, they come together in a ternary (tertiary) complex (FIG. 49).

Sandwich Assay in Solution

In certain non-limiting embodiments, sandwiches are formed in solution for deoxycorticosterone and serotonin and fluorescence read-outs are measured (FIG. 48A-C). In this assay, a primary aptamer is in solution and a secondary aptamer, which is also in solution, is turned in structure-switching fluorescent form, and is interacting with the primary aptamer when the primary aptamer is in complex with the targeted steroid through the release of a quencher-labeled competitor oligonucleotide.

Sandwich Assay on Plate

In certain non-limiting embodiments, a sandwich assay on plate is performed for serotonin (FIG. 48D-E). In this assay, a primary aptamer is deposited on plate, while a secondary aptamer binds to the primary aptamer when it is in its complex with ligand (in this case serotonin).

In certain non-limiting embodiments, the foregoing assays can be used to detect and/or quantitate any analyte of interest, and can be particularly advantageous over existing methods in detecting and/or quantitating analytes that have a molecular weight less than about 1000 Daltons or less than about 500 Daltons or less than about 200 Daltons, including but not limited to steroid compounds, such as but not limited to cortisol, aldosterone, dehydroepiandrosterone, progesterone, testosterone; glucose; amino acids such as but not limited to phenylalanine, leucine, isoleucine, valine, citrulline, tyrosine, alanine; pharmaceutical compounds, vitamins, toxins, neurotransmitters (e.g., catecholamines, serotonin), peptides (vasopressin, oxytocin, angiotensin, natriuretic peptides, glucagon, insulin and others), antibiotics and antifungal compounds (e.g., aminoglucosides), macrocyclic immunosuppresants, or lipids or lipid complexes.

In certain non-limiting embodiments, these assays can also be applied to larger molecules, with molecular weight above 1000 Daltons.

### 6. EXAMPLE 1—ISOLATION OF PRIMARY APTAMERS

The present example provides methods for isolating primary aptamers (A^{P}) and examples of their structures.

The isolation of primary aptamers was performed using the SELEX process. Primary aptamers were isolated by solution-phase selection, as this method has inherent advantages for small molecules, such as higher affinity and ease of screening of aptamers (Table 1). Non-limiting examples are provided in FIG. 10-46.

The method was based on attaching a biotinylated strand complementary (C_{B}) to one of the PCR primers to agarose-streptavidin (FIG. 7) and attaching sequences from a library (e.g., but not limited to, N₈-N₁₀₀) through complementary interactions to C_{B} of a primer. Two primers on library, 5′- and 3′-, were also partially complementary; all members of the library which interacted with a target in a way that favors stem formation between complementary region of these primers were released from the agarose by displacing the complementary nucleotide (C_{B}), and were used in PCR amplification. This created an enriched pool of potential aptamers.

Fluorescent sensors were directly obtained from this selection, by substituting biotin with dabcyl and attaching fluorescein to the aptamer (FIG. 6, C_{D}), confirming that aptamers bind, determining their K_{d} (this was a competitive assay, so half-response is shifted away from the K_{d}⁸⁰, and C_{D} was present in an excess), and establishing selectivity.

Primary aptamers were isolated according to the aforementioned method for:
1. D-Glucose (SEQ ID NO:1);
2. L-Phenylalanine (SEQ ID NOS: 2-4);
3. Hydrocortisone (SEQ ID NOS: 5-7);
4. Dehydroisoandrosterone and Deoxycorticosterone 21-glucoside (DOG) (SEQ ID NOS: 8-12);
5. Testosterone (SEQ ID NOS: 13-17);
6. Sphingosine-1-phosphate (SEQ ID NO: 18);
7. Dopamine (SEQ ID NOS: 19-23);
8. Serotonin (SEQ ID NOS: 24-30);
9. Melatonine (SEQ ID NOS: 31-32); and
10. L-Tyrosine (SEQ ID NO: 33).

The disclosed primary aptamers bound to their target analyte and in their structure-switching formats they responded to its presence by an increase in fluorescence (FIG. 10-46 and FIG. 47A-D).

TABLE 1
Primary Aptamer Sequences.

| | | SEQ ID |
|---|---|---|
| Target | Primary Aptamer Sequence | NO: |
| Glucose | CTCTCGGGACGACCGTGTGTGTTGCTCTGTAACAGTGTCCATTGTCG | 1 |
| | TCCC | |
| Phenylalanine | CTCTCGGGACGACCGCGTTTCCCAAGAAAGCAAGTATTGGTTGGTCG | 2 |
| | TCCC | |
| | CTCTCGGGACGACCGGTGGGGGTTCTTTTTCAGGGGAGGTACGGTCG | 3 |
| | TCCC | |
| | CTCTCGGGACGACGAGGCTGGATGCATTCGCCGGATGTTCGATGTCG | 4 |
| | TCCC | |
| Hydrocortisone | CTCTCGGGACGACGCCCGCATGTTCCATGGATAGTCTTGACTAGTCG | 5 |
| | TCCC | |
| | CTCTCGGGACGACTAGCGTATGCGCCAGAAGTATACGAGGATAGTC | 6 |
| | GTCCC | |
| | CTCTCGGGACGACGCCAGAAGTTTACGAGGATATGGTAACATAGTC | 7 |
| | GTCCC | |
| Dehydro- | CTCTCGGGACGACGGGGATTTTCCCAATTGGTTCTTTCAATTTAGTCG | 8 |
| isoandrosterone and | TCCC | |
| Deoxycorticosterone | | |
| Dehydro- | CTCTCGGGACGACGGGGGTGGCATAGGGTAGGCTAGGGTCACTGTC | 9 |
| isoandrosterone | GTCCC | |
| | CTCTCGGGACGACGTGGCTAGGTAGGTTGCATGCGGCATAGGGGTC | 10 |
| | GTCCC | |
| | CTCTCGGGACGACGTGACGGTGTGTAGTTGGGTTGTGGCAGGAGTCG | 11 |
| | TCCC | |
| Deoxycorticosterone | CTCTCGGGACGACCCGGATTTTCCGAGTGGAACTAGCTGTGGCGGTC | 12 |
| | GTCCC | |
| Testosterone | CTCTCGGGACGACGGGATGTCCGGGGTACGGTGGTTGCAGTTCGTCG | 13 |
| | TCCC | |
| | CTCTCGGGACGACCAGGTGCCATTAGCGTCAGTGTGCTACGATGTCG | 14 |
| | TCCC | |
| | CTCTCGGGACGACCCGTTCGATCTAACCCTTGTTAGCCGTGATGTCG | 15 |
| | TCCC | |
| | CTCTCGGGACGACCCCTTCGATCTTCAACCAAAGCCGTTGGATGTCG | 16 |
| | TCCC | |
| | CTCTCGGGACGACGGGTGGTCATTGAGTGGTCTTAGGCAGGTAGTCG | 17 |
| | TCCC | |
| Sphingosine-1- | CTCTCGGGACGACGTGGTGTGGGAGAAAGAATTTTCATTGGGGTAG | 18 |
| phosphate | GGGGTCGTCCC | |
| Dopamine | CTCTCGGGACGACCACTTCAGACGCTCAACGTTTGGGGAGGCACGG | 19 |
| | CAGGTCGTCCC | |
| | CTCTCGGGACGACGGGGAGGAGTTAGCATGACGGCAACTTTAGTAC | 20 |
| | TTCGTCGTCCC | |
| | CTCTCGGGACGACGCCAGTTTGAAGGTTCGTTCGCAGGTGTGGAGTG | 21 |
| | ACGTCGTCCC | |
| | CTCTCGGGACGACTGCAGCCTGGGGTTGTGGGGGGTAGGGGAGGTC | 22 |
| | TGAGTCGTCCC | |
| | CTCTCGGGACGACCACACAGAGGCACAACTCGCAGGAGCAAAGCGG | 23 |
| | CAGGTCGTCCC | |
| Serotonin | CTCTCGGGACGACAGGGGCATATATAGTCTAGGGTTTGGTGTGGGTA | 24 |
| | GTGTCGTCCC | |
| | CTCTCGGGACGACTGGTAGGCAGATAGGGGAAGCTGATTCGATGCG | 25 |
| | TGGGTCGTCCC | |
| | CTCTCGGGACGACTGGTAGGCAGCAGGGGAAGTAGGCGTGTCCTCG | 26 |
| | TGGGTCGTCCC | |
| | CTCTCGGGACGACCAGTAGGGGATCCACAGTGAGGGGTTTGTATGG | 27 |
| | GTGGTCGTCCC | |
| | CTCTCGGGACGACTGGTAGGCAACAGGGGAAGGGAGTTCTGCGTAC | 28 |
| | GTGGGTCGTCCC | |
| | CTCTCGGGACGACGGAGGTGGTGTCTTGGACAGTGGTATTCGCAGTT | 29 |
| | GCGTCGTCCC | |
| | CTCTCGGGACGACAGAGACGGGGTGCTTACTTGGTTCAGGGGAGTC | 30 |
| | GACGTCGTCCC | |
| Melatonin | CTCTCGGGACGACAGCCAAGGTCGTAAGGTACGGTCAGTGTACTCG | 31 |
| | GTTGTCGTCCC | |
| | CTCTCGGGACGACGTCTTGGGGGTGGTGGGTTTGGCTGGTACTTAGG | 32 |
| | GCGTCGTCCC | |
| Tyrosine | CTCTCGGGACGACGGCCCGATCTCAGAGTAGTCGTCCC | 33 |

The sequences of the primary aptamers that were isolated for serotonin in their presumed secondary structure, as shown with the complementary oligonucleotide (C) that was used in selection (C_{B}) or fluorescence sensing (C_{D}), and the presumed core pocket (as shown in FIG. 10-46) are included in Table 2.

TABLE 2
Sequences of Primary Aptamers for serotonin in their presumed secondary structure,
the complementary oligonucleotides used in their selection, and their presumed core pockets.
For Primary Aptamers isolated for Serotonin comprising the nucleotide sequence of SEQ ID NO: 24

| | |
|---|---|
| Primary Aptamer | CTCTCGGGACGACAGGGGCATATATAGTCTAGGGTTTGGTGTGGGTAGTGTCGT |
| | CCC (SEQ ID NO: 37) |
| Complementary | TGTCGTCCCGAGAG (SEQ ID NO: 38) |
| oligonucleotide | |
| Core Pocket | NAGGGGCATATATAGTCTAGGGTTTGGTGTGGGTAGTN, where N can be any one of |
| | A, T, G, or C (SEQ ID NO: 39) |

For Primary Aptamers isolated for Serotonin comprising the nucleotide sequence of SEQ ID NO: 25

| | |
|---|---|
| Primary Aptamer | CTCTCGGGACGACTGGTAGGCAGATAGGGGAAGCTGATTCGATGCGTGGGTCGT |
| | CCC (SEQ ID NO: 40) |
| Complementary | GTCGTCCCGAGAG (SEQ ID NO: 41) |
| oligonucleotide | |
| Core Pocket | NTGGTAGNNNGATAGGGNNNNGCTGANNNGANNNGTGGN, where N can be any |
| | one of A, T, G, or C (SEQ ID NO: 42) |

For Primary Aptamers isolated for Serotonin comprising the nucleotide sequence of SEQ ID NO: 26

| | |
|---|---|
| Primary Aptamer | CTCTCGGGACGACTGGTAGGCAGCAGGGGAAGTAGGCGTGTCCTCGTGGGTCGT |
| | CCC (SEQ ID NO: 43) |
| Complementary | GTCGTCCCGAGAG (SEQ ID NO: 44) |
| oligonucleotide | |
| Core Pocket | NTGGTAGGCAGCAGGGGAAGTAGGCGTGTCCTCGTGGN, where N can be any one |
| | of A, T, G, or C (SEQ ID NO: 45), |

For Primary Aptamers isolated for Serotonin comprising the nucleotide sequence of SEQ ID NO: 27

| | |
|---|---|
| Primary Aptamer | CTCTCGGGACGACCAGTAGGGGATCCACAGTGAGGGGTTTGTATGGGTGGTCGT |
| | CCC (SEQ ID NO: 46) |
| Complementary | GGTCGTCCCGAGAG (SEQ ID NO: 47) |
| oligonucleotide | |
| Core Pocket | NAGTAGGGGANNNCAGTGAGGGGTTTGTANNNNTN, where N can be any one of A, |
| | T, G, or C (SEQ ID NO: 48) |

For Primary Aptamers isolated for Serotonin comprising the nucleotide sequence of SEQ ID NO: 28

| | |
|---|---|
| Primary Aptamer | CTCTCGGGACGACTGGTAGGCAACAGGGGAAGGGAGTTCTGCGTACGTGGGTC |
| | GTCCC (SEQ ID NO: 49) |
| Complementary | GTCGTCCCGAGAG (SEQ ID NO: 50) |
| oligonucleotide | |
| Core Pocket | NTGGNAGGNAACAGGGGNGGGAGNNCTNCGTNCGTGGN, where N can be any one |
| | of A, T, G, or C (SEQ ID NO: 51) |

For Primary Aptamers isolated for Serotonin comprising the nucleotide sequence of SEQ ID NO: 29

| | |
|---|---|
| Primary Aptamer | CTCTCGGGACGACGGAGGTGGTGTCTTGGACAGTGGTATTCGCAGTTGCGTCGT |
| | CCC (SEQ ID NO: 52) |
| Complementary | CGTCGTCCCGAGAG (SEQ ID NO: 53) |
| oligonucleotide | |
| Core Pocket | NGGAGGTGGNNNNNNNNNNNGTGGTATTCGCAGTTGCN, where N can be any one |
| | of A, T, G, or C (SEQ ID NO: 54) |

For Primary Aptamers isolated for Serotonin comprising the nucleotide sequence of SEQ ID NO: 30

| | |
|---|---|
| Primary Aptamer | CTCTCGGGACGACAGAGACGGGGTGCTTACTTGGTTCAGGGGAGTCGACGTCGT |
| | CCC (SEQ ID NO 55) |
| Complementary | TGTCGTCCCGAGAG (SEQ ID NO: 56) |
| oligonucleotide | |
| Core Pocket | NNAGANNNGGGGTGCTTACTTGGTTCAGGGGANNNGACNN, where N can be any |
| | one of A, T, G, or C (SEQ ID NO: 57) |

### 7. EXAMPLE 2—PSEUDOSANDWICH ASSAYS

In the present example pseudosandwich assays, with fluorescent detection in solution and on plates (ELISA-like format) are provided.

Solution-evolved aptameric sensors were adapted for use in a solid surface-format application, for example, ELISA-type assays. The solution sensor—composed of an analyte and a primary aptamer partially hybridized to a competitor oligonucleotide—was modified using attachment chemistry to enable its anchoring to a solid surface. The surface attachment chemistry used was biotin-streptavidin interaction. In addition, the complementary strand to the competitor oligonucleotide (optimized to bind to the competitor oligonucleotide in the presence of analyte) was modified with a biotin-tag, which was used to capture a “detection” molecule e.g. streptavidin-HRP conjugate. The HRP substrate, TMB, was used for visualization.

Optimization of response of the surface-bound sensor, relative to +/−analyte, was carried out by adjusting parameters including: amount of sensor on surface, attachment chemistry, concentration of complementary competitor oligonucleotide, buffer in each step, washing steps, HRP concentration, and HRP substrate.

### Experimental Procedure for Pseudosandwich Assay—in Solution

The solution buffer had the composition of the SELEX buffer or any other buffer that the sensor was shown to work in. Each sample well contained a final concentration of 50 nM aptamer-C_{Dext} duplex (with a stoichiometric amount of C_{Dext} in relation to aptamer, or in two- or three-fold excess). Selected concentrations of analyte were added, and complement to C_{Dext} was added to a final concentration of 2 μM. Final mixtures were incubated at room temperature for 20 minutes and then the fluorescence signal was measured.

Pseudosandwich assays in solution, where a double helix formation was triggered by an analyte, were performed for:
1. Deoxycorticosterone 21-glucoside (DOG);
2. L-Tyrosine;
3. D-Glucose; and
4. L-Phenylalanine

The primary aptamers formed a complex with C_{Dext} and in the presence of C_{Dext}^{comp} produced an increase in fluorescence in the presence of their target analytes (FIG. 47A-D).

If the C_{Dext} was deposited on plates, the double helix remained attached to the plate when the rest of solution was removed and it could be extensively washed.

### Experimental Procedure for Pseudosandwich Assay—in Solid State

Deoxycorticosterone 21-glucoside (DOG) sensor ELISA on streptavidin-coated plates was performed as following: 151.5 pmoles (6.6 μL×30 μM) of DOG sensor in PBS pH 7.4 buffer solution, was added per well of the streptavidin-coated ELISA plate (Thermo, binding capacity 5 pmoles) containing 100 μL of PBS buffer, to give a final sensor concentration of 1.44 μM. The mixture was incubated at room temperature for 5 minutes. The solution was then removed from the well and the well washed thoroughly eight times with PBS buffer. Next, 100 μL of TRIS pH 7.4 buffer (composed of 20 mM TRIS, 140 mM NaCl, 5 mM KCl, and 2 mM MgCl₂) was added per well, followed by 2 μL of 2 mM DOG dissolved in DMSO, to give a final Deoxycorticosterone 21-glucoside (DOG) concentration of 50 μM. The single-stranded complement containing the biotin tag was then add (2 μL of 100 μM stock) to give a final concentration of 1.79 μM. The mixture was incubated at room temperature of 20 minutes. The solution was then removed and the wells washed thoroughly 8× with PBS buffer. 100 μL of PBS containing 1% BSA and 8000-fold diluted HRP-STV conjugate was then added per well, and incubated for 5 minutes at room temperature. The solution was removed and wells were washed thoroughly 8× with PBS buffer. 100 μL of TMB/H₂O₂ substrate was then added to each well and the reaction progression was monitored on a plate reader at 652 nm.

Results for Deoxycorticosterone 21-glucoside (DOG) concentration dependence on a streptavidin-ELISA 96-well plate are shown in FIG. 47E. Eight wells were coated with aptamer sensor and exposed to various concentrations of Deoxycorticosterone 21-glucoside (DOG) analyte, followed by tagging with HRP enzyme, and quantified by adding TMB substrate, which is oxidized to a blue product by H₂O₂/HRP. A time course of the oxidation of TMB proportional to the amount of HRP enzyme bound in each well, as well as a concentration dependence were measured (FIG. 47E).

Phenylalanine (Phe) sensor ELISA on streptavidin-coated plates was performed as following: The aforementioned procedure was carried out for the Phe sensor, except the 20 minute incubation step was carried out in 20 mM HEPES pH 7.5, 1 M NaCl, 10 mM MgCl₂, 5 mM KCl buffer. Phe concentrations used were 0-200 μM from a 2 mM stock solution in water. Results for Phe concentration dependence on a streptavidin-ELISA 96-well plate are shown in FIG. 47F.

### 8. EXAMPLE 3—ISOLATION OF SECONDARY APTAMERS

The present example discloses methods for isolating secondary aptamers (A^{S}) and their structure.

The isolation of secondary aptamers was performed using the SELEX process. Two methods for selecting secondary aptamers (A^{s}) were performed, which can also be combined in some cases (FIG. 9:

(1) A Solid-State Selection.

A target aptamer (A^{p}) was attached to a matrix (e.g., beads), incubated with a library (e.g., but not limited to, prestructured or unstructured N₂₀₋₁₀₀) in the presence of target analyte L to isolate aptamer candidates with affinity for A^{p}*L complex (FIG. 9A). These oligonucleotides were PCR-amplified, single-stranded species regenerated, and then used in the next selection cycle. The process was repeated until convergence is reached, pools cloned and sequenced, leading to A^{s} candidates. The counter-selection was performed by elimination of binders to A^{P} in the absence of L, ensuring binding to the complex.

(2) A Solution-Phase Selection.

The process in solution-phase used pre-structured library to enable formation of stem between primers attached to matrix via complementary oligonucleotides, and A^{s} candidates were selected because their loop was closed through binding to A^{p} and they get released from the solid surface (FIG. 9B). Counter-selection in this case was against A^{p} without ligand, and ligand itself.

Secondary aptamers were isolated using the aforementioned methods for:
1. Deoxycorticosterone (SEQ ID NOS: 34-35);
2. Serotonin (SEQ ID NOS: 36, 59, and 60);

The secondary aptamers bound to the primary aptamers, when the latter is in the complex with their target analytes (FIG. 48).

The nucleotide sequences of the secondary aptamers are shown in Table 3.

TABLE 3
Secondary Aptamer Sequences.

| | | SEQ ID |
|---|---|---|
| Target | Secondary Aptamer Sequence | NO: |
| Deoxy- | GTCATGTGGCGCCTCGACCCCAGCCTTGGTAGCTGTTGGCCACACAA | 290 |
| corticosterone | GAGCACATGAC | |
| | GTCATGTGACGAGACAAGGAGAACGGAAGGCGACCGGATAAATCG | 291 |
| | GATATCACATGAC | |
| Serotonin | GTCATGTGGATTGTGACTTGCCCACAACGATTTTGCCAAACATGCAT | 36 |
| | AGCCACATGAC | |
| | CATGTGGATTGTGACTTGCCCACAACGATTTTGCCAAACATGCATAG | 60 |
| | CCACATG | |

Secondary aptamers for serotonin were isolated based on nucleotide sequences of primary aptamer for serotonin comprising: CGACTGGTAGGCAGATAGGGGAAGCTGATTCGATGCGTGGGTCG (SEQ ID NO: 58), which was derived from the nucleotide sequence SEQ ID NO: 25 (FIG. 48A-C). Secondary aptamers were isolated for serotonin comprising the following nucleotide sequence: TCTGTGTCATGTGGATTGTGACTTGCCCACAACGATTTTGCCAAACATGCATA GCCACATGAC (SEQ ID NO: 59), in their presumed secondary structure (FIG. 48A-C).

Isolated primary and secondary aptamers were used to perform sandwich assays as described in Example 4. Complementary oligonucleotides comprising the following nucleotide sequence: CACATGACACAGA (SEQ ID NO: 61) were used in fluorescence sensing.

### 9. EXAMPLE 4—SANDWICH ASSAYS

In the present example, examples of sandwich assays, with fluorescent detections and ELISA-like formats are provided.

In the pseudosandwich assay described above, there were no true sandwich interactions (thus, ‘pseudo-sandwich’) and ligand*aptamer interactions were “translated” into a presence of a double helix allowing a format similar to non-competitive sandwich ELISAs. However, the true sandwich requires two aptamers (similar to two antibodies) binding to one molecule at the same time without competing with each other. This can be difficult for small molecules, simply because they lack two epitopes.

An alternate approach is depicted in FIG. 8 and FIG. 48, wherein aptamers against aptamer*ligand complexes or secondary aptamer binding primary aptamers when in complexes with ligands have been generated. The advantage of this method was that it is a true sandwich which could lead to better sensitivity. Aptamers against aptamer*ligand complexes acted as sophisticated switches with finely tuned complementarity; upon sensing ligand by one of them, they came together in a ternary (tertiary) complex.

### Experimental Procedure for Sandwich Assay—in Solution

The solution buffer had the composition of the SELEX buffer or any other buffer that the sensor was shown to work in. The buffer used for the measurement of serotonin was PBS buffer including 2 mM MgCl₂. For the measurement of deoxycorticosterone and testosterone, the buffer consisted of 20 mM HEPES, 1 M NaCl, 10 mM MgCl₂, and 5 mM KCl (pH 7.5).

Separate solutions were prepared in parallel: (1) Four times concentrated primary aptamer (A^{P}) was prepared in buffer and incubated for >5 min in a water bath heated to boiling until use for unfolding secondary structure, then mixed with four times concentrated analyte (L) and incubated >30 min. e.g. 20 μL of 4× concentrated A^{P} (e.g. 10 μM) and 20 μL of 4× concentrated serotonin solution (e.g. 800 μM). The final concentration of serotonin was 400 μM and of the complex A^{P}-serotonin was 5 μM. This is the 2× concentration of Ap-serotonin complex solution. (2) Two times concentrated secondary aptamer (A^{S}) solution was prepared. FAM fluorescent conjugated A^{S} (100 nM) and its dabcyl-quencher strand were mixed, the dabcyl-quencher strand was in three to five-fold excess for the sufficient quenching. This mixture was incubated for >5 min in a water bath heated to boiling—used to promote unfolding of secondary structure and hybridization with the quencher strand. 40 μL of each of the separately prepared solutions (1) and (2) were mixed and incubated for 40 min. Then 75 μL of the mixed solution was taken for measurement. The fluorescent signal was measured. Each sample contained a final concentration of 2.5-5 μM of primary aptamer (A^{P}), 50 nM of secondary aptamer A^{S}, 150 nM-250 nM of dabcyl quencher strand, and the indicated concentrations of analyte on the plots.

### Sandwich Assay—in Solid State

First, ligand (L) and primary aptamer (Ap) solution were prepared. 400 μL of 2× concentrated Bio-TEG conjugated primary aptamer (0.6 pmole/μL) was prepared in buffer, and incubated in the boiling water for >5 min until use. Two times concentrated serotonin solution (e.g. 400 μM in buffer, 50 μL) was prepared, then mixed with 50 μL ligand and 50 μL of A^{P} solution and incubated >30 min. The final concentration of serotonin was 200 μM and of the complex A^{P}-serotonin was 30 pmole per well. Second, a Bio-TEG modified secondary aptamer solution was prepared in bulk and 100 μL were applied (0.5 pmole/μl) per well. This solution was incubated for >5 min in a water bath heated to boiling and was allowed to cool until use. After preparing these solutions, the ELISA assay was carried out. The ELISA plate wells were washed with PBS (+2 mM MgCl2) buffer, 2 times, with soaking 5 min between washes. The A^{S} solution (100 μL) was added to the wells, then incubated for ˜20 min at room temperature to allow biotin binding to the plate. The solution was then removed and the wells were washed thoroughly 8 times with the same buffer. Then, 100 μL of [L*A^{P}] solution was added to the wells and incubated for >30 min at room temperature. The solution was then removed and the wells were washed thoroughly 8 times with the same buffer. 100 μL of PBS containing 1% BSA and 10000-fold diluted HRP-STV conjugate was then added per well, and incubated for 5 minutes at room temperature. The solution was removed and wells washed thoroughly 10 times with the same buffer. Directly after this wash, 100 μL of TMB/H₂O₂ substrate was then added to each well and the reaction progression monitored on a plate reader at 370 or 652 nm.

Fluorescence read-outs when sandwiches are formed in solution (FIG. 48A-C) were performed for the following analytes:
1. Deoxycorticosterone 21-glucoside (DOG); and
2. Serotonin.

In this assay, a primary aptamer is in solution and a secondary aptamer, which is also in solution, was turned in structure-switching fluorescent form, and was interacting with the primary aptamer when the primary aptamer was in complex with the targeted steroid through the release of a quencher-labeled competitor oligonucleotide (FIG. 48A-C).

A sandwich assay on plate was performed for the following analyte:
1. Serotonin. In this assay, a primary aptamer was deposited on plate, while a secondary aptamer bound to the primary aptamer when it was in its complex with serotonin (FIG. 48D-E).

### 10. EXAMPLE 5—ANTI-APTAMER ASSAY

400 nM FAM-aptamer solution and four-times concentrated Iowa Black-complement strand solution are prepared separately in buffer (20 mM HEPES, 1 M NaCl, 10 mM MgCl2, 5 mM KCl, pH 7.5). These solutions were annealed separately by incubating in boiling water for 5 min, and cooled down at room temperature for ˜30 min. Meanwhile, a two-times concentrate of each aptamer's target solutions were prepared; the final concentration is indicated in each plot. After the 30 min incubation period of the aptamer and complement strand solutions, 18.75 ul of the aptamer solution is added first to the 384-well plate, then 37.5 ul of the target solution is added, and then immediately added is 18.75 ul of complementary strand solution. Without incubation, the fluorescent measurement was started and read every 5 min at room temperature for >8 hours by using Fluorescence plate reader (Victor II microplate reader, PerkinElmer). The final concentration of aptamer is 100 nM, the concentration of complementary strand is 100 nM for all except CS aptamer (1000 nM) in 75 μL reaction volume. The final concentration of target/ligand is indicated in each plot.

Each aptamer and its complementary strand is listed in below. The results are shown in FIG. 3A-L.

### (A) DOG Aptamer/Complementary (100 nM: 100 nM)

-DOGS.2_sht/DOGS.2_sht_anti:
(SEQ ID NO: 292)
CGA CCC GGA TTT TCC GAG TGG AAC TAG CTG TGG CGG
TCG/36-FAM/;
(SEQ ID NO: 293)
/5IABkFQ/CGA CCG CCA CAG CTA GTT CCA CTC GGA AAA
TCC GGG TCG
-DOGS.2_Full/DOGS.2_Full_anti:
(SEQ ID NO: 294)
/56-FAM/CTC TCG GGA CGA CCC GGA TTT TCC GAG TGG
AAC TAG CTG TGG CGG TCG TCC C;
(SEQ ID NO: 295)
GGG ACG ACC GCC ACA GCT AGT TCC ACT CGG AAA ATC
CGG GTC GTC CCG AGA G/3IABkFQ/

### (B) ALD Aptamer/Complementary: (100 nM: 100 nM)

-ALDS.1_sht/ALDS.1_sht_anti:
(SEQ ID NO: 296)
CGA CAG ATA GTT GTT CTT AGC GAT GTT CAG CGT TGT
CG/36-FAM/;
(SEQ ID NO: 297)
/5IABkFQ/CGA CAA CGC TGA ACA TCG CTA AGA ACA ACT
ATC TGT CG
-ALDS.1_Full/ALDS.1_Full_anti:
(SEQ ID NO: 298)
/56-FAM/CTC TCG GGA CGA CAG ATA GTT GTT CTT AGC
GAT GTT CAG CGT TGT CGT CCC;
(SEQ ID NO: 299)
GGG ACG ACA ACG CTG AAC ATC GCT AAG AAC AAC TAT
CTG TCG TCC CGA GAG/3IABkFQ/

### (C) CS Aptamer/Complementary: (100 nM: 1000 nM)

CSS.1_sht/CSS.1_sht_anti:
(SEQ ID NO: 300)
GAC GAC GCC CGC ATG TTC CAT GGA TAG TCT TGA CTA
GTC GTC/36-FAM/;
(SEQ ID NO: 301)
/5IABkFQ/GAC GAC TAG TCA AGA CTA TCC ATG GAA CAT
GCG GGC GTC GTC

### (D) TES Aptamer/Complementary:

TES.1_sht/TES.1_sht_anti:
(SEQ ID NO: 302)
ACG GGA TGT CCG GGG TAC GGT GGT TGC AGT TCG T/36-
FAM/;
(SEQ ID NO: 303)
/5IABkFQ/ACG AAC TGC AAC CAC CGT ACC CCG GAC ATC
CCG T

### (E) Phe-CpRh Aptamer/Complementary:

Phe-CpRh_sht/Phe-CpRh_sht_anti:
(SEQ ID NO: 304)
/56-FAM/CGA CAC AGC GTG AGC CAA CTA ATT AGT GCG
TAT TGT CG;
(SEQ ID NO: 305)
CGA CAA TAC GCA CTA ATT AGT TGG CTC ACG CTG TGT
CG/3IABkFQ/

### (F) Phe Aptamer/Complementary:

Phe_sht/Phe_sht_anti:
(SEQ ID NO: 306)
/GAC CGG TGG GGG TTC TTT TTC AGG GGA GGT ACG GTC/
36-FAM/;
(SEQ ID NO: 307)
/5IABkFQ/GAC CGT ACC TCC CCT GAA AAA GAA CCC CCA
CCG GTC

### 11. EXAMPLE 6—SANDWICH ASSAY

FIG. 49H shows a primary aptamer (A^{P}) that binds to serotonin (SRTNS.1) and a secondary aptamer (SRTN 2nd Apt1: GTG GTT AGT AAC TTG CAC GCC GCC CAA TTG CTA TTC ATG ACA AGC CAC (SEQ ID NO: 251) that binds to an aptamer (primary) with former binding to the latter preferentially when the latter is bound to its ligand. The colored letters with the dot lines indicate the hypothetical partial hybridization region between two aptamers. Those two aptamers are applied to ELISA-like assay through immobilizing the secondary aptamer on the streptavidin-coated plate and the primary in the solution. The spectrophotometric signal increases in proportion to the serotonin analyte. The signal is produced through the biotin conjugated on the SRTNS.1 binds the streptavidin-HRP conjugate, and catalyze the substrate TMB (FIG. 49I). Increase in signal confirms that there is more binding when serotonin is present in increasing concentrations.

FIG. 49 J shows a primary aptamer that binds to serotonin (gold ball) and a secondary aptamer (A^{S}) (SRTN 2^{nd} Apt.2: TCT GTG TCA TGT GGA TTG TGA CTT GCC CAC AAC GAT TTT GCC AAA CAT GCA TAG CCA CAT GAC (SEQ ID NO: 252) that binds to an aptamer (primary) with former binding to the latter preferentially when the latter is bound to its ligand. The colored letters with the dot lines indicate the partial hybridization region between two aptamers. SRTN 2^{nd} Apt.2 (F conjugated strand, ‘F’ indicates fluorescein) is shown in a format that was based on a competition between ligand and complementary oligonucleotide carrying a quencher (D, dabcyl). The quencher strand (grey C_{D}) first hybridizes with the 5′ end of the aptamer, through the secondary conformational change upon target (SRTNS.1+serotonin) binding. This quencher is released from the aptamer strand and results to the fluorescent signal increase. Red line higher than blue line indicates that complementary oligonucleotide with a quencher (C_{D}) is being displaced when (LOWER) either primary aptamer (A^{P}) is present (i.e., that binding occurs when primary aptamer to the ligand already present was added) or when (UPPER) serotonin to solution when primary aptamer is already present was added (FIG. 49K).

FIG. 49L shows an ELISA assay with the trimmed secondary aptamer (SRTN 2^{nd} Apt1) on the plate and primary aptamer (SRTNS.1) in solution with amplification through HRP-conjugated to streptavidin, streptavidin-HRP conjugate, and catalyze the substrate TMB. Red line shows that there is more binding when serotonin is present (FIG. 49M). In certain non-limiting embodiments, reverse format in which primary and secondary aptamers are present are both possible.

FIG. 49N shows a primary aptamer (A^{P}) that binds to the DOG (DOGS.2), a secondary aptamer (A^{S}) that binds to an aptamer (primary) with former binding to the latter preferentially when the latter is bound to its ligand (DOG 2nd Apt.1: TCT GTG TCA TGT GGC GCC TCG ACC CCA GCC TTG GTA GCT GTT GGC CAC ACA AGA GCA CAT GAC (SEQ ID NO: 253). The colored letters with the dot lines indicate the partial hybridization region between two aptamers. DOG 2nd Apt.2 (F conjugated strand, ‘F’ indicates fluorescein) is shown in a format that was based on a competition between ligand and complementary oligonucleotide carrying a quencher (D, dabcyl). The quencher strand (grey) first hybridizes with the 5′ end of the aptamer, through the secondary conformational change upon target (DOGS.2+DOG) binding, this quencher is released from the aptamer strand. It results the fluorescent signal increase. (TOP) shows that secondary aptamer binds to primary aptamer when we add DOG (steroid concentration on X-axis) and (BOTTOM) shows that secondary aptamer binds to DOG only in the presence of primary aptamer (FIG. 49O).

### 12. EXAMPLE 7—PROTOCOLS FOR SECONDARY APTAMER ELISA (SANDWICH ASSAY)

Secondary Aptamer on the Plate and Primary Aptamer in Solution Method (FIG. 49P).

A biotinylated secondary aptamer is immobilized on a streptavidin-coated plate in appropriate buffer for about 30 minutes at room temperature, and then washed with buffer to remove excess unbound aptamers. A primary aptamer and its target are pre-incubated for 40 min at room temperature and then added to the immobilized secondary aptamer, incubating on the plate for 40 minutes. Streptavidin-HRP, 10000-fold diluted in 100 μL of PBS (w. 1% BSA), is added next, and incubated for 20 mins, then washed thoroughly with PBS. TMB substrate mix is then added and change in absorbance at 370 nm recorded for 45 mins.

Primary Aptamer on the Plate and Secondary Aptamer in Solution” Method (FIG. 49Q).

A biotinylated primary aptamer is immobilized on a streptavidin-coated plate in appropriate buffer for about 30 minutes at room temperature, and then washed with buffer to remove excess unbound aptamers. A target solution is then added to the immobilized primary aptamer and incubated for about 40 min. The secondary aptamer is added to the plate on top of the target solution and incubated about 40 min, then washed with buffer to remove the unbound secondary aptamers. Streptavidin-HRP, 10000-fold diluted in 100 μL of PBS (w. 1% BSA), is added next, and incubated for 20 mins, then washed thoroughly with PBS. TMB substrate mix is then added and change in absorbance at 370 nm recorded for 45 mins.

### 13. REFERENCES

1. Cox, K. L.; Devanarayan, V.; Kriauciunas, A.; Manetta, J.' Montrose, C.; Sittampalam, S. “Immunoassay Methods” in Assay Guidance Manual (Internet), NCBI Resources Bookshelf; last updated: Dec. 24, 2014.
2. Gan, S. D.; Patel, K. R. “Enzyme Immunoassay and Enzyme-Linked Immunosorbent Assay” J Investig Derm 133, e12 (2013)
3. Lequin, R. “Enzyme Immunoassay (EIA)/Enzyme-Linked Immunosorbent Assay (ELISA)” Clinical Chemistry 51(12): 2415-2418 (2005).
4. Fan, M.; He, J. “Recent Progress in Noncompetitive Hapten Immunoassays: A review” Chapter 5 in Trends in Immunolabelled and Related Techniques, www.intechopen.com, accessed at: Jun. 1, 2015.
5. Wilson, R. “Sensitivity and Specificity: Twin Goals of Proteomics Assays” Expert Rev Proteomics 10(2): 135-149 (2013).
6. Dong, J.-X.; Xu, C.; Wang, H.' Xiao, Z.-L.; Gee, S. J.; Li, Z.-F.; Wang, F.; Wu, W.-J.; Shen, Y.-D.; Yang, J.-Y.; Sun, Y.-M.; Hammock, B. D. “Enhanced Sensitive Immunoassay” Noncompetitive Phage AntiImmune Complex Assay for the Determination of Malachite Green and Leucomalachite Green” J Agric Food Chem 62(34): 8752-8758 (2014).
7. Ihara, M.; Suzuki, T.; Kobayashi, N.; Goto, J.; Ueda, H. “Open-Sandwich Enzyme Immunoassay for One-Step Noncompetitive Detection of Corticosteroid 11-Deoxycortisol” Anal Chem 81(20): 8298-8304 (2009).
8. Shen, J.; Li, Y.; Gu, H.; Xia, F.; Zuo, X. “Recent Development of Sandwich Assay based on Nanobiotechnologies for Proteins, Nucleic Acids, Small Molecules, and Ions” Chem Rev 114(15): 7631-7677 (page 7662) (2014).
9. Ozer, A.; Pagano, J. M.; Lis, J. T. “New Technologies Provide Quantum Changes in the Scale, Speed, and Success of SELEX Methods and Aptamer Characterization” Molecular Therapy Nucleic Acids 3: e 183 (2014).
10. McKeague, M.; Derosa, M. C. “Challenges and opportunities for small molecule aptamer development.” J Nucleic Acids, Article ID 748913 (2012).
11. Rohloff, J. C.; Gelinas, A. D.; Jarvis, T. C.; Ochsner, U. A.; Schenider, D. J.; Gold, L.; Janjic, N. “Nucleic Acid Ligands with Protein-like Side Chains: Modified Aptamers and Their Use as Diagnostic and Therapeutic Agents” Molecular Therapy Nucleic Acids 3: e:201 (2014).
12. Ellington, A. D. & Szostak, J. W. “In vitro selection of RNA molecules that bind specific ligands” Nature (London) 346: 818-822 (1990).
13. Tuerk, C. & Gold, L. “Systematic evolution of ligands by exponential enrichment: RNA ligands to bacteriophage T4 DNA polymerase” Science 249(4968):505-510 (1990).
14. Cho, E. J.; Lee, J.-W.; Ellington, A. D. “Applications of Aptamers as Sensors” Annu. Rev. Anal Chem 2:241-264 (2009).
15. Keefe, A. D.; Pai, S.; Ellington, A. “Aptamers as therapeutics” Nat Rev Drug Discovery, 9:537-550 (2010).
16. Yang, K.-A.; Pei, R.; Stefanovic, D.; Stojanovic, M. N. “Optimizing Cros-reactivity with Evolutionary Search for Sensors” J Am Chem Soc 134:1642-1647 (2012).
17. Stoltenburg, R.; Nikolaus, N.; Strehlitz, B. “Capture-SELEX: Selection of DNA Aptamers for Aminoglycoside Antibiotics” J Anal Meth Chem 15697 (2012).
18. Rajendran M.; Ellington, A. D. “Selection of fluorescent aptamer beacons that light up in the presence of zinc” Anal Bioanal Chem 390:1067-75 (2008).
19. Nutiu, R.; Li, Y. “In vitro selection of structure-switching signaling aptamers” Angew Chem Int Ed 44: 1061-1065 (2005).
20. Hu, J.; Easley, C. J.; “A simple and rapid approach for measurement of dissociation constants of DNA aptamers against proteins and small molecules via automated microchip electrophoresis” Analyst 136:3461-3468 (2011).
21. Nutiu, R., and Li, Y. “Structure-switching signaling aptamers” J Am Chem Soc 125:4771-4778 (2003).
22. Vater, A.; Klussmann, S. “Toward third-generation aptamers: Spiegelmers and their therapeutics prospects.” Curr Opin Drug Discov Devel 6: 253-261 (2003).
23. Vater, A.; Klussmann, S. “Turning mirror-image oligonucleotides into drugs: the evolution of Spiegelmer therapeutics” Drug Discovery Today 20(1): 147-155 (2015).
24. Williams, K. P.; Liu, X. N.; Schumacher, T. N.; Lin, H. Y.; Ausiello, D. A.; Kim, P. S.; Bartel, D. P. “Bioactive and Nuclease-resistant L-DNA Ligand of Vasopressin.” Proc Natl Acad Sci. USA, 94(21):11285-11290 (1997).
25. Purschke, W. G.; Eulberg, D.; Buchner, K.; Vonhoff, S.; Klussmann, S. “An L-RNA-based aquaretic agent that inhibits vasopressin in vivo.” Proc Natl Acad Sci USA 103:5173-5178 (2003).
26. Yang, K. A; Barbu, M.; Halim, M.; Pallavi, P.; Kim, B.; Kokpashchikov, D.; Pecic, S.; Taylor, S.; Worgall, T. S.; Stojanovic, M. N. “Recognition and Sensing of Low-Epitope Targets via Ternary Complexes with Oligonucleotides and Synthetic Receptors” Nat Chem. 6:1003-1008 (2014).
27. Ozer, A.; Pagano, J. M.; Lis, J. T. “New Technologies Provide Quantum Changes in the Scale, Speed, and Success of SELEX Methods and Aptamer Characterization” Molecular Therapy Nucleic Acids 3: e 183 (2014).
28. McKeague, M.; Derosa, M. C. “Challenges and opportunities for small molecule aptamer development.” J Nucleic Acids, Article ID 748913 (2012).
29. Rohloff, J. C.; Gelinas, A. D.; Jarvis, T. C.; Ochsner, U. A.; Schenider, D. J.; Gold, L.; Janjic, N. “Nucleic Acid Ligands with Protein-like Side Chains: Modified Aptamers and Their Use as Diagnostic and Therapeutic Agents” Molecular Therapy Nucleic Acids 3: e:201 (2014).
30. Ellington, A. D. & Szostak, J. W. “In vitro selection of RNA molecules that bind specific ligands” Nature (London) 346: 818-822 (1990).
31. Tuerk, C. & Gold, L. “Systematic evolution of ligands by exponential enrichment: RNA ligands to bacteriophage T4 DNA polymerase” Science 249(4968):505-510 (1990).
32. Robertson, D. L.; Joyce, G. F. “Selection in vitro of an RNA enzyme that specifically cleaves single stranded DNA” Nature 344:467-468 (1990).
33. Cho, E. J.; Lee, J.-W.; Ellington, A. D. “Applications of Aptamers as Sensors” Annu. Rev. Anal Chem 2:241-264 (2009).
34. Keefe, A. D.; Pai, S.; Ellington, A. “Aptamers as therapeutics” Nat Rev Drug Discovery, 9:537-550 (2010).
35. Anslyn, E. “Supramolecular Analytical Chemistry” J Org Chem 72(3):687-699 (2007).
36. Gennady V.; Oshovsky, D.; Reinhoudt, D. V.; Verboom, W. “Supramolecular Chemistry in Water” Angew. Chem. Int. Ed. 46(14): 2366-2393 (2007).
37. Jonathan W. Steed (Editor-in-Chief), Philip A. Gale (Editor-in-Chief) “Supramolecular Chemistry: From Molecules to Nanomaterials” Wiley, 8 Volume Set (2012).
38. Steed, J. W.; Atwood, J. L.; Supramolecular Chemistry, 2nd Edition, Willey, (2009).
39. Pedersen, C. J. “Cyclic polyethers and their complexes with metal salts” J Am Chem Soc 89(26):7017-7036 (1967).
40. Kyba, E. P.; Siegel, M. G.; Sousa, L. R.; Sogah, G. D. Y.; Cram, D. J. “Chiral, hinged, and functionalized multiheteromacrocycles” J Am Chem Soc 95:2691-2692 (1973).
41. Dietrich, B.; Lehn, J.-M.; Sauvage, J.-P. “Diaza-polyoxa-macrocycles et macrobicycles” Tet Lett 2885-2888 (1969).
42. Costa J. B.; Andreiev A. I.; Dieckmann T. “Thermodynamics and kinetics of adaptive binding in the malachite green RNA aptamer” Biochemistry 24; 52(38):6575-83 (2013).
43. Hermann, T.; Patel, D. J. “Adaptive Recognition by Nucleic Acids” Science 287:820-825 (2000).
44. Xia, T.; Yuan, J.; Fang, X.; Conformational dynamics of an ATP-binding DNA aptamer: a single molecule study. J Phys Chem B 117(48): 14994-50003 (2013).
45. Vater, A.; Klussmann, S. “Toward third-generation aptamers: Spiegelmers and their therapeutics prospects.” Curr Opin Drug Discov Devel 6: 253-261 (2003).
46. Vater, A.; Klussmann, S. “Turning mirror-image oligonucleotides into drugs: the evolution of Spiegelmer therapeutics” Drug Discovery Today 20(1): 147-155 (2015).
47. Williams, K. P.; Liu, X. N.; Schumacher, T. N.; Lin, H. Y.; Ausiello, D. A.; Kim, P. S.; Bartel, D. P. “Bioactive and Nuclease-resistant L-DNA Ligand of Vasopressin.” Proc Natl Acad Sci. USA, 94(21):11285-11290 (1997).
48. Purschke, W. G.; Eulberg, D.; Buchner, K.; Vonhoff, S.; Klussmann, S. “An L-RNA-based aquaretic agent that inhibits vasopressin in vivo.” Proc Natl Acad Sci USA 103:5173-5178 (2003).
49. Kato, T.; Yano, K.; Ikebukuro, K.; and Karube, I. “Interaction of three-way DNA junctions with steroids” Nucleic Acids Res 28:1963-1968 (2000).
50. Stojanovic, M. N.; Green, E. G.; Semova, S.; Nikic, D. B.; Landry, D. W. “Cross-reactive arrays based on three-way junctions” J Am Chem Soc 125:6085-6089 (2003).
51. Yang, K.-A.; Pei, R.; Stefanovic, D.; Stojanovic, M. N. “Optimizing Cros-reactivity with Evolutionary Search for Sensors” J Am Chem Soc 134:1642-1647 (2012).
52. Iskander, K. N.; Osuchowski, M. F.; Stearns-Kurosava, D. J.; Stepien, D.; Valentine, C.; Remick, D. G. “Sepsis: multiple abnormalities, heterogenous responses, and evolving understanding” Phys. Rev. 93(3): 1247-1288 (2013).
53. Benedict, C. R.; Rose, J. A. “Arterial norepinephrine changes in patients with septic shock” Circ Shock 38(3): 165-172 (1992).
54. Wilson, M. F.; Brackett, D. J.; Tompkins, P, Benjamin, B.; Archer, L. T.; Hinshaw, L. B. “Elevated plasma vasopressin concentrations during endotoxin and E. coli shock” Adv Shock Res 6:15-26 (1981).
55. Landry D. W., Levin H. R., Gallant E. M., Ashton R. C., Seo S., D'Alessandro D., Oz M. C., Oliver J. A. “Vasopressin deficiency contributes to the vasodilation of septic shock” Circulation 95: 1122-1125 (1997).
56. Lin, I. Y.; Ma, H. P.; Lin, A. C.; Chong, C. F.; Lin, C. M.; Wang, T. “Low plasma vasopressin/norepinephrine ratio predicts septic shock” Am J Emrg Med 23(6): 718-724 (2005).
57. Sam, S.; Corbridge, T. C.; Mokhlesi, B.; Comellas, A. P.; Molitch, M. E. “Cortisol levels and mortality in severe sepsis” Clin Endorinol (Oxf) 60(1): 29-35 (2004).
58. Bendel, S.; Karlsson, S.; Pettila, V.; Loisa, P.; Varpula, M.; Ruokonen, E.; Finnsepsis Study Group “Freel cortisol in sepsis and septic shock” Anesth Analq 106(6): 1813-1819 (2008).
59. Annane, D.; Maximi, V.; Ibrahim, F.; Alvarez, J. C.; Abe, E.; Boudou, P. “Diagnosis of Adrenal Insufficiency in Severe Sepsis and Septic Shock” Am J Respir Crit Care Med 174:1319-1326, 2006.
60. Moares, R. B.; Freidman, G.; Viana, M. V.; Tonietto, T.; Saltz, H.; Czepielewski, M. A. “Aldosterone secretion in patients with septic shock: a prospective study.” Arq Bras Endorcinol Metabol 57(8): 636-641 (2013).
61. Salgado, D. R.; Rocco, J. R.; Silva, E.; Vincent, J. L. “Modulation of the renin-angiotensin-aldosterone system in sepsis: a new therapeutic approach?” Expert Opin Ther Targets 14(1): 11-20 (2010).
62. Zhang, W.; Chen, X.; Huang, L.; Lu, N.; Zhou, L.; Wu, G.; Chen, Y. “Severe sepsis: Low expression of renin-angiotensin system is associated with poor prognosis” Exper Therap Med 7(5): 1342-1348 (2014).
63. Sato, Y.; Fujiwara, H.; Takatsu, Y. “Cardiac troponin and heart failure in the era of high-sensitivity assays” J. Cardiology 60(3): 160-167 (2012).
64. Sherwood, M. W.; Newby, K. “High-Sensitivity Troponin Assays: Evidence, Indications, and Reasonable Use” J Am Heath Assoc. 3: e000403 (2014).
65. Cox, K. L.; D evanarayan, V.; Kriauciunas, A.; Manetta, J.' Montrose, C.; Sittampalam, S. “Immunoassay Methods” in Assay Guidance Manual (Internet), NCBI Resources Bookshelf; last updated: Dec. 24, 2014.
66. Gan, S. D.; Patel, K. R. “Enzyme Immunoassay and Enzyme-Linked Immunosorbent Assay” J Investig Derm 133, e12 (2013).
67. Lequin, R. “Enzyme Immunoassay (EIA)/Enzyme-Linked Immunosorbent Assay (ELISA)” Clinical Chemistry 51(12): 2415-2418 (2005).
68. Fan, M.; He, J. “Recent Progress in Noncompetitive Hapten Immunoassays: A review” Chapter 5 in Trends in Immunolabelled and Related Techniques, www.intechopen.com, accessed at: Jun. 1, 2015.
69. Wilson, R. “Sensitivity and Specificity: Twin Goals of Proteomics Assays” Expert Rev Proteomics 10(2): 135-149 (2013).
70. De Lemos, J. A.; Morrow, D. A.; “Brain Natriuretic Peptide Measurement in Acute Coronary Syndromes” Circulation 106: 2686-2870 (2002).
71. Osborne, S. E.; Ellington, E. D. “Nucleic acid selection and the challenge of combinatorial chemistry” Chemical Reviews 97 (2): 349-370 (1997).
72. Vant-Hull B., Gold L, Zichi D A. “Theoretical principles of in vitro selection using combinatorial nucleic acid libraries” Curr Protoc Nucleic Acid Chem. Chapter 9: Unit 9.1 (2000).
73. Dong, J.-X.; Xu, C.; Wang, H.′ Xiao, Z.-L.; Gee, S. J.; Li, Z.-F.; Wang, F.; Wu, W.-J.; Shen, Y.-D.; Yang, J.-Y.; Sun, Y.-M.; Hammock, B. D. “Enhanced Sensitive Immunoassay” Noncompetitive Phage AntiImmune Complex Assay for the Determination of Malachite Green and Leucomalachite Green” J Agric Food Chem 62(34): 8752-8758 (2014).
74. Ihara, M.; Suzuki, T.; Kobayashi, N.; Goto, J.; Ueda, H. “Open-Sandwich Enzyme Immunoassay for One-Step Noncompetitive Detection of Corticosteroid 11-Deoxycortisol” Anal Chem 81(20): 8298-8304 (2009).
75. Shen, J.; Li, Y.; Gu, H.; Xia, F.; Zuo, X. “Recent Development of Sandwich Assay based on Nanobiotechnologies for Proteins, Nucleic Acids, Small Molecules, and Ions” Chem Rev 114(15): 7631-7677 (page 7662) (2014).
76. Stojanovic, M. N., de Prada, P., Landry, D. W. “Self-assembling aptameric sensors” J Am Chem Soc, 122:11547-11548 (2000).
77. Chandra, M.; Silverman, S. K. “DNA and RNA Can Be Equally Efficient Catalysts for Carbon-Carbon Bond Formation”, J Am Chem Soc 130:2936-2937 (2008).
78. Pinheiro, V. B.; Holliger, P. “Towards XNA nanotechnology: new materials from synthetic genetic polymers” Trends Biotechnology 32(6): 321-328 (2014).
79. Santoro, S. W.; Joyce, G. F.; Sakthivel, K.; Gramatikova, S.; Barbas, C. F. “RNA cleavage by a DNA enzyme with extended chemical functionality” J Am Chem Soc 122:2433-2439 (2000).
80. Perrin D. M.; Garestier, T.; Helene, C. “Bridging the gap between proteins and nucleic acids: a metal-independent RNAseA mimic with two protein-like functionalities” J Am Chem Soc 123:1556-1563 (2001).
81. Kimoto, M.; Yamashige, R.; Matsunaga, K-I.; Yokoyama, S.; Hirao, I. “Generation of high-affinity DNA aptamers using an expanded genetic alphabet” Nature Biotechnol 31, 453-457 (2013).
82. Imaizumi, Y.; Kasahare, Y.; Fujita, H.; Kitadume, S.; Ozaki, H.; Endoh, T.; Kuwhara, M.; Sugimoto, N. “Efficacy of base-modification on target binding of small molecule DNA aptamers” J Am Chem Soc 135, 9412-9419 (2013).
83. Brukner, I.; El-Ramahi, R.; Gorska-Flipot, I.; Krajinovic, M.; Labuda, D. “An in vitro selection scheme for oligonucleotide probes to discriminate between closely related DNA sequences.” Nucleic Acids Res. 35(9): e66, (2007), and literature cited therein.
84. Brukner, I.; Krajinovic, M.; Dascal, A.; Labuda, D. “A protocol for the in vitro selection of specific oligonucleotide probes for high-resolution DNA typing.” Nat Protoc 2(11):2807-14 (2007).
85. Ayel, E.; Escudé, C. “In vitro selection of oligonucleotides that bind double-stranded DNA in the presence of triplex-stabilizing agents.” Nucleic Acids Res 38(5): e31 (2010), and literature cited therein.
86. Darfeuille, F.; S. Reigadas; J. Hansen; H. Orum; C. Di Primo; J. Toulme (2006). “Aptamers targeted to an RNA hairpin show improved specificity compared to that of complementary oligonucleotides.” Biochemistry 45 (39):12076-12082.
87. Durand, G.; Lisi, S.; Ravelet, C.; Dausse, E.; Peyrin, E.; Toulme, J.-J. “Riboswitches Based on Kissing Complexes for the Detection of Small Ligands” Angew Chem Int Ed 53(27): 6942-6945 (2014).
88. Luense, C. E.; Michlewski, G.; Hopp, C. S.; Rentmeister, A.; Caceres, J. F.; Famulok, M.; Mayer, G. “An aptamer targeting the apical-loop domain modulates pri-miRNA processing” Angew Chem Int Ed 49(27):4674-4677, (2010).
89. Butcher, S. E.; Pyle, A. M. “The Molecular Interactions that Stabilize RNA Tertiary Structure: RNA Motifs, Patterns, and Networks” Acc. Chem. Res. 44(12):1302-1311 (2011).
90. Abraham, M.; Dror, O.; Nussinov, R.; Wolfson, H. J. “Analysis and classification of RNA tertiary structures” RNA 14(11):2274-2289 (2008).
91. Butcher, S. E.; Heckman, J. E.; Burke, J. M. “Reconstitution of hairpin ribozyme activity following separation of functional domains”. Journal Biol Chem 270 (50): 29648-29651 (1995).
92. Doherty, E A; Doudna J A “Ribozyme structures and mechanisms”. Annu. Rev. Biophys. Biomol. Struct. 30: 457-475 (2001).
93. Rinker, S.; Ke, Y.; Liu, Y.; Chhabra, R.; Yan, H. “Self-assembled DNA nanostructures for distance dependent multivalent ligand-protein binding” Nature Nanotech 3:418-422 (2008).
94. Corbett, P. T.; Leclaire, J.; Vial, L.; West, K. R.; Wietor, J.-L.; Sanders, J. K. M.; Otto, S. “Dynamic combinatorial chemistry”. Chem Rev 106 (9):3652-3711 (2006).
95. Wang Y.; Rando R. R. “Specific binding of aminoglycoside antibiotics to RNA” Chemistry & Biology 2:281-290 (1995).
96. Stojanovic, M. N.; de Prada, P.; Landry, D. W. “Fluorescent Sensors based on aptamer self-assembly” J Am Chem Soc 122: 9678-9679 (2000).
97. Stojanovic, M. N.; de Prada, P.; Landry, D. W. “Aptamer-based folding fluorescent sensor for cocaine”, J Am Chem Soc 123: 4928-4929, (2001).
98. Stojanovic, M. N.; Landry, D. W. “Aptamer-based colorimetric sensor for cocaine” J Am Chem Soc 124: 9678-9679 (2002).
99. Stojanovic, M. N. Kolpashchikov, D. M. “Modular allosteric sensors” J Am Chem Soc 126: 9266-9270, (2004).
100. Pei, R.; Shen, A; Olah, M.; Stefanovic, D.; Worgall, T.; Stojanović, M. N. “High-resolution crossreactive array for alkaloids” Chem Commun. (Camb); (22):3193-5 (2009).
101. Green, E. G.; Olah, M. J.; Abramova, T.; Williams, L.; Stefanović, D.; Worgall, T.; Stojanović, M. N. “Rational Approach to Minimal Cross-reactive Arrays” J Am Chem Soc 128: 15278-15282 (2006).
102. Stoltenburg, R.; Nikolaus, N.; Strehlitz, B. “Capture-SELEX: Selection of DNA Aptamers for Aminoglycoside Antibiotics” J Anal Meth Chem 15697 (2012).
103. Rajendran M.; Ellington, A. D. “Selection of fluorescent aptamer beacons that light up in the presence of zinc” Anal Bioanal Chem 390:1067-75 (2008).
104. Nutiu, R.; Li, Y. “In vitro selection of structure-switching signaling aptamers”
Angew Chem Int Ed 44: 1061-1065 (2005).
105. Hu, J.; Easley, C. J.; “A simple and rapid approach for measurement of dissociation constants of DNA aptamers against proteins and small molecules via automated microchip electrophoresis” Analyst 136:3461-3468 (2011).
106. Nutiu, R., and Li, Y. “Structure-switching signaling aptamers” J Am Chem Soc 125:4771-4778 (2003).
107. Nguyen, T. H.; Pei, R.; Landry, D.; Stojanovic, M.; Lin. Q “Label-free microfluidic characterization of temperature dependent biomolecular interactions,” Biomicrofluid 5:34118-341187 (2011).
108. Nguyen, T. H.; Pei, R.; Landry, D.; Stojanovic, M.; Lin. Q “Microfluidic Aptameric Affinity Sensing of Vasopressin for Clinical Diagnostic and Therapeutic Applications,” Sensors and Actuators B: Chemical 154: 59-66 (2011).
109. Nguyen, T. H.; Pei, R.; Landry, D.; Stojanovic, M.; Lin. Q. “An Aptameric Microfluidic System for Specific Purification, Enrichment and Mass Spectrometric Detection of Biomolecules,” J of Microelectromechanical Syst 18(6): 1198-1207 (2009).
110. Hilton, J. P.; Olsen, T.; Kim, J.; Zhu, J.; Nguyen, T. H.; Barbu, M.; Pei, R.; Stojanovic, M.; Lin, Q. “Isolation of thermally sensitive protein-binding oligonucleotides on a microchip” Microfluidics and Nanofluidics, 19(4):795-804 (2015).
111. Linnet, K. “Necessary sample size for method comparison studies based on regression analysis” Clin Chem 45:882-894 (1999).
112. Magari, R. T. “Evaluating Agreement Between Two Analytical Methods in Clinical Chemistry” Clin Chem Lab Med 38: 1021-1025 (2000).
113. Linnet, K. “Performance of Deming regression analysis in case of misspecified analytic error ratio in method comparison studies” Clin Chem 44:1024-1031 (1998).
114. Dewitte, K.; Fierens, C.; Stockl, D.; Thienport, L. M. “Application of the Bland-Altman plot for interpretation of method-comparison studies: a critical investigation of its practice” Clin Chem 48:799-801 (2002).
115. Yang, L.; Flint Beal, M. “Determination of Neurotransmitter Levels in Models of Parkinson's Disease by HPLC-ECD” in Giovanni Manfredi and Hibiki Kawamata (eds.), Neurodegeneration: Methods and Protocols, Methods in Molecular Biology., vol. 793: 401-415, 2011.
116. Kennedy, B.; Ziegler, M. G. “A more sensitive and specific radioenzymatic assay for catecholamines” Life Sci 47(23):2143-53 (1990).
117. “http://www.abnova.com/products/products detail. asp?catalog id=KA1887” and other vendors, accessed on Mar. 2, 2016.
118. Clark, J. J.; Sandberg, S. G.; Wanat, M. J.; Gan, J. O.; Home, E. A.; Hart, A. S.; Akers, C. A. et al. “Chronic microsensors for longitudinal subsecond dopamine detection in behaving animals” Nat. Methods 7(2):126-129 (2010).
119. Mannironi, C.; Di Nardo, A.; Fruscoloni, P.; Tocchini-Valentini, G. P. “In vitro selection of dopamine RNA ligands” Biochem 36(32): 9726-9734 (1997).
120. Simpson, N.; Maffei, A.; Freeby, M.; Burroughs, S.; Freyberg, Z.; Javitch, J.; Leibel, R. L.; Harris, P. E. “Dopamine-mediated autocrine inhibitory circuit regulating human insulin secretion in vitro” Mol Endocrinol (10):1757-72 (2012).
121. Hauser, N. C.; Martinez, R.; Jacob, A.; Rupp, S.; Hoheisel, J. D.; Matysiak, S. “Utilising the left helical conformation of L-DNA for analyzing different marker types on a single universal microarrays platform” Nucleic Acids Res 34(18): 501-5111 (2006).
122. Hampshire, A. J.; Rusling, D. A.; Broughton-Head, V. J.; Fox, K. R. “Footprinting: A method for determining the sequence selectivity, affinity, and kinetics of DNA binding ligands” Methods 42(2): 128-140 (2007).
123. Regulski, E. E.; Breaker, R. R. “In-Line Probing Analysis of Riboswitches” Methods in Molecular Biology 419: 53-75, Totowa, N.J. (2008).
124. Franksson, G.; Anggard, E. “The Plasma Protein Binding of Amphetamine, Catecholamines and Related Compounds” Acta Pharm Tox 28(3):209-214 (2009).
125. Appendix. Normal Hormone Reference Ranges. Greenspan's Basic & Clinical Endocrinology. Ninth Edition. the McGraw-Hill Companies (2011).
126. Elias, A. N.; Nosratola, D.; Vaziri, M. D.; Maksy, M. “Plasma Norepinephrine, Epinephrine, and Dopamine Levels in End-Stage Renal Disease” Arch Intern Med 145:101301015 (1985).
127. Plunker, J. J.; Reeves, J. D.; Ngo, L.; Bellows, W., Shafer, S. L.; Roache, G.; Howse, J.; Herskowitz, A.; Mangano, D. T. et al “Urine and Plasma Catecholamine and Cortisol Concentrations after Myocardial Revascularization” Anest 86:785-796 (1997).
128. Zuker, M. “Mfold web server for nucleic acid folding and hybridization prediction” Nucleic Acids Res 31 (13), 3406-15, (2003).
129. Zadeh, J. N., Steenberg, C. D.; Bois, J. S.; Wolfe, B. R.; Khan, A. R.; Pierce, M. B.; Dirks, R. M.; Pierce, N. A. “NUPACK: analysis and design of nucleic acid systems” J Comp Chem 32:170-173 (2011).
130. (http://www.enzolifesciences.com/ADI-900-017A/arg8-vasopressin-elisa-kit/) and other similar kits, accessed on Mar. 3, 2015.
131. (https://www.buhlmannlabs.ch/products-solutions/special-products/vasopressin-adh/) and similar kits, accessed on Mar. 3, 2015.
132. Zhang, D.; Rios, D. R.; Tam, V. H.; Chow, D. S. “Development and validation of a highly sensitive LC-MS/MS assay for the quantification of arginine vasopressin in human plasma and urine: Application in preterm neonates and child” J Pharm Biomed Anal 96:67-73 (2014).
133. Bassi, E.; Park, M.; Azavedo, L. C. P. “Therapeutic Strategies for High-Dose Vasopressor-Dependent Shock” volume 2013: ID 654708, 10 pages (2013).
134. Raff, H.; Findling, J. W. “Aldosterone control in critically ill patients: ACTH, metoclopramide, and atrial natriuretric peptide” Crit Care Med 18(9):915-920 (1997).
135. Dick, M.; Dasta, J. F.; Choban, P. S.; Sinha, R.; Flancbaum, L. “Serum Aldosterone Concentrations and Urine Output in Oliguric Intensive Care Unit Patients Receiving Low-Dose Dopamine” Ann Pharmacotherapy 28:837-840 (1994).
136. Lichtarowicz-Krynska, E. J.; Cole, T. J.; Camacho-Hubner, C.; Britto, J.; Levin, M.; Klein, N.; Aynsley-Green, A. “Circulating Aldosterone Levels are Unexpectedly Low in Children with Actute Meningococcal Disease” J Clin Endocrin Met 89(3):1410-1414 (2004).
137. Meurant, G. “The Adrenal Cortex” chapter in Molecular and Cell Endocrinology in Principles of Medical Biology, v. 10A, p. 241 (1997).
138. Folan, M. M.; Stone, R. A.; Pittenger, A. L.; Pittenger, A. L.; Stofferl, J. A.; Hess, M. M.; Kroboth, P. D. “Dehydroepiisoandreosterone, dehydroepiisoandrosterone-sulfate, and cortisol concentrations in intensive care unit patients” Crit Care Med 20(5):965-970 (2001).
139. Vd Berghe, G.; d Zegher, F.; Wouters, P.; Schetz, M.; Verwaest, C.; Ferdinande, P.; Lauwers, P. “Dehydroepiandrosterone sulphate in critical illness: effect of dopamine” Clin Endocrin 43:457-463 (1995).

Various references are cited herein, the contents of which are hereby incorporated by reference in their entireties.

1. An assay for testing a sample for the presence and/or amount of an analyte of interest comprising contacting at least a portion of the sample with effective amounts of (1) a primary aptamer comprising a core sequence that binds to the analyte and (2) an anti-aptamer which is complementary to at least a portion of the primary aptamer, wherein the primary aptamer and/or anti-aptamer comprise a detectable moiety(ies) which detect whether the primary aptamer and anti-aptamer are bound to each other or unbound; and wherein a primary aptamer bound to the analyte does not bind to its anti-aptamer.

2. The assay of claim 1 wherein the primary aptamer comprises a fluorescent label.

3. The assay of claim 1, where the anti-aptamer comprises a quencher moiety.

4. The assay of claim 1, where the anti-aptamer is complementary to at least 85 percent of the primary aptamer

5. The assay of claim 4, where the anti-aptamer is complementary to at least 95 percent of the primary aptamer.

6. The assay of claim 1, where the analyte is selected from the group consisting of glucose, hydrocortisone, phenylalanine, dehydroisoandrosterone, deoxycortisone, testosterone, aldosterone, dopamine, sphingosine-1-phosphate, serotonin, melatonin, tyrosine, tobramycin, amikacin, methylene blue, ammonium, boronic acid, and epinephrine.

7. A method of detecting or measuring the presence or amount of an analyte of interest in a sample, comprising (i) contacting at least a portion of a sample with effective amounts of a primary aptamer and an anti-aptamer that is complementary to at least a portion of the primary aptamer, said primary aptamer and/or anti-aptamer comprising a moiety which allows the amount of primary aptamer bound to analyte to be detected and/or measured, under conditions that would permit duplex formation between the primary aptamer and anti-aptamer if target analyte were not present; and (ii) detecting the amount of primary aptamer that is not bound to anti-aptamer.

8. The method of claim 7 wherein the primary aptamer comprises a fluorescent label.

9. The method of claim 7, where the anti-aptamer comprises a quencher moiety.

10. The method of claim 7, where the anti-aptamer is complementary to at least 85 percent of the primary aptamer

11. The method of claim 10, where the anti-aptamer is complementary to at least 95 percent of the primary aptamer.

12. The method of claim 7, where the analyte is selected from the group consisting of glucose, hydrocortisone, phenylalanine, dehydroisoandrosterone, deoxycortisone, testosterone, aldosterone, dopamine, sphingosine-1-phosphate, serotonin, melatonin, tyrosine, tobramycin, amikacin, methylene blue, ammonium, boronic acid, epinephrine, creatinine, and vasopressin.

13. A method of detecting or measuring the presence or amount of an analyte of interest in a sample, comprising (i) contacting at least a portion of the sample with effective amounts of (a) a primary aptamer comprising a fluorescent label and (b) an anti-aptamer, comprising a moiety that quenches fluorescence of said fluorescent label if primary aptamer and anti-aptamer are bound together in a duplex, under conditions that would permit duplex formation between primary aptamer and anti-aptamer to occur if analyte were not present; and (ii) detecting the amount of fluorescence.

14. The method of claim 13 wherein the primary aptamer comprises a fluorescent label.

15. The method of claim 13, where the anti-aptamer comprises a quencher moiety.

16. The method of claim 13, where the anti-aptamer is complementary to at least 85 percent of the primary aptamer

17. The method of claim 16, where the anti-aptamer is complementary to at least 95 percent of the primary aptamer.

18. The method of claim 13, where the analyte is selected from the group consisting of glucose, hydrocortisone, phenylalanine, dehydroisoandrosterone, deoxycortisone, testosterone, aldosterone, dopamine, sphingosine-1-phosphate, serotonin, melatonin, tyrosine, tobramycin, amikacin, methylene blue, ammonium, boronic acid, epinephrine, creatinine and vasopressin.

19. (canceled)

20. (canceled)

21. (canceled)

22. (canceled)

23. (canceled)

24. (canceled)

25. The method of claim 7, wherein the primary aptamer comprises a core sequence that binds to the analyte and a portion complementary to a sensor oligonucleotide, and the method further comprises contacting at least a portion of the sample with an effective amounts of a sensor oligonucleotide one or more of which is bound to a detectable moiety(ies) which can detect whether the primary aptamer and sensor oligonucleotide are bound to each other or whether primary aptamer is bound to analyte.

26. (canceled)

27. (canceled)

28. (canceled)

29. (canceled)

30. (canceled)

31. (canceled)

32. (canceled)

33. (canceled)

34. The method of claim 7, wherein the primary aptamer comprises a core sequence that binds to the analyte and a portion that, when primary aptamer is bound to analyte, binds to a secondary “sandwich” aptamer, and the method further comprises contacting at least a portion of the sample with an effective amounts of a secondary “sandwich” aptamer; one or more of which is bound to a detectable moiety(ies) which can detect whether the primary aptamer and secondary “sandwich” aptamer are bound to each other or unbound.

35. (canceled)

36. (canceled)

37. (canceled)

38. (canceled)

39. (canceled)

40. (canceled)

41. (canceled)
